(19) United States
(12) Patent Application Publication
(10) Pub. No.: US 2010/0305124 A1
(43) Pub. Date: Dec. 2, 2010

(54) Acylated Aminopyridine and Aminopyridazine Insecticides

(75) Inventors: Martin FUSSLEIN, Dusseldorf (DE); Ernst Rudolf Gesing, Erkrath (DE); Markus Heil, Leichlingen (DE); Bernd Alig, Konigswinter (DE); Eva-Maria Franken, Limonest (FR); Olga Malsam, Rosrath (DE); Arnd Voerste, Koln (DE); Koichi Araki, Ibaraki (JP); Norio Sasaki, Ibaraki (JP); Sachio Kudo, Ibaraki (JP); Eiichi Shimojo, Tochigi (JP); Teruyuki Ichihara, Tochigi (JP); Masashi Ataka, Tochigi (JP); Katsuhiko Shibuya, Tochigi (JP)

Correspondence Address: STERNE, KESSLER, GOLDSTEIN & FOX P.L.L.C., 1100 NEW YORK AVENUE, N.W., WASHINGTON, DC, 20005, US

(73) Assignee: BAYER CROPSCIENCE AG, Monheim (DE)

(21) Appl. No.: 12/753,185

(22) Filed: Apr. 2, 2010

(30) Foreign Application Priority Data
Apr. 3, 2009 (EP) 09157253.7

(51) Int. Cl. A01N 43/84; C07D 401/14; C07D 401/12; C07D 413/14; A01N 43/40; A01N 43/54; A01N 43/66; A01P 7/04; A01P 7/02; A01P 5/00; A01P 9/00

(52) U.S. Cl. 514/235.5; 546/256; 546/261; 544/316; 544/300; 544/219; 544/131; 514/333; 514/335; 514/274; 514/241

(57) ABSTRACT

The present invention relates to aminopyridine and aminopyridazine derivatives of the general formula (I)

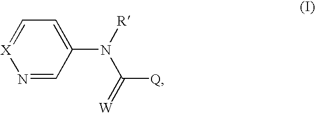

in which R¹, X, W and Q have the definitions given in the description, to a number of processes for preparing them, and to their use as insecticides.

The present invention relates to new pesticides, to a number of processes for preparing them and to their use as active ingredients, more particular their use as insecticides.

Described in the literature are certain 3-substituted pyridines for use as insecticides; see, for example, EP 314615 or WO 2009/027393. Surprisingly it has now been found that certain acylated aminopyridines and aminopyridazines possess strong insecticidal properties.

The present invention accordingly provides aminopyridine and aminopyridazine derivatives of the general formula (I),

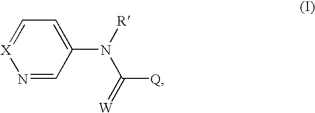

in which

### R¹ is

hydrogen or is optionally singly or multiply and identically or differently substituted C₁-C₆-alkyl, C₂-C₆-alkenyl, C₂-C₆-alkynyl or C₃-C₆-cycloalkyl,
it being possible for the substituents to be selected independently of one another from halogen, cyano, nitro, hydroxyl, C₁-C₆-alkyl, C₁-C₆-alkoxy, C₃-C₆-cycloalkyl, C₁-C₆-haloalkoxy, C₁-C₆-alkylthio, aryl, hetaryl, arylalkyl and hetarylalkyl,
it being possible for the substituents aryl, hetaryl, arylalkyl and hetarylalkyl to be optionally substituted singly or multiply and identically or differently by halogen, cyano, nitro, hydroxyl, C₁-C₆-alkyl, C₂-C₆-alkenyl, C₂-C₆-alkynyl, C₃-C₆-cycloalkyl, C₁-C₆-alkoxy, C₁-C₆-haloalkyl, C₁-C₆-haloalkoxy or C₁-C₆-alkylthio,
X is CH or N,
Q is an aromatic 5-6-membered ring Q which is optionally substituted by 1 to 3 heteroatoms from the series N, S and O and which may optionally be substituted by the radicals R²-R⁴ and the radical A-Y independently of one another,

### if X is N

Q is an aromatic 6-membered ring Q which is optionally substituted by 1 to 3 heteroatoms from the series N, S and O and which may optionally be substituted by the radicals R²-R⁴ and the radical A-Y independently of one another; or is an aromatic 5-membered ring Q which is optionally substituted by 1 to 3 heteroatoms from the series N, S and O, not more than one of the heteroatoms being N, and which may optionally be substituted by the radicals R²-R⁴ and the radical A-Y independently of one another;
   or stands for the following ring systems selected from Q₈ₐ-Q_{8d}

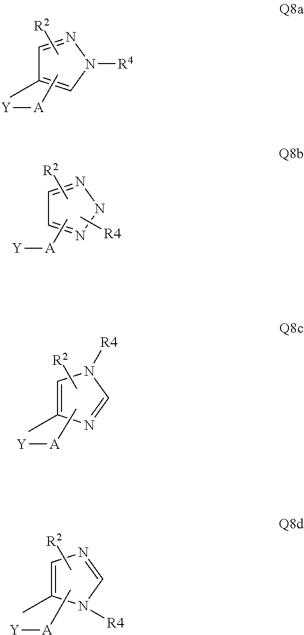

### if X is CH,

W is O or S,
R² and R³ independently of one another are hydrogen, hydroxyl, halogen or nitro, or are optionally singly or multiply, identically or differently substituted amino, C₁-C₆-alkyl, C₁-C₆-alkoxy, C₁-C₆-alkoxy-C₁-C₆-alkyl, C₂-C₆-alkenyl, C₂-C₆-alkynyl or C₃-C₆-cycloalkyl, it being possible for the substituents to be selected independently of one another from halogen, C₁-C₆-alkyl, C₁-C₆-alkylcarbonyl, C₁-C₆-alkoxy, C₁-C₆-haloalkoxy, C₃-C₆-cycloalkyl, C₃-C₆-cycloalkylcarbonyl, cyano, nitro, hydroxyl, C₁-C₆-alkylthio, C₁-C₆-alkylsulphinyl, C₁-C₆-alkylsulphonyl, (C₁-C₆-alkoxy)carbonyl, C₁-C₆-alkylamino, C₃-C₆-cycloalkylamino, (C₁-C₆-alkyl)C₃-C₆-cycloalkylamino or aryl, hetaryl, which may optionally be singly or multiply and identically or differently substituted by halogen, cyano, nitro, hydroxyl, C₁-C₆-alkyl, C₁-C₆-haloalkyl, C₂-C₆-alkenyl, C₂-C₆-alkynyl, C₃-C₆-cycloalkyl, C₁-C₆-alkoxy, C₁-C₆-haloalkoxy, C₁-C₆-alkylthio, C₁-C₆-alkylsulphinyl and/or C₁-C₆-alkylsulphonyl,
R² and R³ may likewise together form a 5- to 7-membered heteroaromatic ring which contains 1-3 heteroatoms from the series N, S and O and which may optionally be singly or multiply substituted by C₁-C₆-alkyl,
R⁴ is hydrogen or is optionally singly or multiply, identically or differently substituted C₁-C₆-alkyl, C₁-C₆-alkoxy-C₁-C₆-alkyl, C₂-C₆-alkenyl, C₂-C₆-alkynyl, C₃-C₆-cycloalkyl, aryl or hetaryl, it being possible for the substituents to be selected independently of one another from halogen, C₁-C₆-alkyl, C₁-C₆-alkylcarbonyl, C₁-C₆-alkoxy, C₁-C₆-haloalkoxy, C₃-C₆-cycloalkyl, C₃-C₆-cycloalkylcarbonyl, cyano, nitro, hydroxyl, C₁-C₆-alkylthio, C₁-C₆-alkylsulphinyl, C₁-C₆-alkylsulphonyl, (C₁-C₆-alkoxy)carbonyl, C₁-C₆-alkylamino, C₃-C₆-cycloalkylamino, (C₁-C₆-alkyl)C₃-C₆-cycloalkylamino, aryl or hetaryl, which may optionally be singly or multiply and identically or differently substituted by halogen, cyano, nitro, hydroxyl, C₁-C₆-alkyl, C₁-C₆-haloalkyl, C₂-C₆-alkenyl, C₂-C₆-alkynyl, C₃-C₆-cycloalkyl, C₁-C₆-alkoxy, C₁-C₆-haloalkoxy C₁-C₆-alkylthio, C₁-C₆-alkylsulphinyl and/or C₁-C₆-alkylsulphonyl,
A is a direct bond or C₁-C₆-alkylene, C₁-C₆-alkyleneoxy, oxy(C₁-C₆)-alkylene, thio(C₁-C₆)-alkylene, C₁-C₆-alkylenethio, —O—, —S—, C₁-C₄-alkylenecarboxy, carboxy(C₁-C₄)-alkylene, —NR⁵, —C═N—O—, C₁-C₄-alkylenecarbamido, carbamido(C₁-C₄)-alkylene, —NR⁵(C═O)O—, NR⁵(SO₂)—, —NR⁵(C═O)NR⁵— or —(SO₂)—,
R⁵ is C₁-C₆-alkyl, C₃-C₆-cycloalkyl or hydrogen,
Y is hydrogen, optionally singly or multiply, identically or differently substituted C₁-C₆-alkyl, C₃-C₆-cycloalkyl, an optionally singly or multiply, identically or differently substituted, 5-10-membered, optionally fused ring system containing 0-4 heteroatoms, it being possible for the heteroatoms to be selected from the series N, S and O,
   it being possible for the substituents to be selected from halogen, nitro, cyano, from optionally singly or multiply, identically or differently substituted amino, C₁-C₆-alkyl, C₁-C₆-alkoxy, C₁-C₆-alkoxy-C₁-C₆-alkyl, C₂-C₆-alkenyl, C₂-C₆-alkynyl, C₃-C₆-cycloalkyl, aryl, hetaryl, C₁-C₆-arylalkyl, C₁-C₆-hetarylalkyl, aryloxy, hetaryloxy, sulphonyl, C₁-C₆-thioalkyl and/or C₁-C₆-carboxyalkyl,
      it being possible for the substituents to be selected independently of one another from halogen, C₁-C₆-alkyl, C₁-C₆-alkylcarbonyl, C₁-C₆-haloalkyl, C₁-C₆-alkoxy, C₁-C₆-haloalkoxy, C₃-C₆-cycloalkyl, C₃-C₆-cycloalkylcarbonyl, cyano, nitro, C₁-C₆-alkylthio, C₁-C₆-alkylsulphinyl, C₁-C₆-alkylsulphonyl, (C₁-C₆-alkoxy)carbonyl, C₂-C₆-alkenyl, C₂-C₆-alkynyl, C₁-C₆-alkylamino, C₃-C₆-cycloalkylamino, (C₁-C₆-alkyl)C₃-C₆-cycloalkylamino and/or di(C₁-C₄)alkylamino,
      and also salts of compounds of the formula (I), and their use for controlling animal pests.

The compounds of the formula (I) may possibly be present in different polymorphic forms or as a mixture of different polymorphic forms. Not only the pure polymorphs but also the polymorph mixtures are subject matter of the invention and can be used in accordance with the invention.

The compounds of the formula (I) comprise any possible diastereomers or enantiomers.

A general definition of the aminopyridines and aminopyridazines is provided by the formula (I). Preferred definitions of radicals in the formulae given above and below are specified below. These definitions apply equally to the end products of the formula (I) and to all intermediates.

Considered preferably, more preferably and very preferably are compounds of the formula (I), and also a method of controlling pests that uses the compounds of the formula (I), where

R¹ preferably is
hydrogen, or is optionally singly or multiply, identically or differently substituted C₁-C₄-alkyl, C₂-C₄-alkenyl, C₂-C₄-alkynyl or C₃-C₄-cycloalkyl,
it being possible for the substituents to be selected independently of one another from halogen, cyano, nitro, hydroxyl, C₁-C₄-alkyl, C₁-C₄-alkoxy, C₃-C₄-cycloalkyl, C₁-C₄-haloalkoxy, aryl, hetaryl, arylalkyl, hetarylalkyl,
it being possible for the substituents aryl, hetaryl, arylalkyl and hetarylalkyl to be optionally singly or multiply, identically or differently substituted by halogen, cyano, nitro, hydroxyl, C₁-C₄-alkyl, C₂-C₄-alkenyl, C₂-C₄-alkynyl, C₃-C₄-cycloalkyl, C₁-C₄-alkoxy, C₁-C₄-haloalkyl or C₁-C₄-haloalkoxy,
R¹ more preferably is
hydrogen, or is optionally singly or multiply, identically or differently substituted C₁-C₄-alkyl, C₂-C₄-alkenyl or C₂-C₄-alkynyl,
it being possible for the substituents to be selected independently of one another from halogen, C₁-C₄-alkyl, aryl, arylalkyl,
it being possible for the substituents aryl, arylalkyl to be optionally singly or multiply, identically or differently substituted by halogen, C₁-C₄-alkyl, C₁-C₄-haloalkyl and C₁-C₄-haloalkoxy,
R¹ very preferably is
hydrogen, methyl, ethyl, propyl, allyl or propargyl, or optionally methyl-, halogen-, C₁-C₄-haloalkyl- or C₁-C₄-haloalkoxy-substituted benzyl,
Q preferably is a ring selected from the group Q1 to Q14

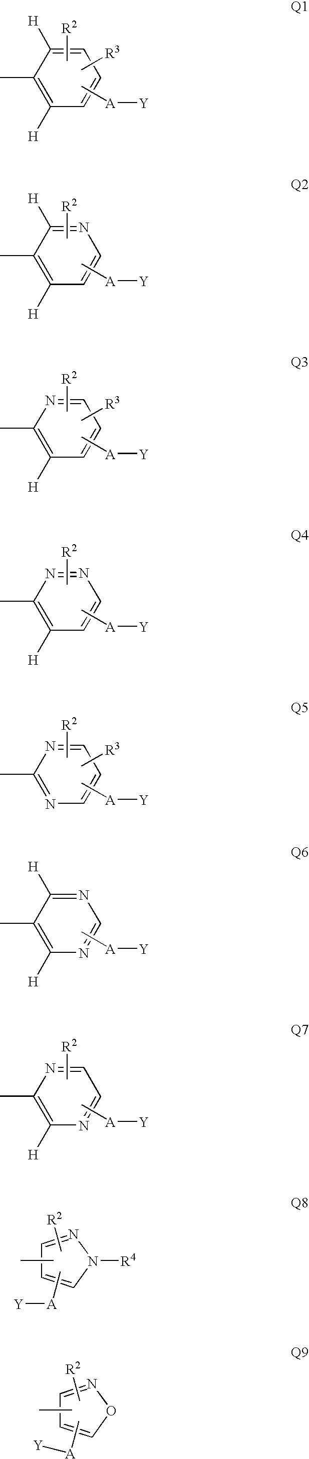

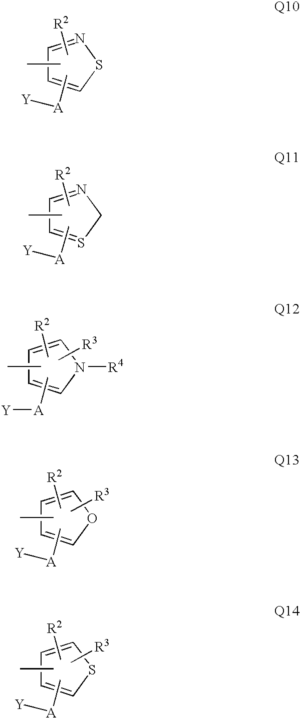

### if X is N

Q more preferably is one of the following rings

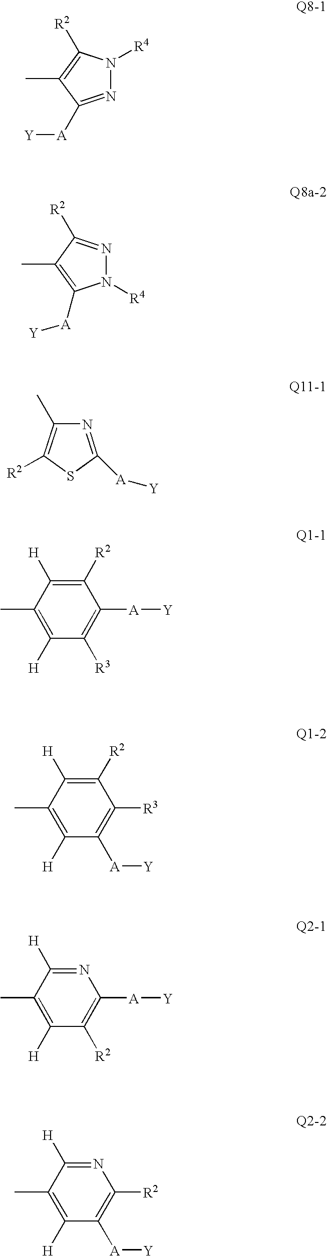

### if X is N

Q very preferably is one of the following rings

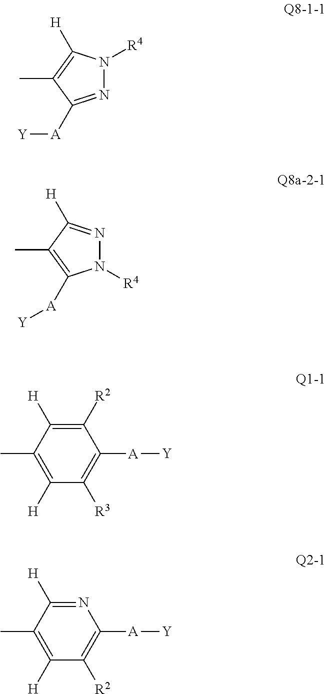

### if X is N

Q preferably is a ring selected from the group Q1 to Q14

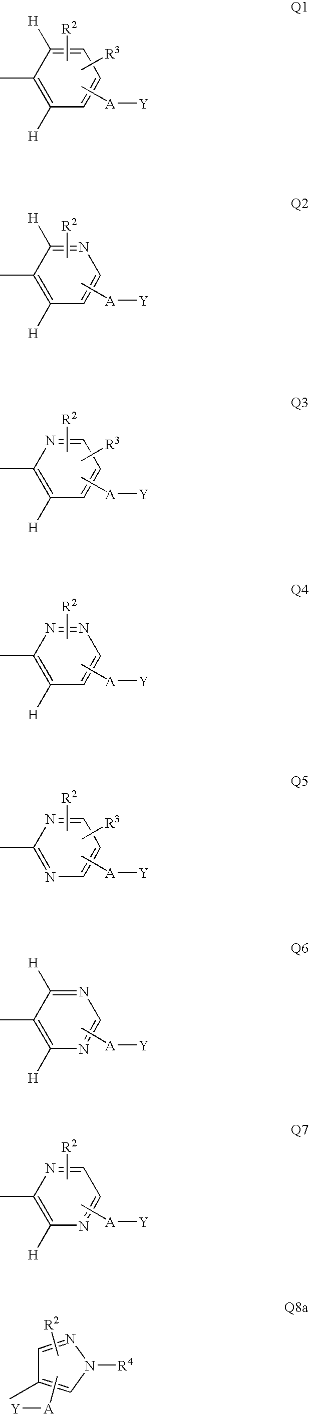

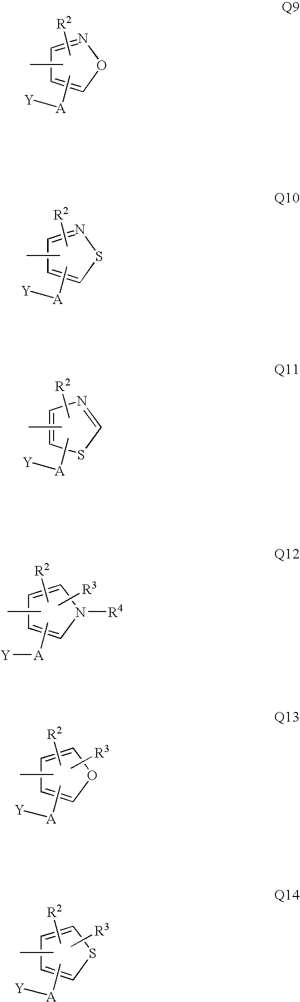

### if X is CH

Q more preferably is one of the following rings

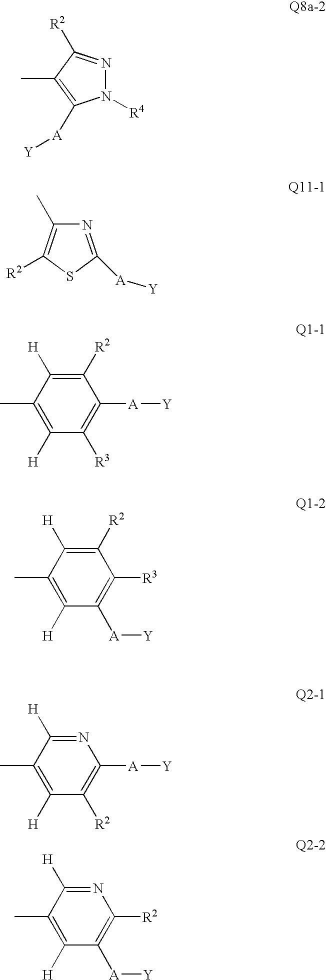

### if X is CH

Q very preferably is one of the following rings

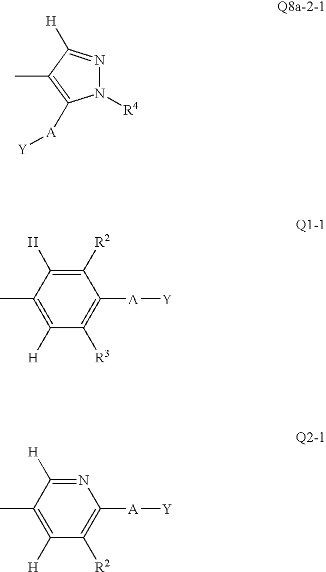

### if X is CH

R² and R³ independently of one another preferably are hydrogen, hydroxyl, halogen, nitro, or are optionally singly or multiply, identically or differently substituted amino, C₁-C₄-alkyl, C₁-C₄-alkoxy, C₁-C₄-alkoxy-C₁-C₄-alkyl, C₂-C₄-alkenyl, C₂-C₄-alkynyl or C₃-C₄-cycloalkyl, it being possible for the substituents to be selected independently of one another from halogen, C₁-C₄-alkyl, C₁-C₄-alkylcarbonyl, C₁-C₄-alkoxy, C₁-C₄-haloalkoxy, C₃-C₄-cycloalkyl, C₃-C₄-cycloalkylcarbonyl, cyano, nitro, hydroxyl, C₁-C₄-alkylthio, C₁-C₄-alkylsulphinyl, C₁-C₄-alkylsulphonyl, (C₁-C₄-alkoxy)carbonyl, C₁-C₄-alkylamino, C₃-C₄-cycloalkylamino, (C₁-C₄-alkyl)C₃-C₄-cycloalkylamino or aryl, hetaryl, which may optionally be singly or multiply and identically or differently substituted by halogen, cyano, nitro, hydroxyl, C₁-C₄-alkyl, C₁-C₄-haloalkyl, C₂-C₄-alkenyl, C₂-C₄-alkynyl, C₃-C₄-cycloalkyl, C₁-C₄-alkoxy, C₁-C₄-haloalkoxy, C₁-C₄-alkylthio, C₁-C₄-alkylsulphinyl, C₁-C₄-alkylsulphonyl,
R² and R³ likewise preferably may together form a 5-membered, optionally singly or multiply C₁-C₆-alkyl-substituted, N-containing, heteroaromatic ring,
R² and R³ independently of one another more preferably are hydrogen, halogen, nitro, or are optionally singly or multiply, identically or differently substituted amino, C₁-C₄-alkyl, C₁-C₄-alkoxy, C₁-C₄-alkoxy-C₁-C₄-alkyl or C₃-C₄-cycloalkyl, it being possible for the substituents to be selected independently of one another from halogen, C₁-C₄-alkyl, C₁-C₄-alkoxy, C₁-C₄-haloalkoxy, C₃-C₄-cycloalkyl, cyano and nitro,
R² and R³ independently of one another very preferably are hydrogen, halogen, nitro, amino, C₁-C₄-alkyl, C₁-C₄-haloalkyl, C₁-C₄-alkoxy or C₁-C₄-alkoxy-C₁-C₄-alkyl.
R⁴ preferably is hydrogen or is optionally singly or multiply, identically or differently substituted, C₁-C₄-alkyl, C₁-C₄-alkoxy-C₁-C₄-alkyl, C₂-C₄-alkenyl, C₂-C₄-alkynyl, C₃-C₄-cycloalkyl, aryl, hetaryl, it being possible for the substituents to be selected independently of one another from halogen, C₁-C₄-alkyl, C₁-C₄-alkoxy, C₁-C₄-haloalkoxy, C₃-C₄-cycloalkyl, cyano, nitro, C₁-C₄-alkylthio, (C₁-C₆-alkoxy)carbonyl or aryl, hetaryl, which may be optionally singly or multiply and identically or differently substituted by halogen, cyano, nitro, C₁-C₄-alkyl, C₁-C₄-haloalkyl, C₃-C₄-cycloalkyl, C₁-C₄-alkoxy and/or C₁-C₄-haloalkoxy,
R⁴ more preferably is hydrogen or is optionally singly or multiply, identically or differently substituted C₁-C₄-alkyl, C₁-C₄-alkoxy-C₁-C₄-alkyl, C₂-C₄-alkenyl, C₂-C₄-alkynyl, C₃-C₄-cycloalkyl, aryl, hetaryl, it being possible for the substituents to be selected independently of one another from halogen, C₁-C₄-alkyl, C₁-C₄-alkoxy, C₁-C₄-haloalkoxy, C₃-C₄-cycloalkyl, cyano, nitro, (C₁-C₄-alkoxy)carbonyl or aryl, hetaryl, which may be optionally singly or multiply and identically or differently substituted by halogen, cyano, nitro, C₁-C₄-alkyl, C₁-C₄-haloalkyl, C₁-C₄-alkoxy and/or C₁-C₄-haloalkoxy,
R⁴ very preferably is hydrogen or is optionally singly or multiply, identically or differently substituted C₁-C₄-alkyl, C₁-C₄-alkoxy-C₁-C₄-alkyl, C₂-C₄-alkenyl, C₃-C₄-cycloalkyl, aryl, hetaryl, it being possible for the substituents to be selected independently of one another from halogen, C₁-C₄-alkyl, C₁-C₄-alkoxy, or aryl, hetaryl, which may be optionally singly or multiply and identically or differently substituted by halogen and/or C₁-C₄-haloalkyl,
A preferably is a direct bond or is C₁-C₄-alkylene, C₁-C₄-alkyleneoxy, oxy(C₁-C₄)-alkylene, thio(C₁-C₄)-alkylene, C₁-C₄-alkylenethio, —O—, —S—, C₁-C₄-alkylenecarboxy, carboxy(C₁-C₄)-alkylene, —NR⁵, —C═N—O—, —NR⁵(C═O)O—, NR⁵(SO₂)—, —NR⁵(C═O)NR⁵—, optionally R⁵-substituted carbamido, amidocarbonyl, C₁-C₄-alkylenecarbamido, carbamido(C₁-C₄)-alkylene, —(SO₂)—,
A more preferably is a direct bond or is C₁-C₄-alkylene, —CH₂—O—, CH₂—S—, —O—, —S—, —O—CH₂, —S—CH₂—, —C(═O)O—, OC(═O)—, —NR—, —C═N—O—, —R⁵NC(═O)—, —(CH₂)R⁵NC(═O)—, —C(═O)NR⁵—, —NR⁵(C═O)O—, NR⁵(SO₂)—, —NR⁵(C═O)NR⁵— or —(SO₂)—,
A very preferably is a direct bond or is —CH₂—, —CH₂—O—, CH₂—S—, —O—, —S—, —O—CH₂—, —S—CH₂—, —C(═O)O—, OC(═O)—, —NR⁵—, —C═N—O—, —R⁵NC(═O)—, —C(═O)NR⁵—, —(CH₂)R⁵NC(═O)—, —NR⁵(C═O)O—, NR⁵(SO₂)—, —NR⁵(C═O)NR⁵—, —(SO₂)—,
R⁵ preferably is C₁-C₄-alkyl, C₃-C₆-cycloalkyl or hydrogen,
R⁵ more preferably is methyl, ethyl, cyclopentyl, isobutyl or hydrogen,
Y preferably is hydrogen, is optionally singly or multiply and identically or differently substituted C₁-C₄-alkyl or C₃-C₄-cycloalkyl, or is an optionally singly or multiply and identically or differently substituted, 5-6-membered ring system containing 0-4 heteroatoms, it being possible for the heteroatoms to be selected from the series N and O, or is naphthalene or quinoline,
it being possible for the substituents to be selected from halogen, nitro and cyano, from optionally singly or multiply, identically or differently substituted amino, C₁-C₄-alkyl, C₁-C₄-alkoxy, C₁-C₄-alkoxy-C₁-C₄-alkyl, C₂-C₄-alkenyl, C₂-C₄-alkynyl, C₃-C₄-cycloalkyl, aryl, hetaryl, C₁-C₄-arylalkyl, C₁-C₄-hetarylalkyl, aryloxy, hetaryloxy, sulphonyl, C₁-C₄-thioalkyl and C₁-C₄-carboxyalkyl,
it being possible for the substituents to be selected independently of one another from halogen, C₁-C₄-alkyl, C₁-C₄-alkylcarbonyl, C₁-C₄-haloalkyl, C₁-C₄-alkoxy, C₁-C₄-haloalkoxy, C₃-C₄-cycloalkyl, C₃-C₄-cycloalkylcarbonyl, cyano, nitro, (C₁-C₄-alkoxy)carbonyl, C₁-C₄-alkylthio and di(C₁-C₄)alkylamino,
Y more preferably is optionally singly or multiply, identically or differently substituted C₁-C₄-alkyl or C₃-C₄-cycloalkyl, or is an optionally singly or multiply, identically or differently substituted, 5-6-membered ring containing 0-3 nitrogen atoms, or is naphthalene or quinoline,
it being possible for the substituents to be selected from halogen, nitro and cyano, from optionally singly or multiply, identically or differently substituted amino, C₁-C₄-alkyl, C₁-C₄-alkoxy, C₁-C₄-alkoxy-C₁-C₄-alkyl, C₃-C₄-cycloalkyl, aryl, hetaryl, C₁-C₄-arylalkyl, C₁-C₄-hetarylalkyl, aryloxy, hetaryloxy, sulphonyl, C₁-C₄-thioalkyl and C₁-C₄-carboxyalkyl,
it being possible for the substituents to be selected independently of one another from halogen, C₁-C₄-alkyl, C₁-C₄-haloalkyl, C₁-C₄-alkoxy, C₁-C₄-haloalkoxy, C₁-C₄-alkylthio, C₃-C₄-cycloalkyl and di(C₁-C₄)alkylamino,
Y very preferably is optionally singly or multiply, identically or differently substituted C₁-C₄-alkyl or C₃-C₄-cycloalkyl, or is optionally singly or multiply, identically or differently substituted phenyl, pyridyl, pyrimidinyl, pyrazyl, triazolyl, pyrrolidinyl, morpholinyl or piperidinyl,
it being possible for the substituents to be selected from halogen, nitro and cyano, from optionally singly or multiply, identically or differently substituted C₁-C₄-alkyl, C₁-C₄-alkoxy, phenyl, triazole, phenyloxy, sulphonyl, C₁-C₄-thioalkyl and C₁-C₄-carboxyalkyl,
it being possible for the substituents to be selected independently of one another from halogen, C₁-C₄-alkyl, C₁-C₄-haloalkyl, C₁-C₄-alkoxy, C₁-C₄-haloalkoxy, C₁-C₄-alkylthio, C₃-C₄-cycloalkyl and dimethylamino,
X preferably and more preferably is CH or N,
X very preferably is CH,
X likewise very preferably is N,
W preferably is O or S,
W more preferably and very preferably is O.

The individual general, preferred, more preferred and very preferred definitions stated above for the substituents R¹-R⁵, X, W and A, Y and Q may be combined arbitrarily with one another.

The present invention preferably also provides the lithium, sodium, potassium, magnesium, calcium, ammonium, C₁-C₄-alkylammonium, di(C₁-C₄-alkyl)ammonium, tri(C₁-C₄-alkyl)ammonium, tetra(C₁-C₄-alkyl)ammonium, tri(C₁-C₄-alkyl)sulphonium, C₅- or C₆-cycloalkylammonium, di(C₁-C₂-alkyl)benzylammonium and tri(C₁-C₂-alkyl)benzylammonium salts of compounds of the formula (I) in which R¹, X, W and A have the above general, preferred, more preferred and especially preferred definitions, and which may be prepared by processes that are generally customary.

The present compounds of the general formula (I) may possibly have a chiral carbon atom.

In accordance with the rules of Cahn, Ingold and Prelog (CIP rules), these substituents may have both an (R) and an (S) configuration.

Embraced by the present invention are compounds of the general formula (I) both having (S) and having (R) configuration at the respective chiral carbon atoms; in other words, the present invention encompasses the compounds of the general formula (I) in which the carbon atoms in question have in each case independently of one another

(1) an (R) configuration; or
(2) an (S) configuration.

If there are two more centres of chirality in the compounds of the general formula (I), any desired combinations of the configurations of the chiral centres are possible, i.e. such that

(1) one chiral centre has the (R) configuration and the other chiral centre has the (S) configuration;
(2) one chiral centre has the (R) configuration and the other chiral centre has the (R) configuration; and
(3) one chiral centre has the (S) configuration and the other chiral centre has the (S) configuration.

Embraced in accordance with the invention, therefore, are more particularly the following compounds of the formulae (I-1), (I-2) and (I-3)

(I-1) RS compound
(I-2) RR compound
(I-3) SS compound.

With good plant compatibility, favourable toxicity to warm-blooded animals and good environmental compatibility, the active ingredients according to the invention are suitable for protecting plants and plant organs, for increasing harvest yields, improving the quality of the harvested material and for controlling animal pests, in particular insects, arachnids, helminths, nematodes and molluscs which are encountered in agriculture, in horticulture, in animal husbandry, in forests, in gardens and leisure facilities, in the protection of stored products and of materials, and also in the hygiene sector. They can preferably be used as crop protection agents. They are effective against normally sensitive and resistant species and also against all or some stages of development. The aforementioned pests include:

From the order of the Anoplura (Phthiraptera), for example Damalinia spp., Haematopinus spp., Linognathus spp., Pediculus spp., Trichodectes spp.

From the class of the Arachnida, for example Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vacates lycopersici.

From the class of the Bivalva, for example Dreissena spp.

From the order of the Chilopoda, for example Geophilus spp., Scutigera spp.

From the order of the Coleoptera, for example Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zea-landica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnosterna consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chryso-cephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogoderma spp., Tychius spp., Xylotrechus spp., Zabrus spp.

From the order of the Collembola, for example Onychiurus armatus.

From the order of the Dermaptera, for example Forficula auricularia.

From the order of the Diplopoda, for example Blaniulus guttulatus.

From the order of the Diptera, for example Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp., Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa, Wohlfahrtia spp.

From the class of the Gastropoda, for example Anion spp., Biomphalaria spp., Bulinus spp., Deroceras spp., Galba spp., Lymnaea spp., Oncomelania spp., Succinea spp.

From the class of the helminths, for example Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp., Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

Furthermore, protozoans, such as Eimeria, can be controlled.

From the order of the Heteroptera, for example Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodnius spp., Sahlbergella singularis, Scotinophora spp., Stephanitis nashi, Tibraca spp., Triatoma spp.

From the order of the Homoptera, for example Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma piri, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Doralis spp., Drosicha spp., Dysaphis spp., Dysmicoccus spp., Empoasca spp., Eriosoma spp., Erythroneura spp., Euscelis bilobatus, Geococcus coffeae, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolii.

From the order of the Hymenoptera, for example Diprion spp., Hoplocampa spp., Lasius spp., Monomorium pharaonis, Vespa spp.

From the order of the Isopoda, for example Armadillidium vulgare, Oniscus asellus, Porcellio scaber.

From the order of the Isoptera, for example Reticulitermes spp., Odontotermes spp.

From the order of the Lepidoptera, for example Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Chematobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.

From the order of the Orthoptera, for example Acheta domesticus, Blatta orientalis, Blattella germanica, Gryllotalpa spp., Leucophaea maderae, Locusta spp., Melanoplus spp., Periplaneta americana, Schistocerca gregaria.

From the order of the Siphonaptera, for example Ceratophyllus spp., Xenopsylla cheopis.

From the order of the Symphyla, for example Scutigerella immaculata.

From the order of the Thysanoptera, for example Baliothrips biformis, Enneothrips flavens, Frankliniella spp., Heliothrips spp., Hercinothrips femoralis, Kakothrips spp., Rhipiphorothrips cruentatus, Scirtothrips spp., Taeniothrips cardamoni, Thrips spp.

From the order of the Thysanura, for example Lepisma saccharina.

The phytoparasitic nematodes include, for example, Anguina spp., Aphelenchoides spp., Belonoaimus spp., Bursaphelenchus spp., Ditylenchus dipsaci, Globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans, Xiphinema spp.

The compounds of the formula (I) can be used, if appropriate, in certain concentrations or at certain application rates also as herbicides, safeners, growth regulators or agents for improving plant properties, or as microbicides, for example as fungicides, antimycotics, bactericides, viricides (including agents against viroids) or as agents against MLO (Mycoplasma-like organisms) and RLO (Rickettsia-like organisms). If appropriate, they can also be used as intermediates or precursors for the synthesis of further active ingredients.

The active ingredients can be converted to customary formulations, such as solutions, emulsions, wettable powders, water- and oil-based suspensions, powders, dusts, pastes, soluble powders, soluble granules, granules for scattering, suspension-emulsion concentrates, natural substances impregnated with active ingredient, synthetic substances impregnated with active ingredient, fertilizers, and also microencapsulations in polymeric substances.

These formulations are produced in a known manner, e.g. by mixing the active ingredients with extenders, i.e. liquid solvents and/or solid carriers, optionally using surface-active agents, i.e. emulsifiers and/or dispersants and/or foam-generating agents. The production of the formulations takes place either in suitable plants or before or during application.

Auxiliaries that can be used are substances which are suitable for imparting particular properties to the agent itself and/or preparations derived therefrom (e.g. spray liquors, seed dressings), such as certain technical properties and/or also particular biological properties. Suitable typical auxiliaries are: extenders, solvents and carriers.

Suitable extenders are, for example, water, polar and nonpolar organic chemical liquids, for example from the classes of the aromatic and nonaromatic hydrocarbons (such as paraffins, alkylbenzenes, alkylnaphthalenes, chlorobenzenes), the alcohols and polyols (which, if appropriate, can also be substituted, etherified and/or esterified), the ketones (such as acetone, cyclohexanone), esters (also fats and oils) and (poly)ethers, the simple and substituted amines, amides, lactams (such as N-alkylpyrrolidones) and lactones, the sulphones and sulphoxides (such as dimethyl sulphoxide).

If water is used as extender, it is also possible for example to use organic solvents as auxiliary solvents. Suitable liquid solvents are essentially: aromatics, such as xylene, toluene, or alkyl-naphthalenes, chlorinated aromatics and chlorinated aliphatic hydrocarbons, such as chlorobenzenes, chloroethylenes or methylene chloride, aliphatic hydrocarbons, such as cyclohexane or paraffins, e.g. petroleum fractions, mineral and vegetable oils, alcohols, such as butanol or glycol, and ethers and esters thereof, ketones, such as acetone, methyl ethyl ketone, methyl isobutyl ketone or cyclohexanone, strongly polar solvents, such as dimethyl sulphoxide, and also water.

Suitable solid carriers are:

e.g. powdered natural minerals, such as kaolins, clay earths, talc, chalk, quartz, attapulgite, montmorillonite or diatomaceous earth, and powdered synthetic minerals, such as highly disperse silica, alumina and silicates; suitable solid carriers for granules are: e.g. crushed and fractionated natural rocks such as calcite, marble, pumice, sepiolite and dolomite, and also synthetic granules of inorganic and organic meals, and granules of organic material such as paper, sawdust, coconut shells, corn cobs and tobacco stalks; suitable emulsifiers and/or foam-generating agents are: e.g. nonionogenic and anionic emulsifiers, such as polyoxyethylene fatty acid esters, polyoxyethylene fatty alcohol ethers, e.g. alkylaryl polyglycol ethers, alkylsulphonates, alkyl sulphates, arylsulphonates and protein hydrolysates. Suitable dispersants are nonionic and/or ionic substances, e.g. from the classes of the alcohol POE and/or POP ethers, acid and/or POP POE esters, alkylaryl and/or POP POE ethers, fat and/or POP POE adducts, POE and/or POP polyol derivatives, POE and/or POP sorbitan or sugar adducts, alkyl or aryl sulphates, sulphonates and phosphates or the corresponding PO ether adducts. Further suitable oligomers or polymers, e.g. starting from vinylic monomers, of acrylic acid, of EO and/or PO alone or in combination with e.g. (poly)alcohols or (poly)amines. It is also possible to use lignin and its sulphonic acid derivatives, simple and modified celluloses, aromatic and/or aliphatic sulphonic acids, and adducts thereof with formaldehyde.

In the formulations it is possible to use adhesives such as carboxymethylcellulose, natural and synthetic polymers in the form of powders, granules or latices, such as gum Arabic, polyvinyl alcohol, polyvinyl acetate, and also natural phospholipids, such as cephalins and lecithins and synthetic phospholipids.

It is possible to use dyes such as inorganic pigments, e.g. iron oxide, titanium oxide, Prussian blue, and organic dyes, such as alizarin dyes, azo dyes and metallophthalocyanine dyes, and trace nutrients such as salts of iron, manganese, boron, copper, cobalt, molybdenum and zinc.

Further additives may be fragrances, mineral or vegetable optionally modified oils, waxes and nutrients (including trace nutrients), such as salts of iron, manganese, boron, copper, cobalt, molybdenum and zinc.

Furthermore, stabilizers may also be present, such as low-temperature stabilizers, preservatives, oxidation inhibitors, light stabilizers or other agents that improve the chemical and/or physical stability.

The formulations generally comprise between 0.01 and 98% by weight of active ingredient, preferably between 0.5 and 90%.

The active ingredient may be present in its standard commercial formulations, and also in the application forms prepared from these formulations, in a mixture with other active ingredients, such as insecticides, attractants, sterilizers, bactericides, acaricides, nematicides, fungicides, growth-regulating substances, herbicides, safeners, fertilizers, semiochemicals or else with agents for improving plant properties.

When used as insecticides in their standard commercial formulations, and also in the application forms prepared from these formulations, the active ingredients according to the invention may also be present in a mixture with synergistics. Synergistics are compounds by which the effect of the active ingredients is increased without the added synergistic itself having to be active.

When used as insecticides in their standard commercial formulations, and also in the application forms prepared from these formulations, the active ingredients according to the invention may also be present in mixtures with inhibitors which reduce the degradation of the active ingredient following application in the environment of the plant, on the surface of plant parts or in plant tissues.

The active ingredient content of the application forms prepared from the standard commercial formulations can vary within wide ranges. The active ingredient concentration of the application forms can be from 0.00000001 to 95% by weight of active ingredient, preferably between 0.00001 and 1% by weight.

Application takes place in a customary manner adapted to the application forms.

According to the invention, all plants and plant parts can be treated. Plants are to be understood here as meaning all plants and plant populations such as desired and undesired wild plants or crop plants (including naturally occurring crop plants). Crop plants may be plants which can be obtained by conventional cultivation and optimization methods or by biotechnological and genetic engineering methods or combinations of these methods, including the transgenic plants and including the plant cultivars protectable or nonprotectable by plant breeders' rights. Plant parts are to be understood as meaning all parts and organs of the plants above and below the ground, such as shoot, leaf, flower and root, examples which may be mentioned being leaves, needles, stalks, stems, flowers, fruit bodies, fruits and seed material, and also roots, tubers and rhizomes. The plant parts also include harvested material, and also vegetative and generative propagation material, for example cuttings, tubers, rhizomes, offshoots and seed material.

Treatment according to the invention of the plants and plant parts with the active ingredients takes place directly or through contact with their surroundings, habitat or storage space by the customary treatment methods, for example by immersion, spraying, evaporation, fogging, scattering, painting on, injection, pouring on and, in the case of propagation material, in particular in the case of seed material, also by applying one or more coats.

As already mentioned above, according to the invention it is possible to treat all plants and their parts. In one preferred embodiment, wild plant species or plant varieties and plant cultivars which have been obtained by conventional biological cultivation methods, such as hybridization or protoplast fusion, and their parts are treated. In a further preferred embodiment, transgenic plants and plant cultivars which have been obtained by genetic engineering methods optionally in combination with conventional methods (genetically modified organisms) and their parts are treated. The terms “parts” or “parts of plants” or “plant parts” have been explained above.

Plants which are treated particularly preferably according to the invention are those of the plant cultivars which are in each case commercially available or in use. Plant cultivars are understood as meaning plants with new properties (“traits”) which have been cultivated either by conventional cultivation, by mutagenesis or by recombinant DNA techniques. These may be cultivars, biotypes and genotypes.

Depending on the plant species or plant cultivars, their location and growth conditions (soils, climate, vegetation period, nutrition), the treatment according to the invention may also result in superadditive (“synergistic”) effects. Thus, for example, reduced application rates and/or widening of the activity spectrum and/or increasing the effect of the substances and compositions that can be used according to the invention, better plant growth, increased tolerance to high or low temperatures, increased tolerance to drought or to salt content in the water or soil, increased flowering performance, facilitated harvesting, accelerated maturation, higher harvest yields, higher quality and/or higher nutritional value of the harvested products, better storability and/or processability of the harvested products are possible, which exceed the effects which were actually expected.

The preferred transgenic (genetically engineered) plants or plant cultivars to be treated according to the invention include all of those plants which, as a result of the genetic modification, have received genetic material which confers particular advantageous valuable properties (“traits”) to these plants. Examples of such properties are better plant growth, increased tolerance to high or low temperatures, increased tolerance to drought and to salt content in the water or soil, increased flowering performance, facilitated harvesting, accelerated maturation, higher harvest yields, higher quality and/or higher nutritional value of the harvested products, better storability and/or processability of the harvested products. Further and particularly emphasized examples of such properties are an increased defence of the plants against animal and microbial pests, such as against insects, mites, phytopathogenic fungi, bacteria and/or viruses, and an increased tolerance of the plants to certain herbicidal active ingredients. Examples of transgenic plants are the important crop plants, such as cereals (wheat, rice), corn, soybeans, potato, sugarbeet, tomatoes, peas and other vegetable varieties, cotton, tobacco, oilseed rape, and fruit plants (with the fruits apples, pears, citrus fruits and grapes), with particular emphasis on corn, soybeans, potato, cotton, tobacco and oil seed rape. Properties (“traits”) which are especially emphasized are the increased defence of the plants against insects, arachnids, nematodes and slugs and snails, as a result of toxins being formed in the plants, in particular those which are generated in the plants by the genetic material from Bacillus Thuringiensis (e.g. by the genes CryIA(a), CryIA(b), CryIA(c), CryIIA, CryIIIA, CryIIIB2, Cry9c Cry2Ab, Cry3Bb and CryIF and combinations thereof), (hereinbelow “Bt plants”). Further particularly emphasized properties (“traits”) are the increased defence of plants against fungi, bacteria and viruses through systematic acquired resistance (SAR), systemin, phytoalexins, elicitors and resistance genes and correspondingly expressed proteins and toxins. Also particularly emphasized properties (“traits”) are the increased tolerance of the plants to certain herbicidal active ingredients, for example imidazolinones, sulphonylureas, glyphosate or phosphinotricin (e.g. “PAT” gene). The genes that in each case confer the desired properties (“traits”) may also be present in the transgenic plants in combinations with one another. Examples of “Bt plants” that may be mentioned are corn cultivars, cotton cultivars, soybean cultivars and potato cultivars which are sold under the trade names YIELD GARD® (e.g. corn, cotton, soybeans), KnockOut® (e.g. corn), StarLink® (e.g. corn), Bollgard® (cotton), Nucotn® (cotton) and NewLeaf® (potato). Examples of herbicide-tolerant plants that may be mentioned are corn cultivars, cotton cultivars and soybean cultivars which are sold under the trade names Roundup Ready® (tolerance to glyphosate, for example corn, cotton, soybeans), Liberty Link® (tolerance to phosphinotricin, e.g. oil seed rape), IMF® (tolerance to imidazolinones) and STS® (tolerance to sulphonylureas, for example corn). Herbicide-resistant plants (plants cultivated in a conventional manner for herbicide tolerance) that may be mentioned are also the cultivars sold under the name Clearfield® (e.g. corn). Naturally, these statements also apply to plant cultivars having these genetic properties (“traits”) or genetic properties (“traits”) still to be developed, which plant cultivars will be developed and/or marketed in the future.

The plants listed can be treated particularly advantageously according to the invention with the compounds of the general formula I or the active ingredient mixtures according to the invention. The preferred ranges stated above for the active ingredients or mixtures also apply to the treatment of these plants. Particular emphasis may be given to the treatment of plants with the compounds or mixtures specifically listed in the present text.

The active ingredients according to the invention are not only effective against plant, hygiene and stored-product pests, but also in the veterinary medicine sector against animal parasites (ectoparasites and endoparasites), such as ixodid ticks, argasid ticks, scab mites, trombiculid mites, flies (stinging and sucking), parasitic fly larvae, lice, hair lice, bird lice and fleas. These parasites include:

From the order of the Anoplurida, for example Haematopinus spp., Linognathus spp., Pediculus spp., Phtirus spp., Solenopotes spp.

From the order of the Mallophagida and the sub-orders Amblycerina and Ischnocerina, for example Trimenopon spp., Menopon spp., Trinoton spp., Bovicola spp., Werneckiella spp., Lepikentron spp., Damalina spp., Trichodectes spp., Felicola spp.

From the order Diptera and the sub-orders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp.

From the order of the Siphonapterida, for example Pulex spp., Ctenocephalides spp., Xenopsylla spp., Ceratophyllus spp.

From the order of the Heteropterida, for example Cimex spp., Triatoma spp., Rhodnius spp., Panstrongylus spp.

From the order of the Blattarida, for example Blatta orientalis, Periplaneta americana, Blattela germanica, Supella spp.

From the sub-class of the Acari (Acarina) and the orders of the Meta- and Mesostigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp., Varroa spp.

From the order of the Actinedida (Prostigmata) and Acaridida (Astigmata), for example Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.

The active ingredients of the formula (I) according to the invention are also suitable for controlling arthropods which attack agricultural livestock, such as, for example, cattle, sheep, goats, horses, pigs, donkeys, camels, buffalos, rabbits, chickens, turkeys, ducks, geese, honeybees, other domestic animals, such as, for example, dogs, cats, cage birds, aquarium fish, and so-called experimental animals, such as, for example, hamsters, guinea pigs, rats and mice. By controlling these arthropods, the aim is to reduce deaths and decreased performances (in the case of meat, milk, wool, hides, eggs, honey etc.), so that more economical and simpler animal keeping is made possible by using the active ingredients according to the invention.

In the veterinary sector and in animal keeping, the active ingredients according to the invention are used in a known manner by enteral administration, in the form, for example, of tablets, capsules, drinks, drenches, granules, pastes, boli, the feed-through method, suppositories, by parenteral administration, such as, for example, by means of injections (intramuscular, subcutaneous, intravenous, intraperitonal and the like), implants, by nasal application, by dermal application in the form, for example, of immersion or bathing (dipping), spraying, pouring-on and spotting-on, washing, dusting, and also with the help of shaped bodies that contain active ingredient, such as collars, ear tags, tail marks, limb bands, halters, marking devices etc.

When used for livestock, poultry, domestic animals etc., the active ingredients of the formula (I) can be used as formulations (for example powders, emulsions, flowables) which comprise the active ingredients in an amount of from 1 to 80% by weight, directly or after dilution by a factor of 100 to 10 000, or they can be used in the form of a chemical bath.

Furthermore, it has been found that the compounds according to the invention exhibit a high insecticidal effect against insects which destroy industrial materials.

The following insects may be mentioned by way of example and preference, but without any limitation:

beetles, such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec. Tryptodendron spec. Apate monachus, Bostrychus capucins, Heterobostrychus brunneus, Sinoxylon spec. Dinoderus minutus;
Dermapterans, such as Sirex juvencus, Urocerus gigas, Urocerus gigas taignus, Urocerus augur;
Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus;
Bristletails, such as Lepisma saccharina.

Industrial materials are to be understood as meaning, in the present context, inanimate materials, such as, preferably, plastics, adhesives, sizes, papers and boards, leather, wood, wood processing products and coatings.

The ready-to-use compositions can, if appropriate, also comprise further insecticides and, if appropriate, also one or more fungicides.

The compounds according to the invention can equally be used for protecting against fouling of objects, in particular of hulls, screens, nets, buildings, moorings and signalling systems which come into contact with salt water or brackish water.

Furthermore, the compounds according to the invention can be used alone or in combinations with other active ingredients as antifouling composition.

The active ingredients are also suitable for controlling animal pests in household protection, hygiene protection and stored-product protection, in particular insects, arachnids and mites, which are encountered in enclosed spaces, such as, for example, dwellings, factory halls, offices, vehicle cabins and the like. They can be used for controlling these pests alone or in combination with other active ingredients and auxiliaries in domestic insecticide products. They are effective against sensitive and resistant species and against all stages of development. These pests include:

From the order of the Scorpionidea, for example Buthus occitanus.

From the order of the Acarina, for example Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.

From the order of the Araneae, for example Aviculariidae, Araneidae.

From the order of the Opiliones, for example Pseudoscorpiones chelifer, Pseudoscorpiones cheiridium, Opiliones phalangium.

From the order of the Isopoda, for example Oniscus asellus, Porcellio scaber.

From the order of the Diplopoda, for example Blaniulus guttulatus, Polydesmus spp.

From the order of the Chilopoda, for example Geophilus spp.

From the order of the Zygentoma, for example Ctenolepisma spp., Lepisma saccharina, Lepismodes inquilinus.

From the order of the Blattaria, for example Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example Acheta domesticus.

From the order of the Dermaptera, for example Forficula auricularia.

From the order of the Isoptera, for example Kalotermes spp., Reticulitermes spp.

From the order of the Psocoptera, for example Lepinatus spp., Liposcelis spp.

From the order of the Coleoptera, for example Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa.

From the order of the Lepidoptera, for example Achroia grisella, Galleria mellonella, Plodia interpunctella, Tinea cloacella, Tinea pellionella, Tineola bisselliella.

From the order of the Siphonaptera, for example Ctenocephalides canis, Ctenocephalides felis, Pulex irritans, Tunga penetrans, Xenopsylla cheopis.

From the order of the Hymenoptera, for example Camponotus herculeanus, Lasius fuliginosus, Lasius niger, Lasius umbratus, Monomorium pharaonis, Paravespula spp., Tetramorium caespitum.

From the order of the Anoplura, for example Pediculus humanus capitis, Pediculus humanus corporis, Pemphigus spp., Phylloera vastatrix, Phthirus pubis.

From the order of the Heteroptera, for example Cimex hemipterus, Cimex lectularius, Rhodinus prolixus, Triatoma infestans.

They are used in the domestic insecticide sector alone or in combination with other suitable active ingredients, such as phosphoric acid esters, carbamates, pyrethroids, neonicotinoids, growth regulators or active ingredients from other known insecticide classes.

Application takes place in aerosols, pressureless spray products, for example pump sprays and atomizer sprays, automatic fogging systems, foggers, foams, gels, evaporator products with evaporator tablets made of cellulose or polymer, liquid evaporators, gel and membrane evaporators, propeller-driven evaporators, energy-free or passive evaporation systems, moth papers, moth bags and moth gels, as granules or dusts, in baits for spreading or in bait stations.

### DESCRIPTION OF THE PROCESSES AND INTERMEDIATES

As an example and elucidation, the preparation of compounds of the formula (I) is described in the following formula schemes. Reference may also be made at this point to the Preparation Examples.

The compounds according to the invention of the formula (I) with W═O and R¹═H or methyl are obtained by formula scheme 1. The compounds according to the invention of the formula (I) with W═S can be obtained from them by reaction with a sulphurizing reagent such as Lawesson's reagent.

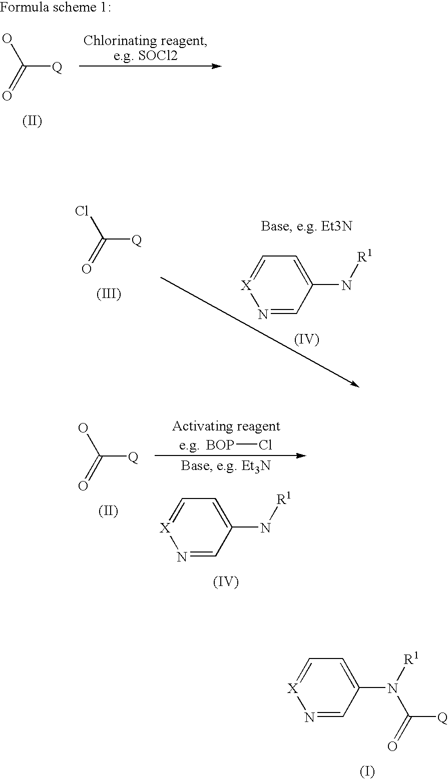

The carboxylic acids of the formula (II) are either converted into their acid chlorides (III) by reaction with a chlorinating reagent such as thionyl chloride, for example, and subsequently reacted with the amines of the formula (IV) with R¹═H or methyl, in the presence of an auxiliary base such as triethylamine, to give the compounds according to the invention of the formula (I), or first reacted with an activating reagent such as BOP-Cl, in the presence of an auxiliary base such as triethylamine, and thereafter reacted with the amines of the formula (IV) with R¹═H or methyl to give the compounds according to the invention of the formula (I).

The aminopyridines of the formula (IV) and aminopyridazines with R¹═H or methyl, required for preparing the compounds according to the invention of the formula (I), have been described.

For 3-aminopyridine, see Chemische Berichte 1936, 1534; for 4-aminopyridazine, see Pharmaceutical Bulletin 4, 1956, 137, 497; for 4-methylaminopyridazine, see Journal of the Chemical Society C, 1967, 23, 2473, and, for 3-methylaminopyridine, see Journal of the Chemical Society 1957, 442.

The compounds according to the invention of the formula (I) with W═O and R¹≠H or methyl are obtained by formula scheme 2.

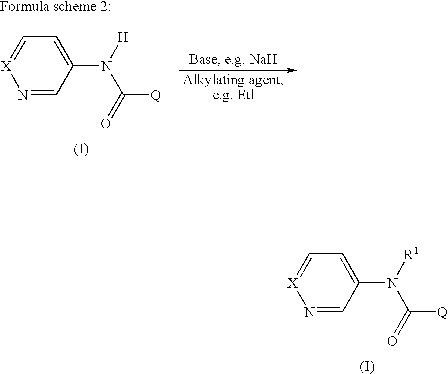

The compounds according to the invention of the formula (I) with W═O and R¹≠H or methyl are reacted, in the presence of an auxiliary base such as sodium hydride, for example, with an alkylating agent such as ethyl iodide, for example.

The compounds according to the invention of the formula (I) with W═S can be obtained thereafter by reaction with a sulphurizing reagent such as Lawesson's reagent.

The carboxylic acids of the formula (II) that are required for preparing the compounds according to the invention of the formula (I) can be prepared by methods that are known in principle, or are known.

For 3-difluoromethyl-1-methyl-1H-pyrazole-4-carboxylic acids, see, for example, WO2005123690.

For 1-methyl-3-trifluoromethyl-1H-pyrazole-4-carboxylic acid, see, for example, U.S. Pat. No. 5,438,070.

For 1-ethyl-3-trifluoromethyl-1H-pyrazole-4-carboxylic acid, see, for example, U.S. Pat. No. 5,480,587.

For 3-cyclopropyl-1-methyl-1H-pyrazole-4-carboxylic acid, see, for example, JP03190862.

For 3-methoxy-1-methyl-1H-pyrazole-4-carboxylic acid, see, for example, US2007/196406.

For 3-(chlorodifluoromethyl)-1-methyl-1H-pyrazole-4-carboxylic acid, see, for example, U.S. Pat. No. 5,223,526.

For 5-methyl-1-(2,2,2-trifluoroethyl)-1H-pyrazole-4-carboxylic acid, see, for example, EP1176140.

For 1,3-dimethyl-1H-pyrazole-4-carboxylic acid, see, for example, EP1176140.

For 3-(4,6-dimethoxypyrimidin-2-yloxy)benzoic acid, see, for example, WO2004100946.

For 4-(4,6-dimethylpyrimidin-2-yloxy)benzoic acid, see, for example, DE3602016.

For 3-bromo-4-methoxybenzoic acid, see, for example, Liebigs Annalen, 1989, 863.

The particularly preferred carboxylic acids of the formula (II) with Q8-1 (formula (II-1), and Q8a-2 (formula II-2) can be prepared in general by formula scheme 3.

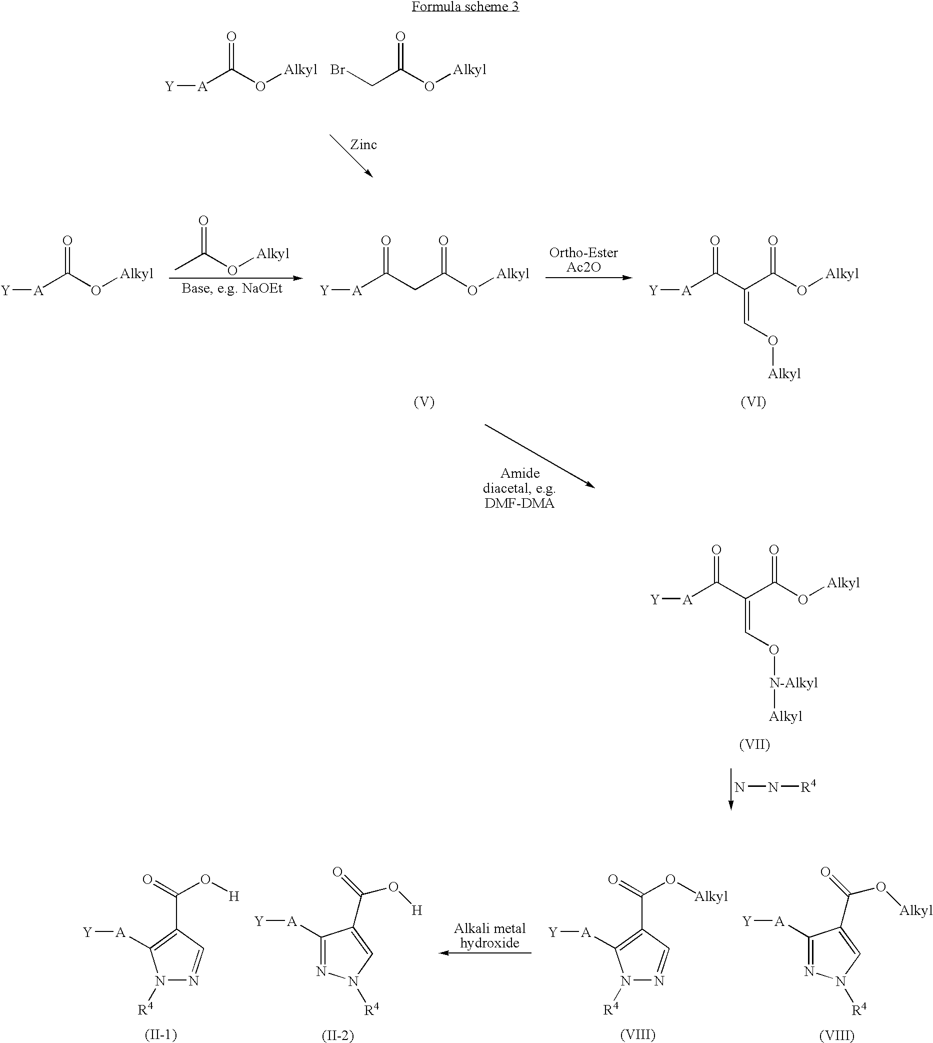

The beta-ketocarboxylic esters of the formula (V) are obtained either by reaction of a carboxylic ester with alkyl acetate in the presence of an auxiliary base such as sodium ethoxide—in this regard, see, for example, Journal of the American Chemical Society 1953, 3152 or Tetrahedron 2005, 2169 or Tetrahedron Letters 2001, 2689—or by formation of a zinc enolate from a bromoacetate and reaction with a carboxylic ester—in this regard, see Bioorganic and Medicinal Chemistry 2004, 233, or Journal of the American Chemical Society 1954, 5391. The esters of the formula (V) are then reacted with ortho-esters in the presence of acetic anhydride to give the enol ethers of the formula (VI)—in this regard, see Journal of the American Chemical Society 1951, 3684—or with amide diacetals such as, for example, DMF dimethyl acetal, to give the dialkyleneamines of the formula (VII) in this regard see, for example, Bioorganic and Medicinal Chemistry Letters, 2005, 4370. From the compounds of the formula (VI) and (VII), the pyrazolecarboxylic esters of the formula (VIII) are then obtained, by reaction with hydrazines, and can be converted by reaction with a base such as alkali metal hydroxide into carboxylic acids of the formula (II).

The thiazolecarboxylic acids of the formula (II-3) with Q11-1 are known or can be prepared by methods known in principle by formula scheme 4.

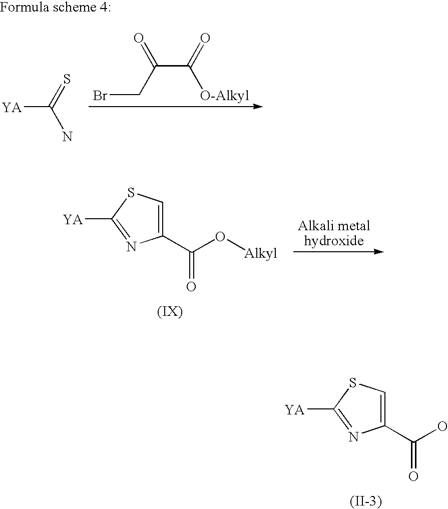

Reaction of thioamides with bromopyruvates gives the thiazolecarboxylic esters of the formula (IX); the thioamides required can be obtained, for example, by reaction of the corresponding nitriles with ammonium sulphide. The carboxylic acids of the formula (II-3) are obtained from the carboxylic esters of the formula (IX) by reaction with alkali metal hydroxide.

The compounds according to the invention of the formula (I) with Q1 may likewise be obtained by reaction of compounds of the formula (I) with a reactive group F such as, for example, chlorine or bromine in accordance with formula scheme 5. The required compounds of the formula (I) with a reactive group F can be prepared by formula scheme 1.

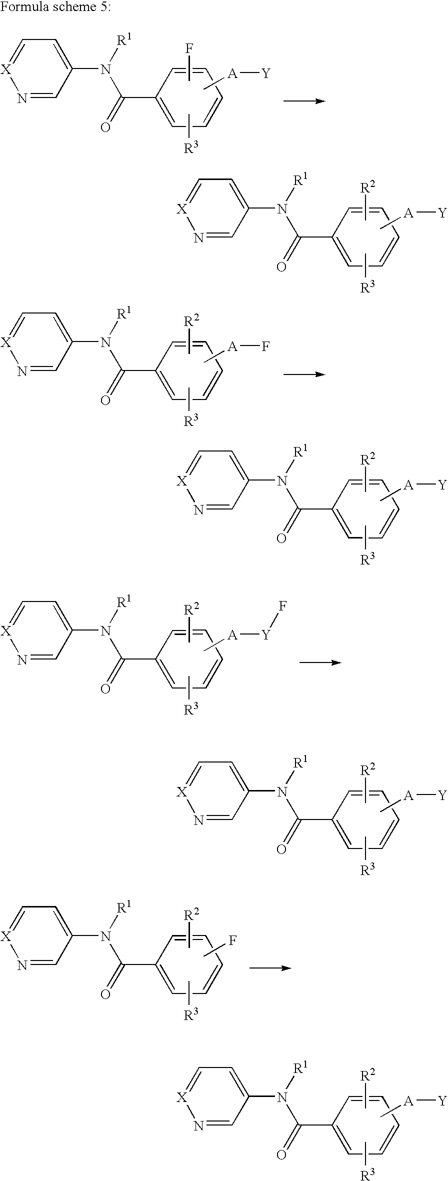

The compounds according to the invention of the formula (I) with Q2 may be obtained likewise by reaction of compounds of the formula (I) with a reactive group F such as, for example, chlorine or bromine by formula scheme 6. The required compounds of the formula (I) with a reactive group F can be prepared by formula scheme 1.

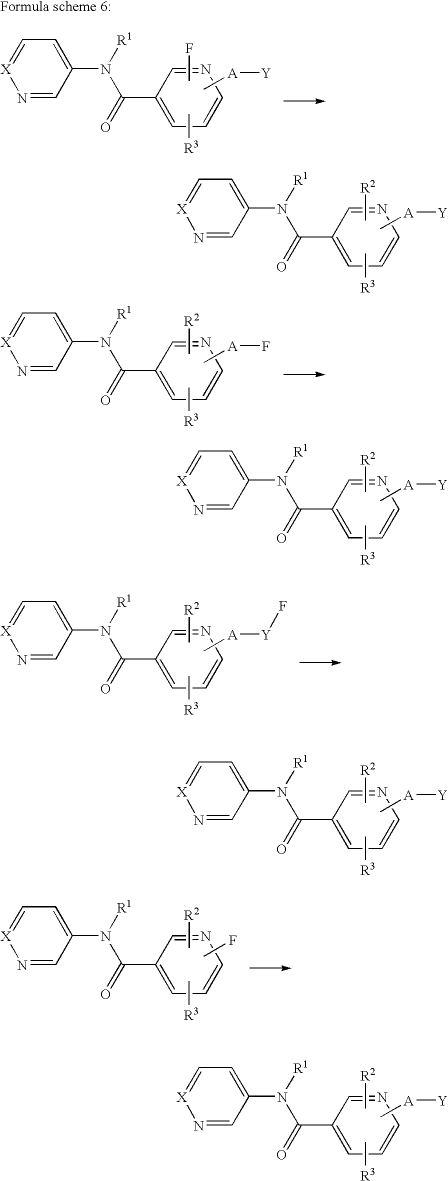

For a more precise explanation, see the reactions given by way of example in formula schemes 7, 8 and 9.

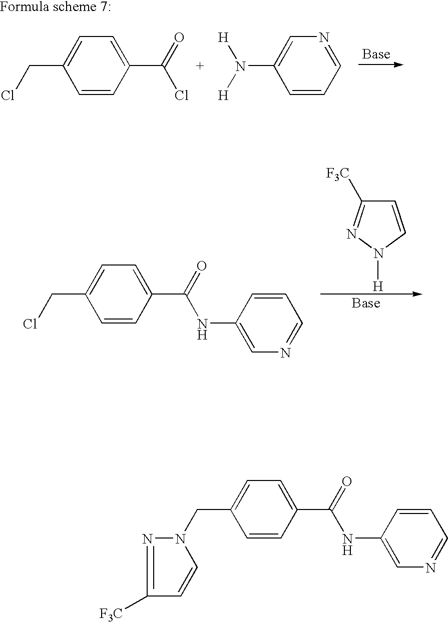

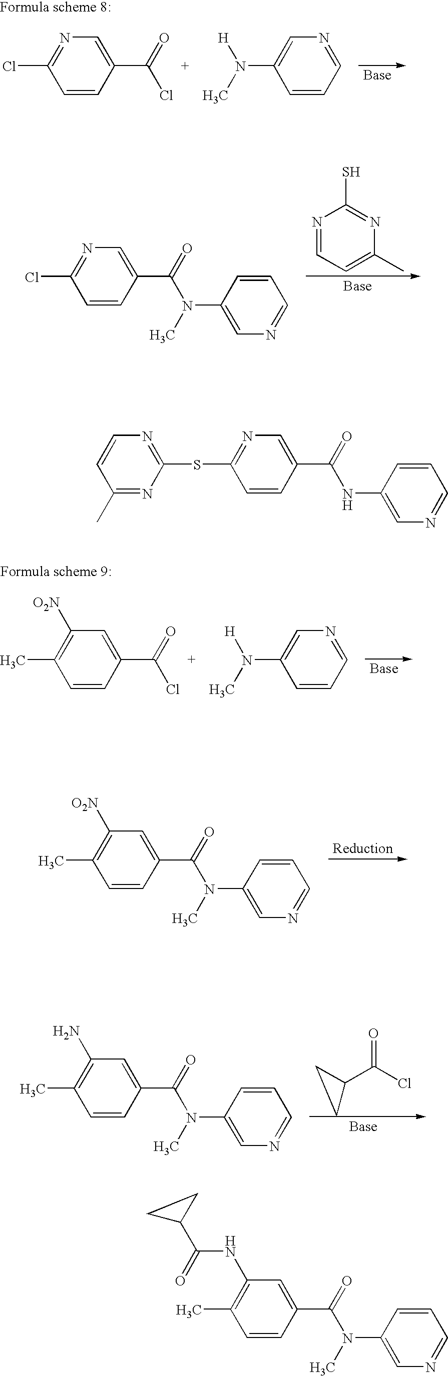

The Preparation Examples and Use Examples which follow provide an illustration of the invention, without limiting it.

### PREPARATION EXAMPLES

### Example 1

### Stage 1

### Methylpyridin-3-ylamine

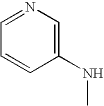

An amount of 100.1 g (633 mmol) of 3-bromopyridine was stirred with 160 ml (1280 mmol) of 8M methylamine in ethanol, 4 g (27.5 mmol) of 8-hydroxyquinoline and 2.2 g (11.5 mmol) of copper(I) iodide in an autoclave at 120° for 16 hours. The mixture was filtered with suction over sand/silica gel and concentrated, aqueous citric acid and cyclohexane were added to the residue, the aqueous phase was saturated with sodium chloride, admixed with dilute aqueous sodium hydroxide solution to a pH of 10 and extracted 6 times with ethyl acetate, and the combined organic phases were dried over MgSO₄ and evaporated. The residue was distilled in a bulb tube under a membrane pump vacuum.

Yield: 46.8 g (63% of theory), 1H-NMR (CD3CN) 2.75 (s, 3H), 4.3 (br, 1H), 6.85 (m, 1H), 7.05 (m, 1H), 7.8 (m, 1H), 7.95 (m, 1H)

### Stage 2

### Ethyl 2-[1-ethoxymethylidene]-4,4,4-trifluoro-3-oxobutyrate

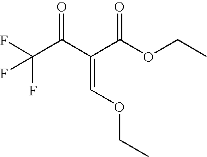

An amount of 101 g (548.5 mmol) of ethyl 4,4,4-trifluoro-3-oxobutyrate, 164 g (1106 mmol) of triethyl orthoformate and 114.1 g (1118 mmol) of acetic anhydride were stirred in a distillation apparatus at 120° C. for 1 hour, and then the temperature was increased to 140° C. over the course of 30 minutes. Partial cooling was followed by distillation under a membrane pump vacuum.

Yield: 103.6 g (75% of theory) 1H-NMR (CD3CN) 1.2-1.4 (m, 6H), 4.2-4.4 (m, 4H), 7.75-7.95 (1H)

### Stage 3

### Ethyl 1-pyrimidin-2-yl-5-trifluoromethyl-1H-pyrazole-4-carboxylate

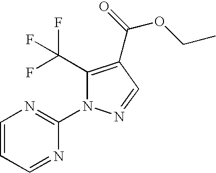

An amount of 6.5 g (27 mmol) of ethyl 2-[1-ethoxymethylidene]-4,4,4-trifluoro-3-oxobutyrate was dissolved in ethanol/dioxane and the solution was admixed with 2.99 g (27.1 mmol) of 2-hydrazinopyrimidine with ice-bath cooling. The mixture was stirred at RT for 16 hours and then heated at reflux for a further 1.5 hours, and then evaporated, and the residue was purified by chromatography (cyclohexane/acetone).

Yield: 6.08 g (77% of theory) 1H-NMR (D6-DMSO) 1.3 (t, 3H), 4.35 (q, 2H), 7.85 (t, 1H), 8.4 (s, 1H), 9.1 (d, 2H)

### Stage 4

### Pyrimidin-2-yl-5-trifluoromethyl-1H-pyrazole-4-carboxylic acid

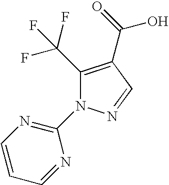

An amount of 5.89 g (20.5 mmol) of ethyl 1-pyrimidin-2-yl-5-trifluoromethyl-1H-pyrazole-4-carboxylate was dissolved in 50 ml of tetrahydrofuran and admixed with aqueous sodium hydroxide solution. Dilution with 100 ml of ethanol caused a precipitate to form. Aqueous citric acid was added, dilution was carried out with water and ethyl acetate, aqueous sodium chloride was added, and the mixture was extracted 4 times with ethyl acetate. The combined organic phases were dried over MgSO₄ and concentrated by evaporation.

Yield: 5.07 g (88% of theory), 1H-NMR (D6-DMSO) 7.75 (m, 1H), 8.25 (m, 1H), 9.05 (m, 2H).

### Stage 5

### 1-Pyrimidin-2-yl-5-trifluoromethyl-1H-pyrazole-4-carboxylic acid methylpyridin-3-ylamide (I-268)

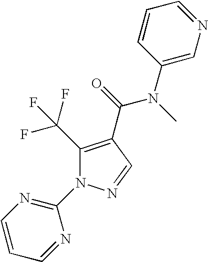

An amount of 1 g (3.8 mmol) of 1-pyrimidin-2-yl-5-trifluoromethyl-1H-pyrazole-4-carboxylic acid was admixed in 40 ml of dichloromethane with 1.5 g (11.6 mmol) of diisopropylethylamine and 1 g (4 mmol) of BOP-Cl and the mixture was stirred for 1 hour and then admixed with 0.68 g (5.8 mmol) of methylpyridin-3-ylamine and stirred for 16 hours. Water was added and the organic phase was separated off, dried and concentrated by evaporation. The residue was purified by chromatography (cyclohexane/acetone).

Yield: 1 g (74% of theory), 1H-NMR see products list

### Example 2

### Stage 1

### 3-Bromo-4-methoxy-N-methyl-N-pyridin-3-ylbenzamide

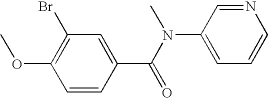

An amount of 5 g (22 mmol) of 3-bromo-4-methoxybenzoic acid was dissolved in 50 ml of dichloromethane with 8.3 g (65 mmol) of diisospropylethylamine, and the solution was admixed with 5.8 g (23 mmol) of BOP-Cl and then with 2.5 g (24 mmol) of methylpyridin-3-ylamine and stirred for 2 days. The mixture was concentrated, the residue was admixed with water and aqueous sodium chloride and extracted three times with ethyl acetate, and the combined organic phases were dried and concentrated by evaporation. The residue was purified by chromatography on silica gel (cyclohexane/acetone).

Yield: 5.49 g (74% of theory), ¹H-NMR (D6-DMSO) 3.35 (s, 3H), 3.8 (s, 3H), 6.95 (d, 1H), 7.25 (m, 1H), 7.35 (m, 1H), 7.5 (m, 1H), 7.7 (m, 1H), 8.35 (m, 2H)

### Stage 2

### 4-Methoxy-N-methyl-3-pyrazol-1-yl-N-pyridin-3-ylbenzamide (I-280)

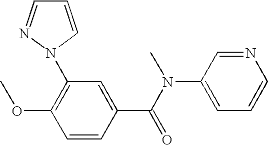

An amount of 0.8 g (2 mmol) of (3-bromo-4-methoxy-N-methyl-N-pyridin-3-ylbenzamide, 1 g (15 mmol) of pyrazole, 2 g (15 mmol) of potassium carbonate, 38 mg (0.2 mmol) of copper(I) iodide and 36 mg (0.24 mmol) of 8-hydroxyquinoline were stirred in 20 ml of DMF with 5 drops of polyethylene glycol 400 under argon at 140° for 15 hours and then concentrated. The residue was admixed with aqueous citric acid, aqueous sodium hydroxide solution and aqueous sodium chloride and extracted at a pH of 9 with ethyl acetate. The organic phase was dried and concentrated by evaporation. The residue was purified by chromatography on silica gel (cyclohexane/acetone).

Yield: 0.25 g (32% of theory) 1H-NMR, see products list.

### Example 3

### Stage 1

### 4-(Chloromethyl)-N-(pyridin-3-yl)benzenecarboxamide

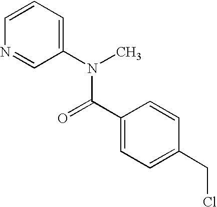

An amount of 1.22 g (6.4 mmol) of 4-chloromethylbenzoyl chloride in dichloromethane was added to 0.7 g (6.4 mmol) of methylpyridin-3-ylamine in pyridine/dichlormethane, with ice-bath cooling. Water and the dichloromethane were added and the organic phase was concentrated. The crude product obtained was reacted further directly.

### Stage 2

### N-Methyl-N-(pyridin-3-yl)-4-{[3-(trifluoromethyl)-1H-pyrazol-1-yl]methyl}benzenecarboxamide (I-27)

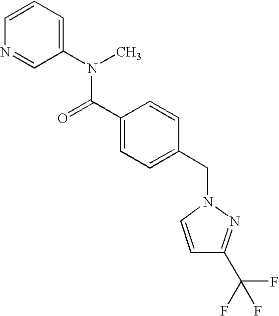

An amount of 0.25 g (0.9 mmol) of 4-(chloromethyl)-N-(pyridin-3-yl)benzenecarboxamide and 3-trifluoromethylpyrazole were stirred with 0.17 g (1.2 mmol) of potassium carbonate in 3 ml of DMF at 65° for 2 hours and then admixed with water/MTBE. The organic phase was concentrated by evaporation and the residue was purified by chromatography.

1H-NMR see products list.

### Example 4

### Stage 1

### 6-Chloro-N-methyl-N-(pyridin-3-yl)pyridine-3-carboxamide

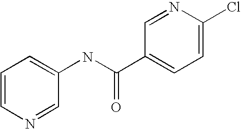

An amount of 5 g (31.7 mmol) of 6-chloronicotinic acid was admixed in 50 ml of dichloromethane with a catalytic amount of dimethylformamide and 10 ml (38 mmol) of oxalyl chloride and the mixture was stirred for 2 hours and concentrated. The residue was admixed with 50 ml of dichloromethane and admixed with 2.98 g (31.75 mmol) of 3-aminopyridine and 6.6 ml of triethylamine, with ice-bath cooling. The mixture was stirred at RT and then admixed with water, the resulting precipitate was filtered off, the filtrate was extracted with dichloromethane, and the organic phase was dried and concentrated by evaporation. The residue was purified by chromatography on silica gel.

Yield: 1.62 g (21% of theory).

### Stage 2

### 6-(3-Chlorophenoxy)-N-pyridin-3-ylnicotinamide (I-83)

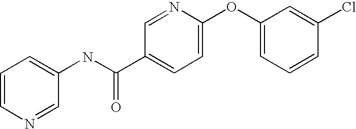

An amount of 250 mg (1.07 mmol) of 6-chloro-N-methyl-N-(pyridin-3-yl)pyridine-3-carboxamide was stirred with 137.5 mg (1.07 mmol) of 3-chlorophenol and 177 mg (1.28 mmol) of potassium carbonate in 10 ml of DMF at 100° C. for 3 hours, and then left to stand at 120° C. for 4 hours. After the batch had cooled, it was admixed with water and then ethyl acetate and the organic phase was dried and concentrated by evaporation. The residue was purified by chromatography on silica gel.

Yield: 230 mg (65% of theory). 1H-NMR see products list.

### Example 5

### Stage 1

### 4,N-Dimethyl-3-nitro-N-pyridin-3-ylbenzamide

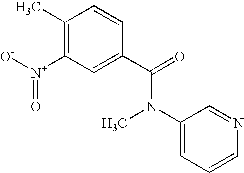

An amount of 1.1 g (5.51 mmol) of 4-methyl-3-nitrobenzoyl chloride was dissolved in 10 ml of chloroform and the solution, with ice-bath cooling, was admixed with 715 mg (6.6 mmol) of methylpyridin-3-ylamine in 10 ml of chloroform with 1 ml of triethylamine and stirred at RT for 16 hours. It was diluted with chloroform and then water and the organic phase was washed with dilute acetic acid and water, dried over MgSO₄ and concentrated by evaporation.

Yield: 1.6 g (101% of theory). 1H-NMR (D6-DMSO) 2.4 (s, 3H), 3.4 (s, 3H), 7.35 (m, 2H), 7.45 (d, 1H), 7.7 (d, 1H), 7.85 (s, 1H), 8.4 (m, 2H)

### Stage 2

### 3-Amino-4,N-dimethyl-N-pyridin-3-ylbenzamide

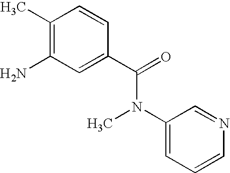

An amount of 1.5 g (5.2 mmol) of 4,N-dimethyl-3-nitro-N-pyridin-3-ylbenzamide was dissolved in 70 ml of ethanol and the solution was admixed over the course of 2 hours with 3.9 g (20 mmol) of tin(II) chloride in 65 ml of EtOH and heated under reflux for 2 hours. The mixture was concentrated by evaporation, admixed with water and aqueous sodium hydroxide solution to a pH of 9 and extracted with ethyl acetate, and the organic phase was dried over MgSO₄ and concentrated by evaporation.

Yield: 0.95 g (71% of theory). 1H-NMR (D6-DMSO): 2 (s, 3H), 3.3 (s, 3H), 4.7 (s, 2H), 6.25 (d, 1H), 6.65 (s, 1H), 6.75 (d, 1H), 7.25 (m, 1H), 7.6 (m, 1H), 8.3 (m, 2H)

### Stage 3

### 3-Isobutyrylamino-4,N-dimethyl-N-pyridin-3-ylbenzamide (I-300)

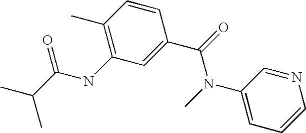

An amount of 100 mg (0.4 mmol) of 3-amino-4,N-dimethyl-N-pyridin-3-ylbenzamide in 1 ml of chloroform with 52 mg (0.5 mmol) of triethylamine was added to a solution of 42 mg (0.4 mmol) of isobutanoyl chloride in 1.5 ml of chloroform (1 mmol) EDC in solution in 2 ml of dioxane, with ice-bath cooling. Stirring was carried out at RT for 16 hours and 50° C. for a further 16 hours. The mixture was diluted with chloroform and water and the organic phase was dried over MgSO₄ and concentrated by evaporation.

Yield: 89 mg (64% of theory) 1H-NMR (D6-DMSO) 1.05 (m, 6H), 2.2 (s, 3H), 2.6 (sep, 1H), 3.35 (s, 3H), 6.9 (d, 1H), 7.05 (d, 1H), 7.3 (m, 1H), 7.4 (s, 1H), 7.65 (m, 1H), 8.3 (m, 2H), 8.9 (s, 1H)

The compounds of the general formula (I) described in Table 1 are obtained in accordance with or in analogy to the synthesis examples described above:

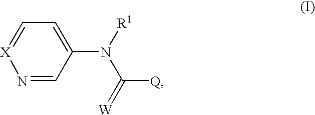

where the radicals R¹, Q, X and W have the definitions stated in Table 1.

TABLE 1

| (I-) | Structure | MS | 1H-NMR |
|---|---|---|---|
| 1 | 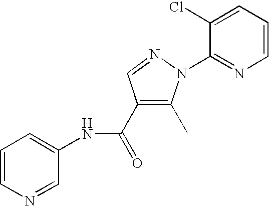 | | (D6-DMSO) δ: 9.95 ppm (NH); 8.88(d, 1 H); 8.63-8.62(m, 1 H); 8.33(s, 1 H); 8.29-8.26(m, 2H); 8.14-8.11(m, 1 H); 7.71-7.68(m, 1 H); 7.37- 7.34(m, 1 H); 2.41(s, CH3). |
| 2 | 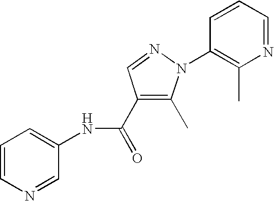 | | (D6-DMSO) δ: 9.91 ppm (NH); 8.63-8.62(m, 1H); 8.12(s, 1 H); 7.98(s, 1 H); 7.80-7.77(m, 2H); 7.44-7.41(m, 2 H); 2.29(s, CH3); 2.18 (s, CH3). |
| 3 | 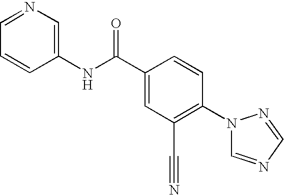 | | (D6-DMSO) δ: 7.41-7.44(1H, m), 8.08-8.11(1H, m), 8.18-8.20(1H, m), 8.34-8.35(1H, m), 8.42- 8.43(2H, m), 8.65(1H, d, J = 1.5 Hz), 8.93(1H, d, J = 2.0 Hz), 9.31(1H, s), 10.71(1H, s). |
| 4 | 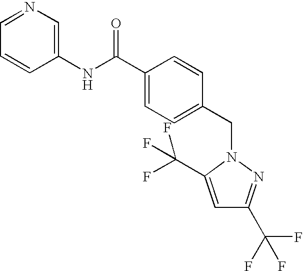 | | (CDCl3) δ: 5.55(2H, s), 6.95(1H, s), 7.29-7.34 (3H, m), 7.86-7.89(2H, m), 7.99-8.02(1H, m), 8.26-8.29(1H, m), 8.38-8.40(1H, m), 8.65-8.66 (1H, m). |
| 5 | 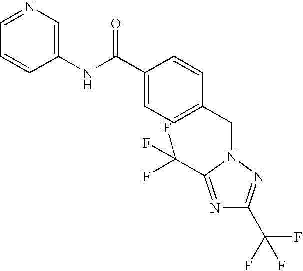 | | (CDCl3) δ: 5.60(2H, s), 7.31-7.34(1H, m), 7.43- 7.45(2H, m), 7.91-7.93(2H, m), 8.12-8.15(1H, m), 8.25-8.28(1H, m), 8.37-8.39(1H, m), 8.66- 8.67(1H, m). |
| 6 | 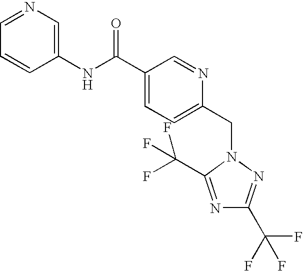 | | (CDCl3) δ: 5.77(2H, s), 7.33-7.36(2H, m), 8.25- 8.28(2H, m), 8.38-8.40(2H, m), 8.68(1H, d, J = 2.4 Hz), 9.04(1H, s). |
| 7 | 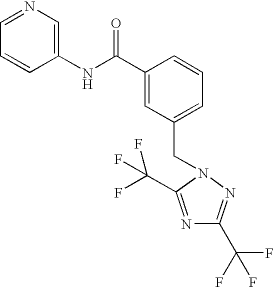 | | (CDCl3) δ: 5.59(2H, s), 7.16-7.34(3H, m), 7.52- 7.53(2H, m), 7.90-7.93(2H, m), 8.25-8.28(1H, m), 8.36-8.39(2H, m), 8.67-8.67(1H, m). |
| 8 | 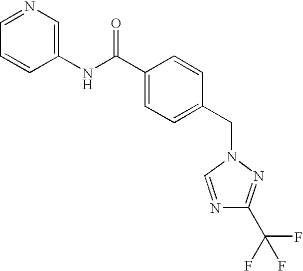 | | (CDCl3) δ: 5.45(2H, s), 7.39-7.41(3H, m), 7.88- 8.03(3H, m), 8.16(1H, s), 8.26-8.29(1H, m), 8.39-8.41(1H, m), 8.68(1H). |
| 9 | 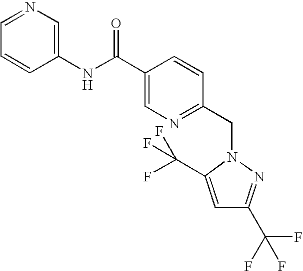 | | (CDCl3) δ: 5.67(2H, d, J = 23.1 Hz), 6.96-7.00 (1H, m), 7.06-7.09(1H, m), 7.32-7.35(1H, m), 8.21-8.26(2H, m), 8.38-8.39(1H, m), 8.46-8.49 (1H, m), 8.67-8.68(1H, m), 9.05-9.06(1H, m). |
| 10 | 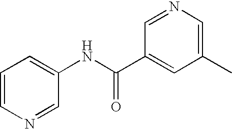 | | (CDCl3) δ: 2.38(3H, s), 7.27-7.29(1H), 8.03 (1H), 8.29-8.30(2H), 8.52(1H), 8.71(1H), 8.91 (1H). |
| 11 | 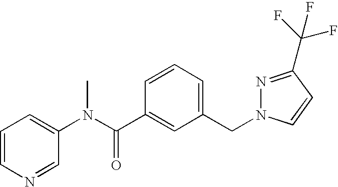 | | (CDCl3) δ: 3.50(5H, s), 5.22(3H, s), 6.51(2H, d, J = 2.0 Hz), 7.17-7.30(13H, m), 8.25(1H, d, J = 2.2 Hz), 8.35-8.37(1H, m). |
| 12 | 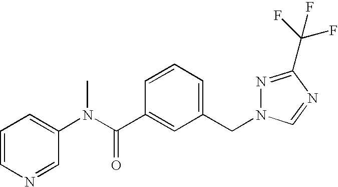 | | (CDCl3) δ: 3.56(3H, t, J = 2.4 Hz), 4.47(2H, t, J = 2.4 Hz), 7.22-7.43(6H, m), 8.39-8.40(1H, m), 8.43-8.44(1H, m). |
| 13 | 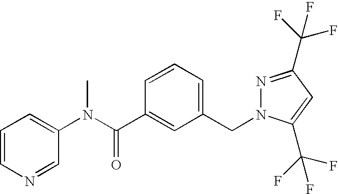 | | (CDCl3) δ: 3.50(3H, s), 5.33(2H, s), 6.90(1H, s), 7.17-7.25(6H, m), 8.27-8.28(1H, m), 8.34- 8.35(1H, m). |
| 14 | 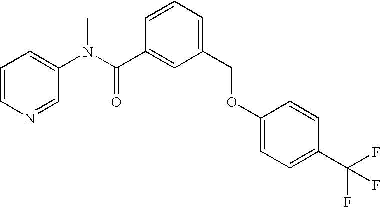 | | (CDCl3) δ: 3.52(3H, s), 4.99(2H, s), 6.90-6.93 (2H, m), 7.13-7.16(1H, m), 7.23-7.27(2H, m), 7.31-7.38(3H, m), 7.51-7.54(2H, m), 8.33-8.35 (2H, m). |
| 15 | 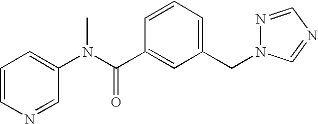 | | (CDCl3) δ: 3.50(3H, s), 5.22(2H, s), 7.14-7.33 (6H, m), 7.88(1H, s), 7.94(1H, s), 8.29(1H, d, J = 2.4 Hz), 8.39(1H, dd, J = 4.8, 1.5 Hz). |
| 16 | 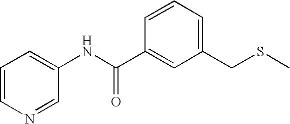 | | (CDCl3) δ: 1.98(3H, s), 3.72(2H, s), 7.30-7.33 (1H, m), 7.45-7.50(2H, m), 7.76-7.80(1H, m), 7.84-7.85(1H, m), 8.29-8.36(2H, m), 8.47(1H, s), 8.68-8.69(1H, m). |
| 17 | 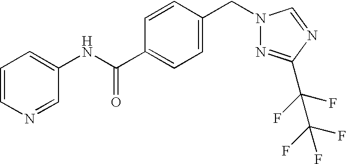 | | (CDCl3) δ: 5.50(2H, s), 7.33-7.36(1H, m), 7.43 (2H, d, J = 8.4 Hz), 7.93(3H, d, J = 8.4 Hz), 8.21 (1H, s), 8.26-8.29(1H, m), 8.40-8.42(1H, m), 8.67-8.68(1H, m). |
| 18 | 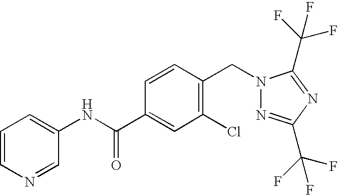 | | (CDCl3) δ: 5.73(2H, s), 7.08-7.11(1H, m), 7.32- 7.34(1H, m), 7.78-7.81(1H, m), 8.01-8.01(1H, m), 8.24-8.27(1H, m), 8.37-8.39(1H, m), 8.46 (1H, br s), 8.67-8.68(1H, m). |
| 19 | 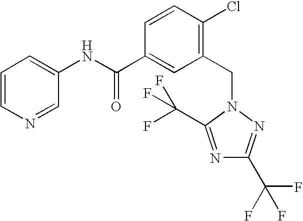 | | (CDCl3) δ: 5.72(2H, s), 7.33-7.36(1H, m), 7.61 (1H, d, J = 8.2 Hz), 7.24-7.75(1H, m), 7.85(1H, d, J = 8.3 Hz), 7.92(1H, s), 8.22-8.25(1H, m), 8.41-8.42(1H, m), 8.65-8.66(1H, m). |
| 20 | 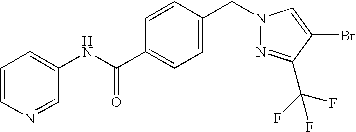 | | (CDCl3) δ: 5.37(2H, s), 7.30-7.36(3H, m), 7.49 (1H, s), 7.87-7.91(2H, m), 8.23-8.25(2H, m), 8.37-8.39(1H, m), 8.67-8.68(1H, m). |
| 21 | 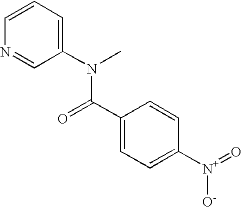 | | (CDCl3) δ: 3.53(3H, s), 7.25(1H, dd), 7.44(2H, d), 8.07(2H, d), 8.33(1H, d), 8.43(1H, dd). |
| 22 | 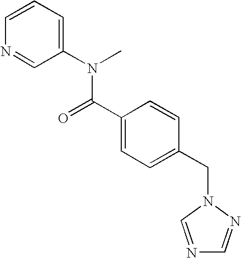 | | (CDCl3) δ: 3.51(3H, s), 5.28(2H, s), 7.06-7.09 (2H, m), 7.19-7.23(1H, m), 7.27-7.31(2H, m), 7.37-7.38(1H, m), 7.96(1H, s), 8.01(1H, s), 8.32-8.33(1H, m), 8.39-8.41(1H, m). |
| 23 | 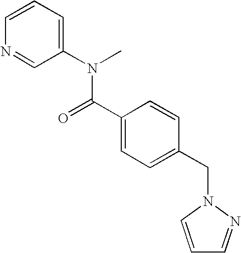 | | (CDCl3) δ: 3.51(3H, s), 5.26(2H, s), 6.27-6.27 (1H, m), 6.99(2H, d, J = 8.4 Hz), 7.17-7.39(6H, m), 7.52-7.53(1H, m), 8.33-8.34(1H, m), 8.38- 8.39(1H, m). |
| 24 | 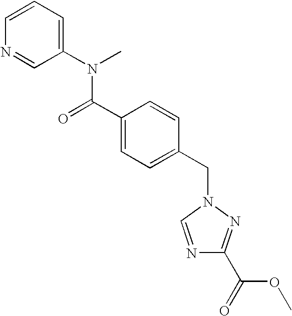 | | (CDCl3) δ: 3.51(3H, s), 3.99(3H, s), 5.36(2H, s), 7.10-7.13(2H, m), 7.20-7.23(1H, m), 7.30- 7.31(2H, m), 7.38-7.38(1H, m), 8.08(1H, s), 8.32-8.33(1H, m), 8.39-8.41(1H, m). |
| 25 | 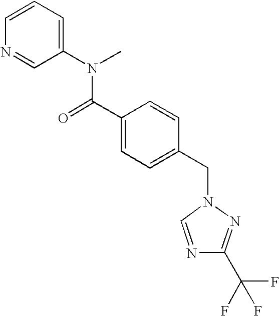 | | (CDCl3) δ: 3.52(3H, s), 5.32(2H, s), 7.11-7.14 (2H, m), 7.21-7.24(1H, m), 7.32-7.38(3H, m), 8.04(1H, s), 8.32-8.33(1H, m), 8.40-8.41(1H, m). |
| 26 | 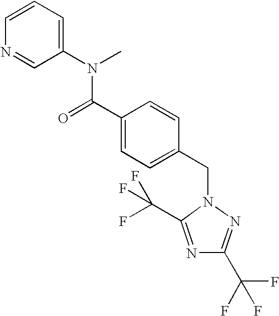 | | (CDCl3) δ: 3.51(3H, s), 5.45(2H, s), 7.14-7.36 (6H, m), 8.33-8.34(1H, m), 8.39-8.40(1H, m). |
| 27 | 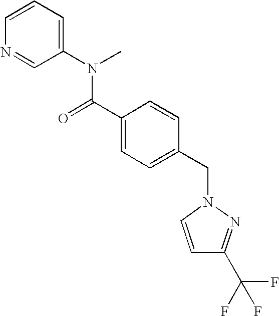 | | (CDCl3) δ: 3.51(3H, s), 5.28(2H, s), 6.53(1H, t), 7.04(2H, d), 7.18-7.33(4H, m), 7.38-7.38 (1H, m), 8.32(1H, d), 8.39(1H, dd). |
| 28 | 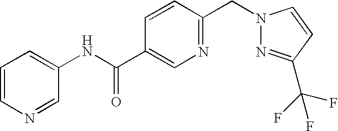 | | (CDCl3) δ: 5.46(2H, s), 6.56(1H, s), 6.95-7.05 (2H, m), 7.59(1H, s), 7.90-7.94(1H, m), 8.15 (2H, dd), 8.29(1H, d), 8.63(1H, s), 9.01(1H, s), 9.43(1H, s). |
| 29 | 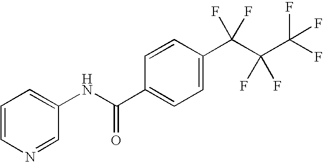 | | (CDCl3) δ: 7.28-7.31(1H, m), 7.67(2H, d, J = 8.4 Hz), 8.02(2H, d, J = 8.6 Hz), 8.26-8.31(2H, m), 8.70-8.70(1H, m), 9.13(1H, s). |
| 30 | 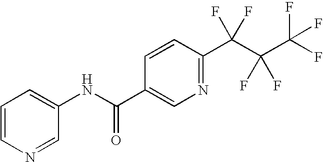 | | (CDCl3) δ: 7.35-7.37(1H, m), 7.83-7.85(1H, m), 8.27-8.30(1H, m), 8.44-8.45(2H, m), 8.67-8.72 (2H, m), 9.25(1H, s). |
| 31 | 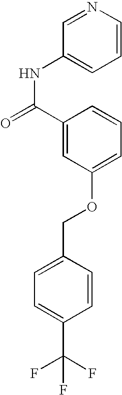 | | (CDCl3) δ: 5.22(2H, s), 7.20-7.21(1H, m), 7.34- 7.37(1H, m), 7.45-7.47(2H, m), 7.56-7.60(3H, m), 7.67-7.70(2H, m), 8.30-8.33(1H, m), 8.42- 8.44(1H, m), 8.69-8.70(1H, m). |
| 32 | 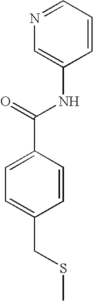 | | (CDCl3) δ: 2.01(3H, s), 3.73(2H, s), 7.32-7.35 (1H, m), 7.44-7.47(2H, m), 7.84-7.87(3H, m), 8.29-8.33(1H, m), 8.39-8.41(1H, m), 8.67-8.67 (1H, m). |
| 33 | 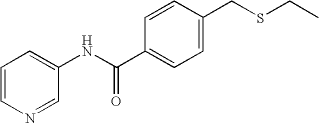 | | (CDCl3) δ: 1.24(3H, t), 2.44(2H, q), 3.78(2H, s), 7.32-7.35(1H, m), 7.44-7.47(2H, m), 7.86- 7.90(3H, m), 8.29-8.32(1H, m), 8.39-8.40(1H, m), 8.66-8.67(1H, m). |
| 34 | 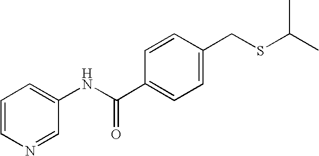 | | (CDCl3) δ: 1.24-1.26(6H, m), 2.77-2.79(1H, m), 3.76(2H, s), 7.25-7.28(1H, m), 7.39-7.42(2H, m), 7.82-7.85(2H, m), 8.25-8.31(3H, m), 8.66- 8.67(1H, m), 8.76(1H, s). |
| 35 | 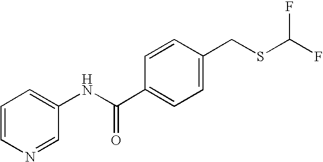 | | (CDCl3) δ: 4.05(2H, s), 6.75(1H, t, J = 56.1 Hz), 7.26-7.29(1H, m), 7.41-7.44(2H, m), 7.84-7.87 (2H, m), 8.25-8.30(2H, m), 8.66-8.67(1H, m), 8.74-8.79(1H, m). |
| 36 | 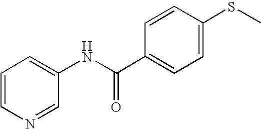 | | (CDCl3) δ: 2.54(3H, s), 7.30-7.33(3H, m), 7.79- 7.83(2H, m), 8.00(1H, s), 8.28-8.30(1H, m), 8.37-8.39(1H, m), 8.66(1H, d, J = 2.6 Hz). |
| 37 | 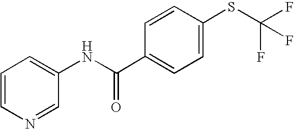 | | (CDCl3) δ: 7.33-7.36(1H, m), 7.78-7.80(2H, m), 7.92-7.94(2H, m), 8.06(1H, s), 8.27-8.30(1H, m), 8.40-8.42(1H, m), 8.68-8.68(1H, m). |
| 38 | 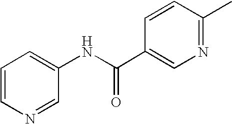 | | (CDCl3) δ: 2.66(3H, s), 7.26-7.37(2H, m), 8.08- 8.14(2H, m), 8.27-8.29(1H, m), 8.41-8.42(1H, m), 8.70(1H, d, J = 2.6 Hz), 9.01(1H, d, J = 2.2 Hz). |
| 39 | 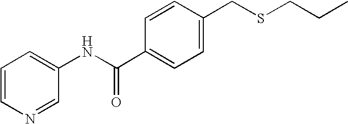 | | (CDCl3) δ: 0.95-0.98(3H, m), 1.56-1.63(2H, m), 2.40(2H, t, J = 7.2 Hz), 3.76(2H, s), 7.32-7.35 (1H, m), 7.46(2H, d, J = 8.4 Hz), 7.84-7.85(3H, m), 8.29-8.33(1H, m), 8.39-8.41(1H, m), 8.67- 8.67(1H, m). |
| 40 | 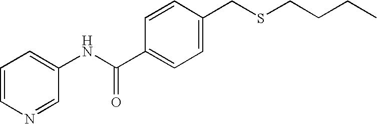 | | (CDCl3) δ: 0.89(3H, t, J = 7.2 Hz), 1.35-1.40 (2H, m), 1.54-1.56(2H, m), 2.42(2H, t, J = 7.3 Hz), 3.75(2H, s), 7.31-7.34(1H, m), 7.45(2H, d, J = 8.4 Hz), 7.84(2H, d, J = 8.4 Hz), 8.04(1H, s), 8.29-8.32(1H, m), 8.38-8.39(1H, m), 8.66-8.67 (1H, m). |
| 41 | 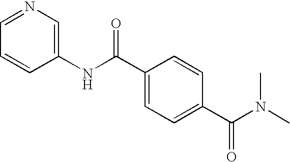 | | (CDCl3) δ: 2.94(3H, s), 3.15(3H, s), 7.29-7.32 (5H, m), 7.81-7.83(2H, m), 8.39-8.42(2H, m), 8.87-8.88(1H, m), 9.39(1H, s). |
| 42 | 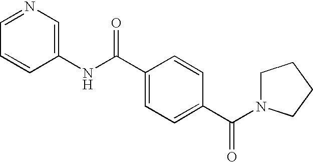 | | (CDCl3) δ: 1.91-1.98(4H, m), 3.35-3.38(2H, m), 3.66-3.68(2H, m), 7.32-7.35(1H, m), 7.41-7.42 (2H, m), 7.84-7.86(2H, m), 8.39-8.44(2H, m), 8.88-8.89(1H, m), 9.12(1H, s). |
| 43 | 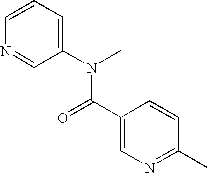 | | (CDCl3) δ: 2.49(3H, s), 3.53(3H, s), 7.01-7.04 (1H, m), 7.23-7.26(2H, m), 7.43-7.46(1H, m), 7.52-7.55(1H, m), 8.34-8.36(2H, m), 8.42-8.44 (1H, m). |
| 44 | 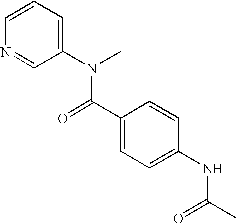 | | (CDCl3) δ: 2.13(3H, s), 3.52(3H, s), 7.20-7.25 (3H, m), 7.33-7.41(3H, m), 7.59(1H, br s), 8.33- 8.33(1H, m), 8.38-8.40(1H, m). |
| 45 | 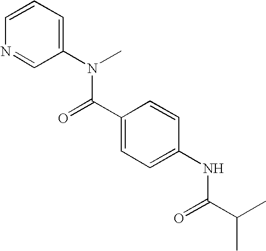 | | (CDCl3) δ: 1.22(6H, d, J = 7.0 Hz), 2.46(1H, d, J = 7.0 Hz), 3.51(3H, s), 7.21-7.26(4H, m), 7.37- 7.40(3H, m), 8.34(1H, d, J = 2.0 Hz), 8.38-8.39 (1H, m). |
| 46 | 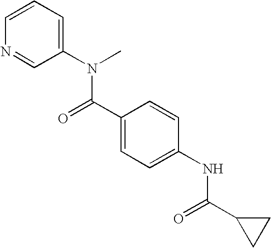 | | (CDCl3) δ: 0.81-0.86(2H, m), 1.04-1.07(2H, m), 1.46-1.48(1H, m), 3.51(3H, s), 7.19-7.25 (3H, m), 7.34-7.40(3H, m), 7.66(1H, s), 8.34 (1H, d, J = 2.0 Hz), 8.38-8.39(1H, m). |
| 47 | 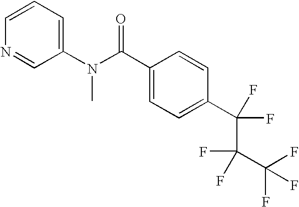 | | (CDCl3) δ: 3.54(3H, s), 7.20-7.22(1H, m), 7.38- 7.43(5H, m), 8.37(1H, d, J = 2.4 Hz), 8.42-8.43 (1H, m). |
| 48 | 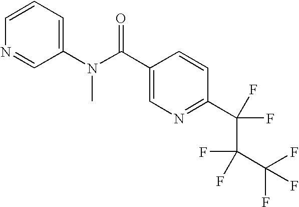 | | (CDCl3) δ: 3.56(3H, s), 7.26-7.28(2H, m), 7.41- 7.44(1H, m), 7.57-7.60(1H, m), 7.84-7.86(1H, m), 8.36(1H, d, J = 2.2 Hz), 8.47-8.49(1H, m), 8.58(1H, s). |
| 49 | 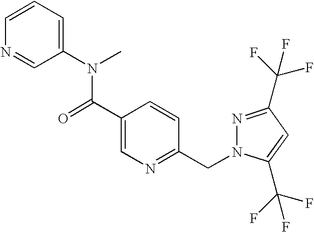 | | (CDCl3) δ: 3.52(3H, s), 5.56(2H, s), 6.81(1H, d, J = 8.1 Hz), 6.95(1H, s), 7.23-7.25(1H, m), 7.37- 7.38(1H, m), 7.61-7.63(1H, m), 8.36(1H, d, J = 2.4 Hz), 8.43-8.45(2H, m). |
| 50 | 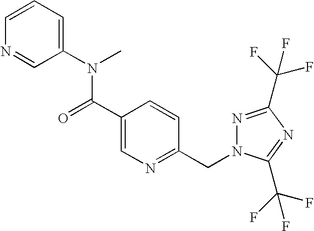 | | (CDCl3) δ: 3.53(3H, s), 5.61(2H, s), 7.05-7.08 (1H, m), 7.24-7.26(1H, m), 7.40-7.42(1H, m), 7.66-7.69(1H, m), 8.33-8.34(1H, m), 8.40-8.41 (1H, m), 8.45-8.46(1H, m). |
| 51 | 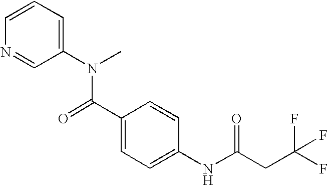 | | (CDCl3) δ: 3.20(2H, q, J = 10.4 Hz), 3.50(3H, s), 7.14(2H, d, J = 8.8 Hz), 7.24-7.28(3H, m), 7.44-7.47(1H, m), 8.28(1H, d, J = 2.4 Hz), 8.40 (1H, dd , J = 4.8, 1.3 Hz), 9.15(1H, s). |
| 52 | 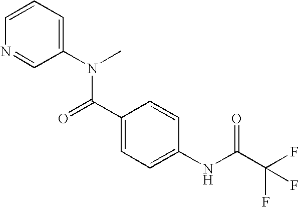 | | (CDCl3) δ: 3.53(3H, s), 7.21-7.33(5H, m), 7.40- 7.44(3H, m), 8.13(1H, s), 8.32(1H, d, J = 2.2 Hz), 8.41(1H, dd, J = 4.7, 1.6 Hz). |
| 53 | 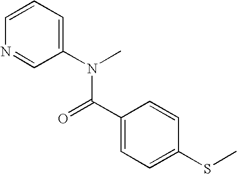 | | (CDCl3) δ: 2.41(3H, s), 3.50(3H, s), 7.01-7.04 (2H, m), 7.18-7.23(3H, m), 7.37-7.38(1H, m), 8.36-8.39(2H, m). |
| 54 | 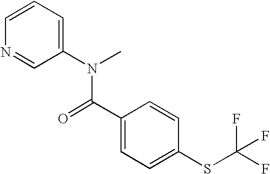 | | (CDCl3) δ: 3.53(3H, s), 7.19-7.23(1H, m), 7.33- 7.35(3H, m), 7.50(2H, d, J = 8.2 Hz), 8.36(1H, d, J = 2.4 Hz), 8.42(1H, dd, J = 4.8, 1.5 Hz). |
| 55 | 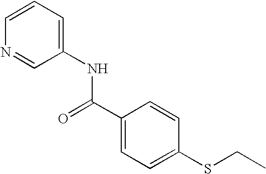 | | (CDCl3) δ: 1.38(3H, t, J = 14.8 Hz), 3.04(2H, q, J = 7.4 Hz), 7.31-7.39(3H, m), 7.79-7.83(3H, m), 8.28-8.31(1H, m), 8.39(1H, dd, J = 4.8, 1.5 Hz), 8.66(1H, d, J = 2.7 Hz). |
| 56 | 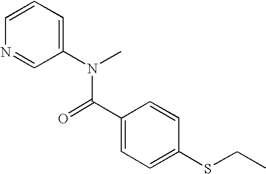 | | (CDCl3) δ: 1.28(3H, t), 2.90(2H, q), 3.51(3H, s), 7.07-7.09(2H, m), 7.18-7.21(3H, m), 7.37- 7.39(1H, m), 8.36-8.39(2H, m). |
| 57 | 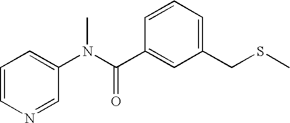 | | (CDCl3) δ: 1.77(3H, s), 3.51(2H, s), 3.52(3H, s), 7.17-7.24(5H, m), 7.40-7.42(1H, m), 8.34- 8.37(2H, m). |
| 58 | 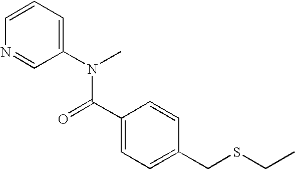 | | (CDCl3) δ: 1.16(3H, t), 2.33(2H, q), 3.51(3H, s), 3.62(2H, s), 7.14-7.28(5H, m), 7.38(1H, dq), 8.35-8.38(2H, m). |
| 59 | 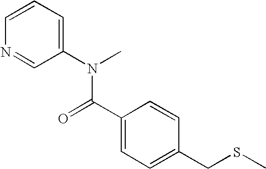 | | (CDCl3) δ: 1.89(3H, s), 3.51(3H, s), 3.57(2H, s), 7.19-7.23(5H, m), 7.38(1H, dt, J = 5.8, 2.2 Hz), 8.35-8.38(2H, m). |
| 60 | 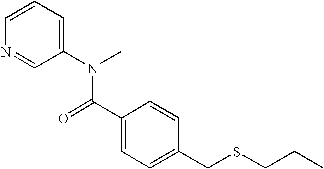 | | (CDCl3) δ: 0.91(3H, t), 1.49-1.52(2H, m), 2.28 (2H, t), 3.52(3H, s), 3.60(2H, s), 7.14-7.26(5H, m), 7.35-7.39(1H, m), 8.36-8.38(2H, m). |
| 61 | 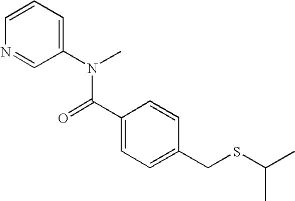 | | (CDCl3) δ: 1.19(6H, dd), 2.66-2.69(1H, m), 3.52(3H, s), 3.65(2H, s), 7.18-7.25(7H, m), 7.35-7.39(1H, m), 8.36-8.38(2H, m). |
| 62 | 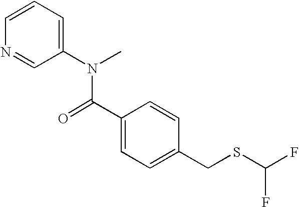 | | (CDCl3) δ: 3.52(3H, s), 3.92(2H, s), 6.67(1H, t), 7.20-7.26(5H, m), 7.36(1H, q), 8.35(1H, d), 8.40(1H, dd). |
| 63 | 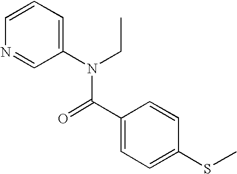 | | (CDCl3) δ: 1.23(3H, t), 2.42(3H, s), 3.99(2H, q), 7.02(2H, d), 7.18-7.22(3H, m), 7.35-7.37 (1H, m), 8.34(1H, d), 8.41(1H, dd). |
| 64 | 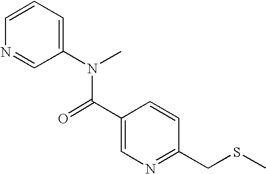 | | (CDCl3) δ: 1.96(2H, s), 3.53(3H, s), 3.71(2H, s), 7.24-7.27(2H, m), 7.43(1H, dt), 7.62(1H, dd), 8.34(1H, d), 8.39(1H, d), 8.44(1H, dd). |
| 65 | 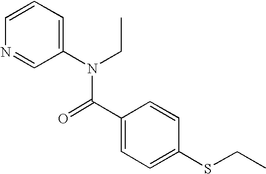 | | (CDCl3) δ: 1.21-1.30(6H, m), 2.90(2H, q, J = 7.4 Hz), 3.98(2H, q, J = 7.1 Hz), 7.07(2H, d, J = 4.2 Hz), 7.17-7.22(3H, m), 7.34-7.37(1H, m), 8.34(1H, d, J = 2.6 Hz), 8.40(1H, dd, J = 4.8, 1.5 Hz). |
| 66 | 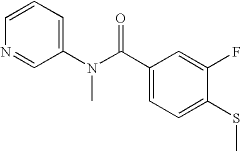 | | (CDCl3) δ: 2.42(3H, s), 3.51(3H, s), 7.01(2H, d), 7.23-7.25(2H, m), 7.39-7.42(1H, m), 8.35 (1H, d), 8.43(1H, dd). |
| 67 | 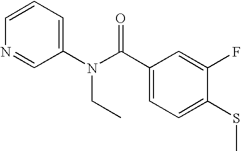 | | (CDCl3) δ: 1.23(3H, t), 2.40(3H, s), 3.98(2H, q), 6.98-7.01(3H, m), 7.25(1H, dd), 7.41(1H, ddd), 8.33(1H, d), 8.44(1H, dd). |
| 68 | 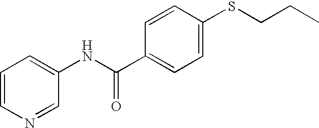 | | (CDCl3) δ: 1.07(3H, t), 1.73-1.76(2H, m), 2.99 (2H, t), 7.31-7.36(4H, m), 7.79(2H, d), 8.30(1H, d), 8.40(1H, dd), 8.66(1H, d). |
| 69 | 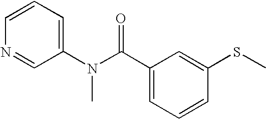 | | (CDCl3) δ: 2.35(3H, s), 3.53(3H, s), 6.99(1H, d), 7.09(1H, t), 7.17-7.20(3H, m), 7.39(1H, dt), 8.36(1H, d), 8.40(1H, dd). |
| 70 | 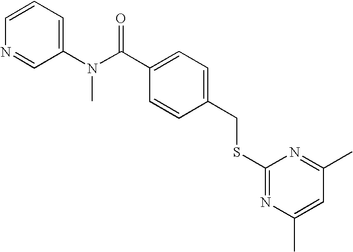 | | (CDCl3) δ: 2.37(6H, s), 3.50(3H, s), 4.30(2H, s), 6.67(1H, s), 7.16-7.35(6H, m), 8.37(2H). |
| 71 | 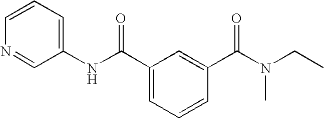 | | (CDCl3) δ: 1.14-1.25(3H, m), 2.92-3.10(3H), 3.36-3.54(2H), 7.27-7.34(1H, m), 7.41-7.43(2H, m), 7.85-7.90(2H, m), 8.37-8.41(2H, m), 8.83 (1H), 9.54(1H, s). |
| 72 | 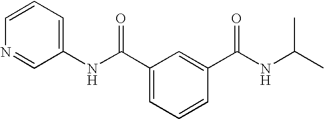 | | (acetone-d6) δ: 1.34(6H, d), 4.30-4.32(1H, m), 7.47(1H), 7.71(1H), 8.09(1H), 8.17(1H), 8.29 (1H), 8.45(2H), 8.96(1H), 9.03(1H). |
| 73 | 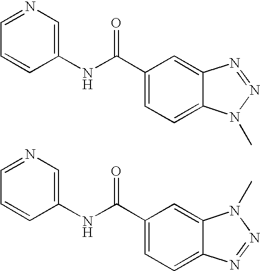 | | (CDCl3) δ: 4.28(6H), 7.28-7.33(2H, m), 7.55 (1H), 7.83(1H), 7.98(1H), 8.11(1H), 8.19(1H), 8.34-8.36(4H, m), 8.70(1H), 8.76(1H), 8.90 (1H), 9.30(1H), 9.77(1H). |
| 74 | 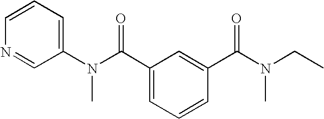 | | (CDCl3) δ: 1.09-1.21(3H), 2.70-3.01(5H), 3.53 (3H, s), 7.19-7.41(6H, m), 8.38-8.40(2H, m). |
| 75 | 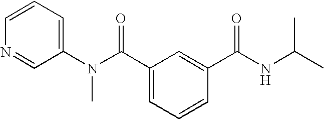 | | (CDCl3) δ: 1.25(6H, d), 3.53(3H, s), 4.21-4.25 (1H, m), 5.88-5.90(1H, m), 7.19-7.31(4H, m), 7.39-7.42(1H, m), 7.70-7.74(2H, m), 8.36(1H, d), 8.40(1H, dd). |
| 76 | 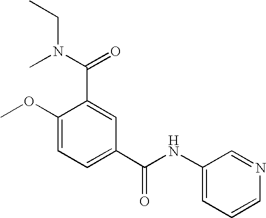 | | (CDCl3) δ: 1.12-1.24(3H, m), 2.81(2H), 3.16 (3H, d), 3.60(3H), 6.80-6.83(1H), 7.27-7.30(1H, m), 7.73(1H, dd), 7.83-7.87(1H), 8.33-8.36(2H), 8.85-8.86(1H), 9.46(1H). |
| 77 | 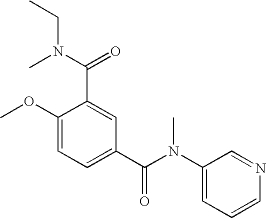 | | (CDCl3) δ: 0.91-1.28(3H, m), 2.96-2.99(3H, m), 3.50(3H, s), 3.78(3H, s), 6.74(1H, dd), 7.16- 7.39(4H, m), 8.36-8.40(2H, m). |
| 78 | 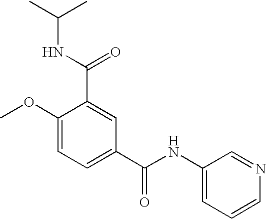 | | (CDCl3) δ: 1.14(6H, d), 4.01(3H), 4.07-4.15 (1H, m), 7.04-7.07(1H, m), 7.26-7.28(1H, m), 7.62(1H, d), 8.15(1H), 8.29-8.33(2H, m), 8.83- 8.84(1H, m), 8.87-8.88(1H, m), 9.63(1H, s). |
| 79 | 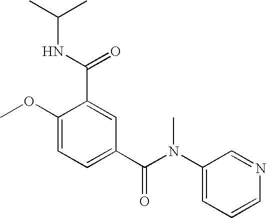 | | (CDCl3) δ: 7.09-7.12(1H, m), 7.32-7.41(2H, m), 7.58-7.59(1H, m), 8.00(1H, br s), 8.24-8.51(5H, m), 8.83-8.91(2H, m). |
| 80 | 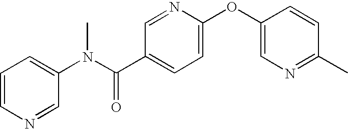 | | (CDCl3) δ: 2.57(3H, s), 3.53(3H, s), 6.81-6.84 (1H, m), 7.16-7.47(4H, m), 7.67-7.70(1H, m), 8.01-8.02(1H, m), 8.29-8.42(3H, m). |
| 81 | 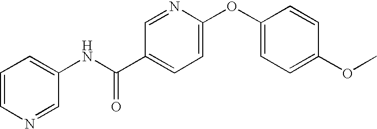 | | (CDCl3) δ: 3.83(3H, s), 6.94-7.11(4H, m), 7.33- 7.36(1, m), 8.02(1H, br s), 8.19-8.43(4H, m), 8.66-8.71(2H, m). |
| 82 | 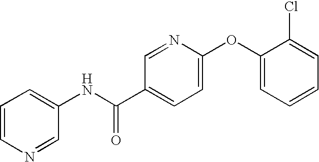 | | (CDCl3) δ: 7.04(1H, d, J = 8.6 Hz), 7.28-7.40 (5H, m), 8.19-8.37(3H, m), 8.66-8.67(2H, m), 8.80-8.84(1H, m). |
| 83 | 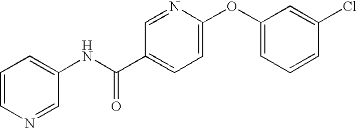 | | (CDCl3) δ: 7.06-7.08(2H, m), 7.19-7.26(2H, m), 7.32-7.40(2H, m), 7.88(1H, br s), 8.24-8.27(2H, m), 8.41-8.42(1H, m), 8.67-8.69(2H, m). |
| 84 | 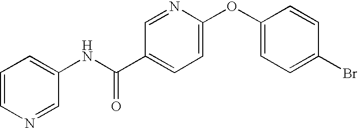 | | (CDCl3) δ: 7.05-7.08(3H, m), 7.30-7.37(2H, m), 7.54-7.57(1H, m), 7.80(1H, br s), 8.21-8.27(2H, m), 8.41-8.43(1H, m), 8.67-8.67(2H, m). |
| 85 | 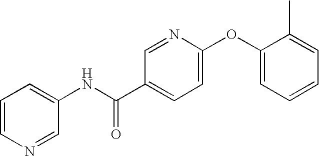 | | (CDCl3) δ: 2.17(3H, s), 6.98-7.36(6H, m), 7.84 (1H, br s), 8.21-8.42(3H, m), 8.67-8.67(2H, m). |
| 86 | 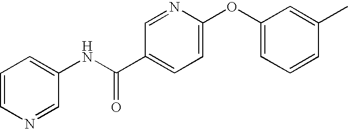 | | (CDCl3) δ: 2.39(3H, s), 6.98-7.07(3H, m), 7.26- 7.36(3H, m), 7.92(1H, br s), 8.25-8.37(3H, m), 8.67-8.70(2H, m). |
| 87 | 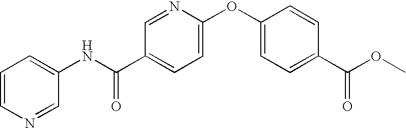 | | (CDCl3) δ: 3.93(3H, s), 7.05-7.08(1H, m), 7.31- 7.54(3H, m), 7.86-7.92(2H, m), 8.08(2H, br s), 8.21-8.44(3H, m), 8.66-8.71(2H, m). |
| 88 | 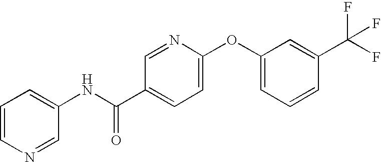 | | (CDCl3) δ: 3.93(3H, s), 7.05-7.08(1H, m), 7.31- 7.54(3H, m), 7.82-7.83(1H, m), 7.93-7.95(1H, m), 8.08(1H, br s), 8.22-8.28(2H, m), 8.40-8.43 (1H, m), 8.67-8.70(2H, m). |
| 89 | 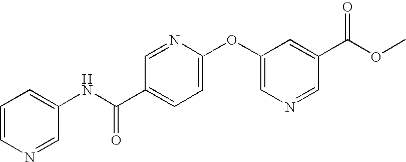 | | (CDCl3) δ: 7.08-7.10(1H, m), 7.32-7.59(5H, m), 8.02-8.06(1H, m), 8.24-8.42(3H, m), 8.67- 8.68(2H, m). |
| 90 | 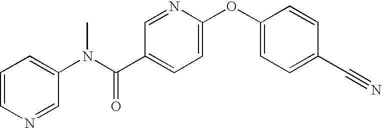 | | (CDCl3) δ: 3.52(3H, s), 6.84-6.87(1H, m), 7.16- 7.74(7H, m), 8.02-8.05(1H, m), 8.32-8.48(2H, m). |
| 91 | 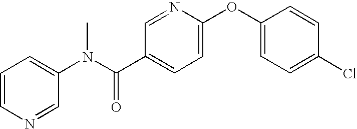 | | (CDCl3) δ: 3.53(3H, s), 6.77-6.80(1H, m), 7.00- 7.03(2H, m), 7.23-7.48(4H, m), 7.67-7.69(1H, m), 8.04-8.04(1H, m), 8.34-8.43(2H, m). |
| 92 | 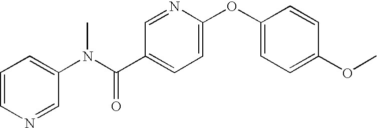 | | (CDCl3) δ: 3.51(3H, s), 3.79(3H, s), 6.71-7.08 (5H, m), 7.20-7.47(1H, m), 7.61-7.65(1H, m), 8.05-8.06(1H, m), 8.32-8.48(1H, m). |
| 93 | 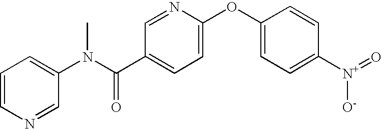 | | (CDCl3) δ: 3.53(3H, s), 6.86-6.91(1H, m), 7.17- 7.77(4H, m), 8.05-8.48(5H, m). |
| 94 | 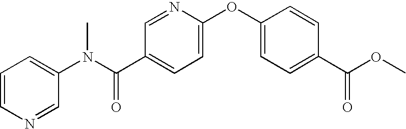 | | (CDCl3) δ: 3.53(3H, s), 3.90(3H, s), 6.79-6.82 (1H, m), 7.20-7.48(5H, m), 7.68-7.72(1H, m), 7.90-8.04(2H, m), 8.35-8.48(2H, m). |
| 95 | 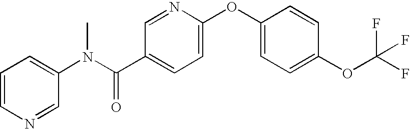 | | (CDCl3) δ: 3.50(3H, s), 6.78-6.81(1H, m), 7.07- 7.70(7H, m), 8.13-8.38(3H, m). |
| 96 | 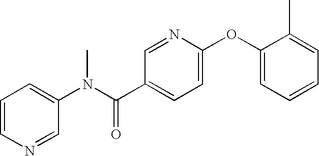 | | (CDCl3) δ: 2.06(3H, s), 3.50(3H, s), 6.73(1H, dd, J = 8.6, 0.7 Hz), 7.14-7.30(6H, m), 7.66-7.69 (1H, m), 8.03-8.03(1H, m), 8.35-8.42(2H, m). |
| 97 | 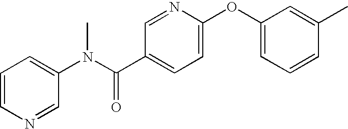 | | (CDCl3) δ: 2.34(3H, s), 3.52(3H, s), 6.78-6.98 (4H, m), 7.23-7.30(2H, m), 7.43-7.46(1H, m), 7.64-7.67(1H, m), 8.07-8.07(1H, m), 8.35-8.43 (2H, m). |
| 98 | 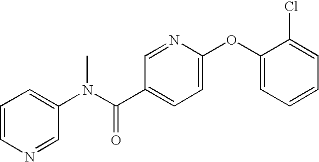 | | (CDCl3) δ: 3.52(3H, s), 6.84(1H, d, J = 8.6 Hz), 7.23-7.36(6H, m), 7.69(1H, dd, J = 8.5, 2.5 Hz), 8.03(1H, d, J = 2.4 Hz), 8.40(2H, dt, J = 20.0, 7.5 Hz). |
| 99 | 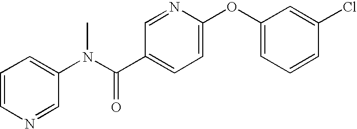 | | (CDCl3) δ: 3.52(3H, s), 6.79(1H, d, J = 8.6 Hz), 6.96-7.03(1H, m), 7.09-7.10(1H, m), 7.21-7.29 (3H, m), 7.43-7.47(1H, m), 7.67-7.70(1H, m), 8.06-8.07(1H, m), 8.34-8.34(1H, m), 8.44-8.46 (1H, m). |
| 100 | 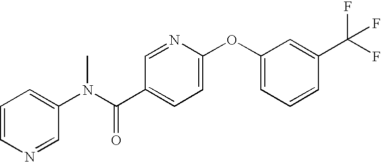 | | (CDCl3) δ: 3.52(3H, s), 6.81-6.84(1H, m), 7.25- 7.72(7H, m), 8.05-8.06(1H, m), 8.34-8.34(1H, m), 8.44-8.46(1H, m). |
| 101 | 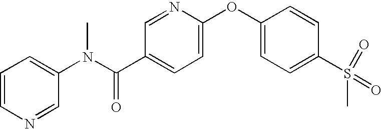 | | (CDCl3) δ: 3.06(3H, s), 3.53(3H, s), 6.86-6.89 (1H, m), 7.23-7.50(4H, m), 7.71-7.24(1H, m), 7.93-8.08(3H, m), 8.35-8.45(2H, m). |
| 102 | 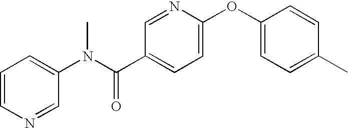 | | (CDCl3) δ: 3.52(3H, s), 6.84(1H, d, J = 8.6 Hz), 7.23-7.36(6H, m), 7.68-7.70(1H, m), 8.02-8.03 (1H, m), 8.36-8.43(2H, m). |
| 103 | 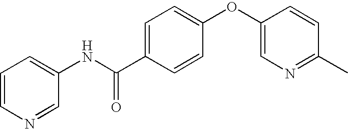 | | (CDCl3) δ: 2.59(3H, s), 6.96-7.43(6H, m), 7.88- 7.90(2H, m), 8.03(1H, br s), 8.27-8.40(2H, m), 8.67-8.68(1H, m). |
| 104 | 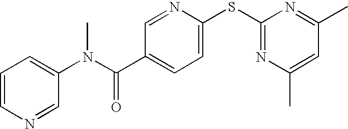 | | (CDCl3) δ: 2.39(6H, s), 3.53(3H, s), 6.79(1H, s), 7.25-7.42(2H, m), 7.61-7.66(1H, m), 7.81- 7.84(1H, m), 8.38-8.50(3H, m). |
| 105 | 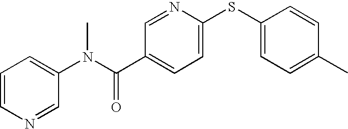 | | (CDCl3) δ: 2.39(3H, s), 3.49(3H, s), 6.64-6.66 (1H, m), 7.20-7.44(7H, m), 8.24-8.46(3H, m). |
| 106 | 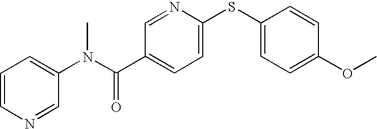 | | (CDCl3) δ: 3.53(3H, s), 3.83(3H, s), 6.59-6.66 (1H, m), 6.95-6.98(2H, m), 7.20-7.56(5H, m), 8.27-8.44(3H, m). |
| 107 | 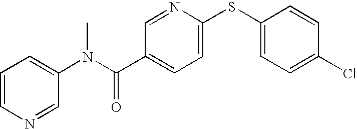 | | (CDCl3) δ: 3.52(3H, s), 6.72-6.75(1H, m), 7.20- 7.49(7H, m), 8.26-8.33(2H, m), 8.44-8.48(1H, m). |
| 108 | 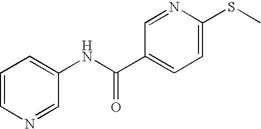 | | (CDCl3) δ: 2.62(3H, s), 7.29-7.35(2H, m), 7.97- 8.49(4H, m), 8.67-8.70(1H, m), 8.91-8.93(1H, m). |
| 109 | 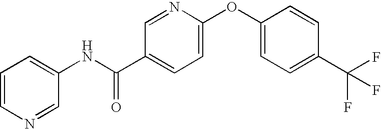 | | (CDCl3) δ: 7.09-7.12(1H, m), 7.30-7.34(3H, m), 7.69-7.72(2H, m), 8.00(1H, br s), 8.18-8.41(3H, m), 8.67-8.69(2H, m). |
| 110 | 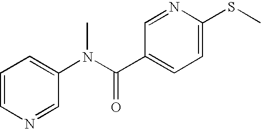 | | (CDCl3) δ: 2.48(3H, s), 3.51(3H, s), 7.00-7.03 (1H, m), 7.20-7.45(3H, m), 8.33-8.45(3H, m). |
| 111 | 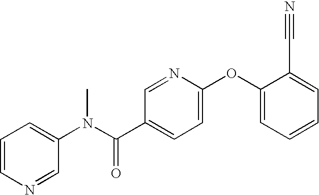 | | (CDCl3) δ: 3.53(3H, s), 6.93-6.96(1H, m), 7.18- 7.73(7H, m), 8.02-8.06(1H, m), 8.42-8.47(2H, m). |
| 112 | 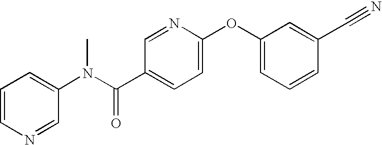 | | (CDCl3) δ: 3.52(3H, s), 6.83-6.86(1H, m), 7.26- 7.73(7H, m), 8.03-8.04(1H, m), 8.36-8.45(2H, m). |
| 113 | 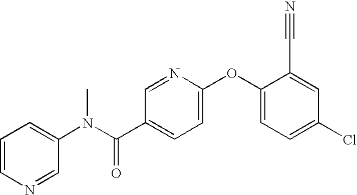 | | (CDCl3) δ: 3.51(3H, s), 6.94-6.96(1H, m), 7.19- 7.73(5H, m), 8.03-8.04(1H, m), 8.24-8.48(3H, m). |
| 114 | 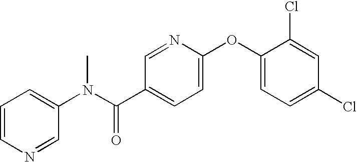 | | (CDCl3) δ: 3.52(3H, s), 6.85-6.88(1H, m), 7.17- 7.36(5H, m), 7.69-7.72(1H, m), 7.99-8.00(1H, m), 8.35-8.43(2H, m). |
| 115 | 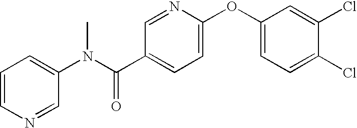 | | (CDCl3) δ: 3.52(3H, s), 6.81-6.95(2H, m), 7.21- 7.48(4H, m), 7.68-7.71(1H, m), 8.04-8.04(1H, m), 8.35-8.44(2H, m). |
| 116 | 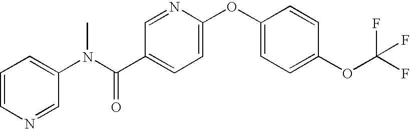 | | (CDCl3) δ: 3.53(3H, s), 6.81-6.84(1H, m), 7.08- 7.48(4H, m), 7.59-7.74(3H, m), 8.06-8.48(3H, m). |
| 117 | 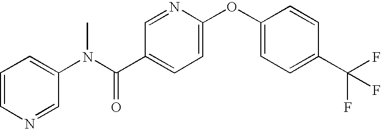 | | (CDCl3) δ: 3.52(3H, s), 6.82-6.85(1H, m), 7.17- 7.49(4H, m), 7.66-7.70(3H, m), 8.05-8.06(1H, m), 8.32-8.48(2H, m). |
| 118 | 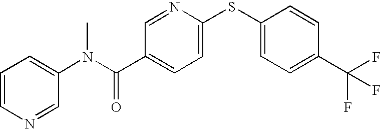 | | (CDCl3) δ: 3.53(3H, s), 6.84-6.88(1H, m), 7.25- 7.67(7H, m), 8.29-8.45(3H, m). |
| 119 | 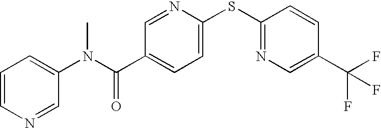 | | (CDCl3) δ: 3.53(3H, s), 72.3-7.27(2H, m), 7.45- 7.59(4H, m), 7.79-7.81(1H, m), 8.35-8.48(3H, m), 8.72-8.72(1H, m). |
| 120 | 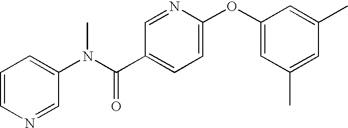 | | (CDCl3) δ: 2.30(6H, s), 3.52(3H, s), 6.71-6.80 (4H, m), 7.24-7.67(3H, m), 8.07-8.08(1H, m), 8.36-8.43(2H, m). |
| 121 | 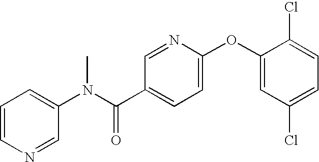 | | (CDCl3) δ: 3.52(3H, s), 6.85-6.88(1H, m), 7.14- 7.45(5H, m), 7.69-7.72(1H, m), 8.02-8.02(1H, m), 8.37-8.44(2H, m). |
| 122 | 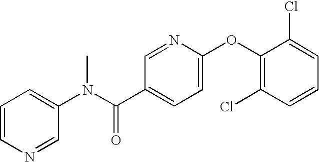 | | (CDCl3) δ: 3.50(3H, s), 6.91-6.94(1H, m), 7.09- 7.43(5H, m), 7.69-7.73(1H, m), 7.99-8.00(1H, m), 8.38-8.46(2H, m). |
| 123 | 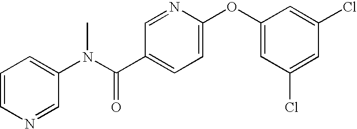 | | (CDCl3) δ: 3.52(3H, s), 6.80-6.82(1H, m), 6.99- 7.03(2H, m) ,7.19-7.48(3H, m), 7.68-7.71(1H, m), 8.06-8.07(1H, m), 8.36-8.45(2H, m). |
| 124 | 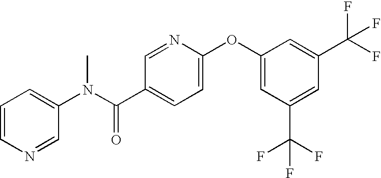 | | (CDCl3) δ: 3.55(3H, s), 6.87-6.90(1H, m), 7.26- 7.31(1H, m), 7.45-7.75(5H, m), 8.04-8.05(1H, m), 8.36-8.45(2H, m). |
| 125 | 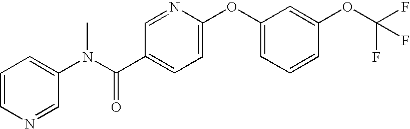 | | (CDCl3) δ: 3.52(3H, s), 6.79-6.82(1H, m), 7.00- 7.05(3H, m), 7.31-7.43(4H, m), 7.68-7.71(1H, m), 8.06-8.07(1H, m), 8.36-8.44(2H, m). |
| 126 | 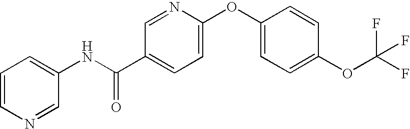 | | (CDCl3) δ: 7.05-7.36(6H, m), 7.92(1H, br s), 8.24-8.26(2H, m), 8.40-8.42(1H, m), 8.66-8.69 (2H, m). |
| 127 | 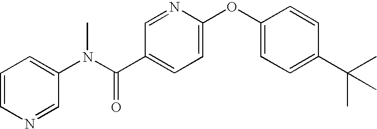 | | (CDCl3) δ: 1.31(9H, s), 3.49(3H, s), 6.72-6.75 (1H, m), 6.97-7.01(2H, m), 7.23-7.67(5H, m), 8.08-8.09(1H, m), 8.36-8.43(2H, m). |
| 128 | 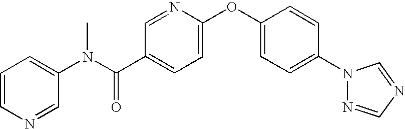 | | (CDCl3) δ: 3.52(3H, s), 6.83-6.86(1H, m), 7.21- 7.30(3H, m), 7.46-7.49(1H, m), 7.68-7.71(3H, m), 8.07-8.09(2H, m), 8.32-8.51(3H, m). |
| 129 | 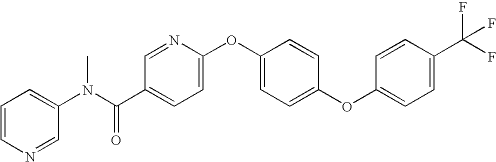 | | (CDCl3) δ: 3.52(3H, s), 6.78-6.80(1H, m), 7.03- 7.11(4H, m), 7.27-7.29(3H, m), 7.49-7.65(4H, m), 8.08-8.09(1H, m), 8.36-8.44(2H, m). |
| 130 | 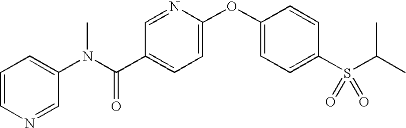 | | (CDCl3) δ: 2.75(6H, s), 3.53(3H, s), 6.84-6.86 (1H, m), 7.22-7.49(4H, m), 7.71-7.79(3H, m), 8.08-8.09(1H, m), 8.36-8.45(2H, m). |
| 131 | 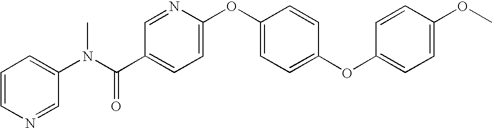 | | (CDCl3) δ: 1.27(3H, t, J = 18.8 Hz), 3.99(2H, q, J = 7.1 Hz), 6.76-6.79(1H, m), 7.06-7.47(6H, m), 7.66-7.69(1H, m), 8.04-8.05(1H, m), 8.32- 8.44(1H, m). |
| 132 | 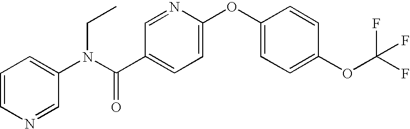 | | (CDCl3) δ: 0.97(3H, t, J = 20.1 Hz), 1.61-1.72 (2H, m), 3.89(2H, q, J = 15.2 Hz), 6.77-6.80(1H, m), 7.06-7.29(5H, m), 7.43-7.46(1H, m), 7.65- 7.67(1H, m), 8.03-8.04(1H, m), 8.30-8.45(2H, m). |
| 133 | 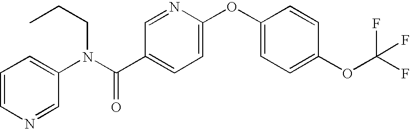 | | (CDCl3) δ: 4.52-4.54(2H, m), 5.18-5.22(2H, m), 5.89-6.02(1H, m), 6.78-6.81(1H, m), 7.06-7.28 (5H, m), 7.43-7.46(1H, m), 7.68-7.71(1H, m), 8.07-8.07(1H, m), 8.32-8.42(2H, m). |
| 134 | 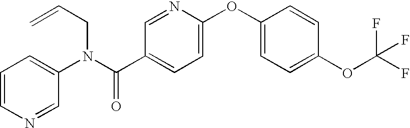 | | (CDCl3) δ: 4.51-4.54(2H, m), 5.15- 5.24(2H, m), 5.91-6.00(1H, m), 6.79(1H, d), 7.07- 7.28(5H, m), 7.44-8.45(5H, m) |
| 135 | 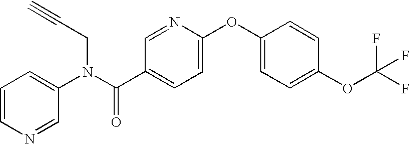 | | (CDCl3) δ: 2.30(1H, t, J = 2.5 Hz), 4.68(2H, d, J = 2.6 Hz), 6.78-6.81(1H, m), 7.08-7.33(5H, m), 7.56-7.75(2H, m), 8.09-8.10(1H, m), 8.41-8.49 (2H, m). |
| 136 | 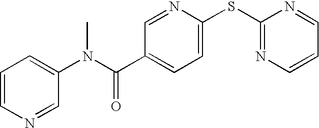 | | (CDCl3) δ: 3.51(3H, s), 7.20-7.30(5H, m), 7.42- 7.46(1H, m), 7.60-7.62(1H, m), 8.24-8.48(3H, m). |
| 137 | 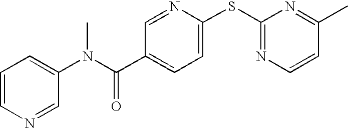 | | (CDCl3) δ: 2.44(3H, s), 3.52(3H, s), 6.91-6.92 (1H, m), 7.24-7.27(1H, m), 7.43-7.46(1H, m), 7.67-7.81(2H, m), 8.39-8.44(4H, m). |
| 138 | 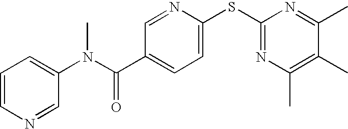 | | (CDCl3) δ: 2.20(3H, s), 2.41(6H, s), 3.51(3H, s), 7.23-7.45(2H, m), 7.61-7.64(1H, m), 7.76- 7.78(1H, m), 8.37-8.44(3H, m). |
| 139 | 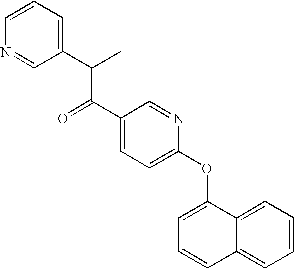 | | (CDCl3) δ: 3.52(4H, s), 6.80-6.83(1H, m), 7.17- 7.52(6H, m), 7.67-7.88(4H, m), 8.03-8.04(1H, m), 8.35-8.42(2H, m). |
| 140 | 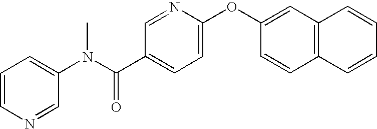 | | (CDCl3) δ: 3.51(3H, s), 6.80-6.83(1H, m), 7.19- 7.87(10H, m), 8.07-8.07(1H, m), 8.36-8.43(2H, m). |
| 141 | 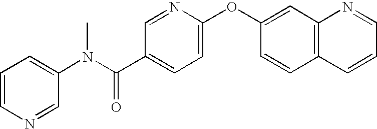 | | (CDCl3) δ: 3.52(3H, s), 6.88-6.91(1H, m), 7.29- 7.38(4H, m), 7.73-7.82(3H, m), 8.07-8.08(2H, m), 8.38-8.44(2H, m), 8.89-8.91(1H, m). |
| 142 | 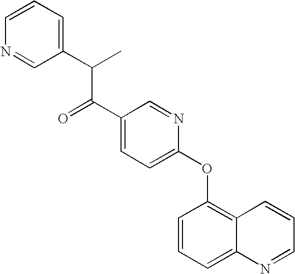 | | (CDCl3) δ: 3.53(3H, s), 6.88-6.91(1H, m), 7.28- 7.44(4H, m), 7.68-7.74(2H, m), 8.00-8.01(2H, m), 8.20-8.40(3H, m), 8.92-8.93(1H, m). |
| 143 | 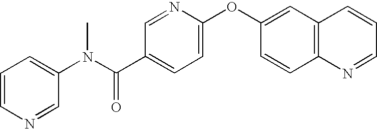 | | (CDCl3) δ: 3.52(3H, s), 6.86-6.89(1H, m), 7.25- 7.52(5H, m), 7.71-7.73(1H, m), 8.06-8.14(3H, m), 8.36-8.44(2H, m), 8.88-8.89(1H, m). |
| 144 | 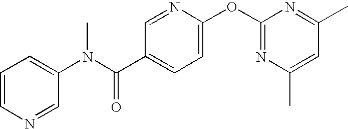 | | (CDCl3) δ: 2.54(6H, s), 3.50(3H, s), 6.30-6.33 (1H, m), 7.01-7.51(4H, m), 7.90-7.95(1H, m), 8.51-8.53(2H, m). |
| 145 | 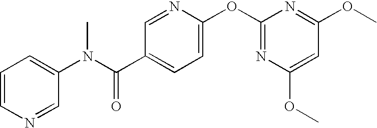 | | (CDCl3) δ: 3.48(3H, s), 3.92(6H, s), 6.06(1H, s), 6.31-6.34(1H, m), 7.05-7.09(1H, m), 7.31- 7.48(2H, m), 7.88-7.91(1H, m), 8.49-8.51(2H, m). |
| 146 | 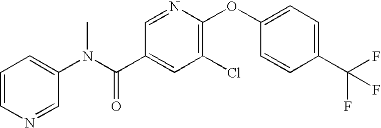 | | (CDCl3) δ: 3.50(3H, s), 7.19-7.33(3H, m), 7.47- 7.84(5H, m), 8.35-8.46(2H, m). |
| 147 | 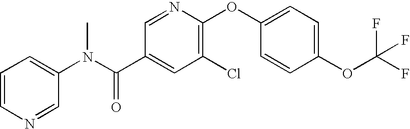 | | (CDCl3) δ: 3.51(3H, s), 7.10-7.50(6H, m), 7.81- 7.82(2H, m), 8.35-8.46(2H, m). |
| 148 | 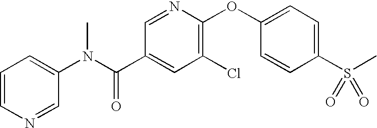 | | (CDCl3) δ: 3.07(3H, s), 3.51(3H, s), 7.26-7.52 (4H, m), 7.86-7.94(4H, m), 8.35-8.47(2H, m). |
| 149 | 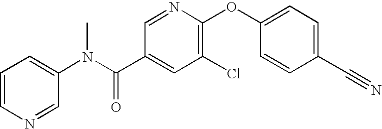 | | (CDCl3) δ: 7.19-7.52(5H, m), 7.67-7.84(4H, m), 8.31-8.32(1H, m), 8.48-8.48(1H, m). |
| 150 | 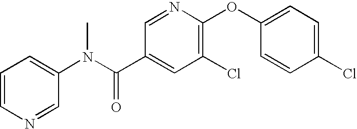 | | (CDCl3) δ: 3.50(3H, s), 7.01-7.05(2H, m), 7.26- 7.50(4H, m), 7.79-7.82(2H, m), 8.32-8.47(2H, m). |
| 151 | 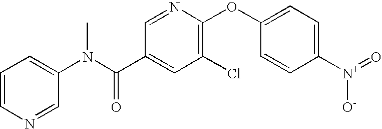 | | (CDCl3) δ: 3.52(3H, s), 7.24-7.52(4H, m), 7.81- 7.86(2H, m), 8.33-8.44(4H, m). |
| 152 | 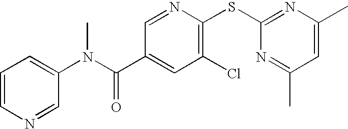 | | (CDCl3) δ: 2.39(6H, s), 3.54(3H, s), 6.80(1H, s), 7.27-7.29(1H, m), 7.42-7.45(1H, m), 7.74- 7.74(1H, m), 8.19-8.20(1H, m), 8.43-8.47(2H, m). |
| 153 | 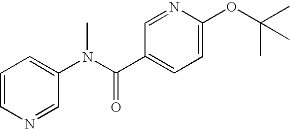 | | (CDCl3) δ: 1.58(9H, s), 3.53(3H, s), 6.46-6.49 (1H, m), 7.37-7.39(1H, m), 7.61-7.64(1H, m), 7.88-7.91(1H, m), 8.52-8.59(2H, m), 8.82-8.83 (1H, m). |
| 154 | 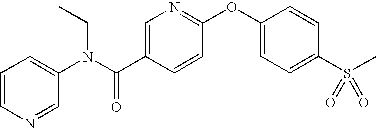 | | (CDCl3) δ: 1.25(3H, t, J = 7.1 Hz), 3.07(3H, s), 4.00(2H, q, J = 7.1 Hz), 6.85-6.87(1H, m), 7.25- 7.30(3H, m), 7.45-7.47(1H, m), 7.70-7.73(1H, m), 7.96-8.04(3H, m), 8.33-8.45(2H, m). |
| 155 | 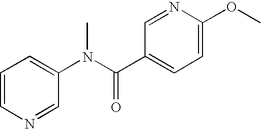 | | (CDCl3) δ: 3.52(3H, s), 3.88(3H, s), 6.55-6.58 (1H, m), 7.25-7.28(1H, m), 7.41-7.53(2H, m), 8.09-8.10(1H, m), 8.37-8.43(2H, m). |
| 156 | 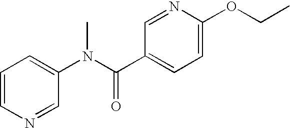 | | (CDCl3) δ: 1.34(3H, t, J = 7.1 Hz), 3.51(3H, s), 4.30(2H, q, J = 7.1 Hz), 6.50-6.55(1H, m), 7.24- 7.26(1H, m), 7.44-7.49(2H, m), 8.08-8.09(1H, m), 8.35-8.45(2H, m). |
| 157 | 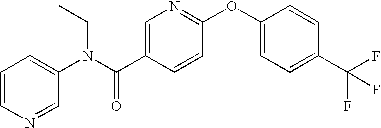 | | (CDCl3) δ: 1.25(3H, t, J = 7.1 Hz), 3.99(2H, q, J = 7.1 Hz), 6.81-6.84(1H, m), 7.16-7.30(3, m), 7.44-7.47(1H, m), 7.64-7.70(3H, m), 8.04-8.05 (1H, m), 8.32-8.44(2H, m). |
| 158 | 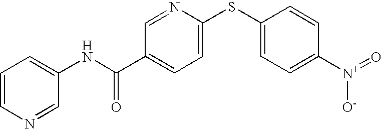 | | (CDCl3) δ: 7.29-7.34(2H, m), 7.73-7.76(3H, m), 7.83(1H, br s), 8.09-8.12(1H, m), 8.24-8.28(2H, m), 8.42-8.44(1H, m), 8.68-8.71(1H, m), 8.90- 8.91(1H, m). |
| 159 | 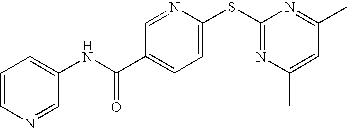 | | (CDCl3) δ: 2.40(6H, s), 6.83(1H, s), 7.31-7.39 (1H, m), 8.03-8.06(1H, m), 8.17-8.40(3H, m), 8.70-8.72(2H, m), 9.01-9.02(1H, m). |
| 160 | 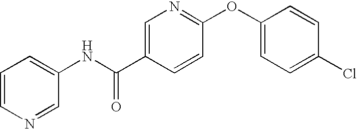 | | (CDCl3) δ: 7.06-7.12(3H, m), 7.40(2H, d, J = 8.4 Hz), 7.79(1H, br s), 8.02-8.10(1H, m), 8.23- 8.25(2H, m), 8.42(1H, d, J = 4.4 Hz), 8.66-8.69 (2H, m). |
| 161 | 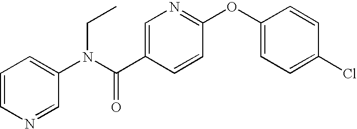 | | (CDCl3) δ: 1.25(3H, t, J = 21.4 Hz), 3.99(2H, q, J = 7.1 Hz), 6.75-6.78(1H, m), 6.99-7.02(2H, m), 7.25-7.46(4H, m), 7.66-7.68(1H, m), 8.02- 8.03(1H, m), 8.32-8.43(2H, m). |
| 162 | 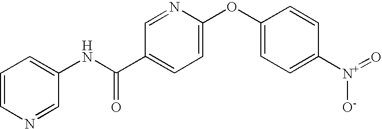 | | (CDCl3) δ: 7.16-7.38(3H, m), 7.79(1H, br s), 8.29-8.40(5H, m), 8.68-8.69(2H, m). |
| 163 | 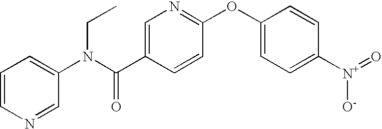 | | (CDCl3) δ: 1.25(3H, t, J = 7.1 Hz), 4.00(2H, q, J = 7.1 Hz), 6.86-6.89(1H, m), 7.20-7.75(5H, m), 8.04-8.48(5H, m). |
| 164 | 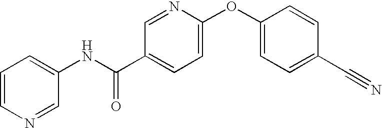 | | (CDCl3) δ: 7.13-7.37(4H, m), 7.72-7.77(3H, m), 8.24-8.44(3H, m), 8.68-8.69(2H, m). |
| 165 | 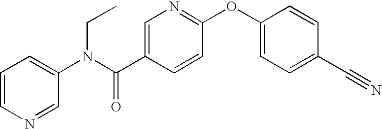 | | (CDCl3) δ: 1.26(3H, s), 4.00(2H, q, J = 7.1 Hz), 6.83-6.86(1H, m), 7.15-7.48(4H, m), 7.67-7.71 (3H, m), 8.03-8.04(1H, m), 8.28-8.29(1H, m), 8.46-8.47(1H, m). |
| 166 | 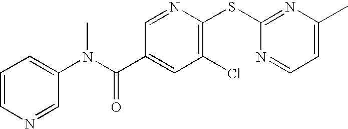 | | (CDCl3) δ: 2.42(3H, s), 3.53(3H, s), 6.91-6.93 (1H, m), 7.26-7.47(2H, m), 7.77-7.77(1H, m), 8.21-8.50(4H, m). |
| 167 | 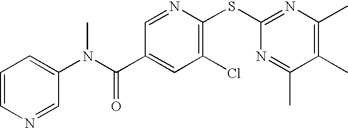 | | (CDCl3) δ: 2.18(3H, s), 2.37(6H, s), 3.52(3H, s), 7.29-7.37(2H, m), 7.68-7.68(1H, m), 8.14- 8.15(1H, m), 8.43-8.47(2H, m). |
| 168 | 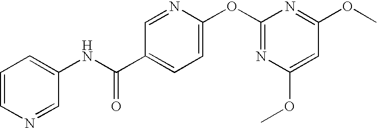 | | (CDCl3) δ: 3.97(6H, s), 6.12(1H, s), 6.68-6.71 (2H, m), 7.27-7.33(3H, m), 8.42-8.61(3H, m). |
| 169 | 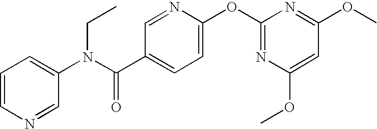 | | (CDCl3) δ: 1.24(3H, t, J = 12.2 Hz), 3.91-3.97 (5H, m), 6.06(1H, s), 6.30-6.33(1H, m), 7.03- 7.07(1H, m), 7.31-7.51(2H, m), 7.89-7.90(1H, m), 8.48-8.51(2H, m). |
| 170 | 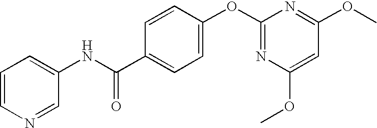 | | (CDCl3) δ: 3.53(3H, s), 7.20-7.46(5H, m), 7.57- 7.63(1H, m), 82.4-8.52(3H, m). |
| 171 | 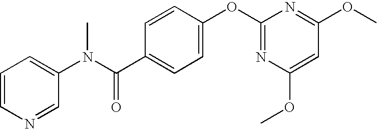 | | (CDCl3) δ: 3.53(3H, s), 3.78(6H, s), 5.77(1H, s), 7.04-7.07(2H, m), 7.17-7.20(1H, m), 7.31- 7.42(3H, m), 8.37-8.38(2H, m). |
| 172 | 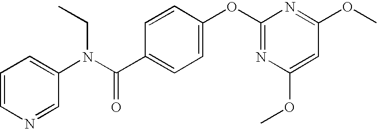 | | (CDCl3) δ: 1.26(3H, t, J = 7.1 Hz), 3.76(6H, s), 4.01(2H, q, J = 7.1 Hz), 5.78(1H, s), 7.02-7.05 (2H, m), 7.17-7.40(4H, m), 8.35-8.38(2H, m). |
| 173 | 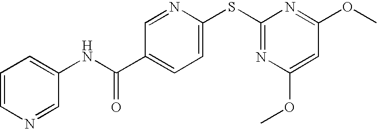 | | (CDCl3) δ: 3.84(6H, s), 5.84(1H, s), 7.26- 7.38(1H, m), 7.92(1H, bs), 8.09-9.06(6H, m) |
| 174 | 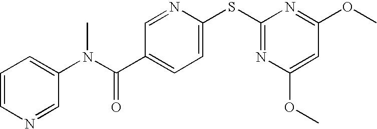 | | (CDCl3) δ: 3.52(3H, s), 3.78(6H, s), 5.78(1H, s), 7.24-72.6(1H, m), 7.45-7.78(3H, m), 8.35- 8.48(3H, m). |
| 175 | 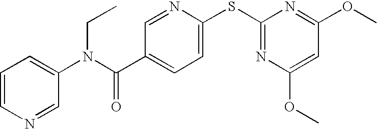 | | (CDCl3) δ: 1.27(3H, t, J = 7.1 Hz), 3.76(6H, s), 4.01(2H, q, J = 7.1 Hz), 5.77(1H, s), 7.21-7.48 (2H, m), 7.64-7.73(2H, m), 8.32-8.46(3H, m). |
| 176 | 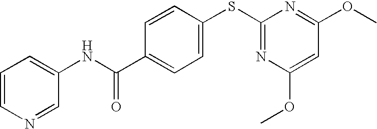 | | (CDCl3) δ: 3.77(6H, s), 5.77(1H, s), 7.32-7.35 (1H, m), 7.75-7.78(2H, m), 7.89-7.92(2H, m), 8.17(1H, br s), 8.31-8.39(2H, m), 8.70-8.70(1H, m). |
| 177 | 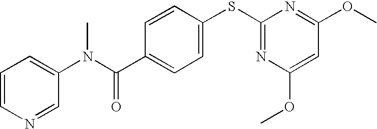 | | (CDCl3) δ: 3.54(3H, s), 3.71(6H, s), 5.74(1H, s), 7.19-7.33(3H, m), 7.40-7.51(3H, m), 8.35- 8.39(2H, m). |
| 178 | 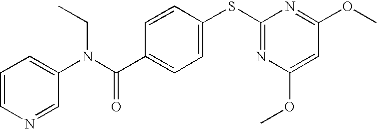 | | (CDCl3) δ: 1.26(3H, t, J = 7.1 Hz), 3.68(6H, s), 4.00(2H, q, J = 7.1 Hz), 5.71(1H, s), 7.19-7.49 (6H, m), 8.33-8.39(2H, m). |
| 179 | 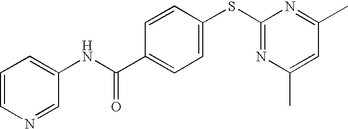 | | (CDCl3) δ: 2.36(6H, s), 6.76(1H, s), 7.33-7.36 (1H, m), 7.78-7.90(5H, m), 8.30-8.33(1H, m), 8.41-8.43(1H, m), 8.68-8.69(1H, m). |
| 180 | 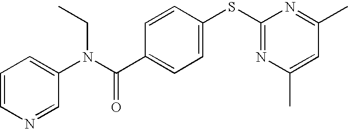 | | (CDCl3) δ: 1.26(3H, t, J = 7.1 Hz), 2.33(6H, s), 4.01(2H, q, J = 7.1 Hz), 6.69(1H, s), 7.19-7.47 (6H, m), 8.38-8.41(2H, m). |
| 181 | 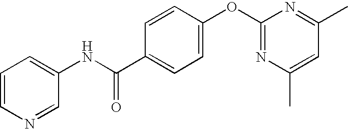 | | (CDCl3) δ: 2.44(6H, s), 6.82(1H, s), 7.26- 8.68(9H, m) |
| 182 | 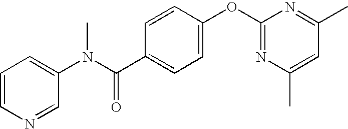 | | (CDCl3) δ: 2.38(6H, s), 3.54(3H, s), 6.76(1H, s), 7.02- 7.40(5H, m), 8.40-8.54(2H, m) |
| 183 | 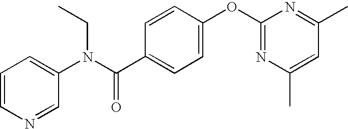 | | (CDCl3) δ: 1.25(3H, t), 2.37(6H, s), 4.00(2H, q), 6.76(1H, s), 7.02- 7.48(6H, m), 8.37-8.43(2H, m) |
| 184 | 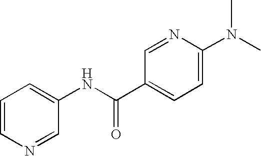 | | (CDCl3) δ: 3.17(6H, s), 6.54(1H, dd, J = 9.0, 0.5 Hz), 7.26-7.32(1H, m), 7.86(1H, br s), 7.97(1H, dd, J = 9.0, 2.6 Hz), 8.25-8.37(2H, m), 8.65-8.71 (2H, m). |
| 185 | 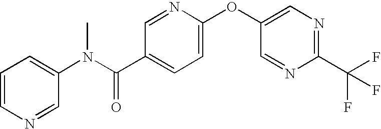 | | (CDCl3) δ: 7.22-7.34(2H, m), 8.18-8.41(4H, m), 8.70-8.88(4H, m). |
| 186 | 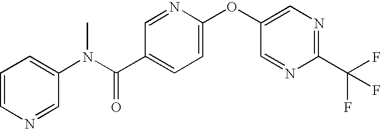 | | (CDCl3) δ: 3.53(3H, s), 6.95-6.97(1H, m), 7.31- 7.47(2H, m), 7.77-8.00(2H, m), 8.33-8.47(2H, m), 8.76(2H, s). |
| 187 | 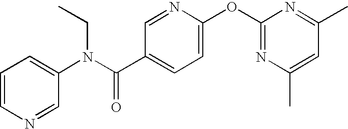 | | (CDCl3) δ: 1.22(3H, q, J = 7.1 Hz), 2.18(6H, s), 3.95(1H, q, J = 7.1 Hz), 6.30(1H, d, J = 9.7 Hz), 7.02-7.09(2H, m), 7.26-7.46(2H, m), 7.93-7.94 (1H, m), 8.51-8.53(2H, m). |
| 188 | 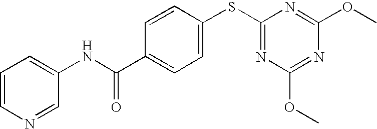 | | (CDCl3) δ: 3.94(6H, s), 7.34-8.70(8H, m) |
| 189 | 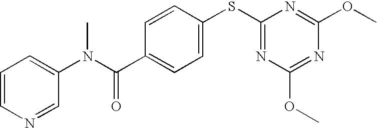 | | (CDCl3) δ: 3.53(3H, s), 3.93(6H, s), 7.20- 8.41(9H, m) |
| 190 | 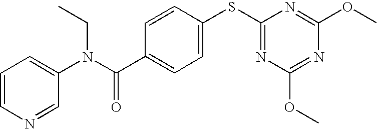 | | (CDCl3) δ: 1.25(3H, t), 3.87(6H, s), 4.02(2H, q), 7.20-8.41(9H, m) |
| 191 | 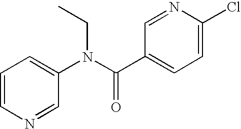 | | (CDCl3) δ: 1.25(3H, t, J = 7.1 Hz), 4.00(2H, q, J = 7.1 Hz), 7.18-7.61(3H, m), 8.26-8.46(3H, m). |
| 192 | 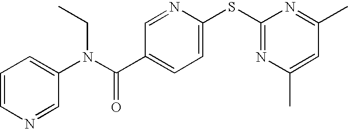 | | (CDCl3) δ: 1.26(3H, t, J = 7.1 Hz), 2.38(6H, s), 4.01(1H, q, J = 7.1 Hz), 6.78(1H, s), 7.24-7.44 (2H, m), 7.66-7.78(2H, m), 8.37-8.47(2H, m). |
| 193 | 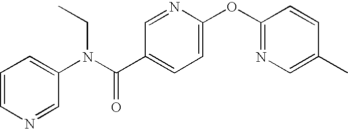 | | (CDCl3) δ: 1.25(3H, q, J = 7.1 Hz), 2.51(3H, s), 3.99(2H, q, J = 7.1 Hz), 6.81(1H, dd, J = 8.6, 0.7 Hz), 7.15-7.70(5H, m), 8.00-8.01(1H, m), 8.29- 8.47(3H, m). |
| 194 | 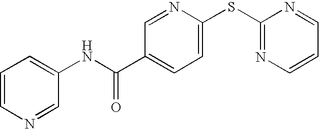 | | (CDCl3) δ: 7.11-7.12(1H, m), 7.35-7.38(1H, m), 7.95(1H, br s), 8.03-8.29(3H, m), 8.47-8.68(4H, m), 9.07(1H, t, J = 1.2 Hz). |
| 195 | 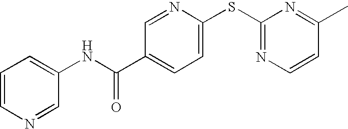 | | (CDCl3) δ: 2.50(3H, s), 6.97(1H, d, J = 5.1 Hz), 7.35-7.38(1H, m), 7.89(1H, br s), 8.05-8.45(5H, m), 8.71(1H, d, J = 2.6 Hz), 9.04(1H, d, J = 2.4 Hz). |
| 196 | 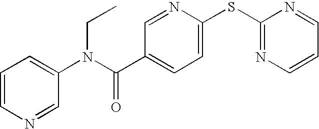 | | (CDCl3) δ: 1.26(3H, t, J = 7.1 Hz), 4.01(2H, q, J = 7.1 Hz), 7.04-7.06(1H, m), 7.25-7.46(2H, m), 7.66-7.79(2H, m), 8.36-8.53(5H, m). |
| 197 | 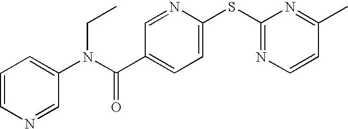 | | (CDCl3) δ: 1.26(3H, t, J = 7.1 Hz), 2.43(3H, s), 4.01(2H, q, J = 7.1 Hz), 6.91(1H, d, J = 5.1 Hz), 7.23-7.80(4H, m), 8.39-8.44(4H, m). |
| 198 | 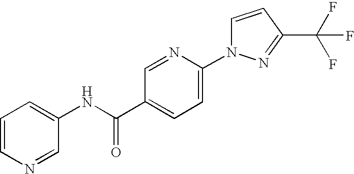 | | (CDCl3) δ: 6.77(1H, d, J = 2.6 Hz), 7.36-7.39 (1H, m), 7.87(1H, br s), 8.18-8.46(4H, m), 8.70- 8.72(2H, m), 8.97-8.98(1H, m). |
| 199 | 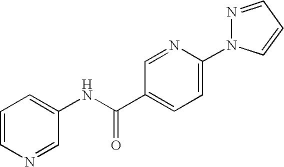 | | (CDCl3/DMSO-d6) δ: 7.30-7.90(2H, m), 8.20- 9.10(8H, m), 10.38(1H, bs) |
| 200 | 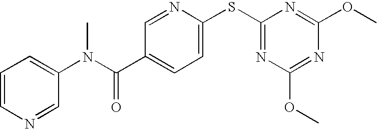 | | (CDCl3) δ: 3.53(3H, s), 3.90(6H, s), 7.26-7.47 (2H, m), 7.71-7.77(2H, m), 8.36-8.46(3H, m). |
| 201 | 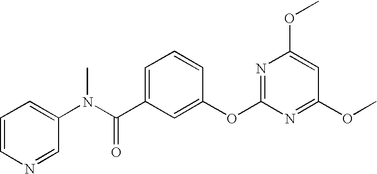 | | (CDCl3) δ: 3.52(3H, s), 3.81(6H, s), 5.77(1H, s), 7.10-7.41(6H, m), 8.35-8.38(2H, m). |
| 202 | 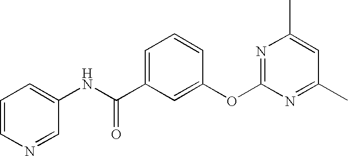 | | (CDCl3) δ: 2.39(6H, s), 6.78(1H, s), 7.26-7.76 (5H, m), 8.16(1H, br s), 8.40-8.58(3H, m). |
| 203 | 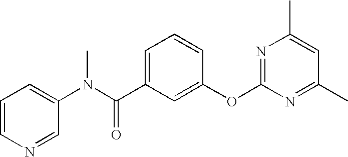 | | (CDCl3) δ: 2.38(6H, s), 3.49(3H, s), 6.78(1H, s), 7.11-7.55(5H, m), 8.40-8.53(3H, m). |
| 204 | 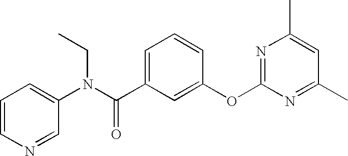 | | (CDCl3) δ: 1.23(3H, t, J = 7.1 Hz), 2.40(6H, s), 3.97(2H, q, J = 7.1 Hz), 6.76(1H, s), 7.09-7.43 (5H, m), 8.35-8.40(3H, m). |
| 205 | 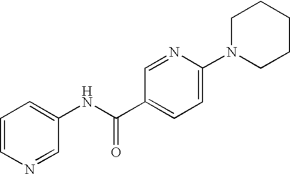 | | (CDCl3) δ: 1.63-1.70(8H, m), 3.67-3.69(4H, m), 6.65(1H, d, J = 8.6 Hz), 7.26-7.37(2H, m), 7.74 (1H, br s), 7.93-7.96(1H, m), 8.19-8.72(4H, m). |
| 206 | 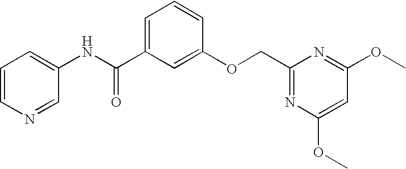 | | (CDCl3) δ: 3.87(6H, s), 5.19(2H, s), 5.95(1H, s), 7.10-7.30(4H, m), 7.82-7.85(3H, m), 8.37- 8.55(3H, m). |
| 207 | 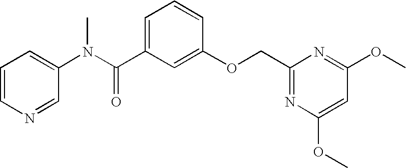 | | (CDCl3) δ: 3.49(3H, s), 3.86(6H, s), 5.06(2H, s), 5.93(1H, s), 6.77-6.81(2H, m), 7.15-7.39(4H, m), 8.35-8.38(2H, m). |
| 208 | 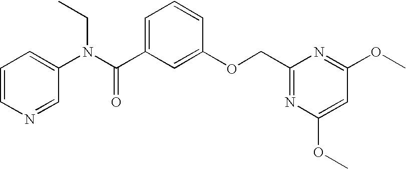 | | (CDCl3) δ: 1.24(3H, t, J = 7.1 Hz), 3.84(6H, s), 3.98(2H, q, J = 7.1 Hz), 5.06(2H, s), 5.93(1H, s), 6.76-6.79(2H, m), 7.16-7.39(4H, m), 8.32- 8.38(2H, m). |
| 209 | 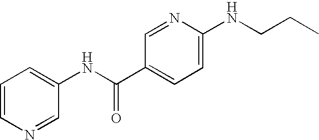 | | (CDCl3) δ: 1.24(3H, t, J = 7.1 Hz), 3.84(6H, s), 3.98(2H, q, J = 7.1 Hz), 5.06(2H, s), 5.93(1H, s), 6.76-6.79(2H, m), 7.16-7.39(4H, m), 8.32- 8.38(2H, m). |
| 210 | 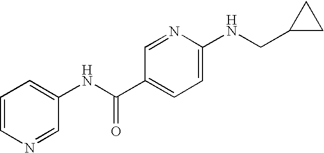 | | (CDCl3) δ: 0.28-0.30(2H, m), 0.56-0.62(2H, m), 1.08-1.13(1H, m), 3.21-3.23(2H, m), 5.11(1H, br s), 6.43(1H, d, J = 8.8 Hz), 7.30-7.33(1H, m), 7.66(1H, br s), 7.94-7.97(1H, m), 8.26-8.37(2H, m), 8.64-8.65(2H, m). |
| 211 | 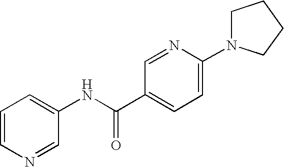 | | (CDCl3) δ: 2.07-2.13(4H, m), 3.52-3.55(4H, m), 6.39-6.42(1H, m), 7.25-7.30(1H, m), 7.76(1H, br s), 7.93-8.71(5H, m). |
| 212 | 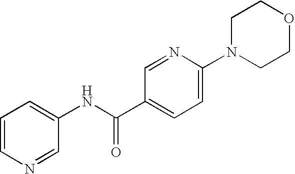 | | (CDCl3) δ: 3.66-3.83(8H, m), 6.66(1H, d, J = 9.2 Hz), 7.26-7.34(1H, m), 7.74(1H, br s), 8.12- 8.60(5H, m). |
| 213 | 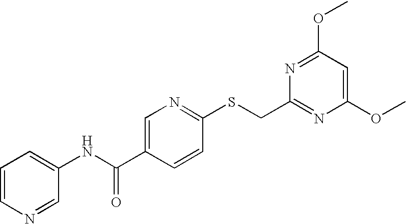 | | (CDCl3) δ: 3.90(6H, s), 4.57(2H, s), 5.91(1H, s), 7.26-7.48(2H, m), 7.84(1H, br s), 8.21-8.68 (5H, m). |
| 214 | 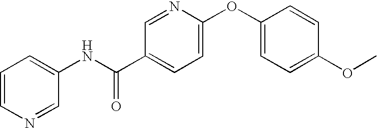 | | (CDCl3) δ: 2.51(3H, s), 7.01-7.36(6H, m), 8.25(1H, bs) 8.24-8.69(5H, m). |
| 215 | 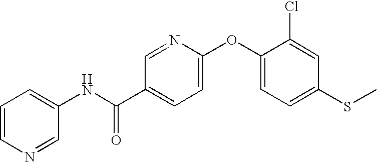 | | (CDCl3) δ: 2.52(3H, s), 7.09-7.36(5H, m), 7.74 (1H, br s), 8.31-8.58(5H, m). |
| 216 | 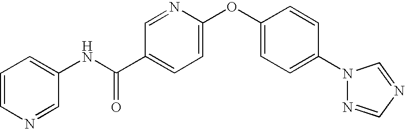 | | (CDCl3/DMSO-d6) ) δ: 7.07-7.35(4H, m), 7.86- 8.84(6H, m), 8.82-8.94(7H, m), 10.32(1H, bs) |
| 217 | 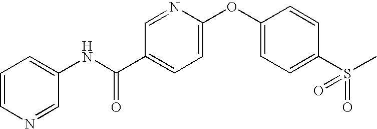 | | (CDCl3) δ: 3.10(2H, s), 7.15(1H, d, J = 8.4 Hz), 7.35-7.37(4H, m), 7.87(1H, br s), 8.00-8.71(7H, m). |
| 218 | 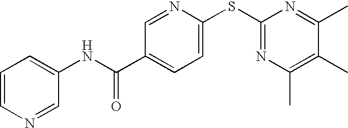 | | (CDCl3) δ: 2.24(3H, s), 2.45(6H, s), 7.30- 7.38(1H, m), 8.00-8.98(7H, m) |
| 219 | 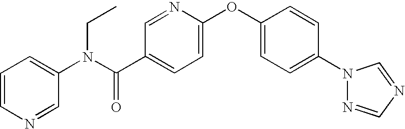 | | (CDCl3) δ: 1.25(3H, q, J = 7.1 Hz), 3.99(2H, q, J = 7.1 Hz), 6.80(1H, dd, J = 8.6, 0.7 Hz), 7.15- 7.71(7H, m), 8.00-8.47(5H, m) |
| 220 | 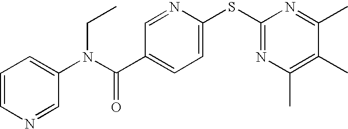 | | (CDCl3) δ: 1.25(3H, t, J = 7.1 Hz), 2.17(3H, s), 2.41(6H, s), 4.00(2H, q, J = 7.1 Hz), 7.45-7.70 (4H, m), 8.36-8.46(3H, m). |
| 221 | 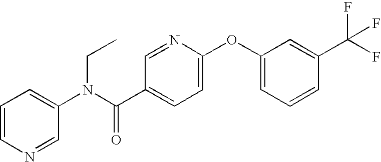 | | (CDCl3) δ: 1.25(3H, q, J = 7.1 Hz), 3.99(2H, q, J = 7.1 Hz), 6.80(1H, dd, J = 8.6, 0.7 Hz), 7.15- 7.71(7H, m), 8.00-8.47(3H, m) |
| 222 | 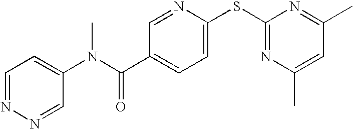 | | (CDCl3) δ: 2.41(6H, s), 3.58(3H, s), 6.83(1H, s), 7.21-7.26(1H, m), 7.70(1H, dd), 7.98(1H, d), 8.52(1H, d), 9.07- 9.09(2H, m) |
| 223 | 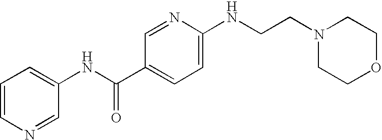 | | (CDCl3) δ: 2.50-2.51(4H, m), 2.64-2.66(2H, m), 3.42-3.46(2H, m), 3.73-3.74(4H, m), 5.60(1H, br s), 6.46(1H, d, J = 9.0 Hz), 7.26-7.33(1H, m), 7.85(1H, br s), 7.94-8.38(3H, m), 8.65-8.66(2H, m). |
| 224 | 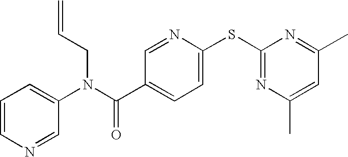 | | (CDCl3) δ: 2.42(6H, s), 4.54(2H, d), 5.91- 6.02(1H, m), 6.78(1H, s), 7.20-8.46(7H, m) |
| 225 | 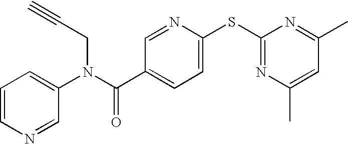 | | (CDCl3) δ: 2.35(1H, t), 2.41(6H, s), 4.71(2H, d), 6.79(1H, s), 7.27-8.52(7H, m) |
| 226 | 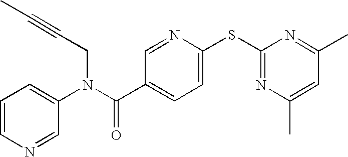 | | (CDCl3) δ: 1.80(3H, t), 2.38(6H, s), 4.63- 4.65(1H, m), 6.78(1H, s), 7.27-8.50(7H, m) |
| 227 | 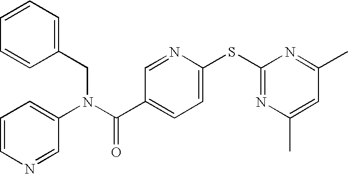 | | (CDCl3) δ: 2.37(6H, s), 5.14(2H, s), 6.78(1H, s), 7.14- 8.41(12H, m) |
| 228 | 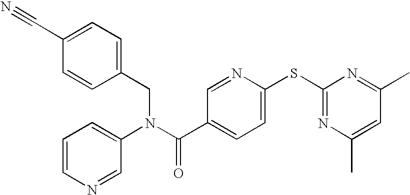 | | (CDCl3) δ: 2.39(6H, s), 5.19(2H, s), 6.79(1H, s), 7.18-8.45(11H, m) |
| 229 | 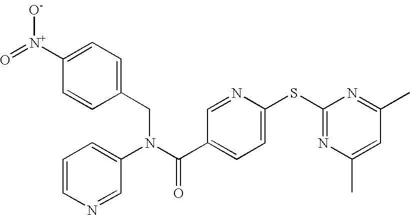 | | (CDCl3) δ: 2.38(6H, s), 5.23(2H, s), 6.79(1H, s), 7.18- 8.45(11H, m) |
| 230 | 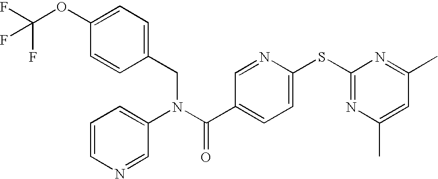 | | (CDCl3) δ: 2.37(6H, s), 5.13(2H, s), 6.78(1H, s), 7.14-8.44(11H, m) |
| 231 | 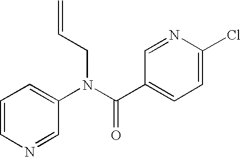 | | (CDCl3) δ: 4.51-4.54(2H, m), 5.17- 5.26(2H, m), 5.88-6.02(1H, m), 7.19-8.47(7H, m) |
| 232 | 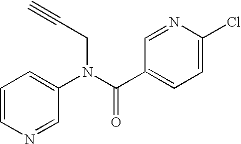 | | (CDCl3) δ: 2.32(1H, t), 4.69(2H, d), 5.88- 6.02(1H, m), 7.20-8.54(7H, m) |
| 233 | 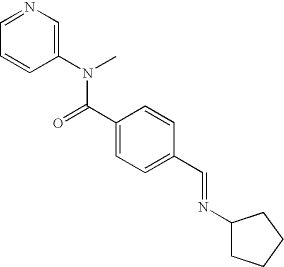 | | (CDCl3) δ: 1.52-1.72(4H, br), 1.72-1.92(4H, br), 3.52(3H, s), 3.64-3.82(1H, m), 7.14-7.46(4H, m), 7.73(2H, d, J = 8.4 Hz), 8.19(1H, s), 8.28- 8.46(2H, m) |
| 234 | 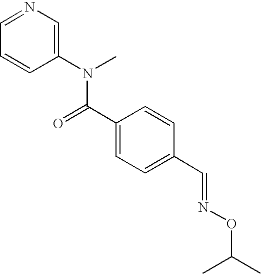 | | (CDCl3) δ: 1.27(6H, d, J = 6.3 Hz), 3.52(3H, s), 4.32-4.48(1H, m), 7.13-7.46(6H, m), 7.94(1H, s), 8.28-8.45(2H, m) |
| 235 | 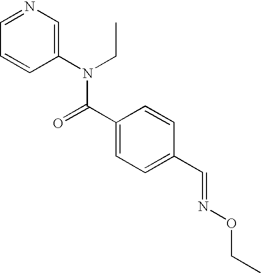 | | (CDCl3) δ: 1.14-1.34(6H, m), 4.00(2H, q, J = 7.2 Hz), 4.19(2H, q, J = 6.9 Hz), 7.13-7.46(8H, m), 7.96(1H, s), 8.29-8.45(2H, m) |
| 236 | 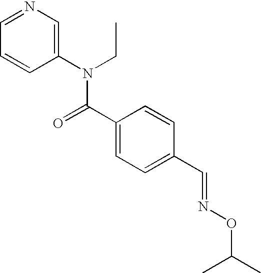 | | (CDCl3) δ: 1.18-1.34(9H, m), 3.99(2H, q, J = 7.2 Hz), 4.32-4.48(1H, m), 7.14-7.46(6H, m), 7.93 (1H, s), 8.30-8.45(2H, m) |
| 237 | 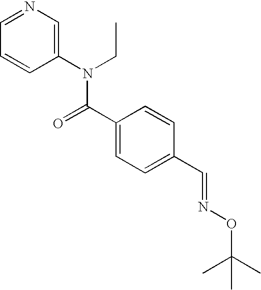 | | (CDCl3) δ: 1.23(3H, t, J = 7.2 Hz), 1.32(9H, s), 4.00(2H, q, J = 7.2 Hz), 7.14-7.45(5H, m), 7.92 (1H, s), 8.30-8.45(2H, m) |
| 238 | 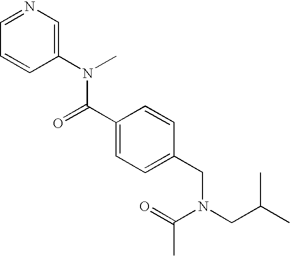 | LC/MS APCI [M + H]+: 340 | (CDCl3) δ: 3.97(3H, s), 7.32-7.39(1H, m), 7.86- 8.02(3H, m), 8.18(2H, d, J = 8.4 Hz), 8.27-8.35 (1H, m), 8.40-8.44(1H, m), 8.70(1H, d) |
| 239 | 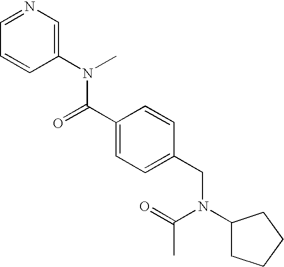 | LC/MS APCI [M + H]+: 352 | |
| 240 | 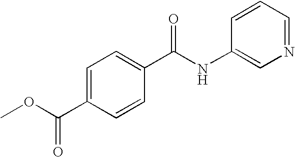 | | (CDCl3) δ: 3.97(3H, s), 7.32-7.39(1H, m), 7.86- 8.02(3H, m), 8.18(2H, d, J = 8.4 Hz), 8.27-8.35 (1H, m), 8.40-8.44(1H, m), 8.70(1H, d) |
| 241 | 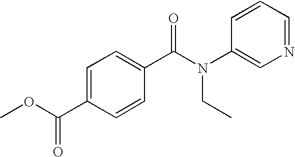 | | (CDCl3) δ: 1.24(3H, t, J = 7.2 Hz), 3.85(3H, s), 4.00(2H, q, J = 7.2 Hz), 7.16-7.44(4H, m), 7.86 (2H, d, J = 6.9 Hz), 8.29-8.45(2H, m) |
| 242 | 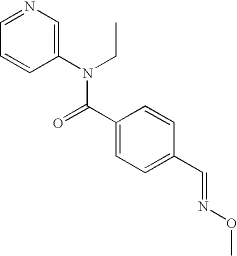 | | (CDCl3) δ: 1.24(3H, t, J = 7.2 Hz), 3.94(3H, s), 3.99(2H, q, J = 7.2 Hz), 7.14-7.48(5H, m), 7.94 (1H, s), 8.29-8.45(2H, m) |
| 243 | 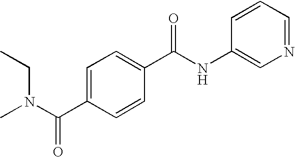 | LC/MS APCI [M + H]+: 284 | |
| 244 | 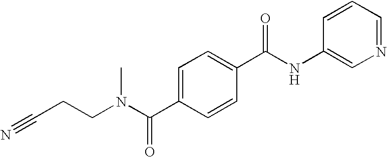 | | (CDCl3) δ: 2.83(2H, br s), 3.13(3H, br s), 3.81 (2H, br), 7.31-7.40(1H, m), 7.42-7.58(3H, m), 7.92(2H, d, J = 8.4 Hz), 8.33-8.44(2H, m), 8.73- 8.84(1H, br) |
| 245 | 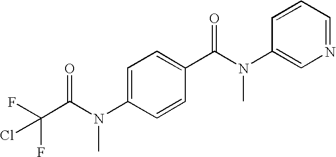 | | (CDCl3) δ: 3.29(3H, br s), 3.53(3H, s), 7.13- 7.40(6H, m), 8.37-8.43(2H, m) |
| 246 | 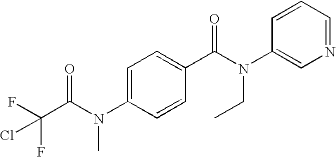 | | (CDCl3) δ: 1.26(3H, t, J = 7.2 Hz), 3.30(3H, br s), 4.01(2H, q, J = 7.2 Hz), 7.05-7.40(6H, m), 8.33- 8.45(2H, m) |
| 247 | 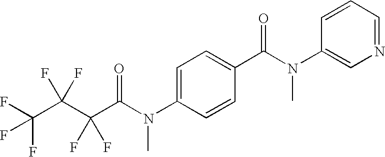 | | (CDCl3) δ: 3.30(3H, br s), 3.54(3H, s), 7.07- 7.40(6H, m), 8.41(2H, d) |
| 248 | 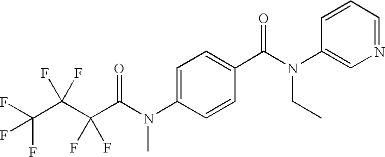 | | (CDCl3) δ: 1.26(3H, t, J = 7.2 Hz), 3.29(3H, br s), 4.01(2H, q, J = 7.2 Hz), 6.98-7.44(6H, m), 8.33- 8.47(2H, m) |
| 249 | 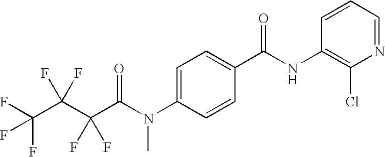 | LC/MS APCI [M + H]+: 458 | |
| 250 | 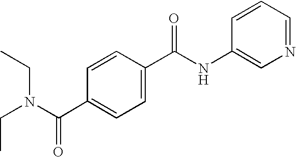 | | (CDCl3) δ: 1.08(3H, br t), 1.26(3H, br t), 3.19 (2H, br q), 3.55(2H, br q), 7.11-7.33(3H, m), 7.65-7.80(2H, m), 8.29-8.4(2H, m), 8.76-8.87 (1H, d), 9.78(1H, s) |
| 251 | 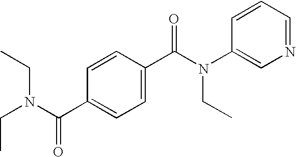 | | (CDCl3) δ: 1.03(3H, br s), 1.21(3H, br s), 1.24 (3H, t, J = 7.2 Hz), 3.22(2H, br s), 3.42(2H, br s), 3.97(2H, q, J = 7.2 Hz), 7.16-7.42(6H, m), 8.36- 8.49(2H, m) |
| 252 | 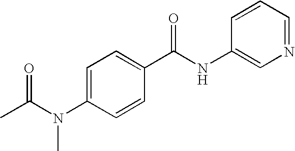 | | (CDCl3) δ: 1.96(3H, br s), 3.32(3H, br), 7.31- 7.40(4H, m), 7.97(2H, d, J = 8.1 Hz), 8.27-8.45 (2H, m), 8.67(1H, d) |
| 253 | 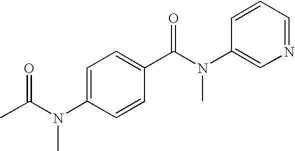 | | (CDCl3) δ: 1.80(3H, br s), 3.20(3H, s), 3.54(3H, s), 7.05(2H, d, J = 8.7 Hz), 7.21-7.46(4H, m), 8.33-8.43(2H, m) |
| 254 | 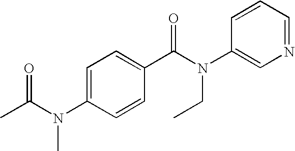 | | (CDCl3) δ: 1.26(3H, t, J = 7.2 Hz), 3.18(3H, s), 4.01(2H, q, J = 7.2 Hz), 7.03(2H, d, J = 8.4 Hz), 8.29-8.47(2H, m) |
| 255 | 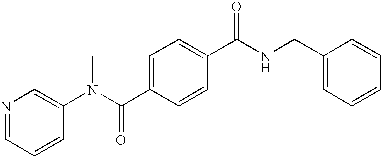 | | (CDCl3) δ: 3.47(3H, s), 4.56(2H, d, J = 5.7 Hz), 6.84-7.02(1H, br), 7.11-7.40(9H, m), 7.62(2H, d, J = 8.4 Hz), 8.22-8.36(2H, m) |
| 256 | 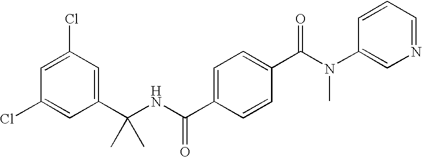 | | (CDCl3) δ: 3.51(3H, s), 6.70(1H, s), 7.16-7.42 (7H, m), 7.59(2H, d, J = 8.4 Hz), 8.29-8.42(2H, m) |
| 257 | 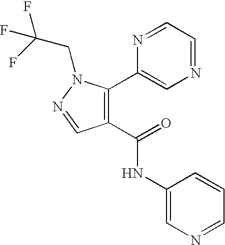 | MS-ESI: [M + H]+ 349 | |
| 258 | 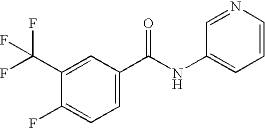 | MS-ESI: [M + H]+ 285 | |
| 259 | 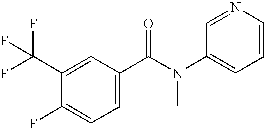 | MS-ESI: [M + H]+ 299 | |
| 260 | 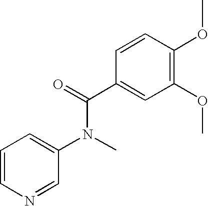 | MS-ESI: [M + H]+ 273 | |
| 261 | 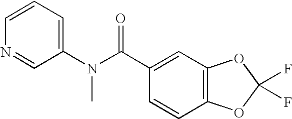 | MS-ESI: [M + H]+ 293 | |
| 262 | 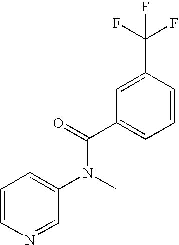 | MS-ESI: [M + H]+ 281 | |
| 263 | 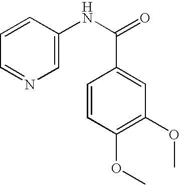 | MS-ESI: [M + H]+ 259 | |
| 264 | 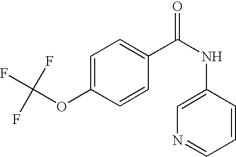 | MS-ESI: [M + H]+ 283 | |
| 265 | 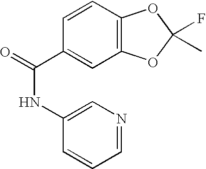 | MS-ESI: [M + H]+ 279 | |
| 266 | 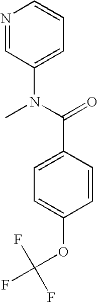 | MS-ESI: [M + H]+ 297 | |
| 267 | 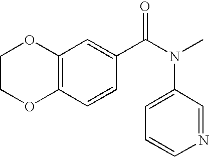 | MS-ESI: [M + H]+ 271 | |
| 268 | 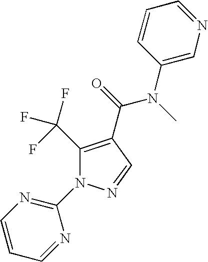 | | (D6-DMSO) δ: 3.4(s, 3H), 7.4(m, 1H), 7.65(m, 1H), 7.7.5(m, 1H), 7.85(m, 1H), 8.45(m, 2H), 8.95(m, 2H) +G289 |
| 269 | 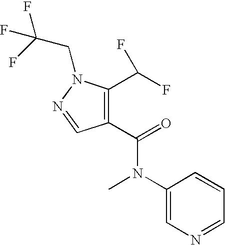 | | (D6-DMSO) δ: 3.4(s, 3H), 5.1(q, 2H), 7.1(s, 1H), 7.36(t, 1H), 7.4(m, 1H), 7.75(m, 1H), 7.95(m, 2H) |
| 270 | 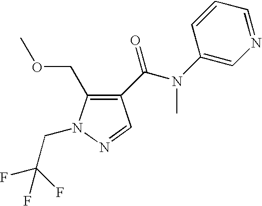 | MS-ESI: [M + H]+ 329 | |
| 271 | 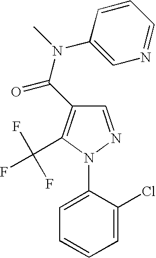 | MS-ESI: [M + H]+ 381 | |
| 272 | 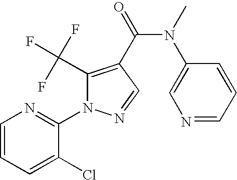 | MS-ESI: [M + H]+ 382 | |
| 273 | 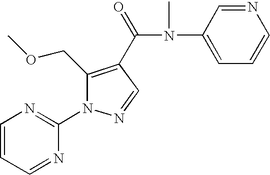 | MS-ESI: [M + H]+ 325 | |
| 274 | 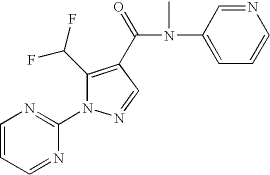 | MS-ESI: [M + H]+ 331 | |
| 275 | 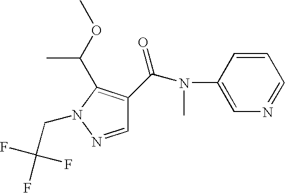 | | (D6-DMSO) δ: 1.4(d, 3H), 3.2(s, 3H), 3.35(s, 3H), 4.9-5.1(m, 3H), 6.9(s, 1H), 7.4(m, 1H), 7.7 (m, 1H), 8.4(m, 2H) |
| 276 | 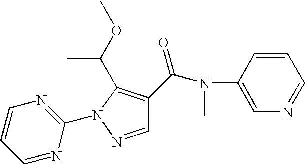 | MS-ESI: [M + H]+ 339 | |
| 277 | 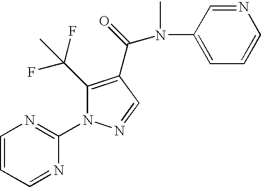 | MS-ESI: [M + H]+ 345 | |
| 278 | 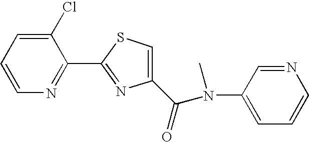 | MS-ESI: [M + H]+ 331 | |
| 279 | 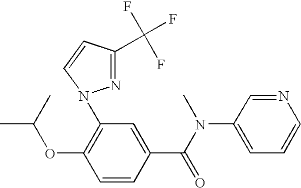 | MS-ESI: [M + H]+ 405 | |
| 280 | 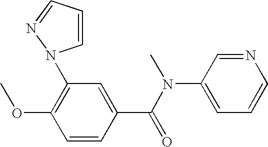 | | (D6-DMSO) δ: 3.4(s, 3H), 3.7(s, 3H), 6.4(m, 1H), 7.1(d, 1H), 7.2(d, 1H), 7.3(m, 1H), 7.2(m, 3H), 8.05(m, 1H), 8.35(m, 2H) |
| 281 | 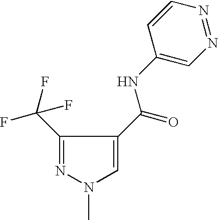 | MS-ESI: [M + H]+ 272 | |
| 282 | 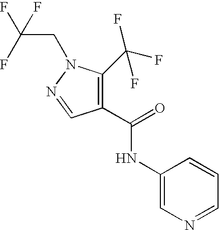 | MS-ESI: [M + H]+ 339 | |
| 283 | 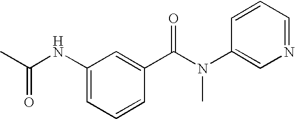 | MS-ESI: [M + H]+ 270 | |
| 284 | 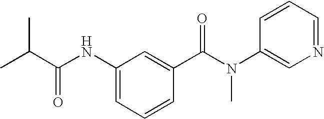 | MS-ESI: [M + H]+ 298 | |
| 285 | 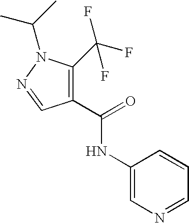 | MS-ESI: [M + H]+ 299 | |
| 286 | 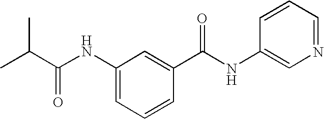 | MS-ESI: [M + H]+ 284 | |
| 287 | 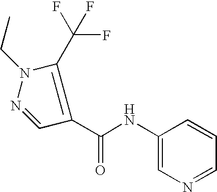 | MS-ESI: [M + H]+ 285 | |
| 288 | 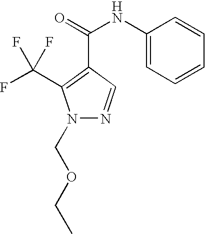 | MS-ESI: [M + H]+ 315 | |
| 289 | 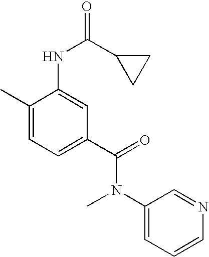 | MS-ESI: [M + H]+ 328 | |
| 290 | 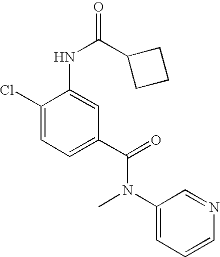 | MS-ESI: [M + H]+ 312 | |
| 291 | 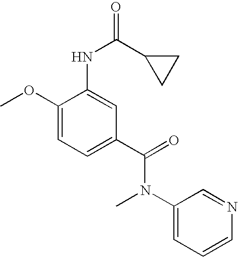 | MS-ESI: [M + H]+ 314 | |
| 292 | 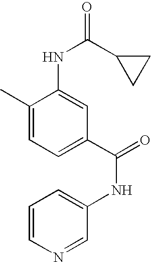 | MS-ESI: [M + H]+ 296 | |
| 293 | 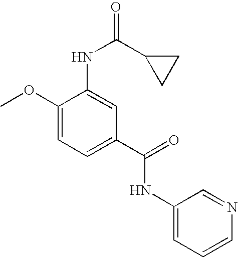 | MS-ESI: [M + H]+ 312 | |
| 294 | 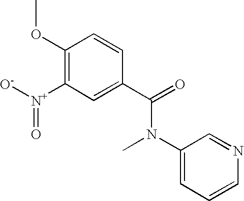 | MS-ESI: [M + H]+ 288 | |
| 295 | 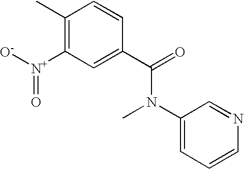 | MS-ESI: [M + H]+ 272 | |
| 296 | 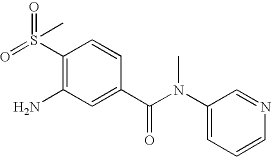 | MS-ESI: [M + H]+ 306 | |
| 297 | 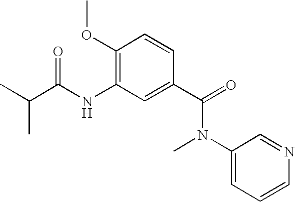 | MS-ESI: [M + H]+ 328 | |
| 298 | 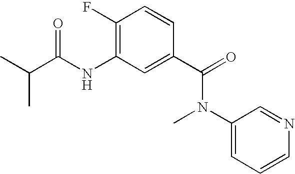 | MS-ESI: [M + H]+ 316 | |
| 299 | 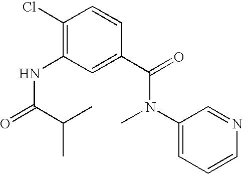 | MS-ESI: [M + H]+ 332 | |
| 300 | 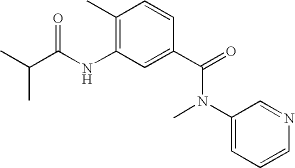 | MS-ESI: [M + H]+ 312 | |
| 301 | 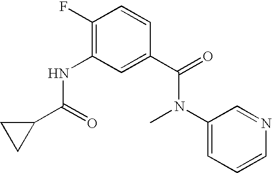 | MS-ESI: [M + H]+ 314 | |
| 302 | 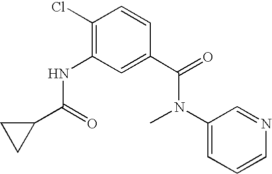 | MS-ESI: [M + H]+ 330 | |
| 303 | 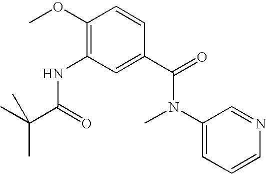 | MS-ESI: [M + H]+ 342 | |
| 304 | 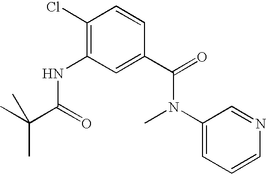 | MS-ESI: [M + H]+ 346 | |
| 305 | 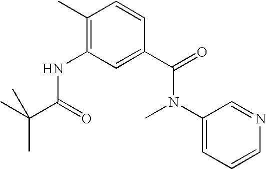 | MS-ESI: [M + H]+ 326 | |
| 306 | 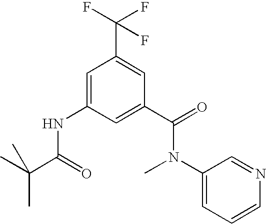 | MS-ESI: [M + H]+ 380 | |
| 307 | 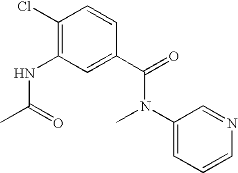 | MS-ESI: [M + H]+ 304 | |
| 308 | 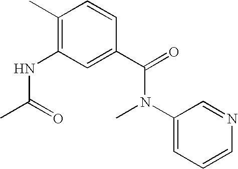 | MS-ESI: [M + H]+ 284 | |
| 309 | 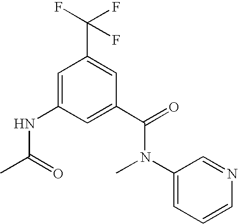 | MS-ESI: [M + H]+ 338 | |
| 310 | 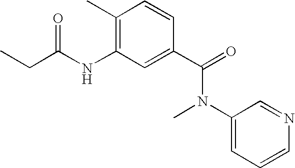 | MS-ESI: [M + H]+ 298 | |
| 311 | 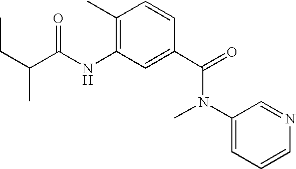 | MS-ESI: [M + H]+ 326 | |
| 312 | 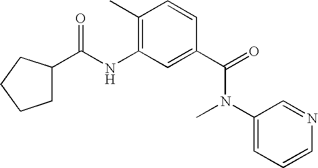 | MS-ESI: [M + H]+ 338 | |
| 313 | 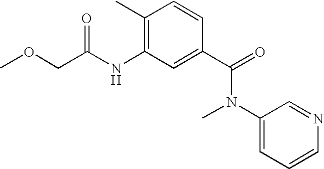 | MS-ESI: [M + H]+ 314 | |
| 314 | 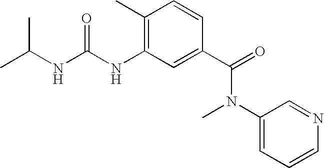 | MS-ESI: [M + H]+ 327 | |
| 315 | 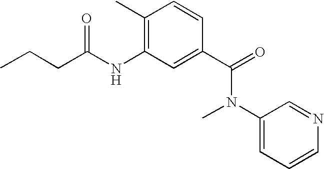 | MS-ESI: [M + H]+ 312 | |
| 316 | 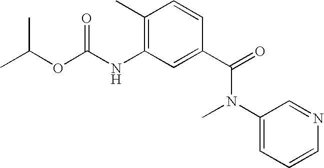 | MS-ESI: [M + H]+ 328 | |
| 317 | 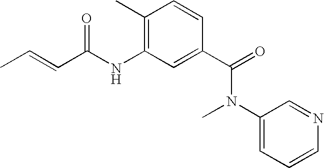 | MS-ESI: [M + H]+ 310 | |
| 318 | 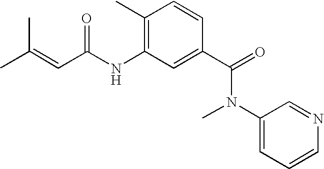 | MS-ESI: [M + H]+ 324 | |
| 319 | 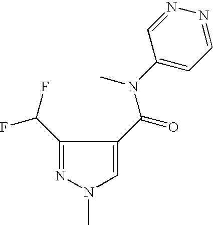 | MS-ESI: [M + H]+ 268 | |
| 320 | 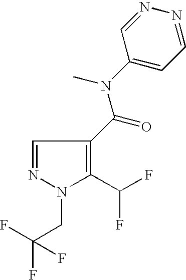 | MS-ESI: [M + H]+ 354 | |
| 321 | 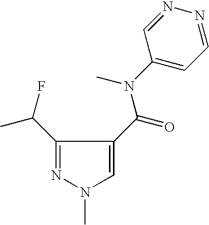 | MS-ESI: [M + H]+ 264 | |
| 322 | 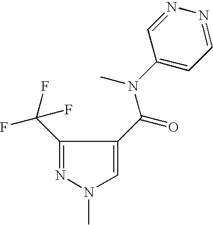 | MS-ESI: [M + H]+ 286 | |
| 323 | 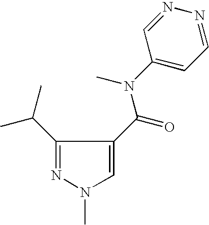 | MS-ESI: [M + H]+ 260 | |
| 324 | 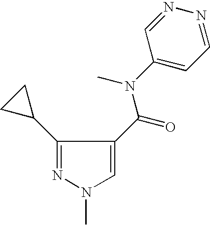 | MS-ESI: [M + H]+ 258 | |
| 325 | 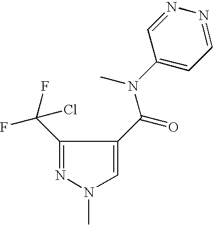 | MS-ESI: [M + H]+ 302 | |
| 326 | 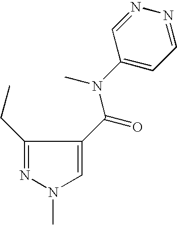 | MS-ESI: [M + H]+ 246 | |
| 327 | 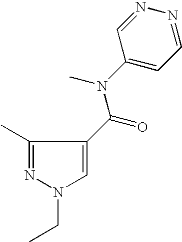 | MS-ESI: [M + H]+ 246 | |
| 328 | 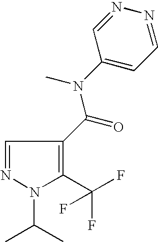 | MS-ESI: [M + H]+ 314 | |
| 329 | 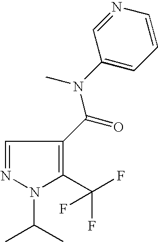 | MS-ESI: [M + H]+ 313 | |
| 330 | 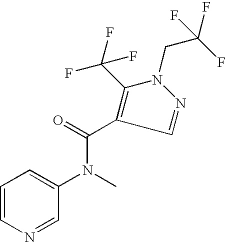 | MS-ESI: [M + H]+ 353 | |
| 331 | 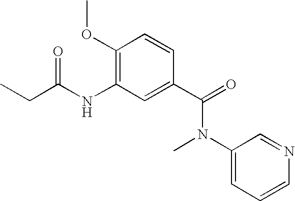 | MS-ESI: [M + H]+ 314 | |
| 332 | 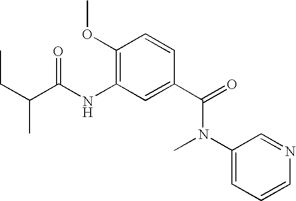 | MS-ESI: [M + H]+ 342 | |
| 333 | 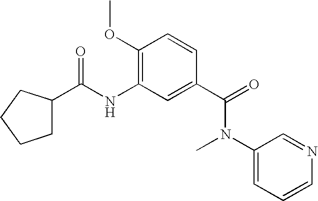 | MS-ESI: [M + H]+ 354 | |
| 334 | 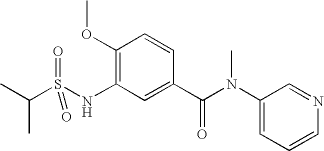 | MS-ESI: [M + H]+ 364 | |
| 335 | 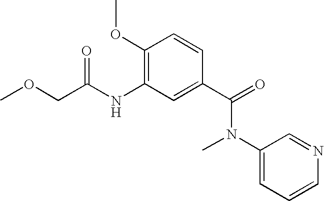 | MS-ESI: [M + H]+ 330 | |
| 336 | 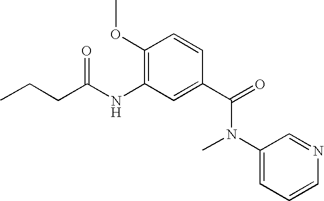 | MS-ESI: [M + H]+ 328 | |
| 337 | 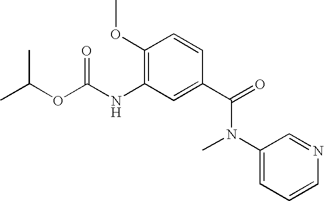 | MS-ESI: [M + H]+ 344 | |
| 338 | 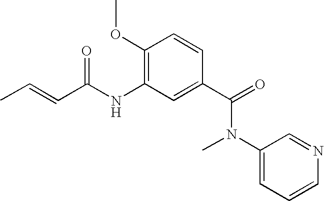 | MS-ESI: [M + H]+ 326 | |

The ¹H-NMR data were determined using a Bruker Avance 400, with tetramethylsilane as reference (0.0 ppm) and the solvents CD₃CN, CDCl₃ and [D₆]-DMSO. The signal splitting was characterized with s=singlet, br. s=broad singlet, d=doublet, t=triplet, q=quartet, m=multiplet, dd=doubled doublet.

The determination of the M+ by LC-MS in the acidic range takes place at a pH of 2.7 with acetonitrile (containing 0.1% formic acid) and water as eluents; linear gradient from 10% acetonitrile to 95% acetonitrile; instrument: Agilent 1100 LC system, Agilent MSD system, HTS PAL.

Further compounds according to the invention of the formula (I) are contained in Table 2.

TABELLE 2

| Number (I-) | 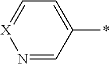 | R¹ | Q |
|---|---|---|---|
| 383 | pyridin-3-yl | H | 6-chloropyridin-3-yl |
| 384 | pyridin-3-yl | H | 3-(trifluoromethyl)phenyl |
| 385 | pyridin-3-yl | H | 4-{[chloro(difluoro)acetyl](methyl)amino}phenyl |
| 386 | pyridin-3-yl | H | 6-{[4-(trifluoromethyl)phenyl]sulphanyl}pyridin-3-yl |
| 387 | pyridin-3-yl | H | 3-fluoro-4-(methylsulphanyl)phenyl |
| 388 | pyridin-3-yl | H | 6-[(methylsulphanyl)methyl]pyridin-3-yl |
| 389 | pyridin-3-yl | H | 1-methyl-3-(trifluoromethyl)-1H-pyrazol-4-yl |
| 390 | pyridin-3-yl | H | 4-(acetylamino)phenyl |
| 391 | pyridin-3-yl | H | 3-[(4,6-dimethoxypyrimidin-2-yl)oxy]phenyl |
| 392 | pyridin-3-yl | H | 4-methyl-3-nitrophenyl |
| 393 | pyridin-3-yl | H | 4-{[(4,6-dimethylpyrimidin-2-yl)sulphanyl]methyl}phenyl |
| 394 | pyridin-3-yl | H | 4-nitrophenyl |
| 395 | pyridin-3-yl | H | 3-[chloro(difluoro)methyl]-1-methyl-1H-pyrazol-4-yl |
| 396 | pyridin-3-yl | H | 3-(methylsulphanyl)phenyl |
| 397 | pyridin-3-yl | H | 2,3-dihydro-1,4-benzodioxin-6-yl |
| 398 | pyridin-3-yl | H | 6-(4-methylphenoxy)pyridin-3-yl |
| 399 | pyridin-3-yl | H | 6-[(6-methylpyridin-3-yl)oxy]pyridin-3-yl |
| 400 | pyridin-3-yl | H | 4-methoxy-3-nitrophenyl |
| 401 | pyridin-3-yl | H | 6-[(4,6-dimethoxy-1,3,5-triazin-2-yl)sulphanyl]pyridin-3-yl |
| 402 | pyridin-3-yl | H | 6-[(4-chlorophenyl)sulphanyl]pyridin-3-yl |
| 403 | pyridin-3-yl | H | 4-[(cyclopropylcarbonyl)amino]phenyl |
| 404 | pyridin-3-yl | H | 3-(acetylamino)phenyl |
| 405 | pyridin-3-yl | CH₃ | 6-chloropyridin-3-yl |
| 406 | pyridin-3-yl | CH₃ | 4-(diethylcarbamoyl)phenyl |
| 407 | pyridin-3-yl | CH₃ | 4-[(4,6-dimethylpyrimidin-2-yl)sulphanyl]phenyl |
| 408 | pyridin-3-yl | CH₃ | 4-(methoxycarbonyl)phenyl |
| 409 | pyridin-3-yl | CH₃ | 4-[(tert-butoxyimino)methyl]phenyl |
| 410 | pyridin-3-yl | ethyl | 3-[(4,6-dimethoxypyrimidin-2-yl)oxy]phenyl |
| 411 | pyridin-3-yl | ethyl | 6-[(4,6-dimethoxy-1,3,5-triazin-2-yl)sulphanyl]pyridin-3-yl |
| 412 | pyridazin-4-yl | CH₃ | 4-[(methylsulphanyl)methyl]phenyl |
| 413 | pyridin-3-yl | H | 1-(2,6-dichloropyridin-3-yl)-5-(trifluoromethyl)-1H-pyrazol-4- |
| | | | yl |
| 414 | pyridin-3-yl | H | 5-(1-methoxyethyl)-1-[3-(trifluoromethyl)pyridin-2-yl]-1H- |
| | | | pyrazol-4-yl |
| 415 | pyridin-3-yl | H | 1-phenyl-1H-pyrazol-4-yl |
| 416 | pyridin-3-yl | H | 2-[5-chloro-3-(trifluoromethyl)pyridin-2-yl]-1,3-thiazol-4-yl |
| 417 | pyridin-3-yl | H | 1-[3-(difluoromethyl)pyridin-2-yl]-1H-pyrazol-4-yl |
| 418 | pyridin-3-yl | H | 5-(trifluoromethyl)-1-[3-(trifluoromethyl)pyridin-2-yl]-1H- |
| | | | pyrazol-4-yl |
| 419 | pyridin-3-yl | H | 1-(2-bromophenyl)-5-(1-methoxyethyl)-1H-pyrazol-4-yl |
| 420 | pyridin-3-yl | H | 2-[3-(1-methoxyethyl)pyridin-2-yl]-1,3-thiazol-4-yl |
| 421 | pyridin-3-yl | H | 5-(1-fluoroethyl)-1-(pyrimidin-2-yl)-1H-pyrazol-4-yl |
| 422 | pyridin-3-yl | H | 1-[2-(trifluoromethyl)phenyl]-1H-pyrazol-4-yl |
| 423 | pyridin-3-yl | H | 1-[3-chloro-2-(trifluoromethyl)phenyl]-5-(methoxymethyl)- |
| | | | 1H-pyrazol-4-yl |
| 424 | pyridin-3-yl | H | 4-[(trifluoroacetyl)amino]phenyl |
| 425 | pyridin-3-yl | H | 6(2,5-dichlorophenoxy)pyridin-3-yl |
| 426 | pyridin-3-yl | H | 2-(pyrimidin-2-yl)-1,3-thiazol-5-yl |
| 427 | pyridin-3-yl | H | 3-(but-2-enoylamino)-4-methoxyphenyl |
| 428 | pyridin-3-yl | H | 4-chloro-3-[(cyclopropylcarbonyl)amino]phenyl |
| 429 | pyridin-3-yl | H | 1-(2,6-dichlorophenyl)-5-methyl-1H-pyrazol-4-yl |
| 430 | pyridin-3-yl | H | 1-(2-chloropyridin-3-yl)-5-(difluoromethyl)-1H-pyrazol-4-yl |
| 431 | pyridin-3-yl | H | 1-(3-chloropyridin-2-yl)-5-(dimethoxymethyl)-1H-pyrazol-4-yl |
| 432 | pyridin-3-yl | H | 5-(difluoromethyl)-1-(2,2,2-trifluoroethyl)-1H-pyrazol-4-yl |
| 433 | pyridin-3-yl | H | 1-(2-bromo-6-chlorophenyl)-5-(dimethoxymethyl)-1H- |
| | | | pyrazol-4-yl |
| 434 | pyridin-3-yl | H | 4-methoxy-3-[3-(pyridin-2-yl)-1H-pyrazol-1-yl]phenyl |
| 435 | pyridin-3-yl | H | 3-chloro-4-{[4-(trifluoromethyl)-1H-pyrazol-1- |
| | | | yl]methyl}phenyl |
| 436 | pyridin-3-yl | H | 4-methyl-3-[(2-methylbutanoyl)amino]phenyl |
| 437 | pyridin-3-yl | H | 4-{[3-(3-chloropyridin-2-yl)-1H-pyrazol-1-yl]methyl}phenyl |
| 438 | pyridin-3-yl | H | 6-{[4-(pyridin-2-yl)-1H-pyrazol-1-yl]methyl}pyridin-3-yl |
| 439 | pyridin-3-yl | H | 4-methoxy-3-[3-(pyrimidin-2-yl)-1H-pyrazol-1-yl]phenyl |
| 440 | pyridin-3-yl | H | 4-{[3-(pyridin-2-yl)-1H-pyrazol-1-yl]methyl}phenyl |
| 441 | pyridin-3-yl | H | 4-chloro-3-[(2,2-dimethylpropanoyl)amino]phenyl |
| 442 | pyridin-3-yl | H | 1-(2-chloropyridin-3-yl)-5-(1-methoxyethyl)-1H-pyrazol-4-yl |
| 443 | pyridin-3-yl | H | 2-(3,5-dichloropyridin-2-yl)-1,3-thiazol-4-yl |
| 444 | pyridin-3-yl | H | 5-chloro-6-(4-cyanophenoxy)pyridin-3-yl |
| 445 | pyridin-3-yl | H | 6-(3,4-dichlorophenoxy)pyridin-3-yl |
| 446 | pyridin-3-yl | H | 6-[(2-chloropyrimidin-4-yl)oxy]pyridin-3-yl |
| 447 | pyridin-3-yl | H | 1-(3-chloropyridin-2-yl)-5-(1-methoxyethyl)-1H-pyrazol-4-yl |
| 448 | pyridin-3-yl | H | 2-[2-(trifluoromethyl)phenyl]-1,3-thiazol-4-yl |
| 449 | pyridin-3-yl | H | 2-(2,6-dichloropyridin-3-yl)-1,3-thiazol-4-yl |
| 450 | pyridin-3-yl | H | 1-[3-(difluoromethyl)pyridin-2-yl]-5-methyl-1H-pyrazol-4-yl |
| 451 | pyridin-3-yl | H | 1-(2,4-dichlorophenyl)-1H-pyrazol-4-yl |
| 452 | pyridin-3-yl | H | 2-[4-chloro-2-(trifluoromethyl)phenyl]-1,3-thiazol-5-yl |
| 453 | pyridin-3-yl | H | 4-[(2-methylpyrimidin-4-yl)oxy]phenyl |
| 454 | pyridin-3-yl | H | 6-{[5-(trifluoromethyl)pyridin-2-yl]sulphanyl}pyridin-3-yl |
| 455 | pyridin-3-yl | H | 4-{[(propan-2-yloxy)imino]methyl}phenyl |
| 456 | pyridin-3-yl | H | 4-methyl-3-{[(propan-2-yloxy)carbonyl]amino}phenyl |
| 457 | pyridin-3-yl | H | 1-[4-fluoro-2-(trifluoromethyl)phenyl]-5-(methoxymethyl)-1H- |
| | | | pyrazol-4-yl |
| 458 | pyridin-3-yl | H | 5-(1,1-difluoroethyl)-1-(pyrimidin-2-yl)-1H-pyrazol-4-yl |
| 459 | pyridin-3-yl | H | 1-(2-chlorophenyl)-5-(1,1-dimethoxyethyl)-1H-pyrazol-4-yl |
| 460 | pyridin-3-yl | H | 1-(3-chloropyridin-2-yl)-5-(1,1-difluoroethyl)-1H-pyrazol-4-yl |
| 461 | pyridin-3-yl | H | 1-(2,6-dichloropyridin-3-yl)-5-(methoxymethyl)-1H-pyrazol-4- |
| | | | yl |
| 462 | pyridin-3-yl | H | 6-(quinolin-6-yloxy)pyridin-3-yl |
| 463 | pyridin-3-yl | H | 3-(4-chloro-1H-pyrazol-1-yl)-4-methoxyphenyl |
| 464 | pyridin-3-yl | H | 2-(2,4-dichlorophenyl)-1,3-thiazol-4-yl |
| 465 | pyridin-3-yl | H | 1-(2-chloropyridin-3-yl)-5-(1,1-difluoroethyl)-1H-pyrazol-4-yl |
| 466 | pyridin-3-yl | H | 6-{4-[4-(methylsulphanyl)phenoxy]phenoxy}pyridin-3-yl |
| 467 | pyridin-3-yl | H | 6-(quinolin-5-yloxy)pyridin-3-yl |
| 468 | pyridin-3-yl | H | 1-[4-chloro-2-(trifluoromethyl)phenyl]-5-(difluoromethyl)-1H- |
| | | | pyrazol-4-yl |
| 469 | pyridin-3-yl | H | 1-[2-(1-methoxyethyl)phenyl]-1H-pyrazol-4-yl |
| 470 | pyridin-3-yl | H | 4-methoxy-3-[(2-methylbutanoyl)amino]phenyl |
| 471 | pyridin-3-yl | H | 6-(2,4-dichlorophenoxy)pyridin-3-yl |
| 472 | pyridin-3-yl | H | 4-{[acetyl(2-methylpropyl)amino]methyl}phenyl |
| 473 | pyridin-3-yl | H | 2-(2,4-dichlorophenyl)-1,3-thiazol-5-yl |
| 474 | pyridin-3-yl | H | 1-(2-bromo-6-chlorophenyl)-5-(difluoromethyl)-1H-pyrazol-4- |
| | | | yl |
| 475 | pyridin-3-yl | H | 4-(propan-2-yloxy)-3-[3-(trifluoromethyl)-1H-pyrazol-1- |
| | | | yl]phenyl |
| 476 | pyridin-3-yl | H | 6-(3,5-dimethylphenoxy)pyridin-3-yl |
| 477 | pyridin-3-yl | H | 1-[4-chloro-2-(trifluoromethyl)phenyl]-5-methyl-1H-pyrazol- |
| | | | 4-yl |
| 478 | pyridin-3-yl | H | 1-(4-chlorophenyl)-1H-pyrazol-4-yl |
| 479 | pyridin-3-yl | H | 2-[2-(difluoromethyl)phenyl]-1,3-thiazol-5-yl |
| 480 | pyridin-3-yl | H | 5-chloro-6-[4-(trifluoromethyl)phenoxy]pyridin-3-yl |
| 481 | pyridin-3-yl | H | 4-methyl-3-(propanoylamino)phenyl |
| 482 | pyridin-3-yl | H | 2-(2-chloropyridin-3-yl)-1,3-thiazol-4-yl |
| 483 | pyridin-3-yl | H | 4-[(2-methylpropanoyl)amino]phenyl |
| 484 | pyridin-3-yl | H | 3-[(2,2-dimethylpropanoyl)amino]-4-methylphenyl |
| 485 | pyridin-3-yl | H | 4-methoxy-3-{[(propan-2-yloxy)carbonyl]amino}phenyl |
| 486 | pyridin-3-yl | H | 1-(2,3-dichlorophenyl)-5-(methoxymethyl)-1H-pyrazol-4-yl |
| 487 | pyridin-3-yl | H | 2-[3-(difluoromethyl)pyridin-2-yl]-1,3-thiazol-5-yl |
| 488 | pyridin-3-yl | H | 3-(methoxymethyl)-1-methyl-1H-pyrazol-4-yl |
| 489 | pyridin-3-yl | H | 2-(3,5-dichloropyridin-2-yl)-1,3-thiazol-5-yl |
| 490 | pyridin-3-yl | H | 1-(2,6-dichloropyridin-3-yl)-5-(difluoromethyl)-1H-pyrazol-4- |
| | | | yl |
| 491 | pyridin-3-yl | H | 6-[(4,6-dimethylpyrimidin-2-yl)oxy]pyridin-3-yl |
| 492 | pyridin-3-yl | H | 5-chloro-6-(4-nitrophenoxy)pyridin-3-yl |
| 493 | pyridin-3-yl | H | 2-[2-(difluoromethyl)phenyl]-1,3-thiazol-4-yl |
| 494 | pyridin-3-yl | H | 6-[(6-chloropyridin-2-yl)oxy]pyridin-3-yl |
| 495 | pyridin-3-yl | H | 3-(butanoylamino)-4-methoxyphenyl |
| 496 | pyridin-3-yl | H | 1-(2-bromophenyl)-5-(trifluoromethyl)-1H-pyrazol-4-yl |
| 497 | pyridin-3-yl | H | 3-{[3-(trifluoromethyl)-1H-pyrazol-1-yl]methyl}phenyl |
| 498 | pyridin-3-yl | H | 5-(difluoromethyl)-1-(2-methylphenyl)-1H-pyrazol-4-yl |
| 499 | pyridin-3-yl | H | 2-[2-(1-methoxyethyl)phenyl]-1,3-thiazol-5-yl |
| 500 | pyridin-3-yl | H | 1-(2-chlorophenyl)-5-(1,1-difluoroethyl)-1H-pyrazol-4-yl |
| 501 | pyridin-3-yl | H | 1-(2-bromo-4-fluorophenyl)-1H-pyrazol-4-yl |
| 502 | pyridin-3-yl | H | 6-[(2-methoxypyrimidin-4-yl)oxy]pyridin-3-yl |
| 503 | pyridin-3-yl | H | 1-[2-(1,1-difluoroethyl)phenyl]-5-methyl-1H-pyrazol-4-yl |
| 504 | pyridin-3-yl | H | 2-(pyrimidin-2-yl)-1,3-thiazol-4-yl |
| 505 | pyridin-3-yl | H | 2-{[3-(trifluoromethyl)-1H-pyrazol-1-yl]methyl}pyridin-4-yl |
| 506 | pyridin-3-yl | H | 1-(2-chloropyridin-3-yl)-5-(trifluoromethyl)-1H-pyrazol-4-yl |
| 507 | pyridin-3-yl | H | 1-(2-chloro-4-fluorophenyl)-1H-pyrazol-4-yl |
| 508 | pyridin-3-yl | H | 4-methoxy-3-[4-(pyridin-2-yl)-1H-pyrazol-1-yl]phenyl |
| 509 | pyridin-3-yl | H | 1-(2-chlorophenyl)-1H-pyrazol-4-yl |
| 510 | pyridin-3-yl | H | 6-(2,6-dichlorophenoxy)pyridin-3-yl |
| 511 | pyridin-3-yl | H | 1-(2-chlorophenyl)-5-methyl-1H-pyrazol-4-yl |
| 512 | pyridin-3-yl | H | 2-[2-(1-methoxyethyl)phenyl]-1,3-thiazol-4-yl |
| 513 | pyridin-3-yl | H | 6-{[6-(1H-pyrazol-1-yl)pyridin-2-yl]oxy}pyridin-3-yl |
| 514 | pyridin-3-yl | H | 2-[4-chloro-2-(trifluoromethyl)phenyl]-1,3-thiazol-4-yl |
| 515 | pyridin-3-yl | H | 4-methoxy-3-(1H-pyrazol-1-yl)phenyl |
| 516 | pyridin-3-yl | H | 3-(acetylamino)-4-methylphenyl |
| 517 | pyridin-3-yl | H | 2-{[4-(trifluoromethyl)-1H-pyrazol-1-yl]methyl}pyridin-4-yl |
| 518 | pyridin-3-yl | H | 1-(2,4-difluorophenyl)-1H-pyrazol-4-yl |
| 519 | pyridin-3-yl | H | 1-(2-methylphenyl)-5-(trifluoromethyl)-1H-pyrazol-4-yl |
| 520 | pyridin-3-yl | H | 2-[3-(difluoromethyl)pyridin-2-yl]-1,3-thiazol-4-yl |
| 521 | pyridin-3-yl | H | 6-(3,5-dichlorophenoxy)pyridin-3-yl |
| 522 | pyridin-3-yl | H | 6-[(4-methylphenyl)sulphanyl]pyridin-3-yl |
| 523 | pyridin-3-yl | H | 1-(2,4-dichlorophenyl)-5-(methoxymethyl)-1H-pyrazol-4-yl |
| 524 | pyridin-3-yl | H | 6-{4-[4-(trifluoromethyl)phenoxy]phenoxy}pyridin-3-yl |
| 525 | pyridin-3-yl | H | 1-(2,6-dichlorophenyl)-5-(trifluoromethyl)-1H-pyrazol-4-yl |
| 526 | pyridin-3-yl | H | 6-(naphthalen-1-yloxy)pyridin-3-yl |
| 527 | pyridin-3-yl | H | 2-(2,6-dichloropyridin-3-yl)-1,3-thiazol-5-yl |
| 528 | pyridin-3-yl | H | 1-[3-chloro-2-(trifluoromethyl)phenyl]-5-(1-methoxyethyl)- |
| | | | 1H-pyrazol-4-yl |
| 529 | pyridin-3-yl | H | 4-methoxy-3-[(methoxyacetyl)amino]phenyl |
| 530 | pyridin-3-yl | H | 3-{[4-(trifluoromethyl)-1H-pyrazol-1-yl]methyl}phenyl |
| 531 | pyridin-3-yl | H | 6-(2-cyanophenoxy)pyridin-3-yl |
| 532 | pyridin-3-yl | H | 3-(dimethoxymethyl)-1-methyl-1H-pyrazol-4-yl |
| 533 | pyridin-3-yl | H | 1-(3-bromopyridin-2-yl)-5-(methoxymethyl)-1H-pyrazol-4-yl |
| 534 | pyridin-3-yl | H | 1-(2-bromo-6-chlorophenyl)-5-(trifluoromethyl)-1H-pyrazol-4- |
| | | | yl |
| 535 | pyridin-3-yl | H | 3-{[4-(trifluoromethyl)phenoxy]methyl}phenyl |
| 536 | pyridin-3-yl | H | 3-(4-bromo-1H-pyrazol-1-yl)-4-methoxyphenyl |
| 537 | pyridin-3-yl | H | 5-methyl-1-(2-methylphenyl)-1H-pyrazol-4-yl |
| 538 | pyridin-3-yl | H | 1-[3-(difluoromethyl)pyridin-2-yl]-5-(methoxymethyl)-1H- |
| | | | pyrazol-4-yl |
| 539 | pyridin-3-yl | H | 2-[2-(methoxymethyl)phenyl]-1,3-thiazol-5-yl |
| 540 | pyridin-3-yl | H | 5-chloro-6-[(4,5,6-trimethylpyrimidin-2-yl)sulphanyl]pyridin- |
| | | | 3-yl |
| 541 | pyridin-3-yl | H | 6-[3,5-bis(trifluoromethyl)phenoxy]pyridin-3-yl |
| 542 | pyridin-3-yl | H | 4-{[4-(pyridin-2-yl)-1H-pyrazol-1-yl]methyl}phenyl |
| 543 | pyridin-3-yl | H | 1-[3-(1,1-difluoroethyl)pyridin-2-yl]-5-methyl-1H-pyrazol-4-yl |
| 544 | pyridin-3-yl | H | 1-(2,6-dichlorophenyl)-5-(difluoromethyl)-1H-pyrazol-4-yl |
| 545 | pyridin-3-yl | H | 1-[4-fluoro-2-(trifluoromethyl)phenyl]-5-methyl-1H-pyrazol-4- |
| | | | yl |
| 546 | pyridin-3-yl | H | 4-chloro-3-[(cyclobutylcarbonyl)amino]phenyl |
| 547 | pyridin-3-yl | H | 2-[2-(methoxymethyl)phenyl]-1,3-thiazol-4-yl |
| 548 | pyridin-3-yl | H | 4-(1H-pyrazol-1-ylmethyl)phenyl |
| 549 | pyridin-3-yl | H | 1-(2-bromophenyl)-5-methyl-1H-pyrazol-4-yl |
| 550 | pyridin-3-yl | H | 1-(2,3-difluorophenyl)-1H-pyrazol-4-yl |
| 551 | pyridin-3-yl | H | 6-{4-[(trifluoromethyl)sulphanyl]phenoxy}pyridin-3-yl |
| 552 | pyridin-3-yl | H | 4-{[3-(pyrimidin-2-yl)-1H-pyrazol-1-yl]methyl}phenyl |
| 553 | pyridin-3-yl | H | 1-(2-chlorophenyl)-5-(difluoromethyl)-1H-pyrazol-4-yl |
| 554 | pyridin-3-yl | H | 5-(difluoromethyl)-1-[2-(trifluoromethyl)phenyl]-1H-pyrazol- |
| | | | 4-yl |
| 555 | pyridin-3-yl | H | 1-[2-(dimethoxymethyl)phenyl]-1H-pyrazol-4-yl |
| 556 | pyridin-3-yl | H | 5-(methoxymethyl)-1-(pyrimidin-2-yl)-1H-pyrazol-4-yl |
| 557 | pyridin-3-yl | H | 6-tert-butoxypyridin-3-yl |
| 558 | pyridin-3-yl | H | 4-[(cyclopentylimino)methyl]phenyl |
| 559 | pyridin-3-yl | H | 1-(3-chlorophenyl)-1H-pyrazol-4-yl |
| 560 | pyridin-3-yl | H | 4-chloro-3-[(2-methylpropanoyl)amino]phenyl |
| 561 | pyridin-3-yl | H | 1-[2-(difluoromethyl)phenyl]-5-(methoxymethyl)-1H-pyrazol- |
| | | | 4-yl |
| 562 | pyridin-3-yl | H | 4-methoxy-3-[4-(pyrimidin-2-yl)-1H-pyrazol-1-yl]phenyl |
| 563 | pyridin-3-yl | H | 1-(2-chlorophenyl)-5-(methoxymethyl)-1H-pyrazol-4-yl |
| 564 | pyridin-3-yl | H | 5-chloro-6-[4-(trifluoromethoxy)phenoxy]pyridin-3-yl |
| 565 | pyridin-3-yl | H | 4-[(tert-butoxyimino)methyl]phenyl |
| 566 | pyridin-3-yl | H | 2-(3-chloropyridin-2-yl)-1,3-thiazol-5-yl |
| 567 | pyridin-3-yl | H | 1-(2-fluorophenyl)-1H-pyrazol-4-yl |
| 568 | pyridin-3-yl | H | 3-[(2,2-dimethylpropanoyl)amino]-5-(trifluoromethyl)phenyl |
| 569 | pyridin-3-yl | H | 1-(3-chlorophenyl)-5-(1,1-dimethoxyethyl)-1H-pyrazol-4-yl |
| 570 | pyridin-3-yl | H | 1-(2,4-dichlorophenyl)-5-(1,1-dimethoxyethyl)-1H-pyrazol-4- |
| | | | yl |
| 571 | pyridin-3-yl | H | 3-[(cyclopropylcarbonyl)amino]-4-fluorophenyl |
| 572 | pyridin-3-yl | H | 6-[4-(dimethylsulphamoyl)phenoxy]pyridin-3-yl |
| 573 | pyridin-3-yl | H | 1-(2,4-difluorophenyl)-5-methyl-1H-pyrazol-4-yl |
| 574 | pyridin-3-yl | H | 3-(acetylamino)-4-chlorophenyl |
| 575 | pyridin-3-yl | H | 4-[(4-bromo-1H-pyrazol-1-yl)methyl]phenyl |
| 576 | pyridin-3-yl | H | 4-methyl-3-[(propan-2-ylcarbamoyl)amino]phenyl |
| 577 | pyridin-3-yl | H | 1-(2,2-diethoxyethyl)-3-(trifluoromethyl)-1H-pyrazol-4-yl |
| 578 | pyridin-3-yl | H | 4-(1H-1,2,4-triazol-1-ylmethyl)phenyl |
| 579 | pyridin-3-yl | H | 5-(methoxymethyl)-1-[2-(trifluoromethyl)phenyl]-1H-pyrazol- |
| | | | 4-yl |
| 580 | pyridin-3-yl | H | 5-chloro-6-[(4,6-dimethylpyrimidin-2-yl)sulphanyl]pyridin-3- |
| | | | yl |
| 581 | pyridin-3-yl | H | 3-[(2,2-dimethylpropanoyl)amino]-4-methoxyphenyl |
| 582 | pyridin-3-yl | H | 2-[2-(trifluoromethyl)pyridin-3-yl]-1,3-thiazol-4-yl |
| 583 | pyridin-3-yl | H | 5-(methoxymethyl)-1-(2,2,2-trifluoroethyl)-1H-pyrazol-4-yl |
| 584 | pyridin-3-yl | H | 4-methoxy-3-(propanoylamino)phenyl |
| 585 | pyridin-3-yl | H | 4-methoxy-3-[(2-methylpropanoyl)amino]phenyl |
| 586 | pyridin-3-yl | H | 6-(quinolin-7-yloxy)pyridin-3-yl |
| 587 | pyridin-3-yl | H | 4-{[3-(trifluoromethyl)-1H-pyrazol-1-yl]methyl}phenyl |
| 588 | pyridin-3-yl | H | 3-fluoro-4-{[4-(trifluoromethyl)-1H-pyrazol-1- |
| | | | yl]methyl}phenyl |
| 589 | pyridin-3-yl | H | 2-[3-(methoxymethyl)pyridin-2-yl]-1,3-thiazol-5-yl |
| 590 | pyridin-3-yl | H | 6-({6-[3-(trifluoromethyl)-1H-pyrazol-1-yl]pyridin-2- |
| | | | yl}oxy)pyridin-3-yl |
| 591 | pyridin-3-yl | H | 3-{[3-(pyridin-2-yl)-1H-pyrazol-1-yl]methyl}phenyl |
| 592 | pyridin-3-yl | H | 1-(2-bromo-4-chlorophenyl)-5-methyl-1H-pyrazol-4-yl |
| 593 | pyridin-3-yl | H | 2-[3-(trifluoromethyl)pyridin-2-yl]-1,3-thiazol-4-yl |
| 594 | pyridin-3-yl | H | 4-(benzylcarbamoyl)phenyl |
| 595 | pyridin-3-yl | H | 4-[(2-chloropyrimidin-4-yl)oxy]phenyl |
| 596 | pyridin-3-yl | H | 1-[3-(trifluoromethyl)pyridin-2-yl]-1H-pyrazol-4-yl |
| 597 | pyridin-3-yl | H | 5-chloro-6-(4-chlorophenoxy)pyridin-3-yl |
| 598 | pyridin-3-yl | H | 3-amino-4-(methylsulphonyl)phenyl |
| 599 | pyridin-3-yl | H | 4-methyl-3-[(2-methylpropanoyl)amino]phenyl |
| 600 | pyridin-3-yl | H | 1-(2,6-dibromophenyl)-5-(trifluoromethyl)-1H-pyrazol-4-yl |
| 601 | pyridin-3-yl | H | 3-{[3-(trifluoromethyl)-1H-1,2,4-triazol-1-yl]methyl}phenyl |
| 602 | pyridin-3-yl | H | 4-[(3,3,3-trifluoropropanoyl)amino]phenyl |
| 603 | pyridin-3-yl | H | 1-(2,6-dichlorophenyl)-5-(dimethoxymethyl)-1H-pyrazol-4-yl |
| 604 | pyridin-3-yl | H | 1-(4-chlorophenyl)-5-(1,1-dimethoxyethyl)-1H-pyrazol-4-yl |
| 605 | pyridin-3-yl | H | 4-({6-[3-(trifluoromethyl)-1H-pyrazol-1-yl]pyridin-2- |
| | | | yl}oxy)phenyl |
| 606 | pyridin-3-yl | H | 1-[2-(1-methoxyethyl)phenyl]-5-methyl-1H-pyrazol-4-yl |
| 607 | pyridin-3-yl | H | 6-(4-chloro-2-cyanophenoxy)pyridin-3-yl |
| 608 | pyridin-3-yl | H | 4-{[4-(trifluoromethyl)-1H-pyrazol-1-yl]methyl}phenyl |
| 609 | pyridin-3-yl | H | 6-methoxypyridin-3-yl |
| 610 | pyridin-3-yl | H | 4-[(ethoxyimino)methyl]phenyl |
| 611 | pyridin-3-yl | H | 4-{[acetyl(cyclopentyl)amino]methyl}phenyl |
| 612 | pyridin-3-yl | H | 3-{[3,5-bis(trifluoromethyl)-1H-pyrazol-1-yl]methyl}phenyl |
| 613 | pyridin-3-yl | H | 1-(2-bromo-4-chlorophenyl)-1H-pyrazol-4-yl |
| 614 | pyridin-3-yl | H | 1-(2-chlorophenyl)-5-(trifluoromethyl)-1H-pyrazol-4-yl |
| 615 | pyridin-3-yl | H | 1-(2,6-dibromophenyl)-5-(difluoromethyl)-1H-pyrazol-4-yl |
| 616 | pyridin-3-yl | H | 6-[(4-methoxyphenyl)sulphanyl]pyridin-3-yl |
| 617 | pyridin-3-yl | H | 1-(2,6-dichlorophenyl)-5-(1-methoxyethyl)-1H-pyrazol-4-yl |
| 618 | pyridin-3-yl | H | 4-[(6-chloropyridin-2-yl)oxy]phenyl |
| 619 | pyridin-3-yl | H | 3-fluoro-4-{[3-(trifluoromethyl)-1H-pyrazol-1- |
| | | | yl]methyl}phenyl |
| 620 | pyridin-3-yl | H | 4-{[2-(3,5-dichlorophenyl)propan-2-yl]carbamoyl}phenyl |
| 621 | pyridin-3-yl | H | 3-(1H-1,2,4-triazol-1-ylmethyl)phenyl |
| 622 | pyridin-3-yl | H | 2-(3-chloropyridin-2-yl)-1,3-thiazol-4-yl |
| 623 | pyridin-3-yl | H | 5-(1-methoxyethyl)-1-(pyrimidin-2-yl)-1H-pyrazol-4-yl |
| 624 | pyridin-3-yl | H | 1-(3-chloropyridin-2-yl)-5-(1,1-dimethoxyethyl)-1H-pyrazol- |
| | | | 4-yl |
| 625 | pyridin-3-yl | H | 1-(2-bromo-4-fluorophenyl)-5-methyl-1H-pyrazol-4-yl |
| 626 | pyridin-3-yl | H | 3-(butanoylamino)-4-methylphenyl |
| 627 | pyridin-3-yl | H | 5-(methoxymethyl)-1-[3-(trifluoromethyl)pyridin-2-yl]-1H- |
| | | | pyrazol-4-yl |
| 628 | pyridin-3-yl | H | 1-(2,6-dichloropyridin-3-yl)-5-(1-methoxyethyl)-1H-pyrazol- |
| | | | 4-yl |
| 629 | pyridin-3-yl | H | 1-[2-(difluoromethyl)phenyl]-5-methyl-1H-pyrazol-4-yl |
| 630 | pyridin-3-yl | H | 2-[2-(trifluoromethyl)phenyl]-1,3-thiazol-5-yl |
| 631 | pyridin-3-yl | H | 2-[2-(trifluoromethyl)pyridin-3-yl]-1,3-thiazol-5-yl |
| 632 | pyridin-3-yl | H | 2-(2-chloropyridin-3-yl)-1,3-thiazol-5-yl |
| 633 | pyridin-3-yl | H | 1-(2,6-dichloropyridin-3-yl)-5-methyl-1H-pyrazol-4-yl |
| 634 | pyridin-3-yl | H | 1-(3-chloropyridin-2-yl)-5-(methoxymethyl)-1H-pyrazol-4-yl |
| 635 | pyridin-3-yl | H | 3-[(cyclopentylcarbonyl)amino]-4-methylphenyl |
| 636 | pyridin-3-yl | H | 6-ethoxypyridin-3-yl |
| 637 | pyridin-3-yl | H | 2-(2,6-dichlorophenyl)-1,3-thiazol-4-yl |
| 638 | pyridin-3-yl | H | 5-chloro-6-[(4-methylpyrimidin-2-yl)sulphanyl]pyridin-3-yl |
| 639 | pyridin-3-yl | H | 1-[4-chloro-2-(trifluoromethyl)phenyl]-5-(1-methoxyethyl)- |
| | | | 1H-pyrazol-4-yl |
| 640 | pyridin-3-yl | H | 6-[3-(trifluoromethoxy)phenoxy]pyridin-3-yl |
| 641 | pyridin-3-yl | H | 5-(difluoromethyl)-1-(pyrimidin-2-yl)-1H-pyrazol-4-yl |
| 642 | pyridin-3-yl | H | 1-(3-bromopyridin-2-yl)-5-(1-methoxyethyl)-1H-pyrazol-4-yl |
| 643 | pyridin-3-yl | H | 4-methyl-3-[(3-methylbut-2-enoyl)amino]phenyl |
| 644 | pyridin-3-yl | H | 1-[3-(methoxymethyl)pyridin-2-yl]-1H-pyrazol-4-yl |
| 645 | pyridin-3-yl | H | 6-(naphthalen-2-yloxy)pyridin-3-yl |
| 646 | pyridin-3-yl | H | 1-(2-chlorophenyl)-5-(1-methoxyethyl)-1H-pyrazol-4-yl |
| 647 | pyridin-3-yl | H | 1-[2-(difluoromethyl)phenyl]-5-(1-methoxyethyl)-1H-pyrazol- |
| | | | 4-yl |
| 648 | pyridin-3-yl | H | 3-[(cyclopentylcarbonyl)amino]-4-methoxyphenyl |
| 649 | pyridin-3-yl | H | 2-[3-(trifluoromethyl)pyridin-2-yl]-1,3-thiazol-5-yl |
| 650 | pyridin-3-yl | H | 4-{[3-(methoxycarbonyl)-1H-1,2,4-triazol-1-yl]methyl}phenyl |
| 651 | pyridin-3-yl | H | 1-[3-(1,1-difluoroethyl)pyridin-2-yl]-1H-pyrazol-4-yl |
| 652 | pyridin-3-yl | H | 6-{[3-(pyridin-2-yl)-1H-pyrazol-1-yl]methyl}pyridin-3-yl |
| 653 | pyridin-3-yl | H | 1-(3-chloropyridin-2-yl)-5-(trifluoromethyl)-1H-pyrazol-4-yl |
| 654 | pyridin-3-yl | H | 2-{[3-(pyridin-2-yl)-1H-pyrazol-1-yl]methyl}pyridin-4-yl |
| 655 | pyridin-3-yl | H | 3-[(methoxyacetyl)amino]-4-methylphenyl |
| 656 | pyridin-3-yl | H | 1-(3-bromopyridin-2-yl)-5-(trifluoromethyl)-1H-pyrazol-4-yl |
| 657 | pyridin-3-yl | H | 4-[(methoxyimino)methyl]phenyl |
| 658 | pyridin-3-yl | H | 1-(pyrimidin-2-yl)-5-(trifluoromethyl)-1H-pyrazol-4-yl |
| 659 | pyridin-3-yl | H | 2-(2-chlorophenyl)-1,3-thiazol-4-yl |
| 660 | pyridin-3-yl | H | 4-methoxy-3-[(propan-2-ylsulphonyl)amino]phenyl |
| 661 | pyridin-3-yl | H | 6-(3-cyanophenoxy)pyridin-3-yl |
| 662 | pyridin-3-yl | H | 4-fluoro-3-[(2-methylpropanoyl)amino]phenyl |
| 663 | pyridin-3-yl | H | 1-(2-bromophenyl)-5-(difluoromethyl)-1H-pyrazol-4-yl |
| 664 | pyridin-3-yl | H | 5-(1-methoxyethyl)-1-(2,2,2-trifluoroethyl)-1H-pyrazol-4-yl |
| 665 | pyridin-3-yl | H | 1-(2,6-dichlorophenyl)-5-(1-fluoroethyl)-1H-pyrazol-4-yl |
| 666 | pyridin-3-yl | H | 3-(acetylamino)-5-(trifluoromethyl)phenyl |
| 667 | pyridin-3-yl | H | 2-[3-(methoxymethyl)pyridin-2-yl]-1,3-thiazol-4-yl |
| 668 | pyridin-3-yl | H | 2-[3-(1-methoxyethyl)pyridin-2-yl]-1,3-thiazol-5-yl |
| 669 | pyridin-3-yl | H | 1-[3-(dimethoxymethyl)pyridin-2-yl]-1H-pyrazol-4-yl |
| 670 | pyridin-3-yl | H | 5-(1-methoxyethyl)-1-(2-methylphenyl)-1H-pyrazol-4-yl |
| 671 | pyridin-3-yl | H | 1-(2-bromo-6-chlorophenyl)-5-(1-methoxyethyl)-1H-pyrazol- |
| | | | 4-yl |
| 672 | pyridin-3-yl | H | 5-chloro-6-[4-(methylsulphonyl)phenoxy]pyridin-3-yl |
| 673 | pyridin-3-yl | H | 1-[3-(1-methoxyethyl)pyridin-2-yl]-1H-pyrazol-4-yl |
| 674 | pyridin-3-yl | H | 6-(4-tert-butylphenoxy)pyridin-3-yl |
| 675 | pyridin-3-yl | H | 1-(2,4-dichlorophenyl)-5-methyl-1H-pyrazol-4-yl |
| 676 | pyridin-3-yl | H | 3-(but-2-enoylamino)-4-methylphenyl |
| 677 | pyridin-3-yl | H | 4-{[4-(pyrimidin-2-yl)-1H-pyrazol-1-yl]methyl}phenyl |
| 678 | pyridin-3-yl | H | 4-{[6-(1H-pyrazol-1-yl)pyridin-2-yl]oxy}phenyl |
| 679 | pyridin-3-yl | H | 4-[(2-methoxypyrimidin-4-yl)oxy]phenyl |
| 680 | pyridin-3-yl | H | 1-[4-chloro-2-(trifluoromethyl)phenyl]-5-(methoxymethyl)- |
| | | | 1H-pyrazol-4-yl |
| 681 | pyridin-3-yl | H | 5-(trifluoromethyl)-1-[2-(trifluoromethyl)phenyl]-1H-pyrazol- |
| | | | 4-yl |
| 682 | pyridin-3-yl | H | 2-[5-chloro-3-(trifluoromethyl)pyridin-2-yl]-1,3-thiazol-5-yl |
| 683 | pyridin-3-yl | H | 4-{[4-(3-chloropyridin-2-yl)-1H-pyrazol-1-yl]methyl}phenyl |
| 684 | pyridin-3-yl | H | 4-[(2,2,3,3,4,4,4-heptafluorobutanoyl)(methyl)amino]phenyl |
| 685 | pyridin-3-yl | H | 1-(2-bromophenyl)-5-(methoxymethyl)-1H-pyrazol-4-yl |
| 686 | pyridin-3-yl | H | 6-[(4-bromo-1H-pyrazol-1-yl)methyl]pyridin-3-yl |
| 687 | pyridin-3-yl | H | 3-chloro-4-{[3-(trifluoromethyl)-1H-pyrazol-1- |
| | | | yl]methyl}phenyl |
| 688 | pyridin-3-yl | H | 6-{[4-(trifluoromethyl)-1H-pyrazol-1-yl]methyl}pyridin-3-yl |
| 689 | pyridin-3-yl | H | 1-(3-bromopyridin-2-yl)-5-(difluoromethyl)-1H-pyrazol-4-yl |
| 690 | pyridin-3-yl | CH₃ | 1-(2,6-dichloropyridin-3-yl)-5-(trifluoromethyl)-1H-pyrazol-4- |
| | | | yl |
| 691 | pyridin-3-yl | CH₃ | 5-(1-methoxyethyl)-1-[3-(trifluoromethyl)pyridin-2-yl]-1H- |
| | | | pyrazol-4-yl |
| 692 | pyridin-3-yl | CH₃ | 1-phenyl-1H-pyrazol-4-yl |
| 693 | pyridin-3-yl | CH₃ | 2-[5-chloro-3-(trifluoromethyl)pyridin-2-yl]-1,3-thiazol-4-yl |
| 694 | pyridin-3-yl | CH₃ | 3-(trifluoromethyl)-1H-pyrazol-4-yl |
| 695 | pyridin-3-yl | CH₃ | 1-[3-(difluoromethyl)pyridin-2-yl]-1H-pyrazol-4-yl |
| 696 | pyridin-3-yl | CH₃ | 5-(trifluoromethyl)-1-[3-(trifluoromethyl)pyridin-2-yl]-1H- |
| | | | pyrazol-4-yl |
| 697 | pyridin-3-yl | CH₃ | 1-(2-bromophenyl)-5-(1-methoxyethyl)-1H-pyrazol-4-yl |
| 698 | pyridin-3-yl | CH₃ | 1-(1,3-thiazol-2-ylmethyl)-3-(trifluoromethyl)-1H-pyrazol-4-yl |
| 699 | pyridin-3-yl | CH₃ | 2-[3-(1-methoxyethyl)pyridin-2-yl]-1,3-thiazol-4-yl |
| 700 | pyridin-3-yl | CH₃ | 5-(1-fluoroethyl)-1-(pyrimidin-2-yl)-1H-pyrazol-4-yl |
| 701 | pyridin-3-yl | CH₃ | 1-[2-(trifluoromethyl)phenyl]-1H-pyrazol-4-yl |
| 702 | pyridin-3-yl | CH₃ | 1-[3-chloro-2-(trifluoromethyl)phenyl]-5-(methoxymethyl)- |
| | | | 1H-pyrazol-4-yl |
| 703 | pyridin-3-yl | CH₃ | 4-[(2-cyanoethyl)(methyl)carbamoyl]phenyl |
| 704 | pyridin-3-yl | CH₃ | 1-benzyl-3-(trifluoromethyl)-1H-pyrazol-4-yl |
| 705 | pyridin-3-yl | CH₃ | 2-(pyrimidin-2-yl)-1,3-thiazol-5-yl |
| 706 | pyridin-3-yl | CH₃ | 4-(dimethylcarbamoyl)phenyl |
| 707 | pyridin-3-yl | CH₃ | 1-(2,6-dichlorophenyl)-5-methyl-1H-pyrazol-4-yl |
| 708 | pyridin-3-yl | CH₃ | 1-(2-chloropyridin-3-yl)-5-(difluoromethyl)-1H-pyrazol-4-yl |
| 709 | pyridin-3-yl | CH₃ | 1-(3-chloropyridin-2-yl)-5-(dimethoxymethyl)-1H-pyrazol-4-yl |
| 710 | pyridin-3-yl | CH₃ | 1-(2-bromo-6-chlorophenyl)-5-(dimethoxymethyl)-1H- |
| | | | pyrazol-4-yl |
| 711 | pyridin-3-yl | CH₃ | 4-methoxy-3-[3-(pyridin-2-yl)-1H-pyrazol-1-yl]phenyl |
| 712 | pyridin-3-yl | CH₃ | 3-chloro-4-{[4-(trifluoromethyl)-1H-pyrazol-1- |
| | | | yl]methyl}phenyl |
| 713 | pyridin-3-yl | CH₃ | 4-{[3-(3-chloropyridin-2-yl)-1H-pyrazol-1-yl]methyl}phenyl |
| 714 | pyridin-3-yl | CH₃ | 6-{[4-(pyridin-2-yl)-1H-pyrazol-1-yl]methyl}pyridin-3-yl |
| 715 | pyridin-3-yl | CH₃ | 4-methoxy-3-[3-(pyrimidin-2-yl)-1H-pyrazol-1-yl]phenyl |
| 716 | pyridin-3-yl | CH₃ | 4-{[3-(pyridin-2-yl)-1H-pyrazol-1-yl]methyl}phenyl |
| 717 | pyridin-3-yl | CH₃ | 1-(2-chloropyridin-3-yl)-5-(1-methoxyethyl)-1H-pyrazol-4-yl |
| 718 | pyridin-3-yl | CH₃ | 2-(3,5-dichloropyridin-2-yl)-1,3-thiazol-4-yl |
| 719 | pyridin-3-yl | CH₃ | 6-(propylamino)pyridin-3-yl |
| 720 | pyridin-3-yl | CH₃ | 6-[(2-chloropyrimidin-4-yl)oxy]pyridin-3-yl |
| 721 | pyridin-3-yl | CH₃ | 1-(3-chloropyridin-2-yl)-5-(1-methoxyethyl)-1H-pyrazol-4-yl |
| 722 | pyridin-3-yl | CH₃ | 2-[2-(trifluoromethyl)phenyl]-1,3-thiazol-4-yl |
| 723 | pyridin-3-yl | CH₃ | 2-(2,6-dichloropyridin-3-yl)-1,3-thiazol-4-yl |
| 724 | pyridin-3-yl | CH₃ | 1-ethyl-5-(trifluoromethyl)-1H-pyrazol-4-yl |
| 725 | pyridin-3-yl | CH₃ | 1-[3-(difluoromethyl)pyridin-2-yl]-5-methyl-I H-pyrazol-4-yl |
| 726 | pyridin-3-yl | CH₃ | 1-(2,4-dichlorophenyl)-1H-pyrazol-4-yl |
| 727 | pyridin-3-yl | CH₃ | 2-[4-chloro-2-(trifluoromethyl)phenyl]-1,3-thiazol-5-yl |
| 728 | pyridin-3-yl | CH₃ | 4-[(2-methylpyrimidin-4-yl)oxy]phenyl |
| 729 | pyridin-3-yl | CH₃ | 6-[(4-nitrophenyl)sulphanyl]pyridin-3-yl |
| 730 | pyridin-3-yl | CH₃ | 1-[4-fluoro-2-(trifluoromethyl)phenyl]-5-(methoxymethyl)-1H- |
| | | | pyrazol-4-yl |
| 731 | pyridin-3-yl | CH₃ | 3-{[3,5-bis(trifluoromethyl)-1H-1,2,4-triazol-1- |
| | | | yl]methyl}phenyl |
| 732 | pyridin-3-yl | CH₃ | 4-{[3,5-bis(trifluoromethyl)-1H-pyrazol-1-yl]methyl}phenyl |
| 733 | pyridin-3-yl | CH₃ | 1-(2-chlorophenyl)-5-(1,1-dimethoxyethyl)-1H-pyrazol-4-yl |
| 734 | pyridin-3-yl | CH₃ | 1-(3-chloropyridin-2-yl)-5-(1,1-difluoroethyl)-1H-pyrazol-4-yl |
| 735 | pyridin-3-yl | CH₃ | 1-(2,6-dichloropyridin-3-yl)-5-(methoxymethyl)-1H-pyrazol-4- |
| | | | yl |
| 736 | pyridin-3-yl | CH₃ | 3-(4-chloro-1H-pyrazol-1-yl)-4-methoxyphenyl |
| 737 | pyridin-3-yl | CH₃ | 2-(2,4-dichlorophenyl)-1,3-thiazol-4-yl |
| 738 | pyridin-3-yl | CH₃ | 1-(2-chloropyridin-3-yl)-5-(1,1-difluoroethyl)-1H-pyrazol-4-yl |
| 739 | pyridin-3-yl | CH₃ | 1-[4-chloro-2-(trifluoromethyl)phenyl]-5-(difluoromethyl)-1H- |
| | | | pyrazol-4-yl |
| 740 | pyridin-3-yl | CH₃ | 1-[2-(1-methoxyethyl)phenyl]-1H-pyrazol-4-yl |
| 741 | pyridin-3-yl | CH₃ | 2-(2,4-dichlorophenyl)-1,3-thiazol-5-yl |
| 742 | pyridin-3-yl | CH₃ | 6-[2-chloro-4-(methylsulphanyl)phenoxy]pyridin-3-yl |
| 743 | pyridin-3-yl | CH₃ | 1-(2-bromo-6-chlorophenyl)-5-(difluoromethyl)-1H-pyrazol-4- |
| | | | yl |
| 744 | pyridin-3-yl | CH₃ | 1-[4-chloro-2-(trifluoromethyl)phenyl]-5-methyl-1H-pyrazol- |
| | | | 4-yl |
| 745 | pyridin-3-yl | CH₃ | 1-(4-chlorophenyl)-1H-pyrazol-4-yl |
| 746 | pyridin-3-yl | CH₃ | 2-[2-(difluoromethyl)phenyl]-1,3-thiazol-5-yl |
| 747 | pyridin-3-yl | CH₃ | 1-ethyl-3-(trifluoromethyl)-1H-pyrazol-4-yl |
| 748 | pyridin-3-yl | CH₃ | 2-(2-chloropyridin-3-yl)-1,3-thiazol-4-yl |
| 749 | pyridin-3-yl | CH₃ | 1-(2,3-dichlorophenyl)-5-(methoxymethyl)-1H-pyrazol-4-yl |
| 750 | pyridin-3-yl | CH₃ | 3-(hydroxymethyl)-1-methyl-1H-pyrazol-4-yl |
| 751 | pyridin-3-yl | CH₃ | 1-(propan-2-yl)-3-(trifluoromethyl)-1H-pyrazol-4-yl |
| 752 | pyridin-3-yl | CH₃ | 2-[3-(difluoromethyl)pyridin-2-yl]-1,3-thiazol-5-yl |
| 753 | pyridin-3-yl | CH₃ | 4-(pyrrolidin-l-ylcarbonyl)phenyl |
| 754 | pyridin-3-yl | CH₃ | 2-(3,5-dichloropyridin-2-yl)-1,3-thiazol-5-yl |
| 755 | pyridin-3-yl | CH₃ | 1-(2,6-dichloropyridin-3-yl)-5-(difluoromethyl)-1H-pyrazol-4- |
| | | | yl |
| 756 | pyridin-3-yl | CH₃ | 2-[2-(difluoromethyl)phenyl]-1,3-thiazol-4-yl |
| 757 | pyridin-3-yl | CH₃ | 1-(difluoromethyl)-3-(trifluoromethyl)-1H-pyrazol-4-yl |
| 758 | pyridin-3-yl | CH₃ | 6-[(6-chloropyridin-2-yl)oxy]pyridin-3-yl |
| 759 | pyridin-3-yl | CH₃ | 1-(2-bromophenyl)-5-(trifluoromethyl)-1H-pyrazol-4-yl |
| 760 | pyridin-3-yl | CH₃ | 5-(difluoromethyl)-1-(2-methylphenyl)-1H-pyrazol-4-yl |
| 761 | pyridin-3-yl | CH₃ | 2-[2-(1-methoxyethyl)phenyl]-1,3-thiazol-5-yl |
| 762 | pyridin-3-yl | CH₃ | 1-(2-chlorophenyl)-5-(1,1-difluoroethyl)-1H-pyrazol-4-yl |
| 763 | pyridin-3-yl | CH₃ | 1-(2-bromo-4-fluorophenyl)-1H-pyrazol-4-yl |
| 764 | pyridin-3-yl | CH₃ | 6-[(cyclopropylmethyl)amino]pyridin-3-yl |
| 765 | pyridin-3-yl | CH₃ | 6-[(2-methoxypyrimidin-4-yl)oxy]pyridin-3-yl |
| 766 | pyridin-3-yl | CH₃ | 1-[2-(1,1-difluoroethyl)phenyl]-5-methyl-1H-pyrazol-4-yl |
| 767 | pyridin-3-yl | CH₃ | 3-{[3,5-bis(trifluoromethyl)-1H-1,2,4-triazol-1-yl]methyl}-4- |
| | | | chlorophenyl |
| 768 | pyridin-3-yl | CH₃ | 6-(morpholin-4-yl)pyridin-3-yl |
| 769 | pyridin-3-yl | CH₃ | 2-(pyrimidin-2-yl)-1,3-thiazol-4-yl |
| 770 | pyridin-3-yl | CH₃ | 2-{[3-(trifluoromethyl)-1H-pyrazol-1-yl]methyl}pyridin-4-yl |
| 771 | pyridin-3-yl | CH₃ | 1-(2-chloropyridin-3-yl)-5-(trifluoromethyl)-1H-pyrazol-4-yl |
| 772 | pyridin-3-yl | CH₃ | 6-(4-bromophenoxy)pyridin-3-yl |
| 773 | pyridin-3-yl | CH₃ | 1-(2-chloro-4-fluorophenyl)-1H-pyrazol-4-yl |
| 774 | pyridin-3-yl | CH₃ | 4-methoxy-3-[4-(pyridin-2-yl)-1H-pyrazol-1-yl]phenyl |
| 775 | pyridin-3-yl | CH₃ | 1-(2-chlorophenyl)-1H-pyrazol-4-yl |
| 776 | pyridin-3-yl | CH₃ | 1-(2-chlorophenyl)-5-methyl-1H-pyrazol-4-yl |
| 777 | pyridin-3-yl | CH₃ | 2-[2-(1-methoxyethyl)phenyl]-1,3-thiazol-4-yl |
| 778 | pyridin-3-yl | CH₃ | 6-{[6-(1H-pyrazol-1-yl)pyridin-2-yl]oxy}pyridin-3-yl |
| 779 | pyridin-3-yl | CH₃ | 2-[4-chloro-2-(trifluoromethyl)phenyl]-1,3-thiazol-4-yl |
| 780 | pyridin-3-yl | CH₃ | 2-{[4-(trifluoromethyl)-1H-pyrazol-1-yl]methyl}pyridin-4-yl |
| 781 | pyridin-3-yl | CH₃ | 1-(2,4-difluorophenyl)-1H-pyrazol-4-yl |
| 782 | pyridin-3-yl | CH₃ | 1-(2-methylphenyl)-5-(trifluoromethyl)-1H-pyrazol-4-yl |
| 783 | pyridin-3-yl | CH₃ | 2-[3-(difluoromethyl)pyridin-2-yl]-1,3-thiazol-4-yl |
| 784 | pyridin-3-yl | CH₃ | 1-(pyrimidin-2-ylmethyl)-3-(trifluoromethyl)-1H-pyrazol-4-yl |
| 785 | pyridin-3-yl | CH₃ | 1-(2,4-dichlorophenyl)-5-(methoxymethyl)-1H-pyrazol-4-yl |
| 786 | pyridin-3-yl | CH₃ | 1-(2,6-dichlorophenyl)-5-(trifluoromethyl)-1H-pyrazol-4-yl |
| 787 | pyridin-3-yl | CH₃ | 2-(2,6-dichloropyridin-3-yl)-1,3-thiazol-5-yl |
| 788 | pyridin-3-yl | CH₃ | 6-{[(4,6-dimethoxypyrimidin-2-yl)methyl]sulphanyl}pyridin- |
| | | | 3-yl |
| 789 | pyridin-3-yl | CH₃ | 1-[3-chloro-2-(trifluoromethyl)phenyl]-5-(1-methoxyethyl)- |
| | | | 1H-pyrazol-4-yl |
| 790 | pyridin-3-yl | CH₃ | 3-{[4-(trifluoromethyl)-1H-pyrazol-1-yl]methyl}phenyl |
| 791 | pyridin-3-yl | CH₃ | 1-(ethoxymethyl)-3-(trifluoromethyl)-1H-pyrazol-4-yl |
| 792 | pyridin-3-yl | CH₃ | 1-(3-bromopyridin-2-yl)-5-(methoxymethyl)-1H-pyrazol-4-yl |
| 793 | pyridin-3-yl | CH₃ | 1-(2-bromo-6-chlorophenyl)-5-(trifluoromethyl)-1H-pyrazol-4- |
| | | | yl |
| 794 | pyridin-3-yl | CH₃ | 1-ethenyl-3-(trifluoromethyl)-1H-pyrazol-4-yl |
| 795 | pyridin-3-yl | CH₃ | 3-(4-bromo-1H-pyrazol-1-yl)-4-methoxyphenyl |
| 796 | pyridin-3-yl | CH₃ | 5-methyl-1-(2-methylphenyl)-1H-pyrazol-4-yl |
| 797 | pyridin-3-yl | CH₃ | 1-[3-(difluoromethyl)pyridin-2-yl]-5-(methoxymethyl)-1H- |
| | | | pyrazol-4-yl |
| 798 | pyridin-3-yl | CH₃ | 2-[2-(methoxymethyl)phenyl]-1,3-thiazol-5-yl |
| 799 | pyridin-3-yl | CH₃ | 1-(ethoxymethyl)-5-(trifluoromethyl)-1H-pyrazol-4-yl |
| 800 | pyridin-3-yl | CH₃ | 4-{[4-(pyridin-2-yl)-1H-pyrazol-1-yl]methyl}phenyl |
| 801 | pyridin-3-yl | CH₃ | 1-[3-(1,1-difluoroethyl)pyridin-2-yl]-5-methyl-1H-pyrazol-4-yl |
| 802 | pyridin-3-yl | CH₃ | 1-(2,6-dichlorophenyl)-5-(difluoromethyl)-1H-pyrazol-4-yl |
| 803 | pyridin-3-yl | CH₃ | 1-[4-fluoro-2-(trifluoromethyl)phenyl]-5-methyl-1H-pyrazol-4- |
| | | | yl |
| 804 | pyridin-3-yl | CH₃ | 2-[2-(methoxymethyl)phenyl]-1,3-thiazol-4-yl |
| 805 | pyridin-3-yl | CH₃ | 1-(2-bromophenyl)-5-methyl-1H-pyrazol-4-yl |
| 806 | pyridin-3-yl | CH₃ | 1-(2,3-difluorophenyl)-1H-pyrazol-4-yl |
| 807 | pyridin-3-yl | CH₃ | 4-{[3-(pentafluoroethyl)-1H-1,2,4-triazol-1-yl]methyl}phenyl |
| 808 | pyridin-3-yl | CH₃ | 4-{[3-(pyrimidin-2-yl)-1H-pyrazol-1-yl]methyl}phenyl |
| 809 | pyridin-3-yl | CH₃ | 1-(2-chlorophenyl)-5-(difluoromethyl)-1H-pyrazol-4-yl |
| 810 | pyridin-3-yl | CH₃ | 5-(difluoromethyl)-1-[2-(trifluoromethyl)phenyl]-1H-pyrazol- |
| | | | 4-yl |
| 811 | pyridin-3-yl | CH₃ | 1-[2-(dimethoxymethyl)phenyl]-1H-pyrazol-4-yl |
| 812 | pyridin-3-yl | CH₃ | 1-(3-chlorophenyl)-1H-pyrazol-4-yl |
| 813 | pyridin-3-yl | CH₃ | 4-(propylsulphanyl)phenyl |
| 814 | pyridin-3-yl | CH₃ | 1-[2-(difluoromethyl)phenyl]-5-(methoxymethyl)-1H-pyrazol- |
| | | | 4-yl |
| 815 | pyridin-3-yl | CH₃ | 4-methoxy-3-[4-(pyrimidin-2-yl)-1H-pyrazol-1-yl]phenyl |
| 816 | pyridin-3-yl | CH₃ | 1-(2-chlorophenyl)-5-(methoxymethyl)-1H-pyrazol-4-yl |
| 817 | pyridin-3-yl | CH₃ | 2-(3-chloropyridin-2-yl)-1,3-thiazol-5-yl |
| 818 | pyridin-3-yl | CH₃ | 1-(2-fluorophenyl)-1H-pyrazol-4-yl |
| 819 | pyridin-3-yl | CH₃ | 6-{[2-(morpholin-4-yl)ethyl]amino}pyridin-3-yl |
| 820 | pyridin-3-yl | CH₃ | 3-formyl-1-methyl-1H-pyrazol-4-yl |
| 821 | pyridin-3-yl | CH₃ | 1-(3-chlorophenyl)-5-(1,1-dimethoxyethyl)-1H-pyrazol-4-yl |
| 822 | pyridin-3-yl | CH₃ | 6-{[5-(methoxycarbonyl)pyridin-3-yl]oxy}pyridin-3-yl |
| 823 | pyridin-3-yl | CH₃ | 3-{[4-(trifluoromethyl)benzyl]oxy}phenyl |
| 824 | pyridin-3-yl | CH₃ | 1-(2,4-dichlorophenyl)-5-(1,1-dimethoxyethyl)-1H-pyrazol-4- |
| | | | yl |
| 825 | pyridin-3-yl | CH₃ | 4-{[4-bromo-3-(trifluoromethyl)-1H-pyrazol-1- |
| | | | yl]methyl}phenyl |
| 826 | pyridin-3-yl | CH₃ | 5-methyl-1-(2-methylpyridin-3-yl)-1H-pyrazol-4-yl |
| 827 | pyridin-3-yl | CH₃ | 1-(2,4-difluorophenyl)-5-methyl-1H-pyrazol-4-yl |
| 828 | pyridin-3-yl | CH₃ | 4-[(4-bromo-1H-pyrazol-1-yl)methyl]phenyl |
| 829 | pyridin-3-yl | CH₃ | 6-(1H-pyrazol-1-yl)pyridin-3-yl |
| 830 | pyridin-3-yl | CH₃ | 5-(methoxymethyl)-1-[2-(trifluoromethyl)phenyl]-1H-pyrazol- |
| | | | 4-yl |
| 831 | pyridin-3-yl | CH₃ | 5-methylpyridin-3-yl |
| 832 | pyridin-3-yl | CH₃ | 2-[2-(trifluoromethyl)pyridin-3-yl]-1,3-thiazol-4-yl |
| 833 | pyridin-3-yl | CH₃ | 3-methyl-1H-pyrazol-4-yl |
| 834 | pyridin-3-yl | CH₃ | 3-fluoro-4-{[4-(trifluoromethyl)-1H-pyrazol-1- |
| | | | yl]methyl}phenyl |
| 835 | pyridin-3-yl | CH₃ | 2-[3-(methoxymethyl)pyridin-2-yl]-1,3-thiazol-5-yl |
| 836 | pyridin-3-yl | CH₃ | 6-({6-[3-(trifluoromethyl)-1H-pyrazol-1-yl]pyridin-2- |
| | | | yl}oxy)pyridin-3-yl |
| 837 | pyridin-3-yl | CH₃ | 3-{[3-(pyridin-2-yl)-1H-pyrazol-1-yl]methyl}phenyl |
| 838 | pyridin-3-yl | CH₃ | 1-(2-bromo-4-chlorophenyl)-5-methyl-1H-pyrazol-4-yl |
| 839 | pyridin-3-yl | CH₃ | 2-[3-(trifluoromethyl)pyridin-2-yl]-1,3-thiazol-4-yl |
| 840 | pyridin-3-yl | CH₃ | 4-[(2-chloropyrimidin-4-yl)oxy]phenyl |
| 841 | pyridin-3-yl | CH₃ | 1-[3-(trifluoromethyl)pyridin-2-yl]-1H-pyrazol-4-yl |
| 842 | pyridin-3-yl | CH₃ | 3-cyano-4-(1H-1,2,4-triazol-1-yl)phenyl |
| 843 | pyridin-3-yl | CH₃ | 1-(2,6-dibromophenyl)-5-(trifluoromethyl)-1H-pyrazol-4-yl |
| 844 | pyridin-3-yl | CH₃ | 5-(pyrazin-2-yl)-1-(2,2,2-trifluoroethyl)-1H-pyrazol-4-yl |
| 845 | pyridin-3-yl | CH₃ | 1-(2,6-dichlorophenyl)-5-(dimethoxymethyl)-1H-pyrazol-4-yl |
| 846 | pyridin-3-yl | CH₃ | 1-(4-chlorophenyl)-5-(1,1-dimethoxyethyl)-1H-pyrazol-4-yl |
| 847 | pyridin-3-yl | CH₃ | 4-({6-[3-(trifluoromethyl)-1H-pyrazol-1-yl]pyridin-2- |
| | | | yl}oxy)phenyl |
| 848 | pyridin-3-yl | CH₃ | 1-[2-(1-methoxyethyl)phenyl]-5-methyl-1H-pyrazol-4-yl |
| 849 | pyridin-3-yl | CH₃ | 1-propyl-3-(trifluoromethyl)-1H-pyrazol-4-yl |
| 850 | pyridin-3-yl | CH₃ | 4-{[4-(trifluoromethyl)-1H-pyrazol-1-yl]methyl}phenyl |
| 851 | pyridin-3-yl | CH₃ | 4-[(ethoxyimino)methyl]phenyl |
| 852 | pyridin-3-yl | CH₃ | 1-(2-bromo-4-chlorophenyl)-1H-pyrazol-4-yl |
| 853 | pyridin-3-yl | CH₃ | 1-(2,6-dibromophenyl)-5-(difluoromethyl)-1H-pyrazol-4-yl |
| 854 | pyridin-3-yl | CH₃ | 1-(2,6-dichlorophenyl)-5-(1-methoxyethyl)-1H-pyrazol-4-yl |
| 855 | pyridin-3-yl | CH₃ | 4-[(6-chloropyridin-2-yl)oxy]phenyl |
| 856 | pyridin-3-yl | CH₃ | 3-fluoro-4-{[3-(trifluoromethyl)-1H-pyrazol-1- |
| | | | yl]methyl}phenyl |
| 857 | pyridin-3-yl | CH₃ | 6-{[3-(trifluoromethyl)-1H-pyrazol-1-yl]methyl}pyridin-3-yl |
| 858 | pyridin-3-yl | CH₃ | 1-(3-chloropyridin-2-yl)-5-(1,1-dimethoxyethyl)-1H-pyrazol- |
| | | | 4-yl |
| 859 | pyridin-3-yl | CH₃ | 1-(2-bromo-4-fluorophenyl)-5-methyl-1H-pyrazol-4-yl |
| 860 | pyridin-3-yl | CH₃ | 5-(methoxymethyl)-1-[3-(trifluoromethyl)pyridin-2-yl]-1H- |
| | | | pyrazol-4-yl |
| 861 | pyridin-3-yl | CH₃ | 6-[3-(trifluoromethyl)-1H-pyrazol-1-yl]pyridin-3-yl |
| 862 | pyridin-3-yl | CH₃ | 1-(2,6-dichloropyridin-3-yl)-5-(1-methoxyethyl)-1H-pyrazol- |
| | | | 4-yl |
| 863 | pyridin-3-yl | CH₃ | 1-[2-(difluoromethyl)phenyl]-5-methyl-1H-pyrazol-4-yl |
| 864 | pyridin-3-yl | CH₃ | 2-[2-(trifluoromethyl)phenyl]-1,3-thiazol-5-yl |
| 865 | pyridin-3-yl | CH₃ | 2-[2-(trifluoromethyl)pyridin-3-yl]-1,3-thiazol-5-yl |
| 866 | pyridin-3-yl | CH₃ | 2-(2-chloropyridin-3-yl)-1,3-thiazol-5-yl |
| 867 | pyridin-3-yl | CH₃ | 1-(2,6-dichloropyridin-3-yl)-5-methyl-1H-pyrazol-4-yl |
| 868 | pyridin-3-yl | CH₃ | 1-(3-chloropyridin-2-yl)-5-(methoxymethyl)-1H-pyrazol-4-yl |
| 869 | pyridin-3-yl | CH₃ | 6-(piperidin-l-yl)pyridin-3-yl |
| 870 | pyridin-3-yl | CH₃ | 2-(2,6-dichlorophenyl)-1,3-thiazol-4-yl |
| 871 | pyridin-3-yl | CH₃ | 1-[4-chloro-2-(trifluoromethyl)phenyl]-5-(1-methoxyethyl)- |
| | | | 1H-pyrazol-4-yl |
| 872 | pyridin-3-yl | CH₃ | 4-[ethyl(methyl)carbamoyl]phenyl |
| 873 | pyridin-3-yl | CH₃ | 1-(3-bromopyridin-2-yl)-5-(1-methoxyethyl)-1H-pyrazol-4-yl |
| 874 | pyridin-3-yl | CH₃ | 1-[3-(methoxymethyl)pyridin-2-yl]-1H-pyrazol-4-yl |
| 875 | pyridin-3-yl | CH₃ | 1-(2-chlorophenyl)-5-(1-methoxyethyl)-1H-pyrazol-4-yl |
| 876 | pyridin-3-yl | CH₃ | 1-[2-(difluoromethyl)phenyl]-5-(1-methoxyethyl)-1H-pyrazol- |
| | | | 4-yl |
| 877 | pyridin-3-yl | CH₃ | 1-(3-chloropyridin-2-yl)-5-methyl-1H-pyrazol-4-yl |
| 878 | pyridin-3-yl | CH₃ | 6-[4-(methylsulphanyl)phenoxy]pyridin-3-yl |
| 879 | pyridin-3-yl | CH₃ | 2-[3-(trifluoromethyl)pyridin-2-yl]-1,3-thiazol-5-yl |
| 880 | pyridin-3-yl | CH₃ | 1-[3-(1,1-difluoroethyl)pyridin-2-yl]-1H-pyrazol-4-yl |
| 881 | pyridin-3-yl | CH₃ | 6-{[3-(pyridin-2-yl)-1H-pyrazol-1-yl]methyl}pyridin-3-yl |
| 882 | pyridin-3-yl | CH₃ | 2-{[3-(pyridin-2-yl)-1H-pyrazol-1-yl]methyl}pyridin-4-yl |
| 883 | pyridin-3-yl | CH₃ | 1-(3-bromopyridin-2-yl)-5-(trifluoromethyl)-1H-pyrazol-4-yl |
| 884 | pyridin-3-yl | CH₃ | 4-[(methoxyimino)methyl]phenyl |
| 885 | pyridin-3-yl | CH₃ | 2-(2-chlorophenyl)-1,3-thiazol-4-yl |
| 886 | pyridin-3-yl | CH₃ | 1-(2-bromophenyl)-5-(difluoromethyl)-1H-pyrazol-4-yl |
| 887 | pyridin-3-yl | CH₃ | 1-(2,6-dichlorophenyl)-5-(1-fluoroethyl)-1H-pyrazol-4-yl |
| 888 | pyridin-3-yl | CH₃ | 6-(pyrrolidin-l-yl)pyridin-3-yl |
| 889 | pyridin-3-yl | CH₃ | 2-[3-(methoxymethyl)pyridin-2-yl]-1,3-thiazol-4-yl |
| 890 | pyridin-3-yl | CH₃ | 2-[3-(1-methoxyethyl)pyridin-2-yl]-1,3-thiazol-5-yl |
| 891 | pyridin-3-yl | CH₃ | 1-[3-(dimethoxymethyl)pyridin-2-yl]-1H-pyrazol-4-yl |
| 892 | pyridin-3-yl | CH₃ | 5-(1-methoxyethyl)-1-(2-methylphenyl)-1H-pyrazol-4-yl |
| 893 | pyridin-3-yl | CH₃ | 1-methyl-1H-benzotriazol-5-yl |
| 894 | pyridin-3-yl | CH₃ | 4-[(butylsulphanyl)methyl]phenyl |
| 895 | pyridin-3-yl | CH₃ | 1H-pyrazol-4-yl |
| 896 | pyridin-3-yl | CH₃ | 1-(2-bromo-6-chlorophenyl)-5-(1-methoxyethyl)-1H-pyrazol- |
| | | | 4-yl |
| 897 | pyridin-3-yl | CH₃ | 1-[3-(1-methoxyethyl)pyridin-2-yl]-1H-pyrazol-4-yl |
| 898 | pyridin-3-yl | CH₃ | 4-[(6-methylpyridin-3-yl)oxy]phenyl |
| 899 | pyridin-3-yl | CH₃ | 3-methoxy-1-methyl-1H-pyrazol-4-yl |
| 900 | pyridin-3-yl | CH₃ | 1-(2,4-dichlorophenyl)-5-methyl-1H-pyrazol-4-yl |
| 901 | pyridin-3-yl | CH₃ | 4-{[4-(pyrimidin-2-yl)-1H-pyrazol-1-yl]methyl}phenyl |
| 902 | pyridin-3-yl | CH₃ | 4-{[6-(1H-pyrazol-1-yl)pyridin-2-yl]oxy}phenyl |
| 903 | pyridin-3-yl | CH₃ | 4-{[3,5-bis(trifluoromethyl)-1H-1,2,4-triazol-1-yl]methyl}-3- |
| | | | chlorophenyl |
| 904 | pyridin-3-yl | CH₃ | 4-[(2-methoxypyrimidin-4-yl)oxy]phenyl |
| 905 | pyridin-3-yl | CH₃ | 1-[4-chloro-2-(trifluoromethyl)phenyl]-5-(methoxymethyl)- |
| | | | 1H-pyrazol-4-yl |
| 906 | pyridin-3-yl | CH₃ | 5-(trifluoromethyl)-1-[2-(trifluoromethyl)phenyl]-1H-pyrazol- |
| | | | 4-yl |
| 907 | pyridin-3-yl | CH₃ | 6-(dimethylamino)pyridin-3-yl |
| 908 | pyridin-3-yl | CH₃ | 2-[5-chloro-3-(trifluoromethyl)pyridin-2-yl]-1,3-thiazol-5-yl |
| 909 | pyridin-3-yl | CH₃ | 4-{[4-(3-chloropyridin-2-yl)-1H-pyrazol-1-yl]methyl}phenyl |
| 910 | pyridin-3-yl | CH₃ | 1-(2-bromophenyl)-5-(methoxymethyl)-1H-pyrazol-4-yl |
| 911 | pyridin-3-yl | CH₃ | 6-[(4-bromo-1H-pyrazol-1-yl)methyl]pyridin-3-yl |
| 912 | pyridin-3-yl | CH₃ | 3-chloro-4-{[3-(trifluoromethyl)-1H-pyrazol-1- |
| | | | yl]methyl}phenyl |
| 913 | pyridin-3-yl | CH₃ | 6-{[4-(trifluoromethyl)-1H-pyrazol-1-yl]methyl}pyridin-3-yl |
| 914 | pyridin-3-yl | CH₃ | 1-(3-bromopyridin-2-yl)-5-(difluoromethyl)-1H-pyrazol-4-yl |
| 915 | pyridin-3-yl | ethyl | 1-(2,6-dichloropyridin-3-yl)-5-(trifluoromethyl)-1H-pyrazol-4- |
| | | | yl |
| 916 | pyridin-3-yl | ethyl | 5-(1-methoxyethyl)-1-[3-(trifluoromethyl)pyridin-2-yl]-1H- |
| | | | pyrazol-4-yl |
| 917 | pyridin-3-yl | ethyl | 1-phenyl-1H-pyrazol-4-yl |
| 918 | pyridin-3-yl | ethyl | 2-[5-chloro-3-(trifluoromethyl)pyridin-2-yl]-1,3-thiazol-4-yl |
| 919 | pyridin-3-yl | ethyl | 3-(trifluoromethyl)-1H-pyrazol-4-yl |
| 920 | pyridin-3-yl | ethyl | 1-[3-(difluoromethyl)pyridin-2-yl]-1H-pyrazol-4-yl |
| 921 | pyridin-3-yl | ethyl | 5-(trifluoromethyl)-1-[3-(trifluoromethyl)pyridin-2-yl]-1H- |
| | | | pyrazol-4-yl |
| 922 | pyridin-3-yl | ethyl | 1-(2-bromophenyl)-5-(1-methoxyethyl)-1H-pyrazol-4-yl |
| 923 | pyridin-3-yl | ethyl | 1-(1,3-thiazol-2-ylmethyl)-3-(trifluoromethyl)-1H-pyrazol-4-yl |
| 924 | pyridin-3-yl | ethyl | 3-(trifluoromethyl)phenyl |
| 925 | pyridin-3-yl | ethyl | 2-[3-(1-methoxyethyl)pyridin-2-yl]-1,3-thiazol-4-yl |
| 926 | pyridin-3-yl | ethyl | 5-(1-fluoroethyl)-1-(pyrimidin-2-yl)-1H-pyrazol-4-yl |
| 927 | pyridin-3-yl | ethyl | 1-[2-(trifluoromethyl)phenyl]-1H-pyrazol-4-yl |
| 928 | pyridin-3-yl | ethyl | 1-[3-chloro-2-(trifluoromethyl)phenyl]-5-(methoxymethyl)- |
| | | | 1H-pyrazol-4-yl |
| 929 | pyridin-3-yl | ethyl | 4-[(2-cyanoethyl)(methyl)carbamoyl]phenyl |
| 930 | pyridin-3-yl | ethyl | 4-[(trifluoroacetyl)amino]phenyl |
| 931 | pyridin-3-yl | ethyl | 1-benzyl-3-(trifluoromethyl)-1H-pyrazol-4-yl |
| 932 | pyridin-3-yl | ethyl | 6-(2,5-dichlorophenoxy)pyridin-3-yl |
| 933 | pyridin-3-yl | ethyl | 2-(pyrimidin-2-yl)-1,3-thiazol-5-yl |
| 934 | pyridin-3-yl | ethyl | 3-(but-2-enoylamino)-4-methoxyphenyl |
| 935 | pyridin-3-yl | ethyl | 4-(dimethylcarbamoyl)phenyl |
| 936 | pyridin-3-yl | ethyl | 4-chloro-3-[(cyclopropylcarbonyl)amino]phenyl |
| 937 | pyridin-3-yl | ethyl | 1-(2,6-dichlorophenyl)-5-methyl-1H-pyrazol-4-yl |
| 938 | pyridin-3-yl | ethyl | 1-(2-chloropyridin-3-yl)-5-(difluoromethyl)-1H-pyrazol-4-yl |
| 939 | pyridin-3-yl | ethyl | 1-(3-chloropyridin-2-yl)-5-(dimethoxymethyl)-1H-pyrazol-4-yl |
| 940 | pyridin-3-yl | ethyl | 5-(difluoromethyl)-1-(2,2,2-trifluoroethyl)-1H-pyrazol-4-yl |
| 941 | pyridin-3-yl | ethyl | 4-[(propan-2-ylsulphanyl)methyl]phenyl |
| 942 | pyridin-3-yl | ethyl | 1-(2-bromo-6-chlorophenyl)-5-(dimethoxymethyl)-1H- |
| | | | pyrazol-4-yl |
| 943 | pyridin-3-yl | ethyl | 4-methoxy-3-[3-(pyridin-2-yl)-1H-pyrazol-1-yl]phenyl |
| 944 | pyridin-3-yl | ethyl | 3-chloro-4-{[4-(trifluoromethyl)-1H-pyrazol-1- |
| | | | yl]methyl}phenyl |
| 945 | pyridin-3-yl | ethyl | 4-methyl-3-[(2-methylbutanoyl)amino]phenyl |
| 946 | pyridin-3-yl | ethyl | 4-{[3-(3-chloropyridin-2-yl)-1H-pyrazol-1-yl]methyl}phenyl |
| 947 | pyridin-3-yl | ethyl | 6-{[4-(pyridin-2-yl)-1H-pyrazol-1-yl]methyl}pyridin-3-yl |
| 948 | pyridin-3-yl | ethyl | 4-methoxy-3-[3-(pyrimidin-2-yl)-1H-pyrazol-1-yl]phenyl |
| 949 | pyridin-3-yl | ethyl | 6-{[4-(trifluoromethyl)phenyl]sulphanyl}pyridin-3-yl |
| 950 | pyridin-3-yl | ethyl | 4-{[3-(pyridin-2-yl)-1H-pyrazol-1-yl]methyl}phenyl |
| 951 | pyridin-3-yl | ethyl | 4-chloro-3-[(2,2-dimethylpropanoyl)amino]phenyl |
| 952 | pyridin-3-yl | ethyl | 1-(2-chloropyridin-3-yl)-5-(1-methoxyethyl)-1H-pyrazol-4-yl |
| 953 | pyridin-3-yl | ethyl | 2-(3,5-dichloropyridin-2-yl)-1,3-thiazol-4-yl |
| 954 | pyridin-3-yl | ethyl | 4-[(methylsulphanyl)methyl]phenyl |
| 955 | pyridin-3-yl | ethyl | 6-(propylamino)pyridin-3-yl |
| 956 | pyridin-3-yl | ethyl | 5-chloro-6-(4-cyanophenoxy)pyridin-3-yl |
| 957 | pyridin-3-yl | ethyl | 6-(3,4-dichlorophenoxy)pyridin-3-yl |
| 958 | pyridin-3-yl | ethyl | 6-[(2-chloropyrimidin-4-yl)oxy]pyridin-3-yl |
| 959 | pyridin-3-yl | ethyl | 1-(3-chloropyridin-2-yl)-5-(1-methoxyethyl)-1H-pyrazol-4-yl |
| 960 | pyridin-3-yl | ethyl | 2-[2-(trifluoromethyl)phenyl]-1,3-thiazol-4-yl |
| 961 | pyridin-3-yl | ethyl | 3-(propan-2-ylcarbamoyl)phenyl |
| 962 | pyridin-3-yl | ethyl | 4-(heptafluoropropyl)phenyl |
| 963 | pyridin-3-yl | ethyl | 2-(2,6-dichloropyridin-3-yl)-1,3-thiazol-4-yl |
| 964 | pyridin-3-yl | ethyl | 1-ethyl-5-(trifluoromethyl)-1H-pyrazol-4-yl |
| 965 | pyridin-3-yl | ethyl | 1-[3-(difluoromethyl)pyridin-2-yl]-5-methyl-1H-pyrazol-4-yl |
| 966 | pyridin-3-yl | ethyl | 1-(2,4-dichlorophenyl)-1H-pyrazol-4-yl |
| 967 | pyridin-3-yl | ethyl | 2-[4-chloro-2-(trifluoromethyl)phenyl]-1,3-thiazol-5-yl |
| 968 | pyridin-3-yl | ethyl | 1-(propan-2-yl)-5-(trifluoromethyl)-1H-pyrazol-4-yl |
| 969 | pyridin-3-yl | ethyl | 4-[(2-methylpyrimidin-4-yl)oxy]phenyl |
| 970 | pyridin-3-yl | ethyl | 6-{[5-(trifluoromethyl)pyridin-2-yl]sulphanyl}pyridin-3-yl |
| 971 | pyridin-3-yl | ethyl | 6-[(4-nitrophenyl)sulphanyl]pyridin-3-yl |
| 972 | pyridin-3-yl | ethyl | 4-methyl-3-{[(propan-2-yloxy)carbonyl]amino}phenyl |
| 973 | pyridin-3-yl | ethyl | 1-[4-fluoro-2-(trifluoromethyl)phenyl]-5-(methoxymethyl)-1H- |
| | | | pyrazol-4-yl |
| 974 | pyridin-3-yl | ethyl | 5-(1,1-difluoroethyl)-1-(pyrimidin-2-yl)-1H-pyrazol-4-yl |
| 975 | pyridin-3-yl | ethyl | 3-{[3,5-bis(trifluoromethyl)-1H-1,2,4-triazol-1- |
| | | | yl]methyl}phenyl |
| 976 | pyridin-3-yl | ethyl | 4-{[3,5-bis(trifluoromethyl)-1H-pyrazol-1-yl]methyl}phenyl |
| 977 | pyridin-3-yl | ethyl | 1-(2-chlorophenyl)-5-(1,1-dimethoxyethyl)-1H-pyrazol-4-yl |
| 978 | pyridin-3-yl | ethyl | 1-(3-chloropyridin-2-yl)-5-(1,1-difluoroethyl)-1H-pyrazol-4-yl |
| 979 | pyridin-3-yl | ethyl | 3-(1-fluoroethyl)-1-methyl-1H-pyrazol-4-yl |
| 980 | pyridin-3-yl | ethyl | 1-(2,6-dichloropyridin-3-yl)-5-(methoxymethyl)-1H-pyrazol-4- |
| | | | yl |
| 981 | pyridin-3-yl | ethyl | 6-(quinolin-6-yloxy)pyridin-3-yl |
| 982 | pyridin-3-yl | ethyl | 3-(4-chloro-1H-pyrazol-1-yl)-4-methoxyphenyl |
| 983 | pyridin-3-yl | ethyl | 2-(2,4-dichlorophenyl)-1,3-thiazol-4-yl |
| 984 | pyridin-3-yl | ethyl | 1-(2-chloropyridin-3-yl)-5-(1,1-difluoroethyl)-1H-pyrazol-4-yl |
| 985 | pyridin-3-yl | ethyl | 6-{4-[4-(methylsulphanyl)phenoxy]phenoxy}pyridin-3-yl |
| 986 | pyridin-3-yl | ethyl | 6-(quinolin-5-yloxy)pyridin-3-yl |
| 987 | pyridin-3-yl | ethyl | 1-[4-chloro-2-(trifluoromethyl)phenyl]-5-(difluoromethyl)-1H- |
| | | | pyrazol-4-yl |
| 988 | pyridin-3-yl | ethyl | 1-[2-(1-methoxyethyl)phenyl]-1H-pyrazol-4-yl |
| 989 | pyridin-3-yl | ethyl | 4-methoxy-3-[(2-methylbutanoyl)amino]phenyl |
| 990 | pyridin-3-yl | ethyl | 6-(2,4-dichlorophenoxy)pyridin-3-yl |
| 991 | pyridin-3-yl | ethyl | 4-{[acetyl(2-methylpropyl)amino]methyl}phenyl |
| 992 | pyridin-3-yl | ethyl | 6-[(methylsulphanyl)methyl]pyridin-3-yl |
| 993 | pyridin-3-yl | ethyl | 2-(2,4-dichlorophenyl)-1,3-thiazol-5-yl |
| 994 | pyridin-3-yl | ethyl | 6-[2-chloro-4-(methylsulphanyl)phenoxy]pyridin-3-yl |
| 995 | pyridin-3-yl | ethyl | 1-(2-bromo-6-chlorophenyl)-5-(difluoromethyl)-1H-pyrazol-4- |
| | | | yl |
| 996 | pyridin-3-yl | ethyl | 4-(propan-2-yloxy)-3-[3-(trifluoromethyl)-1H-pyrazol-1- |
| | | | yl]phenyl |
| 997 | pyridin-3-yl | ethyl | 3-[ethyl(methyl)carbamoyl]phenyl |
| 998 | pyridin-3-yl | ethyl | 1-methyl-3-(trifluoromethyl)-1H-pyrazol-4-yl |
| 999 | pyridin-3-yl | ethyl | 6-(3,5-dimethylphenoxy)pyridin-3-yl |
| 1000 | pyridin-3-yl | ethyl | 1-[4-chloro-2-(trifluoromethyl)phenyl]-5-methyl-1H-pyrazol- |
| | | | 4-yl |
| 1001 | pyridin-3-yl | ethyl | 4-fluoro-3-(trifluoromethyl)phenyl |
| 1002 | pyridin-3-yl | ethyl | 6-[4-(methoxycarbonyl)phenoxy]pyridin-3-yl |
| 1003 | pyridin-3-yl | ethyl | 3-[(cyclopropylcarbonyl)amino]-4-methylphenyl |
| 1004 | pyridin-3-yl | ethyl | 1-(4-chlorophenyl)-1H-pyrazol-4-yl |
| 1005 | pyridin-3-yl | ethyl | 2-[2-(difluoromethyl)phenyl]-1,3-thiazol-5-yl |
| 1006 | pyridin-3-yl | ethyl | 1-ethyl-3-(trifluoromethyl)-1H-pyrazol-4-yl |
| 1007 | pyridin-3-yl | ethyl | 5-chloro-6-[4-(trifluoromethyl)phenoxy]pyridin-3-yl |
| 1008 | pyridin-3-yl | ethyl | 4-methyl-3-(propanoylamino)phenyl |
| 1009 | pyridin-3-yl | ethyl | 2-(2-chloropyridin-3-yl)-1,3-thiazol-4-yl |
| 1010 | pyridin-3-yl | ethyl | 4-[(2-methylpropanoyl)amino]phenyl |
| 1011 | pyridin-3-yl | ethyl | 3-[(2,2-dimethylpropanoyl)amino]-4-methylphenyl |
| 1012 | pyridin-3-yl | ethyl | 4-methoxy-3-{[(propan-2-yloxy)carbonyl]amino}phenyl |
| 1013 | pyridin-3-yl | ethyl | 1-(2,3-dichlorophenyl)-5-(methoxymethyl)-1H-pyrazol-4-yl |
| 1014 | pyridin-3-yl | ethyl | 3-(hydroxymethyl)-1-methyl-1H-pyrazol-4-yl |
| 1015 | pyridin-3-yl | ethyl | 1-(propan-2-yl)-3-(trifluoromethyl)-1H-pyrazol-4-yl |
| 1016 | pyridin-3-yl | ethyl | 2-[3-(difluoromethyl)pyridin-2-yl]-1,3-thiazol-5-yl |
| 1017 | pyridin-3-yl | ethyl | 4-(pyrrolidin-l-ylcarbonyl)phenyl |
| 1018 | pyridin-3-yl | ethyl | 3-(methoxymethyl)-1-methyl-1H-pyrazol-4-yl |
| 1019 | pyridin-3-yl | ethyl | 2-(3,5-dichloropyridin-2-yl)-1,3-thiazol-5-yl |
| 1020 | pyridin-3-yl | ethyl | 1-(2,6-dichloropyridin-3-yl)-5-(difluoromethyl)-1H-pyrazol-4- |
| | | | yl |
| 1021 | pyridin-3-yl | ethyl | 5-chloro-6-(4-nitrophenoxy)pyridin-3-yl |
| 1022 | pyridin-3-yl | ethyl | 2-[2-(difluoromethyl)phenyl]-1,3-thiazol-4-yl |
| 1023 | pyridin-3-yl | ethyl | 1-(difluoromethyl)-3-(trifluoromethyl)-1H-pyrazol-4-yl |
| 1024 | pyridin-3-yl | ethyl | 6-[(6-chloropyridin-2-yl)oxy]pyridin-3-yl |
| 1025 | pyridin-3-yl | ethyl | 3-(butanoylamino)-4-methoxyphenyl |
| 1026 | pyridin-3-yl | ethyl | 4-(acetylamino)phenyl |
| 1027 | pyridin-3-yl | ethyl | 1-(2-bromophenyl)-5-(trifluoromethyl)-1H-pyrazol-4-yl |
| 1028 | pyridin-3-yl | ethyl | 3-{[3-(trifluoromethyl)-1H-pyrazol-1-yl]methyl}phenyl |
| 1029 | pyridin-3-yl | ethyl | 5-(difluoromethyl)-1-(2-methylphenyl)-1H-pyrazol-4-yl |
| 1030 | pyridin-3-yl | ethyl | 2-[2-(1-methoxyethyl)phenyl]-1,3-thiazol-5-yl |
| 1031 | pyridin-3-yl | ethyl | 1-(2-chlorophenyl)-5-(1,1-difluoroethyl)-1H-pyrazol-4-yl |
| 1032 | pyridin-3-yl | ethyl | 1-(2-bromo-4-fluorophenyl)-1H-pyrazol-4-yl |
| 1033 | pyridin-3-yl | ethyl | 6-[(cyclopropylmethyl)amino]pyridin-3-yl |
| 1034 | pyridin-3-yl | ethyl | 6-[(2-methoxypyrimidin-4-yl)oxy]pyridin-3-yl |
| 1035 | pyridin-3-yl | ethyl | 1-[2-(1,1-difluoroethyl)phenyl]-5-methyl-1H-pyrazol-4-yl |
| 1036 | pyridin-3-yl | ethyl | 3-{[3,5-bis(trifluoromethyl)-1H-1,2,4-triazol-1-yl]methyl}-4- |
| | | | chlorophenyl |
| 1037 | pyridin-3-yl | ethyl | 6-(morpholin-4-yl)pyridin-3-yl |
| 1038 | pyridin-3-yl | ethyl | 2-(pyrimidin-2-yl)-1,3-thiazol-4-yl |
| 1039 | pyridin-3-yl | ethyl | 2-{[3-(trifluoromethyl)-1H-pyrazol-1-yl]methyl}pyridin-4-yl |
| 1040 | pyridin-3-yl | ethyl | 1-(2-chloropyridin-3-yl)-5-(trifluoromethyl)-1H-pyrazol-4-yl |
| 1041 | pyridin-3-yl | ethyl | 6-(4-bromophenoxy)pyridin-3-yl |
| 1042 | pyridin-3-yl | ethyl | 4-[(propylsulphanyl)methyl]phenyl |
| 1043 | pyridin-3-yl | ethyl | 1-(2-chloro-4-fluorophenyl)-1H-pyrazol-4-yl |
| 1044 | pyridin-3-yl | ethyl | 4-methoxy-3-[4-(pyridin-2-yl)-1H-pyrazol-1-yl]phenyl |
| 1045 | pyridin-3-yl | ethyl | 1-(2-chlorophenyl)-1H-pyrazol-4-yl |
| 1046 | pyridin-3-yl | ethyl | 6-(2,6-dichlorophenoxy)pyridin-3-yl |
| 1047 | pyridin-3-yl | ethyl | 1-(2-chlorophenyl)-5-methyl-1H-pyrazol-4-yl |
| 1048 | pyridin-3-yl | ethyl | 3-[ethyl(methyl)carbamoyl]-4-methoxyphenyl |
| 1049 | pyridin-3-yl | ethyl | 4-[(ethylsulphanyl)methyl]phenyl |
| 1050 | pyridin-3-yl | ethyl | 2-[2-(1-methoxyethyl)phenyl]-1,3-thiazol-4-yl |
| 1051 | pyridin-3-yl | ethyl | 6-{[6-(1H-pyrazol-1-yl)pyridin-2-yl]oxy}pyridin-3-yl |
| 1052 | pyridin-3-yl | ethyl | 2-[4-chloro-2-(trifluoromethyl)phenyl]-1,3-thiazol-4-yl |
| 1053 | pyridin-3-yl | ethyl | 4-methoxy-3-(1H-pyrazol-1-yl)phenyl |
| 1054 | pyridin-3-yl | ethyl | 3-(acetylamino)-4-methylphenyl |
| 1055 | pyridin-3-yl | ethyl | 2-{[4-(trifluoromethyl)-1H-pyrazol-1-yl]methyl}pyridin-4-yl |
| 1056 | pyridin-3-yl | ethyl | 1-(2,4-difluorophenyl)-1H-pyrazol-4-yl |
| 1057 | pyridin-3-yl | ethyl | 1-(2-methylphenyl)-5-(trifluoromethyl)-1H-pyrazol-4-yl |
| 1058 | pyridin-3-yl | ethyl | 2-[3-(difluoromethyl)pyridin-2-yl]-1,3-thiazol-4-yl |
| 1059 | pyridin-3-yl | ethyl | 6-(3,5-dichlorophenoxy)pyridin-3-yl |
| 1060 | pyridin-3-yl | ethyl | 1-(pyrimidin-2-ylmethyl)-3-(trifluoromethyl)-1H-pyrazol-4-yl |
| 1061 | pyridin-3-yl | ethyl | 6-methylpyridin-3-yl |
| 1062 | pyridin-3-yl | ethyl | 6-[(4-methylphenyl)sulphanyl]pyridin-3-yl |
| 1063 | pyridin-3-yl | ethyl | 1-(2,4-dichlorophenyl)-5-(methoxymethyl)-1H-pyrazol-4-yl |
| 1064 | pyridin-3-yl | ethyl | 6-{4-[4-(trifluoromethyl)phenoxy]phenoxy}pyridin-3-yl |
| 1065 | pyridin-3-yl | ethyl | 1-(2,6-dichlorophenyl)-5-(trifluoromethyl)-1H-pyrazol-4-yl |
| 1066 | pyridin-3-yl | ethyl | 6-(naphthalen-1-yloxy)pyridin-3-yl |
| 1067 | pyridin-3-yl | ethyl | 3-ethyl-1-methyl-1H-pyrazol-4-yl |
| 1068 | pyridin-3-yl | ethyl | 2-(2,6-dichloropyridin-3-yl)-1,3-thiazol-5-yl |
| 1069 | pyridin-3-yl | ethyl | 6-{[(4,6-dimethoxypyrimidin-2-yl)methyl]sulphanyl}pyridin- |
| | | | 3-yl |
| 1070 | pyridin-3-yl | ethyl | 1-[3-chloro-2-(trifluoromethyl)phenyl]-5-(1-methoxyethyl)- |
| | | | 1H-pyrazol-4-yl |
| 1071 | pyridin-3-yl | ethyl | 3,4-dimethoxyphenyl |
| 1072 | pyridin-3-yl | ethyl | 4-methoxy-3-[(methoxyacetyl)amino]phenyl |
| 1073 | pyridin-3-yl | ethyl | 3-{[4-(trifluoromethyl)-1H-pyrazol-1-yl]methyl}phenyl |
| 1074 | pyridin-3-yl | ethyl | 6-(2-cyanophenoxy)pyridin-3-yl |
| 1075 | pyridin-3-yl | ethyl | 3-[(methylsulphanyl)methyl]phenyl |
| 1076 | pyridin-3-yl | ethyl | 3-(dimethoxymethyl)-1-methyl-1H-pyrazol-4-yl |
| 1077 | pyridin-3-yl | ethyl | 1-(ethoxymethyl)-3-(trifluoromethyl)-1H-pyrazol-4-yl |
| 1078 | pyridin-3-yl | ethyl | 1-(3-bromopyridin-2-yl)-5-(methoxymethyl)-1H-pyrazol-4-yl |
| 1079 | pyridin-3-yl | ethyl | 6-(methylsulphanyl)pyridin-3-yl |
| 1080 | pyridin-3-yl | ethyl | 1-(2-bromo-6-chlorophenyl)-5-(trifluoromethyl)-1H-pyrazol-4- |
| | | | yl |
| 1081 | pyridin-3-yl | ethyl | 3-{[4-(trifluoromethyl)phenoxy]methyl}phenyl |
| 1082 | pyridin-3-yl | ethyl | 6-(heptafluoropropyl)pyridin-3-yl |
| 1083 | pyridin-3-yl | ethyl | 1-ethenyl-3-(trifluoromethyl)-1H-pyrazol-4-yl |
| 1084 | pyridin-3-yl | ethyl | 4-methyl-3-nitrophenyl |
| 1085 | pyridin-3-yl | ethyl | 3-(4-bromo-1H-pyrazol-1-yl)-4-methoxyphenyl |
| 1086 | pyridin-3-yl | ethyl | 5-methyl-1-(2-methylphenyl)-1H-pyrazol-4-yl |
| 1087 | pyridin-3-yl | ethyl | 1-[3-(difluoromethyl)pyridin-2-yl]-5-(methoxymethyl)-1H- |
| | | | pyrazol-4-yl |
| 1088 | pyridin-3-yl | ethyl | 6-{[2-(trifluoromethyl)pyrimidin-5-yl]oxy}pyridin-3-yl |
| 1089 | pyridin-3-yl | ethyl | 2-[2-(methoxymethyl)phenyl]-1,3-thiazol-5-yl |
| 1090 | pyridin-3-yl | ethyl | 1-(ethoxymethyl)-5-(trifluoromethyl)-1H-pyrazol-4-yl |
| 1091 | pyridin-3-yl | ethyl | 5-chloro-6-[(4,5,6-trimethylpyrimidin-2-yl)sulphanyl]pyridin- |
| | | | 3-yl |
| 1092 | pyridin-3-yl | ethyl | 6-[3,5-bis(trifluoromethyl)phenoxy]pyridin-3-yl |
| 1093 | pyridin-3-yl | ethyl | 4-{[4-(pyridin-2-yl)-1H-pyrazol-1-yl]methyl}phenyl |
| 1094 | pyridin-3-yl | ethyl | 1-[3-(1,1-difluoroethyl)pyridin-2-yl]-5-methyl-1H-pyrazol-4-yl |
| 1095 | pyridin-3-yl | ethyl | 1-(2,6-dichlorophenyl)-5-(difluoromethyl)-1H-pyrazol-4-yl |
| 1096 | pyridin-3-yl | ethyl | 1-[4-fluoro-2-(trifluoromethyl)phenyl]-5-methyl-1H-pyrazol-4- |
| | | | yl |
| 1097 | pyridin-3-yl | ethyl | 4-chloro-3-[(cyclobutylcarbonyl)amino]phenyl |
| 1098 | pyridin-3-yl | ethyl | 2-[2-(methoxymethyl)phenyl]-1,3-thiazol-4-yl |
| 1099 | pyridin-3-yl | ethyl | 4-(1H-pyrazol-1-ylmethyl)phenyl |
| 1100 | pyridin-3-yl | ethyl | 1-(2-bromophenyl)-5-methyl-1H-pyrazol-4-yl |
| 1101 | pyridin-3-yl | ethyl | 1-(2,3-difluorophenyl)-1H-pyrazol-4-yl |
| 1102 | pyridin-3-yl | ethyl | 6-{4-[(trifluoromethyl)sulphanyl]phenoxy}pyridin-3-yl |
| 1103 | pyridin-3-yl | ethyl | 4-{[3-(pentafluoroethyl)-1H-1,2,4-triazol-1-yl]methyl}phenyl |
| 1104 | pyridin-3-yl | ethyl | 4-{[3-(pyrimidin-2-yl)-1H-pyrazol-1-yl]methyl}phenyl |
| 1105 | pyridin-3-yl | ethyl | 1-(2-chlorophenyl)-5-(difluoromethyl)-1H-pyrazol-4-yl |
| 1106 | pyridin-3-yl | ethyl | 5-(difluoromethyl)-1-[2-(trifluoromethyl)phenyl]-1H-pyrazol- |
| | | | 4-yl |
| 1107 | pyridin-3-yl | ethyl | 6-(4-methoxyphenoxy)pyridin-3-yl |
| 1108 | pyridin-3-yl | ethyl | 1-[2-(dimethoxymethyl)phenyl]-1H-pyrazol-4-yl |
| 1109 | pyridin-3-yl | ethyl | 5-(methoxymethyl)-1-(pyrimidin-2-yl)-1H-pyrazol-4-yl |
| 1110 | pyridin-3-yl | ethyl | 6-tert-butoxypyridin-3-yl |
| 1111 | pyridin-3-yl | ethyl | 4-[(cyclopentylimino)methyl]phenyl |
| 1112 | pyridin-3-yl | ethyl | 1-(3-chlorophenyl)-1H-pyrazol-4-yl |
| 1113 | pyridin-3-yl | ethyl | 3-[(2-methylpropanoyl)amino]phenyl |
| 1114 | pyridin-3-yl | ethyl | 6-{[3,5-bis(trifluoromethyl)-1H-pyrazol-1-yl]methyl}pyridin- |
| | | | 3-yl |
| 1115 | pyridin-3-yl | ethyl | 4-chloro-3-[(2-methylpropanoyl)amino]phenyl |
| 1116 | pyridin-3-yl | ethyl | 4-(propylsulphanyl)phenyl |
| 1117 | pyridin-3-yl | ethyl | 1-[2-(difluoromethyl)phenyl]-5-(methoxymethyl)-1H-pyrazol- |
| | | | 4-yl |
| 1118 | pyridin-3-yl | ethyl | 4-methoxy-3-[4-(pyrimidin-2-yl)-1H-pyrazol-1-yl]phenyl |
| 1119 | pyridin-3-yl | ethyl | 4-{[(4,6-dimethylpyrimidin-2-yl)sulphanyl]methyl}phenyl |
| 1120 | pyridin-3-yl | ethyl | 1-(2-chlorophenyl)-5-(methoxymethyl)-1H-pyrazol-4-yl |
| 1121 | pyridin-3-yl | ethyl | 6-{[3,5-bis(trifluoromethyl)-1H-1,2,4-triazol-1- |
| | | | yl]methyl}pyridin-3-yl |
| 1122 | pyridin-3-yl | ethyl | 5-chloro-6-[4-(trifluoromethoxy)phenoxy]pyridin-3-yl |
| 1123 | pyridin-3-yl | ethyl | 2-(3-chloropyridin-2-yl)-1,3-thiazol-5-yl |
| 1124 | pyridin-3-yl | ethyl | 1-(2-fluorophenyl)-1H-pyrazol-4-yl |
| 1125 | pyridin-3-yl | ethyl | 4-(trifluoromethoxy)phenyl |
| 1126 | pyridin-3-yl | ethyl | 6-(2-chlorophenoxy)pyridin-3-yl |
| 1127 | pyridin-3-yl | ethyl | 3-[(2,2-dimethylpropanoyl)amino]-5-(trifluoromethyl)phenyl |
| 1128 | pyridin-3-yl | ethyl | 6-{[2-(morpholin-4-yl)ethyl]amino}pyridin-3-yl |
| 1129 | pyridin-3-yl | ethyl | 3-formyl-1-methyl-1H-pyrazol-4-yl |
| 1130 | pyridin-3-yl | ethyl | 1-methyl-3-(propan-2-yl)-1H-pyrazol-4-yl |
| 1131 | pyridin-3-yl | ethyl | 1-(3-chlorophenyl)-5-(1,1-dimethoxyethyl)-1H-pyrazol-4-yl |
| 1132 | pyridin-3-yl | ethyl | 4-nitrophenyl |
| 1133 | pyridin-3-yl | ethyl | 6-{[5-(methoxycarbonyl)pyridin-3-yl]oxy}pyridin-3-yl |
| 1134 | pyridin-3-yl | ethyl | 3-{[4-(trifluoromethyl)benzyl]oxy}phenyl |
| 1135 | pyridin-3-yl | ethyl | 1-(2,4-dichlorophenyl)-5-(1,1-dimethoxyethyl)-1H-pyrazol-4- |
| | | | yl |
| 1136 | pyridin-3-yl | ethyl | 4-{[4-bromo-3-(trifluoromethyl)-1H-pyrazol-1- |
| | | | yl]methyl}phenyl |
| 1137 | pyridin-3-yl | ethyl | 3-[chloro(difluoro)methyl]-1-methyl-1H-pyrazol-4-yl |
| 1138 | pyridin-3-yl | ethyl | 3-[(cyclopropylcarbonyl)amino]-4-fluorophenyl |
| 1139 | pyridin-3-yl | ethyl | 6-[4-(dimethylsulphamoyl)phenoxy]pyridin-3-yl |
| 1140 | pyridin-3-yl | ethyl | 5-methyl-1-(2-methylpyridin-3-yl)-1H-pyrazol-4-yl |
| 1141 | pyridin-3-yl | ethyl | 1-(2,4-difluorophenyl)-5-methyl-1H-pyrazol-4-yl |
| 1142 | pyridin-3-yl | ethyl | 3-(methylsulphanyl)phenyl |
| 1143 | pyridin-3-yl | ethyl | 3-(acetylamino)-4-chlorophenyl |
| 1144 | pyridin-3-yl | ethyl | 4-[(4-bromo-1H-pyrazol-1-yl)methyl]phenyl |
| 1145 | pyridin-3-yl | ethyl | 4-methyl-3-[(propan-2-ylcarbamoyl)amino]phenyl |
| 1146 | pyridin-3-yl | ethyl | 1-(2,2-diethoxyethyl)-3-(trifluoromethyl)-1H-pyrazol-4-yl |
| 1147 | pyridin-3-yl | ethyl | 6-(1H-pyrazol-1-yl)pyridin-3-yl |
| 1148 | pyridin-3-yl | ethyl | 4-(1H-1,2,4-triazol-1-ylmethyl)phenyl |
| 1149 | pyridin-3-yl | ethyl | 5-(methoxymethyl)-1-[2-(trifluoromethyl)phenyl]-1H-pyrazol- |
| | | | 4-yl |
| 1150 | pyridin-3-yl | ethyl | 5-methylpyridin-3-yl |
| 1151 | pyridin-3-yl | ethyl | 5-chloro-6-[(4,6-dimethylpyrimidin-2-yl)sulphanyl]pyridin-3- |
| | | | yl |
| 1152 | pyridin-3-yl | ethyl | 3-[(2,2-dimethylpropanoyl)amino]-4-methoxyphenyl |
| 1153 | pyridin-3-yl | ethyl | 2-[2-(trifluoromethyl)pyridin-3-yl]-1,3-thiazol-4-yl |
| 1154 | pyridin-3-yl | ethyl | 3-cyclopropyl-1-methyl-1H-pyrazol-4-yl |
| 1155 | pyridin-3-yl | ethyl | 3-methyl-1H-pyrazol-4-yl |
| 1156 | pyridin-3-yl | ethyl | 5-(methoxymethyl)-1-(2,2,2-trifluoroethyl)-1H-pyrazol-4-yl |
| 1157 | pyridin-3-yl | ethyl | 4-methoxy-3-(propanoylamino)phenyl |
| 1158 | pyridin-3-yl | ethyl | 4-methoxy-3-[(2-methylpropanoyl)amino]phenyl |
| 1159 | pyridin-3-yl | ethyl | 6-(quinolin-7-yloxy)pyridin-3-yl |
| 1160 | pyridin-3-yl | ethyl | 4-{[3-(trifluoromethyl)-1H-pyrazol-1-yl]methyl}phenyl |
| 1161 | pyridin-3-yl | ethyl | 4-{[3,5-bis(trifluoromethyl)-1H-1,2,4-triazol-1- |
| | | | yl]methyl}phenyl |
| 1162 | pyridin-3-yl | ethyl | 3-fluoro-4-{[4-(trifluoromethyl)-1H-pyrazol-1- |
| | | | yl]methyl}phenyl |
| 1163 | pyridin-3-yl | ethyl | 2,3-dihydro-1,4-benzodioxin-6-yl |
| 1164 | pyridin-3-yl | ethyl | 2-[3-(methoxymethyl)pyridin-2-yl]-1,3-thiazol-5-yl |
| 1165 | pyridin-3-yl | ethyl | 6-({6-[3-(trifluoromethyl)-1H-pyrazol-1-yl]pyridin-2- |
| | | | yl}oxy)pyridin-3-yl |
| 1166 | pyridin-3-yl | ethyl | 3-{[3-(pyridin-2-yl)-1H-pyrazol-1-yl]methyl}phenyl |
| 1167 | pyridin-3-yl | ethyl | 1-(2-bromo-4-chlorophenyl)-5-methyl-1H-pyrazol-4-yl |
| 1168 | pyridin-3-yl | ethyl | 2,2-difluoro-1,3-benzodioxo1-5-yl |
| 1169 | pyridin-3-yl | ethyl | 2-[3-(trifluoromethyl)pyridin-2-yl]-1,3-thiazol-4-yl |
| 1170 | pyridin-3-yl | ethyl | 4-(benzylcarbamoyl)phenyl |
| 1171 | pyridin-3-yl | ethyl | 4-[(2-chloropyrimidin-4-yl)oxy]phenyl |
| 1172 | pyridin-3-yl | ethyl | 1-[3-(trifluoromethyl)pyridin-2-yl]-1H-pyrazol-4-yl |
| 1173 | pyridin-3-yl | ethyl | 5-chloro-6-(4-chlorophenoxy)pyridin-3-yl |
| 1174 | pyridin-3-yl | ethyl | 3-cyano-4-(1H-1,2,4-triazol-1-yl)phenyl |
| 1175 | pyridin-3-yl | ethyl | 3-amino-4-(methylsulphonyl)phenyl |
| 1176 | pyridin-3-yl | ethyl | 4-methyl-3-[(2-methylpropanoyl)amino]phenyl |
| 1177 | pyridin-3-yl | ethyl | 1-(2,6-dibromophenyl)-5-(trifluoromethyl)-1H-pyrazol-4-yl |
| 1178 | pyridin-3-yl | ethyl | 3-{[3-(trifluoromethyl)-1H-1,2,4-triazol-1-yl]methyl}phenyl |
| 1179 | pyridin-3-yl | ethyl | 4-[(3,3,3-trifluoropropanoyl)amino]phenyl |
| 1180 | pyridin-3-yl | ethyl | 5-(pyrazin-2-yl)-1-(2,2,2-trifluoroethyl)-1H-pyrazol-4-yl |
| 1181 | pyridin-3-yl | ethyl | 1-(2,6-dichlorophenyl)-5-(dimethoxymethyl)-1H-pyrazol-4-yl |
| 1182 | pyridin-3-yl | ethyl | 1-(4-chlorophenyl)-5-(1,1-dimethoxyethyl)-1H-pyrazol-4-yl |
| 1183 | pyridin-3-yl | ethyl | 4-({6-[3-(trifluoromethyl)-1H-pyrazol-1-yl]pyridin-2- |
| | | | yl}oxy)phenyl |
| 1184 | pyridin-3-yl | ethyl | 4-methoxy-3-(propan-2-ylcarbamoyl)phenyl |
| 1185 | pyridin-3-yl | ethyl | 1-[2-(1-methoxyethyl)phenyl]-5-methyl-1H-pyrazol-4-yl |
| 1186 | pyridin-3-yl | ethyl | 1-ethyl-3-methyl-1H-pyrazol-4-yl |
| 1187 | pyridin-3-yl | ethyl | 6-(4-methylphenoxy)pyridin-3-yl |
| 1188 | pyridin-3-yl | ethyl | 6-(4-chloro-2-cyanophenoxy)pyridin-3-yl |
| 1189 | pyridin-3-yl | ethyl | 1-propyl-3-(trifluoromethyl)-1H-pyrazol-4-yl |
| 1190 | pyridin-3-yl | ethyl | 6-(2-methylphenoxy)pyridin-3-yl |
| 1191 | pyridin-3-yl | ethyl | 4-{[4-(trifluoromethyl)-1H-pyrazol-1-yl]methyl}phenyl |
| 1192 | pyridin-3-yl | ethyl | 6-methoxypyridin-3-yl |
| 1193 | pyridin-3-yl | ethyl | 4-{[acetyl(cyclopentyl)amino]methyl}phenyl |
| 1194 | pyridin-3-yl | ethyl | 3-{[3,5-bis(trifluoromethyl)-1H-pyrazol-1-yl]methyl}phenyl |
| 1195 | pyridin-3-yl | ethyl | 1-(2-bromo-4-chlorophenyl)-1H-pyrazol-4-yl |
| 1196 | pyridin-3-yl | ethyl | 1-(2-chlorophenyl)-5-(trifluoromethyl)-1H-pyrazol-4-yl |
| 1197 | pyridin-3-yl | ethyl | 4-methoxy-3-nitrophenyl |
| 1198 | pyridin-3-yl | ethyl | 1-(2,6-dibromophenyl)-5-(difluoromethyl)-1H-pyrazol-4-yl |
| 1199 | pyridin-3-yl | ethyl | 6-[(4-methoxyphenyl)sulphanyl]pyridin-3-yl |
| 1200 | pyridin-3-yl | ethyl | 1-(2,6-dichlorophenyl)-5-(1-methoxyethyl)-1H-pyrazol-4-yl |
| 1201 | pyridin-3-yl | ethyl | 4-[(6-chloropyridin-2-yl)oxy]phenyl |
| 1202 | pyridin-3-yl | ethyl | 3-fluoro-4-{[3-(trifluoromethyl)-1H-pyrazol-1- |
| | | | yl]methyl}phenyl |
| 1203 | pyridin-3-yl | ethyl | 4-{[(difluoromethyl)sulphanyl]methyl}phenyl |
| 1204 | pyridin-3-yl | ethyl | 6-{[3-(trifluoromethyl)-1H-pyrazol-1-yl]methyl}pyridin-3-yl |
| 1205 | pyridin-3-yl | ethyl | 4-{[2-(3,5-dichlorophenyl)propan-2-yl]carbamoyl}phenyl |
| 1206 | pyridin-3-yl | ethyl | 3-(1H-1,2,4-triazol-1-ylmethyl)phenyl |
| 1207 | pyridin-3-yl | ethyl | 2-(3-chloropyridin-2-yl)-1,3-thiazol-4-yl |
| 1208 | pyridin-3-yl | ethyl | 5-(1-methoxyethyl)-1-(pyrimidin-2-yl)-1H-pyrazol-4-yl |
| 1209 | pyridin-3-yl | ethyl | 1-(3-chloropyridin-2-yl)-5-(1,1-dimethoxyethyl)-1H-pyrazol- |
| | | | 4-yl |
| 1210 | pyridin-3-yl | ethyl | 1-(2-bromo-4-fluorophenyl)-5-methyl-1H-pyrazol-4-yl |
| 1211 | pyridin-3-yl | ethyl | 3-(butanoylamino)-4-methylphenyl |
| 1212 | pyridin-3-yl | ethyl | 5-(methoxymethyl)-1-[3-(trifluoromethyl)pyridin-2-yl]-1H- |
| | | | pyrazol-4-yl |
| 1213 | pyridin-3-yl | ethyl | 6-[3-(trifluoromethyl)-1H-pyrazol-1-yl]pyridin-3-yl |
| 1214 | pyridin-3-yl | ethyl | 6-(3-chlorophenoxy)pyridin-3-yl |
| 1215 | pyridin-3-yl | ethyl | 1-(2,6-dichloropyridin-3-yl)-5-(1-methoxyethyl)-1H-pyrazol- |
| | | | 4-yl |
| 1216 | pyridin-3-yl | ethyl | 1-[2-(difluoromethyl)phenyl]-5-methyl-1H-pyrazol-4-yl |
| 1217 | pyridin-3-yl | ethyl | 2-[2-(trifluoromethyl)phenyl]-1,3-thiazol-5-yl |
| 1218 | pyridin-3-yl | ethyl | 2-[2-(trifluoromethyl)pyridin-3-yl]-1,3-thiazol-5-yl |
| 1219 | pyridin-3-yl | ethyl | 2-(2-chloropyridin-3-yl)-1,3-thiazol-5-yl |
| 1220 | pyridin-3-yl | ethyl | 1-(2,6-dichloropyridin-3-yl)-5-methyl-1H-pyrazol-4-yl |
| 1221 | pyridin-3-yl | ethyl | 1-(3-chloropyridin-2-yl)-5-(methoxymethyl)-1H-pyrazol-4-yl |
| 1222 | pyridin-3-yl | ethyl | 6-[(4-chlorophenyl)sulphanyl]pyridin-3-yl |
| 1223 | pyridin-3-yl | ethyl | 6-(piperidin-l-yl)pyridin-3-yl |
| 1224 | pyridin-3-yl | ethyl | 3-[(cyclopentylcarbonyl)amino]-4-methylphenyl |
| 1225 | pyridin-3-yl | ethyl | 6-ethoxypyridin-3-yl |
| 1226 | pyridin-3-yl | ethyl | 2-(2,6-dichlorophenyl)-1,3-thiazol-4-yl |
| 1227 | pyridin-3-yl | ethyl | 5-chloro-6-[(4-methylpyrimidin-2-yl)sulphanyl]pyridin-3-yl |
| 1228 | pyridin-3-yl | ethyl | 1-[4-chloro-2-(trifluoromethyl)phenyl]-5-(1-methoxyethyl)- |
| | | | 1H-pyrazol-4-yl |
| 1229 | pyridin-3-yl | ethyl | 6-(3-methylphenoxy)pyridin-3-yl |
| 1230 | pyridin-3-yl | ethyl | 4-[ethyl(methyl)carbamoyl]phenyl |
| 1231 | pyridin-3-yl | ethyl | 6-[3-(trifluoromethoxy)phenoxy]pyridin-3-yl |
| 1232 | pyridin-3-yl | ethyl | 5-(difluoromethyl)-1-(pyrimidin-2-yl)-1H-pyrazol-4-yl |
| 1233 | pyridin-3-yl | ethyl | 4-[(cyclopropylcarbonyl)amino]phenyl |
| 1234 | pyridin-3-yl | ethyl | 1-(3-bromopyridin-2-yl)-5-(1-methoxyethyl)-1H-pyrazol-4-yl |
| 1235 | pyridin-3-yl | ethyl | 3-[(cyclopropylcarbonyl)amino]-4-methoxyphenyl |
| 1236 | pyridin-3-yl | ethyl | 4-methyl-3-[(3-methylbut-2-enoyl)amino]phenyl |
| 1237 | pyridin-3-yl | ethyl | 1-[3-(methoxymethyl)pyridin-2-yl]-1H-pyrazol-4-yl |
| 1238 | pyridin-3-yl | ethyl | 6-(naphthalen-2-yloxy)pyridin-3-yl |
| 1239 | pyridin-3-yl | ethyl | 1-(2-chlorophenyl)-5-(1-methoxyethyl)-1H-pyrazol-4-yl |
| 1240 | pyridin-3-yl | ethyl | 1-[2-(difluoromethyl)phenyl]-5-(1-methoxyethyl)-1H-pyrazol- |
| | | | 4-yl |
| 1241 | pyridin-3-yl | ethyl | 1-(3-chloropyridin-2-yl)-5-methyl-1H-pyrazol-4-yl |
| 1242 | pyridin-3-yl | ethyl | 4-{[3-(trifluoromethyl)-1H-1,2,4-triazol-1-yl]methyl}phenyl |
| 1243 | pyridin-3-yl | ethyl | 3-[(cyclopentylcarbonyl)amino]-4-methoxyphenyl |
| 1244 | pyridin-3-yl | ethyl | 1-(2,2,2-trifluoroethyl)-5-(trifluoromethyl)-1H-pyrazol-4-yl |
| 1245 | pyridin-3-yl | ethyl | 6-[4-(methylsulphanyl)phenoxy]pyridin-3-yl |
| 1246 | pyridin-3-yl | ethyl | 2-[3-(trifluoromethyl)pyridin-2-yl]-1,3-thiazol-5-yl |
| 1247 | pyridin-3-yl | ethyl | 4-{[3-(methoxycarbonyl)-1H-1,2,4-triazol-1-yl]methyl}phenyl |
| 1248 | pyridin-3-yl | ethyl | 1-[3-(1,1-difluoroethyl)pyridin-2-yl]-1H-pyrazol-4-yl |
| 1249 | pyridin-3-yl | ethyl | 6-{[3-(pyridin-2-yl)-1H-pyrazol-1-yl]methyl}pyridin-3-yl |
| 1250 | pyridin-3-yl | ethyl | 3-(acetylamino)phenyl |
| 1251 | pyridin-3-yl | ethyl | 1-(3-chloropyridin-2-yl)-5-(trifluoromethyl)-1H-pyrazol-4-yl |
| 1252 | pyridin-3-yl | ethyl | 2-{[3-(pyridin-2-yl)-1H-pyrazol-1-yl]methyl}pyridin-4-yl |
| 1253 | pyridin-3-yl | ethyl | 3-[(methoxyacetyl)amino]-4-methylphenyl |
| 1254 | pyridin-3-yl | ethyl | 1-(3-bromopyridin-2-yl)-5-(trifluoromethyl)-1H-pyrazol-4-yl |
| 1255 | pyridin-3-yl | ethyl | 1-(pyrimidin-2-yl)-5-(trifluoromethyl)-1H-pyrazol-4-yl |
| 1256 | pyridin-3-yl | ethyl | 2-(2-chlorophenyl)-1,3-thiazol-4-yl |
| 1257 | pyridin-3-yl | ethyl | 4-methoxy-3-[(propan-2-ylsulphonyl)amino]phenyl |
| 1258 | pyridin-3-yl | ethyl | 6-(3-cyanophenoxy)pyridin-3-yl |
| 1259 | pyridin-3-yl | ethyl | 4-fluoro-3-[(2-methylpropanoyl)amino]phenyl |
| 1260 | pyridin-3-yl | ethyl | 1-(2-bromophenyl)-5-(difluoromethyl)-1H-pyrazol-4-yl |
| 1261 | pyridin-3-yl | ethyl | 5-(1-methoxyethyl)-1-(2,2,2-trifluoroethyl)-1H-pyrazol-4-yl |
| 1262 | pyridin-3-yl | ethyl | 1-(2,6-dichlorophenyl)-5-(1-fluoroethyl)-1H-pyrazol-4-yl |
| 1263 | pyridin-3-yl | ethyl | 6-(pyrrolidin-l-yl)pyridin-3-yl |
| 1264 | pyridin-3-yl | ethyl | 3-(acetylamino)-5-(trifluoromethyl)phenyl |
| 1265 | pyridin-3-yl | ethyl | 2-[3-(methoxymethyl)pyridin-2-yl]-1,3-thiazol-4-yl |
| 1266 | pyridin-3-yl | ethyl | 2-[3-(1-methoxyethyl)pyridin-2-yl]-1,3-thiazol-5-yl |
| 1267 | pyridin-3-yl | ethyl | 1-[3-(dimethoxymethyl)pyridin-2-yl]-1H-pyrazol-4-yl |
| 1268 | pyridin-3-yl | ethyl | 5-(1-methoxyethyl)-1-(2-methylphenyl)-1H-pyrazol-4-yl |
| 1269 | pyridin-3-yl | ethyl | 1-methyl-1H-benzotriazol-5-yl |
| 1270 | pyridin-3-yl | ethyl | 4-[(butylsulphanyl)methyl]phenyl |
| 1271 | pyridin-3-yl | ethyl | 1H-pyrazol-4-yl |
| 1272 | pyridin-3-yl | ethyl | 1-(2-bromo-6-chlorophenyl)-5-(1-methoxyethyl)-1H-pyrazol- |
| | | | 4-yl |
| 1273 | pyridin-3-yl | ethyl | 5-chloro-6-[4-(methylsulphonyl)phenoxy]pyridin-3-yl |
| 1274 | pyridin-3-yl | ethyl | 1-[3-(1-methoxyethyl)pyridin-2-yl]-1H-pyrazol-4-yl |
| 1275 | pyridin-3-yl | ethyl | 4-[(6-methylpyridin-3-yl)oxy]phenyl |
| 1276 | pyridin-3-yl | ethyl | 3-methoxy-1-methyl-1H-pyrazol-4-yl |
| 1277 | pyridin-3-yl | ethyl | 6-(4-tert-butylphenoxy)pyridin-3-yl |
| 1278 | pyridin-3-yl | ethyl | 1-(2,4-dichlorophenyl)-5-methyl-1H-pyrazol-4-yl |
| 1279 | pyridin-3-yl | ethyl | 3-(but-2-enoylamino)-4-methylphenyl |
| 1280 | pyridin-3-yl | ethyl | 4-{[4-(pyrimidin-2-yl)-1H-pyrazol-1-yl]methyl}phenyl |
| 1281 | pyridin-3-yl | ethyl | 4-{[6-(1H-pyrazol-1-yl)pyridin-2-yl]oxy}phenyl |
| 1282 | pyridin-3-yl | ethyl | 4-{[3,5-bis(trifluoromethyl)-1H-1,2,4-triazol-1-yl]methyl}-3- |
| | | | chlorophenyl |
| 1283 | pyridin-3-yl | ethyl | 4-[(2-methoxypyrimidin-4-yl)oxy]phenyl |
| 1284 | pyridin-3-yl | ethyl | 1-[4-chloro-2-(trifluoromethyl)phenyl]-5-(methoxymethyl)- |
| | | | 1H-pyrazol-4-yl |
| 1285 | pyridin-3-yl | ethyl | 5-(trifluoromethyl)-1-[2-(trifluoromethyl)phenyl]-1H-pyrazol- |
| | | | 4-yl |
| 1286 | pyridin-3-yl | ethyl | 6-(dimethylamino)pyridin-3-yl |
| 1287 | pyridin-3-yl | ethyl | 2-[5-chloro-3-(trifluoromethyl)pyridin-2-yl]-1,3-thiazol-5-yl |
| 1288 | pyridin-3-yl | ethyl | 4-{[4-(3-chloropyridin-2-yl)-1H-pyrazol-1-yl]methyl}phenyl |
| 1289 | pyridin-3-yl | ethyl | 4-[(trifluoromethyl)sulphanyl]phenyl |
| 1290 | pyridin-3-yl | ethyl | 1-(2-bromophenyl)-5-(methoxymethyl)-1H-pyrazol-4-yl |
| 1291 | pyridin-3-yl | ethyl | 6-[(4-bromo-1H-pyrazol-1-yl)methyl]pyridin-3-yl |
| 1292 | pyridin-3-yl | ethyl | 3-chloro-4-{[3-(trifluoromethyl)-1H-pyrazol-1- |
| | | | yl]methyl}phenyl |
| 1293 | pyridin-3-yl | ethyl | 6-{[4-(trifluoromethyl)-1H-pyrazol-1-yl]methyl}pyridin-3-yl |
| 1294 | pyridin-3-yl | ethyl | 1-(3-bromopyridin-2-yl)-5-(difluoromethyl)-1H-pyrazol-4-yl |
| 1295 | pyridazin-4-yl | ethyl | 1-(2,6-dichloropyridin-3-yl)-5-(trifluoromethyl)-1H-pyrazol-4- |
| | | | yl |
| 1296 | pyridazin-4-yl | ethyl | 5-(1-methoxyethyl)-1-[3-(trifluoromethyl)pyridin-2-yl]-1H- |
| | | | pyrazol-4-yl |
| 1297 | pyridazin-4-yl | ethyl | 1-phenyl-1H-pyrazol-4-yl |
| 1298 | pyridazin-4-yl | ethyl | 6-chloropyridin-3-yl |
| 1299 | pyridazin-4-yl | ethyl | 2-[5-chloro-3-(trifluoromethyl)pyridin-2-yl]-1,3-thiazol-4-yl |
| 1300 | pyridazin-4-yl | ethyl | 3-(trifluoromethyl)-1H-pyrazol-4-yl |
| 1301 | pyridazin-4-yl | ethyl | 1-[3-(difluoromethyl)pyridin-2-yl]-1H-pyrazol-4-yl |
| 1302 | pyridazin-4-yl | ethyl | 5-(trifluoromethyl)-1-[3-(trifluoromethyl)pyridin-2-yl]-1H- |
| | | | pyrazol-4-yl |
| 1303 | pyridazin-4-yl | ethyl | 1-(2-bromophenyl)-5-(1-methoxyethyl)-1H-pyrazol-4-yl |
| 1304 | pyridazin-4-yl | ethyl | 1-(1,3-thiazol-2-ylmethyl)-3-(trifluoromethyl)-1H-pyrazol-4-yl |
| 1305 | pyridazin-4-yl | ethyl | 3-(trifluoromethyl)phenyl |
| 1306 | pyridazin-4-yl | ethyl | 2-[3-(1-methoxyethyl)pyridin-2-yl]-1,3-thiazol-4-yl |
| 1307 | pyridazin-4-yl | ethyl | 5-(1-fluoroethyl)-1-(pyrimidin-2-yl)-1H-pyrazol-4-yl |
| 1308 | pyridazin-4-yl | ethyl | 6-(4-cyanophenoxy)pyridin-3-yl |
| 1309 | pyridazin-4-yl | ethyl | 1-[2-(trifluoromethyl)phenyl]-1H-pyrazol-4-yl |
| 1310 | pyridazin-4-yl | ethyl | 1-[3-chloro-2-(trifluoromethyl)phenyl]-5-(methoxymethyl)- |
| | | | 1H-pyrazol-4-yl |
| 1311 | pyridazin-4-yl | ethyl | 4-[(2-cyanoethyl)(methyl)carbamoyl]phenyl |
| 1312 | pyridazin-4-yl | ethyl | 4-[(trifluoroacetyl)amino]phenyl |
| 1313 | pyridazin-4-yl | ethyl | 1-benzyl-3-(trifluoromethyl)-1H-pyrazol-4-yl |
| 1314 | pyridazin-4-yl | ethyl | 6-(2,5-dichlorophenoxy)pyridin-3-yl |
| 1315 | pyridazin-4-yl | ethyl | 2-(pyrimidin-2-yl)-1,3-thiazol-5-yl |
| 1316 | pyridazin-4-yl | ethyl | 3-(but-2-enoylamino)-4-methoxyphenyl |
| 1317 | pyridazin-4-yl | ethyl | 4-(dimethylcarbamoyl)phenyl |
| 1318 | pyridazin-4-yl | ethyl | 4-chloro-3-[(cyclopropylcarbonyl)amino]phenyl |
| 1319 | pyridazin-4-yl | ethyl | 1-(2,6-dichlorophenyl)-5-methyl-1H-pyrazol-4-yl |
| 1320 | pyridazin-4-yl | ethyl | 1-(2-chloropyridin-3-yl)-5-(difluoromethyl)-1H-pyrazol-4-yl |
| 1321 | pyridazin-4-yl | ethyl | 1-(3-chloropyridin-2-yl)-5-(dimethoxymethyl)-1H-pyrazol-4-yl |
| 1322 | pyridazin-4-yl | ethyl | 5-(difluoromethyl)-1-(2,2,2-trifluoroethyl)-1H-pyrazol-4-yl |
| 1323 | pyridazin-4-yl | ethyl | 4-[(propan-2-ylsulphanyl)methyl]phenyl |
| 1324 | pyridazin-4-yl | ethyl | 1-(2-bromo-6-chlorophenyl)-5-(dimethoxymethyl)-1H- |
| | | | pyrazol-4-yl |
| 1325 | pyridazin-4-yl | ethyl | 4-methoxy-3-[3-(pyridin-2-yl)-1H-pyrazol-1-yl]phenyl |
| 1326 | pyridazin-4-yl | ethyl | 3-chloro-4-{[4-(trifluoromethyl)-1H-pyrazol-1- |
| | | | yl]methyl}phenyl |
| 1327 | pyridazin-4-yl | ethyl | 3-[(4,6-dimethoxypyrimidin-2-yl)methoxy]phenyl |
| 1328 | pyridazin-4-yl | ethyl | 4-methyl-3-[(2-methylbutanoyl)amino]phenyl |
| 1329 | pyridazin-4-yl | ethyl | 4-{[chloro(difluoro)acetyl](methyl)amino}phenyl |
| 1330 | pyridazin-4-yl | ethyl | 4-{[3-(3-chloropyridin-2-yl)-1H-pyrazol-1-yl]methyl}phenyl |
| 1331 | pyridazin-4-yl | ethyl | 6-{[4-(pyridin-2-yl)-1H-pyrazol-1-yl]methyl}pyridin-3-yl |
| 1332 | pyridazin-4-yl | ethyl | 4-methoxy-3-[3-(pyrimidin-2-yl)-1H-pyrazol-1-yl]phenyl |
| 1333 | pyridazin-4-yl | ethyl | 6-{[4-(trifluoromethyl)phenyl]sulphanyl}pyridin-3-yl |
| 1334 | pyridazin-4-yl | ethyl | 4-{[3-(pyridin-2-yl)-1H-pyrazol-1-yl]methyl}phenyl |
| 1335 | pyridazin-4-yl | ethyl | 4-chloro-3-[(2,2-dimethylpropanoyl)amino]phenyl |
| 1336 | pyridazin-4-yl | ethyl | 1-(2-chloropyridin-3-yl)-5-(1-methoxyethyl)-1H-pyrazol-4-yl |
| 1337 | pyridazin-4-yl | ethyl | 2-(3,5-dichloropyridin-2-yl)-1,3-thiazol-4-yl |
| 1338 | pyridazin-4-yl | ethyl | 4-[(methylsulphanyl)methyl]phenyl |
| 1339 | pyridazin-4-yl | ethyl | 6-(propylamino)pyridin-3-yl |
| 1340 | pyridazin-4-yl | ethyl | 5-chloro-6-(4-cyanophenoxy)pyridin-3-yl |
| 1341 | pyridazin-4-yl | ethyl | 6-(3,4-dichlorophenoxy)pyridin-3-yl |
| 1342 | pyridazin-4-yl | ethyl | 6-[(2-chloropyrimidin-4-yl)oxy]pyridin-3-yl |
| 1343 | pyridazin-4-yl | ethyl | 1-(3-chloropyridin-2-yl)-5-(1-methoxyethyl)-1H-pyrazol-4-yl |
| 1344 | pyridazin-4-yl | ethyl | 2-[2-(trifluoromethyl)phenyl]-1,3-thiazol-4-yl |
| 1345 | pyridazin-4-yl | ethyl | 3-(propan-2-ylcarbamoyl)phenyl |
| 1346 | pyridazin-4-yl | ethyl | 4-(heptafluoropropyl)phenyl |
| 1347 | pyridazin-4-yl | ethyl | 2-(2,6-dichloropyridin-3-yl)-1,3-thiazol-4-yl |
| 1348 | pyridazin-4-yl | ethyl | 1-ethyl-5-(trifluoromethyl)-1H-pyrazol-4-yl |
| 1349 | pyridazin-4-yl | ethyl | 1-[3-(difluoromethyl)pyridin-2-yl]-5-methyl-1H-pyrazol-4-yl |
| 1350 | pyridazin-4-yl | ethyl | 1-(2,4-dichlorophenyl)-1H-pyrazol-4-yl |
| 1351 | pyridazin-4-yl | ethyl | 3-fluoro-4-(methylsulphanyl)phenyl |
| 1352 | pyridazin-4-yl | ethyl | 2-[4-chloro-2-(trifluoromethyl)phenyl]-1,3-thiazol-5-yl |
| 1353 | pyridazin-4-yl | ethyl | 1-(propan-2-yl)-5-(trifluoromethyl)-1H-pyrazol-4-yl |
| 1354 | pyridazin-4-yl | ethyl | 4-[(2-methylpyrimidin-4-yl)oxy]phenyl |
| 1355 | pyridazin-4-yl | ethyl | 6-{[5-(trifluoromethyl)pyridin-2-yl]sulphanyl}pyridin-3-yl |
| 1356 | pyridazin-4-yl | ethyl | 4-{[(propan-2-yloxy)imino]methyl}phenyl |
| 1357 | pyridazin-4-yl | ethyl | 4-(diethylcarbamoyl)phenyl |
| 1358 | pyridazin-4-yl | ethyl | 6-[(4-nitrophenyl)sulphanyl]pyridin-3-yl |
| 1359 | pyridazin-4-yl | ethyl | 4-methyl-3-{[(propan-2-yloxy)carbonyl]amino}phenyl |
| 1360 | pyridazin-4-yl | ethyl | 1-[4-fluoro-2-(trifluoromethyl)phenyl]-5-(methoxymethyl)-1H- |
| | | | pyrazol-4-yl |
| 1361 | pyridazin-4-yl | ethyl | 5-(1,1-difluoroethyl)-1-(pyrimidin-2-yl)-1H-pyrazol-4-yl |
| 1362 | pyridazin-4-yl | ethyl | 3-{[3,5-bis(trifluoromethyl)-1H-1,2,4-triazol-1- |
| | | | yl]methyl}phenyl |
| 1363 | pyridazin-4-yl | ethyl | 4-{[3,5-bis(trifluoromethyl)-1H-pyrazol-1-yl]methyl}phenyl |
| 1364 | pyridazin-4-yl | ethyl | 1-(2-chlorophenyl)-5-(1,1-dimethoxyethyl)-1H-pyrazol-4-yl |
| 1365 | pyridazin-4-yl | ethyl | 1-(3-chloropyridin-2-yl)-5-(1,1-difluoroethyl)-1H-pyrazol-4-yl |
| 1366 | pyridazin-4-yl | ethyl | 3-(1-fluoroethyl)-1-methyl-1H-pyrazol-4-yl |
| 1367 | pyridazin-4-yl | ethyl | 1-(2,6-dichloropyridin-3-yl)-5-(methoxymethyl)-1H-pyrazol-4- |
| | | | yl |
| 1368 | pyridazin-4-yl | ethyl | 6-(quinolin-6-yloxy)pyridin-3-yl |
| 1369 | pyridazin-4-yl | ethyl | 3-(4-chloro-1H-pyrazol-1-yl)-4-methoxyphenyl |
| 1370 | pyridazin-4-yl | ethyl | 2-(2,4-dichlorophenyl)-1,3-thiazol-4-yl |
| 1371 | pyridazin-4-yl | ethyl | 1-(2-chloropyridin-3-yl)-5-(1,1-difluoroethyl)-1H-pyrazol-4-yl |
| 1372 | pyridazin-4-yl | ethyl | 6-{4-[4-(methylsulphanyl)phenoxy]phenoxy}pyridin-3-yl |
| 1373 | pyridazin-4-yl | ethyl | 6-(quinolin-5-yloxy)pyridin-3-yl |
| 1374 | pyridazin-4-yl | ethyl | 1-[4-chloro-2-(trifluoromethyl)phenyl]-5-(difluoromethyl)-1H- |
| | | | pyrazol-4-yl |
| 1375 | pyridazin-4-yl | ethyl | 1-[2-(1-methoxyethyl)phenyl]-1H-pyrazol-4-yl |
| 1376 | pyridazin-4-yl | ethyl | 4-methoxy-3-[(2-methylbutanoyl)amino]phenyl |
| 1377 | pyridazin-4-yl | ethyl | 6-(2,4-dichlorophenoxy)pyridin-3-yl |
| 1378 | pyridazin-4-yl | ethyl | 4-{[acetyl(2-methylpropyl)amino]methyl}phenyl |
| 1379 | pyridazin-4-yl | ethyl | 6-[(methylsulphanyl)methyl]pyridin-3-yl |
| 1380 | pyridazin-4-yl | ethyl | 2-(2,4-dichlorophenyl)-1,3-thiazol-5-yl |
| 1381 | pyridazin-4-yl | ethyl | 6-[(4,5,6-trimethylpyrimidin-2-yl)sulphanyl]pyridin-3-yl |
| 1382 | pyridazin-4-yl | ethyl | 4-[(4,6-dimethylpyrimidin-2-yl)sulphanyl]phenyl |
| 1383 | pyridazin-4-yl | ethyl | 6-[2-chloro-4-(methylsulphanyl)phenoxy]pyridin-3-yl |
| 1384 | pyridazin-4-yl | ethyl | 1-(2-bromo-6-chlorophenyl)-5-(difluoromethyl)-1H-pyrazol-4- |
| | | | yl |
| 1385 | pyridazin-4-yl | ethyl | 4-(propan-2-yloxy)-3-[3-(trifluoromethyl)-1H-pyrazol-1- |
| | | | yl]phenyl |
| 1386 | pyridazin-4-yl | ethyl | 3-[ethyl(methyl)carbamoyl]phenyl |
| 1387 | pyridazin-4-yl | ethyl | 1-methyl-3-(trifluoromethyl)-1H-pyrazol-4-yl |
| 1388 | pyridazin-4-yl | ethyl | 6-(3,5-dimethylphenoxy)pyridin-3-yl |
| 1389 | pyridazin-4-yl | ethyl | 1-[4-chloro-2-(trifluoromethyl)phenyl]-5-methyl-1H-pyrazol- |
| | | | 4-yl |
| 1390 | pyridazin-4-yl | ethyl | 4-fluoro-3-(trifluoromethyl)phenyl |
| 1391 | pyridazin-4-yl | ethyl | 6-[4-(methoxycarbonyl)phenoxy]pyridin-3-yl |
| 1392 | pyridazin-4-yl | ethyl | 3-[(cyclopropylcarbonyl)amino]-4-methylphenyl |
| 1393 | pyridazin-4-yl | ethyl | 1-(4-chlorophenyl)-1H-pyrazol-4-yl |
| 1394 | pyridazin-4-yl | ethyl | 2-[2-(difluoromethyl)phenyl]-1,3-thiazol-5-yl |
| 1395 | pyridazin-4-yl | ethyl | 1-ethyl-3-(trifluoromethyl)-1H-pyrazol-4-yl |
| 1396 | pyridazin-4-yl | ethyl | 5-chloro-6-[4-(trifluoromethyl)phenoxy]pyridin-3-yl |
| 1397 | pyridazin-4-yl | ethyl | 4-methyl-3-(propanoylamino)phenyl |
| 1398 | pyridazin-4-yl | ethyl | 2-(2-chloropyridin-3-yl)-1,3-thiazol-4-yl |
| 1399 | pyridazin-4-yl | ethyl | 4-[(2-methylpropanoyl)amino]phenyl |
| 1400 | pyridazin-4-yl | ethyl | 3-[(2,2-dimethylpropanoyl)amino]-4-methylphenyl |
| 1401 | pyridazin-4-yl | ethyl | 4-methoxy-3-{[(propan-2-yloxy)carbonyl]amino}phenyl |
| 1402 | pyridazin-4-yl | ethyl | 1-(2,3-dichlorophenyl)-5-(methoxymethyl)-1H-pyrazol-4-yl |
| 1403 | pyridazin-4-yl | ethyl | 3-(hydroxymethyl)-1-methyl-1H-pyrazol-4-yl |
| 1404 | pyridazin-4-yl | ethyl | 1-(propan-2-yl)-3-(trifluoromethyl)-1H-pyrazol-4-yl |
| 1405 | pyridazin-4-yl | ethyl | 2-[3-(difluoromethyl)pyridin-2-yl]-1,3-thiazol-5-yl |
| 1406 | pyridazin-4-yl | ethyl | 4-(pyrrolidin-1-ylcarbonyl)phenyl |
| 1407 | pyridazin-4-yl | ethyl | 3-(methoxymethyl)-1-methyl-1H-pyrazol-4-yl |
| 1408 | pyridazin-4-yl | ethyl | 2-(3,5-dichloropyridin-2-yl)-1,3-thiazol-5-yl |
| 1409 | pyridazin-4-yl | ethyl | 1-(2,6-dichloropyridin-3-yl)-5-(difluoromethyl)-1H-pyrazol-4- |
| | | | yl |
| 1410 | pyridazin-4-yl | ethyl | 6-[(4,6-dimethylpyrimidin-2-yl)oxy]pyridin-3-yl |
| 1411 | pyridazin-4-yl | ethyl | 4-[(4,6-dimethoxy-1,3,5-triazin-2-yl)sulphanyl]phenyl |
| 1412 | pyridazin-4-yl | ethyl | 4-(methylsulphanyl)phenyl |
| 1413 | pyridazin-4-yl | ethyl | 4-(ethylsulphanyl)phenyl |
| 1414 | pyridazin-4-yl | ethyl | 6-[4-(trifluoromethoxy)phenoxy]pyridin-3-yl |
| 1415 | pyridazin-4-yl | ethyl | 5-chloro-6-(4-nitrophenoxy)pyridin-3-yl |
| 1416 | pyridazin-4-yl | ethyl | 2-[2-(difluoromethyl)phenyl]-1,3-thiazol-4-yl |
| 1417 | pyridazin-4-yl | ethyl | 3-(difluoromethyl)-1-methyl-1H-pyrazol-4-yl |
| 1418 | pyridazin-4-yl | ethyl | 1-(difluoromethyl)-3-(trifluoromethyl)-1H-pyrazol-4-yl |
| 1419 | pyridazin-4-yl | ethyl | 6-[(6-chloropyridin-2-yl)oxy]pyridin-3-yl |
| 1420 | pyridazin-4-yl | ethyl | 3-(butanoylamino)-4-methoxyphenyl |
| 1421 | pyridazin-4-yl | ethyl | 4-(acetylamino)phenyl |
| 1422 | pyridazin-4-yl | ethyl | 1-(2-bromophenyl)-5-(trifluoromethyl)-1H-pyrazol-4-yl |
| 1423 | pyridazin-4-yl | ethyl | 6-(4-chlorophenoxy)pyridin-3-yl |
| 1424 | pyridazin-4-yl | ethyl | 3-{[3-(trifluoromethyl)-1H-pyrazol-1-yl]methyl}phenyl |
| 1425 | pyridazin-4-yl | ethyl | 5-(difluoromethyl)-1-(2-methylphenyl)-1H-pyrazol-4-yl |
| 1426 | pyridazin-4-yl | ethyl | 2-[2-(1-methoxyethyl)phenyl]-1,3-thiazol-5-yl |
| 1427 | pyridazin-4-yl | ethyl | 1-(2-chlorophenyl)-5-(1,1-difluoroethyl)-1H-pyrazol-4-yl |
| 1428 | pyridazin-4-yl | ethyl | 1-(2-bromo-4-fluorophenyl)-1H-pyrazol-4-yl |
| 1429 | pyridazin-4-yl | ethyl | 6-[(cyclopropylmethyl)amino]pyridin-3-yl |
| 1430 | pyridazin-4-yl | ethyl | 6-[(2-methoxypyrimidin-4-yl)oxy]pyridin-3-yl |
| 1431 | pyridazin-4-yl | ethyl | 4-(methoxycarbonyl)phenyl |
| 1432 | pyridazin-4-yl | ethyl | 1-[2-(1,1-difluoroethyl)phenyl]-5-methyl-1H-pyrazol-4-yl |
| 1433 | pyridazin-4-yl | ethyl | 3-{[3,5-bis(trifluoromethyl)-1H-1,2,4-triazol-1-yl]methyl}-4- |
| | | | chlorophenyl |
| 1434 | pyridazin-4-yl | ethyl | 6-(morpholin-4-yl)pyridin-3-yl |
| 1435 | pyridazin-4-yl | ethyl | 2-(pyrimidin-2-yl)-1,3-thiazol-4-yl |
| 1436 | pyridazin-4-yl | ethyl | 4-[(4,6-dimethoxypyrimidin-2-yl)oxy]phenyl |
| 1437 | pyridazin-4-yl | ethyl | 2-{[3-(trifluoromethyl)-1H-pyrazol-1-yl]methyl}pyridin-4-yl |
| 1438 | pyridazin-4-yl | ethyl | 1-(2-chloropyridin-3-yl)-5-(trifluoromethyl)-1H-pyrazol-4-yl |
| 1439 | pyridazin-4-yl | ethyl | 6-(4-bromophenoxy)pyridin-3-yl |
| 1440 | pyridazin-4-yl | ethyl | 4-[(propylsulphanyl)methyl]phenyl |
| 1441 | pyridazin-4-yl | ethyl | 6-(pyrimidin-2-ylsulphanyl)pyridin-3-yl |
| 1442 | pyridazin-4-yl | ethyl | 1-(2-chloro-4-fluorophenyl)-1H-pyrazol-4-yl |
| 1443 | pyridazin-4-yl | ethyl | 4-methoxy-3-[4-(pyridin-2-yl)-1H-pyrazol-1-yl]phenyl |
| 1444 | pyridazin-4-yl | ethyl | 1-(2-chlorophenyl)-1H-pyrazol-4-yl |
| 1445 | pyridazin-4-yl | ethyl | 6-(2,6-dichlorophenoxy)pyridin-3-yl |
| 1446 | pyridazin-4-yl | ethyl | 3-[(4,6-dimethoxypyrimidin-2-yl)oxy]phenyl |
| 1447 | pyridazin-4-yl | ethyl | 6-(4-nitrophenoxy)pyridin-3-yl |
| 1448 | pyridazin-4-yl | ethyl | 1-(2-chlorophenyl)-5-methyl-1H-pyrazol-4-yl |
| 1449 | pyridazin-4-yl | ethyl | 3-[ethyl(methyl)carbamoyl]-4-methoxyphenyl |
| 1450 | pyridazin-4-yl | ethyl | 4-[(ethylsulphanyl)methyl]phenyl |
| 1451 | pyridazin-4-yl | ethyl | 2-[2-(1-methoxyethyl)phenyl]-1,3-thiazol-4-yl |
| 1452 | pyridazin-4-yl | ethyl | 6-{[6-(1H-pyrazol-1-yl)pyridin-2-yl]oxy}pyridin-3-yl |
| 1453 | pyridazin-4-yl | ethyl | 2-[4-chloro-2-(trifluoromethyl)phenyl]-1,3-thiazol-4-yl |
| 1454 | pyridazin-4-yl | ethyl | 4-methoxy-3-(1H-pyrazol-1-yl)phenyl |
| 1455 | pyridazin-4-yl | ethyl | 3-(acetylamino)-4-methylphenyl |
| 1456 | pyridazin-4-yl | ethyl | 2-{[4-(trifluoromethyl)-1H-pyrazol-1-yl]methyl}pyridin-4-yl |
| 1457 | pyridazin-4-yl | ethyl | 1-(2,4-difluorophenyl)-1H-pyrazol-4-yl |
| 1458 | pyridazin-4-yl | ethyl | 1-(2-methylphenyl)-5-(trifluoromethyl)-1H-pyrazol-4-yl |
| 1459 | pyridazin-4-yl | ethyl | 2-[3-(difluoromethyl)pyridin-2-yl]-1,3-thiazol-4-yl |
| 1460 | pyridazin-4-yl | ethyl | 6-(3,5-dichlorophenoxy)pyridin-3-yl |
| 1461 | pyridazin-4-yl | ethyl | 1-(pyrimidin-2-ylmethyl)-3-(trifluoromethyl)-1H-pyrazol-4-yl |
| 1462 | pyridazin-4-yl | ethyl | 6-methylpyridin-3-yl |
| 1463 | pyridazin-4-yl | ethyl | 6-[(4-methylphenyl)sulphanyl]pyridin-3-yl |
| 1464 | pyridazin-4-yl | ethyl | 1-(2,4-dichlorophenyl)-5-(methoxymethyl)-1H-pyrazol-4-yl |
| 1465 | pyridazin-4-yl | ethyl | 6-{4-[4-(trifluoromethyl)phenoxy]phenoxy}pyridin-3-yl |
| 1466 | pyridazin-4-yl | ethyl | 1-(2,6-dichlorophenyl)-5-(trifluoromethyl)-1H-pyrazol-4-yl |
| 1467 | pyridazin-4-yl | ethyl | 4-[(4,6-dimethylpyrimidin-2-yl)oxy]phenyl |
| 1468 | pyridazin-4-yl | ethyl | 6-(naphthalen-1-yloxy)pyridin-3-yl |
| 1469 | pyridazin-4-yl | ethyl | 3-ethyl-1-methyl-1H-pyrazol-4-yl |
| 1470 | pyridazin-4-yl | ethyl | 2-(2,6-dichloropyridin-3-yl)-1,3-thiazol-5-yl |
| 1471 | pyridazin-4-yl | ethyl | 6-{[(4,6-dimethoxypyrimidin-2-yl)methyl]sulphanyl}pyridin- |
| | | | 3-yl |
| 1472 | pyridazin-4-yl | ethyl | 1-[3-chloro-2-(trifluoromethyl)phenyl]-5-(1-methoxyethyl)- |
| | | | 1H-pyrazol-4-yl |
| 1473 | pyridazin-4-yl | ethyl | 3,4-dimethoxyphenyl |
| 1474 | pyridazin-4-yl | ethyl | 4-methoxy-3-[(methoxyacetyl)amino]phenyl |
| 1475 | pyridazin-4-yl | ethyl | 3-{[4-(trifluoromethyl)-1H-pyrazol-1-yl]methyl}phenyl |
| 1476 | pyridazin-4-yl | ethyl | 6-(2-cyanophenoxy)pyridin-3-yl |
| 1477 | pyridazin-4-yl | ethyl | 3-[(methylsulphanyl)methyl]phenyl |
| 1478 | pyridazin-4-yl | ethyl | 3-(dimethoxymethyl)-1-methyl-1H-pyrazol-4-yl |
| 1479 | pyridazin-4-yl | ethyl | 1-(ethoxymethyl)-3-(trifluoromethyl)-1H-pyrazol-4-yl |
| 1480 | pyridazin-4-yl | ethyl | 1-(3-bromopyridin-2-yl)-5-(methoxymethyl)-1H-pyrazol-4-yl |
| 1481 | pyridazin-4-yl | ethyl | 6-(methylsulphanyl)pyridin-3-yl |
| 1482 | pyridazin-4-yl | ethyl | 1-(2-bromo-6-chlorophenyl)-5-(trifluoromethyl)-1H-pyrazol-4- |
| | | | yl |
| 1483 | pyridazin-4-yl | ethyl | 3-{[4-(trifluoromethyl)phenoxy]methyl}phenyl |
| 1484 | pyridazin-4-yl | ethyl | 6-(heptafluoropropyl)pyridin-3-yl |
| 1485 | pyridazin-4-yl | ethyl | 1-ethenyl-3-(trifluoromethyl)-1H-pyrazol-4-yl |
| 1486 | pyridazin-4-yl | ethyl | 4-methyl-3-nitrophenyl |
| 1487 | pyridazin-4-yl | ethyl | 3-(4-bromo-1H-pyrazol-1-yl)-4-methoxyphenyl |
| 1488 | pyridazin-4-yl | ethyl | 5-methyl-1-(2-methylphenyl)-1H-pyrazol-4-yl |
| 1489 | pyridazin-4-yl | ethyl | 1-[3-(difluoromethyl)pyridin-2-yl]-5-(methoxymethyl)-1H- |
| | | | pyrazol-4-yl |
| 1490 | pyridazin-4-yl | ethyl | 6-{[2-(trifluoromethyl)pyrimidin-5-yl]oxy}pyridin-3-yl |
| 1491 | pyridazin-4-yl | ethyl | 2-[2-(methoxymethyl)phenyl]-1,3-thiazol-5-yl |
| 1492 | pyridazin-4-yl | ethyl | 1-(ethoxymethyl)-5-(trifluoromethyl)-1H-pyrazol-4-yl |
| 1493 | pyridazin-4-yl | ethyl | 5-chloro-6-[(4,5,6-trimethylpyrimidin-2-yl)sulphanyl]pyridin- |
| | | | 3-yl |
| 1494 | pyridazin-4-yl | ethyl | 6-[3,5-bis(trifluoromethyl)phenoxy]pyridin-3-yl |
| 1495 | pyridazin-4-yl | ethyl | 4-{[4-(pyridin-2-yl)-1H-pyrazol-1-yl]methyl}phenyl |
| 1496 | pyridazin-4-yl | ethyl | 1-[3-(1,1-difluoroethyl)pyridin-2-yl]-5-methyl-1H-pyrazol-4-yl |
| 1497 | pyridazin-4-yl | ethyl | 1-(2,6-dichlorophenyl)-5-(difluoromethyl)-1H-pyrazol-4-yl |
| 1498 | pyridazin-4-yl | ethyl | 1-[4-fluoro-2-(trifluoromethyl)phenyl]-5-methyl-1H-pyrazol-4- |
| | | | yl |
| 1499 | pyridazin-4-yl | ethyl | 4-chloro-3-[(cyclobutylcarbonyl)amino]phenyl |
| 1500 | pyridazin-4-yl | ethyl | 2-[2-(methoxymethyl)phenyl]-1,3-thiazol-4-yl |
| 1501 | pyridazin-4-yl | ethyl | 4-(1H-pyrazol-1-ylmethyl)phenyl |
| 1502 | pyridazin-4-yl | ethyl | 1-(2-bromophenyl)-5-methyl-1H-pyrazol-4-yl |
| 1503 | pyridazin-4-yl | ethyl | 1-(2,3-difluorophenyl)-1H-pyrazol-4-yl |
| 1504 | pyridazin-4-yl | ethyl | 6-{4-[(trifluoromethyl)sulphanyl]phenoxy}pyridin-3-yl |
| 1505 | pyridazin-4-yl | ethyl | 4-{[3-(pentafluoroethyl)-1H-1,2,4-triazol-1-yl]methyl}phenyl |
| 1506 | pyridazin-4-yl | ethyl | 4-{[3-(pyrimidin-2-yl)-1H-pyrazol-1-yl]methyl}phenyl |
| 1507 | pyridazin-4-yl | ethyl | 1-(2-chlorophenyl)-5-(difluoromethyl)-1H-pyrazol-4-yl |
| 1508 | pyridazin-4-yl | ethyl | 5-(difluoromethyl)-1-[2-(trifluoromethyl)phenyl]-1H-pyrazol- |
| | | | 4-yl |
| 1509 | pyridazin-4-yl | ethyl | 6-(4-methoxyphenoxy)pyridin-3-yl |
| 1510 | pyridazin-4-yl | ethyl | 1-[2-(dimethoxymethyl)phenyl]-1H-pyrazol-4-yl |
| 1511 | pyridazin-4-yl | ethyl | 5-(methoxymethyl)-1-(pyrimidin-2-yl)-1H-pyrazol-4-yl |
| 1512 | pyridazin-4-yl | ethyl | 6-tert-butoxypyridin-3-yl |
| 1513 | pyridazin-4-yl | ethyl | 4-[(cyclopentylimino)methyl]phenyl |
| 1514 | pyridazin-4-yl | ethyl | 1-(3-chlorophenyl)-1H-pyrazol-4-yl |
| 1515 | pyridazin-4-yl | ethyl | 3-[(2-methylpropanoyl)amino]phenyl |
| 1516 | pyridazin-4-yl | ethyl | 6-{[3,5-bis(trifluoromethyl)-1H-pyrazol-1-yl]methyl}pyridin- |
| | | | 3-yl |
| 1517 | pyridazin-4-yl | ethyl | 4-chloro-3-[(2-methylpropanoyl)amino]phenyl |
| 1518 | pyridazin-4-yl | ethyl | 4-(propylsulphanyl)phenyl |
| 1519 | pyridazin-4-yl | ethyl | 1-[2-(difluoromethyl)phenyl]-5-(methoxymethyl)-1H-pyrazol- |
| | | | 4-yl |
| 1520 | pyridazin-4-yl | ethyl | 4-methoxy-3-[4-(pyrimidin-2-yl)-1H-pyrazol-1-yl]phenyl |
| 1521 | pyridazin-4-yl | ethyl | 4-{[(4,6-dimethylpyrimidin-2-yl)sulphanyl]methyl}phenyl |
| 1522 | pyridazin-4-yl | ethyl | 1-(2-chlorophenyl)-5-(methoxymethyl)-1H-pyrazol-4-yl |
| 1523 | pyridazin-4-yl | ethyl | 6-{[3,5-bis(trifluoromethyl)-1H-1,2,4-triazol-1- |
| | | | yl]methyl}pyridin-3-yl |
| 1524 | pyridazin-4-yl | ethyl | 5-chloro-6-[4-(trifluoromethoxy)phenoxy]pyridin-3-yl |
| 1525 | pyridazin-4-yl | ethyl | 4-[(tert-butoxyimino)methyl]phenyl |
| 1526 | pyridazin-4-yl | ethyl | 2-(3-chloropyridin-2-yl)-1,3-thiazol-5-yl |
| 1527 | pyridazin-4-yl | ethyl | 1-(2-fluorophenyl)-1H-pyrazol-4-yl |
| 1528 | pyridazin-4-yl | ethyl | 4-(trifluoromethoxy)phenyl |
| 1529 | pyridazin-4-yl | ethyl | 6-(2-chlorophenoxy)pyridin-3-yl |
| 1530 | pyridazin-4-yl | ethyl | 3-[(2,2-dimethylpropanoyl)amino]-5-(trifluoromethyl)phenyl |
| 1531 | pyridazin-4-yl | ethyl | 6-[(4-methylpyrimidin-2-yl)sulphanyl]pyridin-3-yl |
| 1532 | pyridazin-4-yl | ethyl | 6-{[2-(morpholin-4-yl)ethyl]amino}pyridin-3-yl |
| 1533 | pyridazin-4-yl | ethyl | 3-formyl-1-methyl-1H-pyrazol-4-yl |
| 1534 | pyridazin-4-yl | ethyl | 1-methyl-3-(propan-2-yl)-1H-pyrazol-4-yl |
| 1535 | pyridazin-4-yl | ethyl | 1-(3-chlorophenyl)-5-(1,1-dimethoxyethyl)-1H-pyrazol-4-yl |
| 1536 | pyridazin-4-yl | ethyl | 4-nitrophenyl |
| 1537 | pyridazin-4-yl | ethyl | 6-{[5-(methoxycarbonyl)pyridin-3-yl]oxy}pyridin-3-yl |
| 1538 | pyridazin-4-yl | ethyl | 3-{[4-(trifluoromethyl)benzyl]oxy}phenyl |
| 1539 | pyridazin-4-yl | ethyl | 1-(2,4-dichlorophenyl)-5-(1,1-dimethoxyethyl)-1H-pyrazol-4- |
| | | | yl |
| 1540 | pyridazin-4-yl | ethyl | 4-{[4-bromo-3-(trifluoromethyl)-1H-pyrazol-1- |
| | | | yl]methyl}phenyl |
| 1541 | pyridazin-4-yl | ethyl | 3-[chloro(difluoro)methyl]-1-methyl-1H-pyrazol-4-yl |
| 1542 | pyridazin-4-yl | ethyl | 3-[(cyclopropylcarbonyl)amino]-4-fluorophenyl |
| 1543 | pyridazin-4-yl | ethyl | 6-[4-(dimethylsulphamoyl)phenoxy]pyridin-3-yl |
| 1544 | pyridazin-4-yl | ethyl | 5-methyl-1-(2-methylpyridin-3-yl)-1H-pyrazol-4-yl |
| 1545 | pyridazin-4-yl | ethyl | 1-(2,4-difluorophenyl)-5-methyl-1H-pyrazol-4-yl |
| 1546 | pyridazin-4-yl | ethyl | 3-(methylsulphanyl)phenyl |
| 1547 | pyridazin-4-yl | ethyl | 3-(acetylamino)-4-chlorophenyl |
| 1548 | pyridazin-4-yl | ethyl | 4-[(4-bromo-1H-pyrazol-1-yl)methyl]phenyl |
| 1549 | pyridazin-4-yl | ethyl | 4-methyl-3-[(propan-2-ylcarbamoyl)amino]phenyl |
| 1550 | pyridazin-4-yl | ethyl | 1-(2,2-diethoxyethyl)-3-(trifluoromethyl)-1H-pyrazol-4-yl |
| 1551 | pyridazin-4-yl | ethyl | 6-[(4,6-dimethoxypyrimidin-2-yl)oxy]pyridin-3-yl |
| 1552 | pyridazin-4-yl | ethyl | 6-(1H-pyrazol-1-yl)pyridin-3-yl |
| 1553 | pyridazin-4-yl | ethyl | 4-(1H-1,2,4-triazol-1-ylmethyl)phenyl |
| 1554 | pyridazin-4-yl | ethyl | 5-(methoxymethyl)-1-[2-(trifluoromethyl)phenyl]-1H-pyrazol- |
| | | | 4-yl |
| 1555 | pyridazin-4-yl | ethyl | 5-methylpyridin-3-yl |
| 1556 | pyridazin-4-yl | ethyl | 5-chloro-6-[(4,6-dimethylpyrimidin-2-yl)sulphanyl]pyridin-3- |
| | | | yl |
| 1557 | pyridazin-4-yl | ethyl | 3-[(2,2-dimethylpropanoyl)amino]-4-methoxyphenyl |
| 1558 | pyridazin-4-yl | ethyl | 2-[2-(trifluoromethyl)pyridin-3-yl]-1,3-thiazol-4-yl |
| 1559 | pyridazin-4-yl | ethyl | 3-cyclopropyl-1-methyl-1H-pyrazol-4-yl |
| 1560 | pyridazin-4-yl | ethyl | 3-methyl-1H-pyrazol-4-yl |
| 1561 | pyridazin-4-yl | ethyl | 5-(methoxymethyl)-1-(2,2,2-trifluoroethyl)-1H-pyrazol-4-yl |
| 1562 | pyridazin-4-yl | ethyl | 4-methoxy-3-(propanoylamino)phenyl |
| 1563 | pyridazin-4-yl | ethyl | 4-methoxy-3-[(2-methylpropanoyl)amino]phenyl |
| 1564 | pyridazin-4-yl | ethyl | 6-(quinolin-7-yloxy)pyridin-3-yl |
| 1565 | pyridazin-4-yl | ethyl | 4-{[3-(trifluoromethyl)-1H-pyrazol-1-yl]methyl}phenyl |
| 1566 | pyridazin-4-yl | ethyl | 3-[(4,6-dimethylpyrimidin-2-yl)oxy]phenyl |
| 1567 | pyridazin-4-yl | ethyl | 4-{[3,5-bis(trifluoromethyl)-1H-1,2,4-triazol-1- |
| | | | yl]methyl}phenyl |
| 1568 | pyridazin-4-yl | ethyl | 3-fluoro-4-{[4-(trifluoromethyl)-1H-pyrazol-1- |
| | | | yl]methyl}phenyl |
| 1569 | pyridazin-4-yl | ethyl | 2,3-dihydro-1,4-benzodioxin-6-yl |
| 1570 | pyridazin-4-yl | ethyl | 2-[3-(methoxymethyl)pyridin-2-yl]-1,3-thiazol-5-yl |
| 1571 | pyridazin-4-yl | ethyl | 6-({6-[3-(trifluoromethyl)-1H-pyrazol-1-yl]pyridin-2- |
| | | | yl}oxy)pyridin-3-yl |
| 1572 | pyridazin-4-yl | ethyl | 3-{[3-(pyridin-2-yl)-1H-pyrazol-1-yl]methyl}phenyl |
| 1573 | pyridazin-4-yl | ethyl | 1-(2-bromo-4-chlorophenyl)-5-methyl-1H-pyrazol-4-yl |
| 1574 | pyridazin-4-yl | ethyl | 2,2-difluoro-1,3-benzodioxo1-5-yl |
| 1575 | pyridazin-4-yl | ethyl | 2-[3-(trifluoromethyl)pyridin-2-yl]-1,3-thiazol-4-yl |
| 1576 | pyridazin-4-yl | ethyl | 4-(benzylcarbamoyl)phenyl |
| 1577 | pyridazin-4-yl | ethyl | 4-[(2-chloropyrimidin-4-yl)oxy]phenyl |
| 1578 | pyridazin-4-yl | ethyl | 1-[3-(trifluoromethyl)pyridin-2-yl]-1H-pyrazol-4-yl |
| 1579 | pyridazin-4-yl | ethyl | 5-chloro-6-(4-chlorophenoxy)pyridin-3-yl |
| 1580 | pyridazin-4-yl | ethyl | 3-cyano-4-(1H-1,2,4-triazol-1-yl)phenyl |
| 1581 | pyridazin-4-yl | ethyl | 3-amino-4-(methylsulphonyl)phenyl |
| 1582 | pyridazin-4-yl | ethyl | 4-methyl-3-[(2-methylpropanoyl)amino]phenyl |
| 1583 | pyridazin-4-yl | ethyl | 1-(2,6-dibromophenyl)-5-(trifluoromethyl)-1H-pyrazol-4-yl |
| 1584 | pyridazin-4-yl | ethyl | 3-{[3-(trifluoromethyl)-1H-1,2,4-triazol-1-yl]methyl}phenyl |
| 1585 | pyridazin-4-yl | ethyl | 4-[(3,3,3-trifluoropropanoyl)amino]phenyl |
| 1586 | pyridazin-4-yl | ethyl | 5-(pyrazin-2-yl)-1-(2,2,2-trifluoroethyl)-1H-pyrazol-4-yl |
| 1587 | pyridazin-4-yl | ethyl | 1-(2,6-dichlorophenyl)-5-(dimethoxymethyl)-1H-pyrazol-4-yl |
| 1588 | pyridazin-4-yl | ethyl | 1-(4-chlorophenyl)-5-(1,1-dimethoxyethyl)-1H-pyrazol-4-yl |
| 1589 | pyridazin-4-yl | ethyl | 6-[3-(trifluoromethyl)phenoxy]pyridin-3-yl |
| 1590 | pyridazin-4-yl | ethyl | 4-({6-[3-(trifluoromethyl)-1H-pyrazol-1-yl]pyridin-2- |
| | | | yl}oxy)phenyl |
| 1591 | pyridazin-4-yl | ethyl | 4-methoxy-3-(propan-2-ylcarbamoyl)phenyl |
| 1592 | pyridazin-4-yl | ethyl | 1-[2-(1-methoxyethyl)phenyl]-5-methyl-1H-pyrazol-4-yl |
| 1593 | pyridazin-4-yl | ethyl | 1-ethyl-3-methyl-1H-pyrazol-4-yl |
| 1594 | pyridazin-4-yl | ethyl | 4-[(4,6-dimethoxypyrimidin-2-yl)sulphanyl]phenyl |
| 1595 | pyridazin-4-yl | ethyl | 6-(4-methylphenoxy)pyridin-3-yl |
| 1596 | pyridazin-4-yl | ethyl | 6-(4-chloro-2-cyanophenoxy)pyridin-3-yl |
| 1597 | pyridazin-4-yl | ethyl | 1-propyl-3-(trifluoromethyl)-1H-pyrazol-4-yl |
| 1598 | pyridazin-4-yl | ethyl | 6-(2-methylphenoxy)pyridin-3-yl |
| 1599 | pyridazin-4-yl | ethyl | 4-{[4-(trifluoromethyl)-1H-pyrazol-1-yl]methyl}phenyl |
| 1600 | pyridazin-4-yl | ethyl | 6-[(6-methylpyridin-3-yl)oxy]pyridin-3-yl |
| 1601 | pyridazin-4-yl | ethyl | 6-methoxypyridin-3-yl |
| 1602 | pyridazin-4-yl | ethyl | 4-[(ethoxyimino)methyl]phenyl |
| 1603 | pyridazin-4-yl | ethyl | 4-{[acetyl(cyclopentyl)amino]methyl}phenyl |
| 1604 | pyridazin-4-yl | ethyl | 3-{[3,5-bis(trifluoromethyl)-1H-pyrazol-1-yl]methyl}phenyl |
| 1605 | pyridazin-4-yl | ethyl | 1-(2-bromo-4-chlorophenyl)-1H-pyrazol-4-yl |
| 1606 | pyridazin-4-yl | ethyl | 6-[4-(1H-1,2,4-triazol-1-yl)phenoxy]pyridin-3-yl |
| 1607 | pyridazin-4-yl | ethyl | 6-[4-(trifluoromethyl)phenoxy]pyridin-3-yl |
| 1608 | pyridazin-4-yl | ethyl | 1-(2-chlorophenyl)-5-(trifluoromethyl)-1H-pyrazol-4-yl |
| 1609 | pyridazin-4-yl | ethyl | 4-methoxy-3-nitrophenyl |
| 1610 | pyridazin-4-yl | ethyl | 1-(2,6-dibromophenyl)-5-(difluoromethyl)-1H-pyrazol-4-yl |
| 1611 | pyridazin-4-yl | ethyl | 6-[(4-methoxyphenyl)sulphanyl]pyridin-3-yl |
| 1612 | pyridazin-4-yl | ethyl | 1-(2,6-dichlorophenyl)-5-(1-methoxyethyl)-1H-pyrazol-4-yl |
| 1613 | pyridazin-4-yl | ethyl | 4-[(6-chloropyridin-2-yl)oxy]phenyl |
| 1614 | pyridazin-4-yl | ethyl | 3-fluoro-4-{[3-(trifluoromethyl)-1H-pyrazol-1- |
| | | | yl]methyl}phenyl |
| 1615 | pyridazin-4-yl | ethyl | 4-{[(difluoromethyl)sulphanyl]methyl}phenyl |
| 1616 | pyridazin-4-yl | ethyl | 6-{[3-(trifluoromethyl)-1H-pyrazol-1-yl]methyl}pyridin-3-yl |
| 1617 | pyridazin-4-yl | ethyl | 4-{[2-(3,5-dichlorophenyl)propan-2-yl]carbamoyl}phenyl |
| 1618 | pyridazin-4-yl | ethyl | 3-(1H-1,2,4-triazol-1-ylmethyl)phenyl |
| 1619 | pyridazin-4-yl | ethyl | 2-(3-chloropyridin-2-yl)-1,3-thiazol-4-yl |
| 1620 | pyridazin-4-yl | ethyl | 5-(1-methoxyethyl)-1-(pyrimidin-2-yl)-1H-pyrazol-4-yl |
| 1621 | pyridazin-4-yl | ethyl | 1-(3-chloropyridin-2-yl)-5-(1,1-dimethoxyethyl)-1H-pyrazol- |
| | | | 4-yl |
| 1622 | pyridazin-4-yl | ethyl | 1-(2-bromo-4-fluorophenyl)-5-methyl-1H-pyrazol-4-yl |
| 1623 | pyridazin-4-yl | ethyl | 3-(butanoylamino)-4-methylphenyl |
| 1624 | pyridazin-4-yl | ethyl | 5-(methoxymethyl)-1-[3-(trifluoromethyl)pyridin-2-yl]-1H- |
| | | | pyrazol-4-yl |
| 1625 | pyridazin-4-yl | ethyl | 6-[3-(trifluoromethyl)-1H-pyrazol-1-yl]pyridin-3-yl |
| 1626 | pyridazin-4-yl | ethyl | 6-(3-chlorophenoxy)pyridin-3-yl |
| 1627 | pyridazin-4-yl | ethyl | 1-(2,6-dichloropyridin-3-yl)-5-(1-methoxyethyl)-1H-pyrazol- |
| | | | 4-yl |
| 1628 | pyridazin-4-yl | ethyl | 6-[(4,6-dimethylpyrimidin-2-yl)sulphanyl]pyridin-3-yl |
| 1629 | pyridazin-4-yl | ethyl | 1-[2-(difluoromethyl)phenyl]-5-methyl-1H-pyrazol-4-yl |
| 1630 | pyridazin-4-yl | ethyl | 2-[2-(trifluoromethyl)phenyl]-1,3-thiazol-5-yl |
| 1631 | pyridazin-4-yl | ethyl | 2-[2-(trifluoromethyl)pyridin-3-yl]-1,3-thiazol-5-yl |
| 1632 | pyridazin-4-yl | ethyl | 6-[(4,6-dimethoxy-1,3,5-triazin-2-yl)sulphanyl]pyridin-3-yl |
| 1633 | pyridazin-4-yl | ethyl | 2-(2-chloropyridin-3-yl)-1,3-thiazol-5-yl |
| 1634 | pyridazin-4-yl | ethyl | 1-(2,6-dichloropyridin-3-yl)-5-methyl-1H-pyrazol-4-yl |
| 1635 | pyridazin-4-yl | ethyl | 1-(3-chloropyridin-2-yl)-5-(methoxymethyl)-1H-pyrazol-4-yl |
| 1636 | pyridazin-4-yl | ethyl | 6-[(4-chlorophenyl)sulphanyl]pyridin-3-yl |
| 1637 | pyridazin-4-yl | ethyl | 6-(piperidin-l-yl)pyridin-3-yl |
| 1638 | pyridazin-4-yl | ethyl | 3-[(cyclopentylcarbonyl)amino]-4-methylphenyl |
| 1639 | pyridazin-4-yl | ethyl | 6-ethoxypyridin-3-yl |
| 1640 | pyridazin-4-yl | ethyl | 2-(2,6-dichlorophenyl)-1,3-thiazol-4-yl |
| 1641 | pyridazin-4-yl | ethyl | 5-chloro-6-[(4-methylpyrimidin-2-yl)sulphanyl]pyridin-3-yl |
| 1642 | pyridazin-4-yl | ethyl | 1-[4-chloro-2-(trifluoromethyl)phenyl]-5-(1-methoxyethyl)- |
| | | | 1H-pyrazol-4-yl |
| 1643 | pyridazin-4-yl | ethyl | 6-(3-methylphenoxy)pyridin-3-yl |
| 1644 | pyridazin-4-yl | ethyl | 4-[ethyl(methyl)carbamoyl]phenyl |
| 1645 | pyridazin-4-yl | ethyl | 6-[3-(trifluoromethoxy)phenoxy]pyridin-3-yl |
| 1646 | pyridazin-4-yl | ethyl | 5-(difluoromethyl)-1-(pyrimidin-2-yl)-1H-pyrazol-4-yl |
| 1647 | pyridazin-4-yl | ethyl | 4-[(cyclopropylcarbonyl)amino]phenyl |
| 1648 | pyridazin-4-yl | ethyl | 1-(3-bromopyridin-2-yl)-5-(1-methoxyethyl)-1H-pyrazol-4-yl |
| 1649 | pyridazin-4-yl | ethyl | 3-[(cyclopropylcarbonyl)amino]-4-methoxyphenyl |
| 1650 | pyridazin-4-yl | ethyl | 4-methyl-3-[(3-methylbut-2-enoyl)amino]phenyl |
| 1651 | pyridazin-4-yl | ethyl | 1-[3-(methoxymethyl)pyridin-2-yl]-1H-pyrazol-4-yl |
| 1652 | pyridazin-4-yl | ethyl | 4-[acetyl(methyl)amino]phenyl |
| 1653 | pyridazin-4-yl | ethyl | 6-(naphthalen-2-yloxy)pyridin-3-yl |
| 1654 | pyridazin-4-yl | ethyl | 1-(2-chlorophenyl)-5-(1-methoxyethyl)-1H-pyrazol-4-yl |
| 1655 | pyridazin-4-yl | ethyl | 1-[2-(difluoromethyl)phenyl]-5-(1-methoxyethyl)-1H-pyrazol- |
| | | | 4-yl |
| 1656 | pyridazin-4-yl | ethyl | 1-(3-chloropyridin-2-yl)-5-methyl-1H-pyrazol-4-yl |
| 1657 | pyridazin-4-yl | ethyl | 4-{[3-(trifluoromethyl)-1H-1,2,4-triazol-1-yl]methyl}phenyl |
| 1658 | pyridazin-4-yl | ethyl | 3-[(cyclopentylcarbonyl)amino]-4-methoxyphenyl |
| 1659 | pyridazin-4-yl | ethyl | 1-(2,2,2-trifluoroethyl)-5-(trifluoromethyl)-1H-pyrazol-4-yl |
| 1660 | pyridazin-4-yl | ethyl | 6-[4-(methylsulphanyl)phenoxy]pyridin-3-yl |
| 1661 | pyridazin-4-yl | ethyl | 2-[3-(trifluoromethyl)pyridin-2-yl]-1,3-thiazol-5-yl |
| 1662 | pyridazin-4-yl | ethyl | 4-{[3-(methoxycarbonyl)-1H-1,2,4-triazol-1-yl]methyl}phenyl |
| 1663 | pyridazin-4-yl | ethyl | 1-[3-(1,1-difluoroethyl)pyridin-2-yl]-1H-pyrazol-4-yl |
| 1664 | pyridazin-4-yl | ethyl | 6-{[3-(pyridin-2-yl)-1H-pyrazol-1-yl]methyl}pyridin-3-yl |
| 1665 | pyridazin-4-yl | ethyl | 3-(acetylamino)phenyl |
| 1666 | pyridazin-4-yl | ethyl | 1-(3-chloropyridin-2-yl)-5-(trifluoromethyl)-1H-pyrazol-4-yl |
| 1667 | pyridazin-4-yl | ethyl | 2-{[3-(pyridin-2-yl)-1H-pyrazol-1-yl]methyl}pyridin-4-yl |
| 1668 | pyridazin-4-yl | ethyl | 3-[(methoxyacetyl)amino]-4-methylphenyl |
| 1669 | pyridazin-4-yl | ethyl | 1-(3-bromopyridin-2-yl)-5-(trifluoromethyl)-1H-pyrazol-4-yl |
| 1670 | pyridazin-4-yl | ethyl | 4-[(methoxyimino)methyl]phenyl |
| 1671 | pyridazin-4-yl | ethyl | 1-(pyrimidin-2-yl)-5-(trifluoromethyl)-1H-pyrazol-4-yl |
| 1672 | pyridazin-4-yl | ethyl | 2-(2-chlorophenyl)-1,3-thiazol-4-yl |
| 1673 | pyridazin-4-yl | ethyl | 4-methoxy-3-[(propan-2-ylsulphonyl)amino]phenyl |
| 1674 | pyridazin-4-yl | ethyl | 6-(3-cyanophenoxy)pyridin-3-yl |
| 1675 | pyridazin-4-yl | ethyl | 4-fluoro-3-[(2-methylpropanoyl)amino]phenyl |
| 1676 | pyridazin-4-yl | ethyl | 1-(2-bromophenyl)-5-(difluoromethyl)-1H-pyrazol-4-yl |
| 1677 | pyridazin-4-yl | ethyl | 1,3-dimethyl-1H-pyrazol-4-yl |
| 1678 | pyridazin-4-yl | ethyl | 5-(1-methoxyethyl)-1-(2,2,2-trifluoroethyl)-1H-pyrazol-4-yl |
| 1679 | pyridazin-4-yl | ethyl | 1-(2,6-dichlorophenyl)-5-(1-fluoroethyl)-1H-pyrazol-4-yl |
| 1680 | pyridazin-4-yl | ethyl | 6-(pyrrolidin-l-yl)pyridin-3-yl |
| 1681 | pyridazin-4-yl | ethyl | 3-(acetylamino)-5-(trifluoromethyl)phenyl |
| 1682 | pyridazin-4-yl | ethyl | 2-[3-(methoxymethyl)pyridin-2-yl]-1,3-thiazol-4-yl |
| 1683 | pyridazin-4-yl | ethyl | 2-[3-(1-methoxyethyl)pyridin-2-yl]-1,3-thiazol-5-yl |
| 1684 | pyridazin-4-yl | ethyl | 1-[3-(dimethoxymethyl)pyridin-2-yl]-1H-pyrazol-4-yl |
| 1685 | pyridazin-4-yl | ethyl | 5-(1-methoxyethyl)-1-(2-methylphenyl)-1H-pyrazol-4-yl |
| 1686 | pyridazin-4-yl | ethyl | 1-methyl-1H-benzotriazol-5-yl |
| 1687 | pyridazin-4-yl | ethyl | 4-[(butylsulphanyl)methyl]phenyl |
| 1688 | pyridazin-4-yl | ethyl | 1H-pyrazol-4-yl |
| 1689 | pyridazin-4-yl | ethyl | 1-(2-bromo-6-chlorophenyl)-5-(1-methoxyethyl)-1H-pyrazol- |
| | | | 4-yl |
| 1690 | pyridazin-4-yl | ethyl | 5-chloro-6-[4-(methylsulphonyl)phenoxy]pyridin-3-yl |
| 1691 | pyridazin-4-yl | ethyl | 1-[3-(1-methoxyethyl)pyridin-2-yl]-1H-pyrazol-4-yl |
| 1692 | pyridazin-4-yl | ethyl | 6-[(4,6-dimethoxypyrimidin-2-yl)sulphanyl]pyridin-3-yl |
| 1693 | pyridazin-4-yl | ethyl | 4-[(6-methylpyridin-3-yl)oxy]phenyl |
| 1694 | pyridazin-4-yl | ethyl | 3-methoxy-1-methyl-1H-pyrazol-4-yl |
| 1695 | pyridazin-4-yl | ethyl | 6-(4-tert-butylphenoxy)pyridin-3-yl |
| 1696 | pyridazin-4-yl | ethyl | 1-(2,4-dichlorophenyl)-5-methyl-1H-pyrazol-4-yl |
| 1697 | pyridazin-4-yl | ethyl | 3-(but-2-enoylamino)-4-methylphenyl |
| 1698 | pyridazin-4-yl | ethyl | 4-{[4-(pyrimidin-2-yl)-1H-pyrazol-1-yl]methyl}phenyl |
| 1699 | pyridazin-4-yl | ethyl | 4-{[6-(1H-pyrazol-1-yl)pyridin-2-yl]oxy}phenyl |
| 1700 | pyridazin-4-yl | ethyl | 4-{[3,5-bis(trifluoromethyl)-1H-1,2,4-triazol-1-yl]methyl}-3- |
| | | | chlorophenyl |
| 1701 | pyridazin-4-yl | ethyl | 4-[(2-methoxypyrimidin-4-yl)oxy]phenyl |
| 1702 | pyridazin-4-yl | ethyl | 1-[4-chloro-2-(trifluoromethyl)phenyl]-5-(methoxymethyl)- |
| | | | 1H-pyrazol-4-yl |
| 1703 | pyridazin-4-yl | ethyl | 5-(trifluoromethyl)-1-[2-(trifluoromethyl)phenyl]-1H-pyrazol- |
| | | | 4-yl |
| 1704 | pyridazin-4-yl | ethyl | 6-(dimethylamino)pyridin-3-yl |
| 1705 | pyridazin-4-yl | ethyl | 2-[5-chloro-3-(trifluoromethyl)pyridin-2-yl]-1,3-thiazol-5-yl |
| 1706 | pyridazin-4-yl | ethyl | 4-{[4-(3-chloropyridin-2-yl)-1H-pyrazol-1-yl]methyl}phenyl |
| 1707 | pyridazin-4-yl | ethyl | 4-[(2,2,3,3,4,4,4-heptafluorobutanoyl)(methyl)amino]phenyl |
| 1708 | pyridazin-4-yl | ethyl | 4-[(trifluoromethyl)sulphanyl]phenyl |
| 1709 | pyridazin-4-yl | ethyl | 6-[4-(methylsulphonyl)phenoxy]pyridin-3-yl |
| 1710 | pyridazin-4-yl | ethyl | 1-(2-bromophenyl)-5-(methoxymethyl)-1H-pyrazol-4-yl |
| 1711 | pyridazin-4-yl | ethyl | 6-[(4-bromo-1H-pyrazol-1-yl)methyl]pyridin-3-yl |
| 1712 | pyridazin-4-yl | ethyl | 3-chloro-4-{[3-(trifluoromethyl)-1H-pyrazol-1- |
| | | | yl]methyl}phenyl |
| 1713 | pyridazin-4-yl | ethyl | 6-{[4-(trifluoromethyl)-1H-pyrazol-1-yl]methyl}pyridin-3-yl |
| 1714 | pyridazin-4-yl | ethyl | 1-(3-bromopyridin-2-yl)-5-(difluoromethyl)-1H-pyrazol-4-yl |
| 1715 | pyridazin-4-yl | H | 1-(2,6-dichloropyridin-3-yl)-5-(trifluoromethyl)-1H-pyrazol-4- |
| | | | yl |
| 1716 | pyridazin-4-yl | H | 5-(1-methoxyethyl)-1-[3-(trifluoromethyl)pyridin-2-yl]-1H- |
| | | | pyrazol-4-yl |
| 1717 | pyridazin-4-yl | H | 1-phenyl-1H-pyrazol-4-yl |
| 1718 | pyridazin-4-yl | H | 6-chloropyridin-3-yl |
| 1719 | pyridazin-4-yl | H | 2-[5-chloro-3-(trifluoromethyl)pyridin-2-yl]-1,3-thiazol-4-yl |
| 1720 | pyridazin-4-yl | H | 3-(trifluoromethyl)-1H-pyrazol-4-yl |
| 1721 | pyridazin-4-yl | H | 1-[3-(difluoromethyl)pyridin-2-yl]-1H-pyrazol-4-yl |
| 1722 | pyridazin-4-yl | H | 5-(trifluoromethyl)-1-[3-(trifluoromethyl)pyridin-2-yl]-1H- |
| | | | pyrazol-4-yl |
| 1723 | pyridazin-4-yl | H | 1-(2-bromophenyl)-5-(1-methoxyethyl)-1H-pyrazol-4-yl |
| 1724 | pyridazin-4-yl | H | 1-(1,3-thiazol-2-ylmethyl)-3-(trifluoromethyl)-1H-pyrazol-4-yl |
| 1725 | pyridazin-4-yl | H | 3-(trifluoromethyl)phenyl |
| 1726 | pyridazin-4-yl | H | 2-[3-(1-methoxyethyl)pyridin-2-yl]-1,3-thiazol-4-yl |
| 1727 | pyridazin-4-yl | H | 5-(1-fluoroethyl)-1-(pyrimidin-2-yl)-1H-pyrazol-4-yl |
| 1728 | pyridazin-4-yl | H | 6-(4-cyanophenoxy)pyridin-3-yl |
| 1729 | pyridazin-4-yl | H | 1-[2-(trifluoromethyl)phenyl]-1H-pyrazol-4-yl |
| 1730 | pyridazin-4-yl | H | 1-[3-chloro-2-(trifluoromethyl)phenyl]-5-(methoxymethyl)- |
| | | | 1H-pyrazol-4-yl |
| 1731 | pyridazin-4-yl | H | 4-[(2-cyanoethyl)(methyl)carbamoyl]phenyl |
| 1732 | pyridazin-4-yl | H | 4-[(trifluoroacetyl)amino]phenyl |
| 1733 | pyridazin-4-yl | H | 1-benzyl-3-(trifluoromethyl)-1H-pyrazol-4-yl |
| 1734 | pyridazin-4-yl | H | 6-(2,5-dichlorophenoxy)pyridin-3-yl |
| 1735 | pyridazin-4-yl | H | 2-(pyrimidin-2-yl)-1,3-thiazol-5-yl |
| 1736 | pyridazin-4-yl | H | 3-(but-2-enoylamino)-4-methoxyphenyl |
| 1737 | pyridazin-4-yl | H | 4-(dimethylcarbamoyl)phenyl |
| 1738 | pyridazin-4-yl | H | 4-chloro-3-[(cyclopropylcarbonyl)amino]phenyl |
| 1739 | pyridazin-4-yl | H | 1-(2,6-dichlorophenyl)-5-methyl-1H-pyrazol-4-yl |
| 1740 | pyridazin-4-yl | H | 1-(2-chloropyridin-3-yl)-5-(difluoromethyl)-1H-pyrazol-4-yl |
| 1741 | pyridazin-4-yl | H | 1-(3-chloropyridin-2-yl)-5-(dimethoxymethyl)-1H-pyrazol-4-yl |
| 1742 | pyridazin-4-yl | H | 5-(difluoromethyl)-1-(2,2,2-trifluoroethyl)-1H-pyrazol-4-yl |
| 1743 | pyridazin-4-yl | H | 4-[(propan-2-ylsulphanyl)methyl]phenyl |
| 1744 | pyridazin-4-yl | H | 1-(2-bromo-6-chlorophenyl)-5-(dimethoxymethyl)-1H- |
| | | | pyrazol-4-yl |
| 1745 | pyridazin-4-yl | H | 4-methoxy-3-[3-(pyridin-2-yl)-1H-pyrazol-1-yl]phenyl |
| 1746 | pyridazin-4-yl | H | 3-chloro-4-{[4-(trifluoromethyl)-1H-pyrazol-1- |
| | | | yl]methyl}phenyl |
| 1747 | pyridazin-4-yl | H | 3-[(4,6-dimethoxypyrimidin-2-yl)methoxy]phenyl |
| 1748 | pyridazin-4-yl | H | 4-methyl-3-[(2-methylbutanoyl)amino]phenyl |
| 1749 | pyridazin-4-yl | H | 4-{[chloro(difluoro)acetyl](methyl)amino}phenyl |
| 1750 | pyridazin-4-yl | H | 4-{[3-(3-chloropyridin-2-yl)-1H-pyrazol-1-yl]methyl}phenyl |
| 1751 | pyridazin-4-yl | H | 6-{[4-(pyridin-2-yl)-1H-pyrazol-1-yl]methyl}pyridin-3-yl |
| 1752 | pyridazin-4-yl | H | 4-methoxy-3-[3-(pyrimidin-2-yl)-1H-pyrazol-1-yl]phenyl |
| 1753 | pyridazin-4-yl | H | 6-{[4-(trifluoromethyl)phenyl]sulphanyl}pyridin-3-yl |
| 1754 | pyridazin-4-yl | H | 4-{[3-(pyridin-2-yl)-1H-pyrazol-1-yl]methyl}phenyl |
| 1755 | pyridazin-4-yl | H | 4-chloro-3-[(2,2-dimethylpropanoyl)amino]phenyl |
| 1756 | pyridazin-4-yl | H | 1-(2-chloropyridin-3-yl)-5-(1-methoxyethyl)-1H-pyrazol-4-yl |
| 1757 | pyridazin-4-yl | H | 2-(3,5-dichloropyridin-2-yl)-1,3-thiazol-4-yl |
| 1758 | pyridazin-4-yl | H | 4-[(methylsulphanyl)methyl]phenyl |
| 1759 | pyridazin-4-yl | H | 6-(propylamino)pyridin-3-yl |
| 1760 | pyridazin-4-yl | H | 5-chloro-6-(4-cyanophenoxy)pyridin-3-yl |
| 1761 | pyridazin-4-yl | H | 6-(3,4-dichlorophenoxy)pyridin-3-yl |
| 1762 | pyridazin-4-yl | H | 6-[(2-chloropyrimidin-4-yl)oxy]pyridin-3-yl |
| 1763 | pyridazin-4-yl | H | 1-(3-chloropyridin-2-yl)-5-(1-methoxyethyl)-1H-pyrazol-4-yl |
| 1764 | pyridazin-4-yl | H | 2-[2-(trifluoromethyl)phenyl]-1,3-thiazol-4-yl |
| 1765 | pyridazin-4-yl | H | 3-(propan-2-ylcarbamoyl)phenyl |
| 1766 | pyridazin-4-yl | H | 4-(heptafluoropropyl)phenyl |
| 1767 | pyridazin-4-yl | H | 2-(2,6-dichloropyridin-3-yl)-1,3-thiazol-4-yl |
| 1768 | pyridazin-4-yl | H | 1-ethyl-5-(trifluoromethyl)-1H-pyrazol-4-yl |
| 1769 | pyridazin-4-yl | H | 1-[3-(difluoromethyl)pyridin-2-yl]-5-methyl-1H-pyrazol-4-yl |
| 1770 | pyridazin-4-yl | H | 1-(2,4-dichlorophenyl)-1H-pyrazol-4-yl |
| 1771 | pyridazin-4-yl | H | 3-fluoro-4-(methylsulphanyl)phenyl |
| 1772 | pyridazin-4-yl | H | 2-[4-chloro-2-(trifluoromethyl)phenyl]-1,3-thiazol-5-yl |
| 1773 | pyridazin-4-yl | H | 1-(propan-2-yl)-5-(trifluoromethyl)-1H-pyrazol-4-yl |
| 1774 | pyridazin-4-yl | H | 4-[(2-methylpyrimidin-4-yl)oxy]phenyl |
| 1775 | pyridazin-4-yl | H | 6-{[5-(trifluoromethyl)pyridin-2-yl]sulphanyl}pyridin-3-yl |
| 1776 | pyridazin-4-yl | H | 4-{[(propan-2-yloxy)imino]methyl}phenyl |
| 1777 | pyridazin-4-yl | H | 4-(diethylcarbamoyl)phenyl |
| 1778 | pyridazin-4-yl | H | 6-[(4-nitrophenyl)sulphanyl]pyridin-3-yl |
| 1779 | pyridazin-4-yl | H | 4-methyl-3-{[(propan-2-yloxy)carbonyl]amino}phenyl |
| 1780 | pyridazin-4-yl | H | 1-[4-fluoro-2-(trifluoromethyl)phenyl]-5-(methoxymethyl)-1H- |
| | | | pyrazol-4-yl |
| 1781 | pyridazin-4-yl | H | 5-(1,1-difluoroethyl)-1-(pyrimidin-2-yl)-1H-pyrazol-4-yl |
| 1782 | pyridazin-4-yl | H | 3-{[3,5-bis(trifluoromethyl)-1H-1,2,4-triazol-1- |
| | | | yl]methyl}phenyl |
| 1783 | pyridazin-4-yl | H | 4-{[3,5-bis(trifluoromethyl)-1H-pyrazol-1-yl]methyl}phenyl |
| 1784 | pyridazin-4-yl | H | 1-(2-chlorophenyl)-5-(1,1-dimethoxyethyl)-1H-pyrazol-4-yl |
| 1785 | pyridazin-4-yl | H | 1-(3-chloropyridin-2-yl)-5-(1,1-difluoroethyl)-1H-pyrazol-4-yl |
| 1786 | pyridazin-4-yl | H | 3-(1-fluoroethyl)-1-methyl-1H-pyrazol-4-yl |
| 1787 | pyridazin-4-yl | H | 1-(2,6-dichloropyridin-3-yl)-5-(methoxymethyl)-1H-pyrazol-4- |
| | | | yl |
| 1788 | pyridazin-4-yl | H | 6-(quinolin-6-yloxy)pyridin-3-yl |
| 1789 | pyridazin-4-yl | H | 3-(4-chloro-1H-pyrazol-1-yl)-4-methoxyphenyl |
| 1790 | pyridazin-4-yl | H | 2-(2,4-dichlorophenyl)-1,3-thiazol-4-yl |
| 1791 | pyridazin-4-yl | H | 1-(2-chloropyridin-3-yl)-5-(1,1-difluoroethyl)-1H-pyrazol-4-yl |
| 1792 | pyridazin-4-yl | H | 6-{4-[4-(methylsulphanyl)phenoxy]phenoxy}pyridin-3-yl |
| 1793 | pyridazin-4-yl | H | 6-(quinolin-5-yloxy)pyridin-3-yl |
| 1794 | pyridazin-4-yl | H | 1-[4-chloro-2-(trifluoromethyl)phenyl]-5-(difluoromethyl)-1H- |
| | | | pyrazol-4-yl |
| 1795 | pyridazin-4-yl | H | 1-[2-(1-methoxyethyl)phenyl]-1H-pyrazol-4-yl |
| 1796 | pyridazin-4-yl | H | 4-methoxy-3-[(2-methylbutanoyl)amino]phenyl |
| 1797 | pyridazin-4-yl | H | 6-(2,4-dichlorophenoxy)pyridin-3-yl |
| 1798 | pyridazin-4-yl | H | 4-{[acetyl(2-methylpropyl)amino]methyl}phenyl |
| 1799 | pyridazin-4-yl | H | 6-[(methylsulphanyl)methyl]pyridin-3-yl |
| 1800 | pyridazin-4-yl | H | 2-(2,4-dichlorophenyl)-1,3-thiazol-5-yl |
| 1801 | pyridazin-4-yl | H | 6-[(4,5,6-trimethylpyrimidin-2-yl)sulphanyl]pyridin-3-yl |
| 1802 | pyridazin-4-yl | H | 4-[(4,6-dimethylpyrimidin-2-yl)sulphanyl]phenyl |
| 1803 | pyridazin-4-yl | H | 6-[2-chloro-4-(methylsulphanyl)phenoxy]pyridin-3-yl |
| 1804 | pyridazin-4-yl | H | 1-(2-bromo-6-chlorophenyl)-5-(difluoromethyl)-1H-pyrazol-4- |
| | | | yl |
| 1805 | pyridazin-4-yl | H | 4-(propan-2-yloxy)-3-[3-(trifluoromethyl)-1H-pyrazol-1- |
| | | | yl]phenyl |
| 1806 | pyridazin-4-yl | H | 3-[ethyl(methyl)carbamoyl]phenyl |
| 1807 | pyridazin-4-yl | H | 6-(3,5-dimethylphenoxy)pyridin-3-yl |
| 1808 | pyridazin-4-yl | H | 1-[4-chloro-2-(trifluoromethyl)phenyl]-5-methyl-1H-pyrazol- |
| | | | 4-yl |
| 1809 | pyridazin-4-yl | H | 4-fluoro-3-(trifluoromethyl)phenyl |
| 1810 | pyridazin-4-yl | H | 6-[4-(methoxycarbonyl)phenoxy]pyridin-3-yl |
| 1811 | pyridazin-4-yl | H | 3-[(cyclopropylcarbonyl)amino]-4-methylphenyl |
| 1812 | pyridazin-4-yl | H | 1-(4-chlorophenyl)-1H-pyrazol-4-yl |
| 1813 | pyridazin-4-yl | H | 2-[2-(difluoromethyl)phenyl]-1,3-thiazol-5-yl |
| 1814 | pyridazin-4-yl | H | 1-ethyl-3-(trifluoromethyl)-1H-pyrazol-4-yl |
| 1815 | pyridazin-4-yl | H | 5-chloro-6-[4-(trifluoromethyl)phenoxy]pyridin-3-yl |
| 1816 | pyridazin-4-yl | H | 4-methyl-3-(propanoylamino)phenyl |
| 1817 | pyridazin-4-yl | H | 2-(2-chloropyridin-3-yl)-1,3-thiazol-4-yl |
| 1818 | pyridazin-4-yl | H | 4-[(2-methylpropanoyl)amino]phenyl |
| 1819 | pyridazin-4-yl | H | 3-[(2,2-dimethylpropanoyl)amino]-4-methylphenyl |
| 1820 | pyridazin-4-yl | H | 4-methoxy-3-{[(propan-2-yloxy)carbonyl]amino}phenyl |
| 1821 | pyridazin-4-yl | H | 1-(2,3-dichlorophenyl)-5-(methoxymethyl)-1H-pyrazol-4-yl |
| 1822 | pyridazin-4-yl | H | 3-(hydroxymethyl)-1-methyl-1H-pyrazol-4-yl |
| 1823 | pyridazin-4-yl | H | 1-(propan-2-yl)-3-(trifluoromethyl)-1H-pyrazol-4-yl |
| 1824 | pyridazin-4-yl | H | 2-[3-(difluoromethyl)pyridin-2-yl]-1,3-thiazol-5-yl |
| 1825 | pyridazin-4-yl | H | 4-(pyrrolidin-l-ylcarbonyl)phenyl |
| 1826 | pyridazin-4-yl | H | 3-(methoxymethyl)-1-methyl-1H-pyrazol-4-yl |
| 1827 | pyridazin-4-yl | H | 2-(3,5-dichloropyridin-2-yl)-1,3-thiazol-5-yl |
| 1828 | pyridazin-4-yl | H | 1-(2,6-dichloropyridin-3-yl)-5-(difluoromethyl)-1H-pyrazol-4- |
| | | | yl |
| 1829 | pyridazin-4-yl | H | 6-[(4,6-dimethylpyrimidin-2-yl)oxy]pyridin-3-yl |
| 1830 | pyridazin-4-yl | H | 4-[(4,6-dimethoxy-1,3,5-triazin-2-yl)sulphanyl]phenyl |
| 1831 | pyridazin-4-yl | H | 4-(methylsulphanyl)phenyl |
| 1832 | pyridazin-4-yl | H | 4-(ethylsulphanyl)phenyl |
| 1833 | pyridazin-4-yl | H | 6-[4-(trifluoromethoxy)phenoxy]pyridin-3-yl |
| 1834 | pyridazin-4-yl | H | 5-chloro-6-(4-nitrophenoxy)pyridin-3-yl |
| 1835 | pyridazin-4-yl | H | 2-[2-(difluoromethyl)phenyl]-1,3-thiazol-4-yl |
| 1836 | pyridazin-4-yl | H | 3-(difluoromethyl)-1-methyl-1H-pyrazol-4-yl |
| 1837 | pyridazin-4-yl | H | 1-(difluoromethyl)-3-(trifluoromethyl)-1H-pyrazol-4-yl |
| 1838 | pyridazin-4-yl | H | 6-[(6-chloropyridin-2-yl)oxy]pyridin-3-yl |
| 1839 | pyridazin-4-yl | H | 3-(butanoylamino)-4-methoxyphenyl |
| 1840 | pyridazin-4-yl | H | 4-(acetylamino)phenyl |
| 1841 | pyridazin-4-yl | H | 1-(2-bromophenyl)-5-(trifluoromethyl)-1H-pyrazol-4-yl |
| 1842 | pyridazin-4-yl | H | 6-(4-chlorophenoxy)pyridin-3-yl |
| 1843 | pyridazin-4-yl | H | 3-{[3-(trifluoromethyl)-1H-pyrazol-1-yl]methyl}phenyl |
| 1844 | pyridazin-4-yl | H | 5-(difluoromethyl)-1-(2-methylphenyl)-1H-pyrazol-4-yl |
| 1845 | pyridazin-4-yl | H | 2-[2-(1-methoxyethyl)phenyl]-1,3-thiazol-5-yl |
| 1846 | pyridazin-4-yl | H | 1-(2-chlorophenyl)-5-(1,1-difluoroethyl)-1H-pyrazol-4-yl |
| 1847 | pyridazin-4-yl | H | 1-(2-bromo-4-fluorophenyl)-1H-pyrazol-4-yl |
| 1848 | pyridazin-4-yl | H | 6-[(cyclopropylmethyl)amino]pyridin-3-yl |
| 1849 | pyridazin-4-yl | H | 6-[(2-methoxypyrimidin-4-yl)oxy]pyridin-3-yl |
| 1850 | pyridazin-4-yl | H | 4-(methoxycarbonyl)phenyl |
| 1851 | pyridazin-4-yl | H | 1-[2-(1,1-difluoroethyl)phenyl]-5-methyl-1H-pyrazol-4-yl |
| 1852 | pyridazin-4-yl | H | 3-{[3,5-bis(trifluoromethyl)-1H-1,2,4-triazol-1-yl]methyl}-4- |
| | | | chlorophenyl |
| 1853 | pyridazin-4-yl | H | 6-(morpholin-4-yl)pyridin-3-yl |
| 1854 | pyridazin-4-yl | H | 2-(pyrimidin-2-yl)-1,3-thiazol-4-yl |
| 1855 | pyridazin-4-yl | H | 4-[(4,6-dimethoxypyrimidin-2-yl)oxy]phenyl |
| 1856 | pyridazin-4-yl | H | 2-{[3-(trifluoromethyl)-1H-pyrazol-1-yl]methyl}pyridin-4-yl |
| 1857 | pyridazin-4-yl | H | 1-(2-chloropyridin-3-yl)-5-(trifluoromethyl)-1H-pyrazol-4-yl |
| 1858 | pyridazin-4-yl | H | 6-(4-bromophenoxy)pyridin-3-yl |
| 1859 | pyridazin-4-yl | H | 4-[(propylsulphanyl)methyl]phenyl |
| 1860 | pyridazin-4-yl | H | 6-(pyrimidin-2-ylsulphanyl)pyridin-3-yl |
| 1861 | pyridazin-4-yl | H | 1-(2-chloro-4-fluorophenyl)-1H-pyrazol-4-yl |
| 1862 | pyridazin-4-yl | H | 4-methoxy-3-[4-(pyridin-2-yl)-1H-pyrazol-1-yl]phenyl |
| 1863 | pyridazin-4-yl | H | 1-(2-chlorophenyl)-1H-pyrazol-4-yl |
| 1864 | pyridazin-4-yl | H | 6-(2,6-dichlorophenoxy)pyridin-3-yl |
| 1865 | pyridazin-4-yl | H | 3-[(4,6-dimethoxypyrimidin-2-yl)oxy]phenyl |
| 1866 | pyridazin-4-yl | H | 6-(4-nitrophenoxy)pyridin-3-yl |
| 1867 | pyridazin-4-yl | H | 1-(2-chlorophenyl)-5-methyl-1H-pyrazol-4-yl |
| 1868 | pyridazin-4-yl | H | 3-[ethyl(methyl)carbamoyl]-4-methoxyphenyl |
| 1869 | pyridazin-4-yl | H | 4-[(ethylsulphanyl)methyl]phenyl |
| 1870 | pyridazin-4-yl | H | 2-[2-(1-methoxyethyl)phenyl]-1,3-thiazol-4-yl |
| 1871 | pyridazin-4-yl | H | 6-{[6-(1H-pyrazol-1-yl)pyridin-2-yl]oxy}pyridin-3-yl |
| 1872 | pyridazin-4-yl | H | 2-[4-chloro-2-(trifluoromethyl)phenyl]-1,3-thiazol-4-yl |
| 1873 | pyridazin-4-yl | H | 4-methoxy-3-(1H-pyrazol-1-yl)phenyl |
| 1874 | pyridazin-4-yl | H | 3-(acetylamino)-4-methylphenyl |
| 1875 | pyridazin-4-yl | H | 2-{[4-(trifluoromethyl)-1H-pyrazol-1-yl]methyl}pyridin-4-yl |
| 1876 | pyridazin-4-yl | H | 1-(2,4-difluorophenyl)-1H-pyrazol-4-yl |
| 1877 | pyridazin-4-yl | H | 1-(2-methylphenyl)-5-(trifluoromethyl)-1H-pyrazol-4-yl |
| 1878 | pyridazin-4-yl | H | 2-[3-(difluoromethyl)pyridin-2-yl]-1,3-thiazol-4-yl |
| 1879 | pyridazin-4-yl | H | 6-(3,5-dichlorophenoxy)pyridin-3-yl |
| 1880 | pyridazin-4-yl | H | 1-(pyrimidin-2-ylmethyl)-3-(trifluoromethyl)-1H-pyrazol-4-yl |
| 1881 | pyridazin-4-yl | H | 6-methylpyridin-3-yl |
| 1882 | pyridazin-4-yl | H | 6-[(4-methylphenyl)sulphanyl]pyridin-3-yl |
| 1883 | pyridazin-4-yl | H | 1-(2,4-dichlorophenyl)-5-(methoxymethyl)-1H-pyrazol-4-yl |
| 1884 | pyridazin-4-yl | H | 6-{4-[4-(trifluoromethyl)phenoxy]phenoxy}pyridin-3-yl |
| 1885 | pyridazin-4-yl | H | 1-(2,6-dichlorophenyl)-5-(trifluoromethyl)-1H-pyrazol-4-yl |
| 1886 | pyridazin-4-yl | H | 4-[(4,6-dimethylpyrimidin-2-yl)oxy]phenyl |
| 1887 | pyridazin-4-yl | H | 6-(naphthalen-1-yloxy)pyridin-3-yl |
| 1888 | pyridazin-4-yl | H | 3-ethyl-1-methyl-1H-pyrazol-4-yl |
| 1889 | pyridazin-4-yl | H | 2-(2,6-dichloropyridin-3-yl)-1,3-thiazol-5-yl |
| 1890 | pyridazin-4-yl | H | 6-{[(4,6-dimethoxypyrimidin-2-yl)methyl]sulphanyl}pyridin- |
| | | | 3-yl |
| 1891 | pyridazin-4-yl | H | 1-[3-chloro-2-(trifluoromethyl)phenyl]-5-(1-methoxyethyl)- |
| | | | 1H-pyrazol-4-yl |
| 1892 | pyridazin-4-yl | H | 3,4-dimethoxyphenyl |
| 1893 | pyridazin-4-yl | H | 4-methoxy-3-[(methoxyacetyl)amino]phenyl |
| 1894 | pyridazin-4-yl | H | 3-{[4-(trifluoromethyl)-1H-pyrazol-1-yl]methyl}phenyl |
| 1895 | pyridazin-4-yl | H | 6-(2-cyanophenoxy)pyridin-3-yl |
| 1896 | pyridazin-4-yl | H | 3-[(methylsulphanyl)methyl]phenyl |
| 1897 | pyridazin-4-yl | H | 3-(dimethoxymethyl)-1-methyl-1H-pyrazol-4-yl |
| 1898 | pyridazin-4-yl | H | 1-(ethoxymethyl)-3-(trifluoromethyl)-1H-pyrazol-4-yl |
| 1899 | pyridazin-4-yl | H | 1-(3-bromopyridin-2-yl)-5-(methoxymethyl)-1H-pyrazol-4-yl |
| 1900 | pyridazin-4-yl | H | 6-(methylsulphanyl)pyridin-3-yl |
| 1901 | pyridazin-4-yl | H | 1-(2-bromo-6-chlorophenyl)-5-(trifluoromethyl)-1H-pyrazol-4- |
| | | | yl |
| 1902 | pyridazin-4-yl | H | 3-{[4-(trifluoromethyl)phenoxy]methyl}phenyl |
| 1903 | pyridazin-4-yl | H | 6-(heptafluoropropyl)pyridin-3-yl |
| 1904 | pyridazin-4-yl | H | 1-ethenyl-3-(trifluoromethyl)-1H-pyrazol-4-yl |
| 1905 | pyridazin-4-yl | H | 4-methyl-3-nitrophenyl |
| 1906 | pyridazin-4-yl | H | 3-(4-bromo-1H-pyrazol-1-yl)-4-methoxyphenyl |
| 1907 | pyridazin-4-yl | H | 5-methyl-1-(2-methylphenyl)-1H-pyrazol-4-yl |
| 1908 | pyridazin-4-yl | H | 1-[3-(difluoromethyl)pyridin-2-yl]-5-(methoxymethyl)-1H- |
| | | | pyrazol-4-yl |
| 1909 | pyridazin-4-yl | H | 6-{[2-(trifluoromethyl)pyrimidin-5-yl]oxy}pyridin-3-yl |
| 1910 | pyridazin-4-yl | H | 2-[2-(methoxymethyl)phenyl]-1,3-thiazol-5-yl |
| 1911 | pyridazin-4-yl | H | 1-(ethoxymethyl)-5-(trifluoromethyl)-1H-pyrazol-4-yl |
| 1912 | pyridazin-4-yl | H | 5-chloro-6-[(4,5,6-trimethylpyrimidin-2-yl)sulphanyl]pyridin- |
| | | | 3-yl |
| 1913 | pyridazin-4-yl | H | 6-[3,5-bis(trifluoromethyl)phenoxy]pyridin-3-yl |
| 1914 | pyridazin-4-yl | H | 4-{[4-(pyridin-2-yl)-1H-pyrazol-1-yl]methyl}phenyl |
| 1915 | pyridazin-4-yl | H | 1-[3-(1,1-difluoroethyl)pyridin-2-yl]-5-methyl-1H-pyrazol-4-yl |
| 1916 | pyridazin-4-yl | H | 1-(2,6-dichlorophenyl)-5-(difluoromethyl)-1H-pyrazol-4-yl |
| 1917 | pyridazin-4-yl | H | 1-[4-fluoro-2-(trifluoromethyl)phenyl]-5-methyl-1H-pyrazol-4- |
| | | | yl |
| 1918 | pyridazin-4-yl | H | 4-chloro-3-[(cyclobutylcarbonyl)amino]phenyl |
| 1919 | pyridazin-4-yl | H | 2-[2-(methoxymethyl)phenyl]-1,3-thiazol-4-yl |
| 1920 | pyridazin-4-yl | H | 4-(1H-pyrazol-1-ylmethyl)phenyl |
| 1921 | pyridazin-4-yl | H | 1-(2-bromophenyl)-5-methyl-1H-pyrazol-4-yl |
| 1922 | pyridazin-4-yl | H | 1-(2,3-difluorophenyl)-1H-pyrazol-4-yl |
| 1923 | pyridazin-4-yl | H | 6-{4-[(trifluoromethyl)sulphanyl]phenoxy}pyridin-3-yl |
| 1924 | pyridazin-4-yl | H | 4-{[3-(pentafluoroethyl)-1H-1,2,4-triazol-1-yl]methyl}phenyl |
| 1925 | pyridazin-4-yl | H | 4-{[3-(pyrimidin-2-yl)-1H-pyrazol-1-yl]methyl}phenyl |
| 1926 | pyridazin-4-yl | H | 1-(2-chlorophenyl)-5-(difluoromethyl)-1H-pyrazol-4-yl |
| 1927 | pyridazin-4-yl | H | 5-(difluoromethyl)-1-[2-(trifluoromethyl)phenyl]-1H-pyrazol- |
| | | | 4-yl |
| 1928 | pyridazin-4-yl | H | 6-(4-methoxyphenoxy)pyridin-3-yl |
| 1929 | pyridazin-4-yl | H | 1-[2-(dimethoxymethyl)phenyl]-1H-pyrazol-4-yl |
| 1930 | pyridazin-4-yl | H | 5-(methoxymethyl)-1-(pyrimidin-2-yl)-1H-pyrazol-4-yl |
| 1931 | pyridazin-4-yl | H | 6-tert-butoxypyridin-3-yl |
| 1932 | pyridazin-4-yl | H | 4-[(cyclopentylimino)methyl]phenyl |
| 1933 | pyridazin-4-yl | H | 1-(3-chlorophenyl)-1H-pyrazol-4-yl |
| 1934 | pyridazin-4-yl | H | 3-[(2-methylpropanoyl)amino]phenyl |
| 1935 | pyridazin-4-yl | H | 6-{[3,5-bis(trifluoromethyl)-1H-pyrazol-1-yl]methyl}pyridin- |
| | | | 3-yl |
| 1936 | pyridazin-4-yl | H | 4-chloro-3-[(2-methylpropanoyl)amino]phenyl |
| 1937 | pyridazin-4-yl | H | 4-(propylsulphanyl)phenyl |
| 1938 | pyridazin-4-yl | H | 1-[2-(difluoromethyl)phenyl]-5-(methoxymethyl)-1H-pyrazol- |
| | | | 4-yl |
| 1939 | pyridazin-4-yl | H | 4-methoxy-3-[4-(pyrimidin-2-yl)-1H-pyrazol-1-yl]phenyl |
| 1940 | pyridazin-4-yl | H | 4-{[(4,6-dimethylpyrimidin-2-yl)sulphanyl]methyl}phenyl |
| 1941 | pyridazin-4-yl | H | 1-(2-chlorophenyl)-5-(methoxymethyl)-1H-pyrazol-4-yl |
| 1942 | pyridazin-4-yl | H | 6-{[3,5-bis(trifluoromethyl)-1H-1,2,4-triazol-1- |
| | | | yl]methyl}pyridin-3-yl |
| 1943 | pyridazin-4-yl | H | 5-chloro-6-[4-(trifluoromethoxy)phenoxy]pyridin-3-yl |
| 1944 | pyridazin-4-yl | H | 4-[(tert-butoxyimino)methyl]phenyl |
| 1945 | pyridazin-4-yl | H | 2-(3-chloropyridin-2-yl)-1,3-thiazol-5-yl |
| 1946 | pyridazin-4-yl | H | 1-(2-fluorophenyl)-1H-pyrazol-4-yl |
| 1947 | pyridazin-4-yl | H | 4-(trifluoromethoxy)phenyl |
| 1948 | pyridazin-4-yl | H | 6-(2-chlorophenoxy)pyridin-3-yl |
| 1949 | pyridazin-4-yl | H | 3-[(2,2-dimethylpropanoyl)amino]-5-(trifluoromethyl)phenyl |
| 1950 | pyridazin-4-yl | H | 6-[(4-methylpyrimidin-2-yl)sulphanyl]pyridin-3-yl |
| 1951 | pyridazin-4-yl | H | 6-{[2-(morpholin-4-yl)ethyl]amino}pyridin-3-yl |
| 1952 | pyridazin-4-yl | H | 3-formyl-1-methyl-1H-pyrazol-4-yl |
| 1953 | pyridazin-4-yl | H | 1-methyl-3-(propan-2-yl)-1H-pyrazol-4-yl |
| 1954 | pyridazin-4-yl | H | 1-(3-chlorophenyl)-5-(1,1-dimethoxyethyl)-1H-pyrazol-4-yl |
| 1955 | pyridazin-4-yl | H | 4-nitrophenyl |
| 1956 | pyridazin-4-yl | H | 6-{[5-(methoxycarbonyl)pyridin-3-yl]oxy}pyridin-3-yl |
| 1957 | pyridazin-4-yl | H | 3-{[4-(trifluoromethyl)benzyl]oxy}phenyl |
| 1958 | pyridazin-4-yl | H | 1-(2,4-dichlorophenyl)-5-(1,1-dimethoxyethyl)-1H-pyrazol-4- |
| | | | yl |
| 1959 | pyridazin-4-yl | H | 4-{[4-bromo-3-(trifluoromethyl)-1H-pyrazol-1- |
| | | | yl]methyl}phenyl |
| 1960 | pyridazin-4-yl | H | 3-[chloro(difluoro)methyl]-1-methyl-1H-pyrazol-4-yl |
| 1961 | pyridazin-4-yl | H | 3-[(cyclopropylcarbonyl)amino]-4-fluorophenyl |
| 1962 | pyridazin-4-yl | H | 6-[4-(dimethylsulphamoyl)phenoxy]pyridin-3-yl |
| 1963 | pyridazin-4-yl | H | 5-methyl-1-(2-methylpyridin-3-yl)-1H-pyrazol-4-yl |
| 1964 | pyridazin-4-yl | H | 1-(2,4-difluorophenyl)-5-methyl-1H-pyrazol-4-yl |
| 1965 | pyridazin-4-yl | H | 3-(methylsulphanyl)phenyl |
| 1966 | pyridazin-4-yl | H | 3-(acetylamino)-4-chlorophenyl |
| 1967 | pyridazin-4-yl | H | 4-[(4-bromo-1H-pyrazol-1-yl)methyl]phenyl |
| 1968 | pyridazin-4-yl | H | 4-methyl-3-[(propan-2-ylcarbamoyl)amino]phenyl |
| 1969 | pyridazin-4-yl | H | 1-(2,2-diethoxyethyl)-3-(trifluoromethyl)-1H-pyrazol-4-yl |
| 1970 | pyridazin-4-yl | H | 6-[(4,6-dimethoxypyrimidin-2-yl)oxy]pyridin-3-yl |
| 1971 | pyridazin-4-yl | H | 6-(1H-pyrazol-1-yl)pyridin-3-yl |
| 1972 | pyridazin-4-yl | H | 4-(1H-1,2,4-triazol-1-ylmethyl)phenyl |
| 1973 | pyridazin-4-yl | H | 5-(methoxymethyl)-1-[2-(trifluoromethyl)phenyl]-1H-pyrazol- |
| | | | 4-yl |
| 1974 | pyridazin-4-yl | H | 5-methylpyridin-3-yl |
| 1975 | pyridazin-4-yl | H | 5-chloro-6-[(4,6-dimethylpyrimidin-2-yl)sulphanyl]pyridin-3- |
| | | | yl |
| 1976 | pyridazin-4-yl | H | 3-[(2,2-dimethylpropanoyl)amino]-4-methoxyphenyl |
| 1977 | pyridazin-4-yl | H | 2-[2-(trifluoromethyl)pyridin-3-yl]-1,3-thiazol-4-yl |
| 1978 | pyridazin-4-yl | H | 3-cyclopropyl-1-methyl-1H-pyrazol-4-yl |
| 1979 | pyridazin-4-yl | H | 3-methyl-1H-pyrazol-4-yl |
| 1980 | pyridazin-4-yl | H | 5-(methoxymethyl)-1-(2,2,2-trifluoroethyl)-1H-pyrazol-4-yl |
| 1981 | pyridazin-4-yl | H | 4-methoxy-3-(propanoylamino)phenyl |
| 1982 | pyridazin-4-yl | H | 4-methoxy-3-[(2-methylpropanoyl)amino]phenyl |
| 1983 | pyridazin-4-yl | H | 6-(quinolin-7-yloxy)pyridin-3-yl |
| 1984 | pyridazin-4-yl | H | 4-{[3-(trifluoromethyl)-1H-pyrazol-1-yl]methyl}phenyl |
| 1985 | pyridazin-4-yl | H | 3-[(4,6-dimethylpyrimidin-2-yl)oxy]phenyl |
| 1986 | pyridazin-4-yl | H | 4-{[3,5-bis(trifluoromethyl)-1H-1,2,4-triazol-1- |
| | | | yl]methyl}phenyl |
| 1987 | pyridazin-4-yl | H | 3-fluoro-4-{[4-(trifluoromethyl)-1H-pyrazol-1- |
| | | | yl]methyl}phenyl |
| 1988 | pyridazin-4-yl | H | 2,3-dihydro-1,4-benzodioxin-6-yl |
| 1989 | pyridazin-4-yl | H | 2-[3-(methoxymethyl)pyridin-2-yl]-1,3-thiazol-5-yl |
| 1990 | pyridazin-4-yl | H | 6-({6-[3-(trifluoromethyl)-1H-pyrazol-1-yl]pyridin-2- |
| | | | yl}oxy)pyridin-3-yl |
| 1991 | pyridazin-4-yl | H | 3-{[3-(pyridin-2-yl)-1H-pyrazol-1-yl]methyl}phenyl |
| 1992 | pyridazin-4-yl | H | 1-(2-bromo-4-chlorophenyl)-5-methyl-1H-pyrazol-4-yl |
| 1993 | pyridazin-4-yl | H | 2,2-difluoro-1,3-benzodioxo1-5-yl |
| 1994 | pyridazin-4-yl | H | 2-[3-(trifluoromethyl)pyridin-2-yl]-1,3-thiazol-4-yl |
| 1995 | pyridazin-4-yl | H | 4-(benzylcarbamoyl)phenyl |
| 1996 | pyridazin-4-yl | H | 4-[(2-chloropyrimidin-4-yl)oxy]phenyl |
| 1997 | pyridazin-4-yl | H | 1-[3-(trifluoromethyl)pyridin-2-yl]-1H-pyrazol-4-yl |
| 1998 | pyridazin-4-yl | H | 5-chloro-6-(4-chlorophenoxy)pyridin-3-yl |
| 1999 | pyridazin-4-yl | H | 3-cyano-4-(1H-1,2,4-triazol-1-yl)phenyl |
| 2000 | pyridazin-4-yl | H | 3-amino-4-(methylsulphonyl)phenyl |
| 2001 | pyridazin-4-yl | H | 4-methyl-3-[(2-methylpropanoyl)amino]phenyl |
| 2002 | pyridazin-4-yl | H | 1-(2,6-dibromophenyl)-5-(trifluoromethyl)-1H-pyrazol-4-yl |
| 2003 | pyridazin-4-yl | H | 3-{[3-(trifluoromethyl)-1H-1,2,4-triazol-1-yl]methyl}phenyl |
| 2004 | pyridazin-4-yl | H | 4-[(3,3,3-trifluoropropanoyl)amino]phenyl |
| 2005 | pyridazin-4-yl | H | 5-(pyrazin-2-yl)-1-(2,2,2-trifluoroethyl)-1H-pyrazol-4-yl |
| 2006 | pyridazin-4-yl | H | 1-(2,6-dichlorophenyl)-5-(dimethoxymethyl)-1H-pyrazol-4-yl |
| 2007 | pyridazin-4-yl | H | 1-(4-chlorophenyl)-5-(1,1-dimethoxyethyl)-1H-pyrazol-4-yl |
| 2008 | pyridazin-4-yl | H | 6-[3-(trifluoromethyl)phenoxy]pyridin-3-yl |
| 2009 | pyridazin-4-yl | H | 4-({6-[3-(trifluoromethyl)-1H-pyrazol-1-yl]pyridin-2- |
| | | | yl}oxy)phenyl |
| 2010 | pyridazin-4-yl | H | 4-methoxy-3-(propan-2-ylcarbamoyl)phenyl |
| 2011 | pyridazin-4-yl | H | 1-[2-(1-methoxyethyl)phenyl]-5-methyl-1H-pyrazol-4-yl |
| 2012 | pyridazin-4-yl | H | 1-ethyl-3-methyl-1H-pyrazol-4-yl |
| 2013 | pyridazin-4-yl | H | 4-[(4,6-dimethoxypyrimidin-2-yl)sulphanyl]phenyl |
| 2014 | pyridazin-4-yl | H | 6-(4-methylphenoxy)pyridin-3-yl |
| 2015 | pyridazin-4-yl | H | 6-(4-chloro-2-cyanophenoxy)pyridin-3-yl |
| 2016 | pyridazin-4-yl | H | 1-propyl-3-(trifluoromethyl)-1H-pyrazol-4-yl |
| 2017 | pyridazin-4-yl | H | 6-(2-methylphenoxy)pyridin-3-yl |
| 2018 | pyridazin-4-yl | H | 4-{[4-(trifluoromethyl)-1H-pyrazol-1-yl]methyl}phenyl |
| 2019 | pyridazin-4-yl | H | 6-[(6-methylpyridin-3-yl)oxy]pyridin-3-yl |
| 2020 | pyridazin-4-yl | H | 6-methoxypyridin-3-yl |
| 2021 | pyridazin-4-yl | H | 4-[(ethoxyimino)methyl]phenyl |
| 2022 | pyridazin-4-yl | H | 4-{[acetyl(cyclopentyl)amino]methyl}phenyl |
| 2023 | pyridazin-4-yl | H | 3-{[3,5-bis(trifluoromethyl)-1H-pyrazol-1-yl]methyl}phenyl |
| 2024 | pyridazin-4-yl | H | 1-(2-bromo-4-chlorophenyl)-1H-pyrazol-4-yl |
| 2025 | pyridazin-4-yl | H | 6-[4-(1H-1,2,4-triazol-1-yl)phenoxy]pyridin-3-yl |
| 2026 | pyridazin-4-yl | H | 6-[4-(trifluoromethyl)phenoxy]pyridin-3-yl |
| 2027 | pyridazin-4-yl | H | 1-(2-chlorophenyl)-5-(trifluoromethyl)-1H-pyrazol-4-yl |
| 2028 | pyridazin-4-yl | H | 4-methoxy-3-nitrophenyl |
| 2029 | pyridazin-4-yl | H | 1-(2,6-dibromophenyl)-5-(difluoromethyl)-1H-pyrazol-4-yl |
| 2030 | pyridazin-4-yl | H | 6-[(4-methoxyphenyl)sulphanyl]pyridin-3-yl |
| 2031 | pyridazin-4-yl | H | 1-(2,6-dichlorophenyl)-5-(1-methoxyethyl)-1H-pyrazol-4-yl |
| 2032 | pyridazin-4-yl | H | 4-[(6-chloropyridin-2-yl)oxy]phenyl |
| 2033 | pyridazin-4-yl | H | 3-fluoro-4-{[3-(trifluoromethyl)-1H-pyrazol-1- |
| | | | yl]methyl}phenyl |
| 2034 | pyridazin-4-yl | H | 4-{[(difluoromethyl)sulphanyl]methyl}phenyl |
| 2035 | pyridazin-4-yl | H | 6-{[3-(trifluoromethyl)-1H-pyrazol-1-yl]methyl}pyridin-3-yl |
| 2036 | pyridazin-4-yl | H | 4-{[2-(3,5-dichlorophenyl)propan-2-yl]carbamoyl}phenyl |
| 2037 | pyridazin-4-yl | H | 3-(1H-1,2,4-triazol-1-ylmethyl)phenyl |
| 2038 | pyridazin-4-yl | H | 2-(3-chloropyridin-2-yl)-1,3-thiazol-4-yl |
| 2039 | pyridazin-4-yl | H | 5-(1-methoxyethyl)-1-(pyrimidin-2-yl)-1H-pyrazol-4-yl |
| 2040 | pyridazin-4-yl | H | 1-(3-chloropyridin-2-yl)-5-(1,1-dimethoxyethyl)-1H-pyrazol- |
| | | | 4-yl |
| 2041 | pyridazin-4-yl | H | 1-(2-bromo-4-fluorophenyl)-5-methyl-1H-pyrazol-4-yl |
| 2042 | pyridazin-4-yl | H | 3-(butanoylamino)-4-methylphenyl |
| 2043 | pyridazin-4-yl | H | 5-(methoxymethyl)-1-[3-(trifluoromethyl)pyridin-2-yl]-1H- |
| | | | pyrazol-4-yl |
| 2044 | pyridazin-4-yl | H | 6-[3-(trifluoromethyl)-1H-pyrazol-1-yl]pyridin-3-yl |
| 2045 | pyridazin-4-yl | H | 6-(3-chlorophenoxy)pyridin-3-yl |
| 2046 | pyridazin-4-yl | H | 1-(2,6-dichloropyridin-3-yl)-5-(1-methoxyethyl)-1H-pyrazol- |
| | | | 4-yl |
| 2047 | pyridazin-4-yl | H | 6-[(4,6-dimethylpyrimidin-2-yl)sulphanyl]pyridin-3-yl |
| 2048 | pyridazin-4-yl | H | 1-[2-(difluoromethyl)phenyl]-5-methyl-1H-pyrazol-4-yl |
| 2049 | pyridazin-4-yl | H | 2-[2-(trifluoromethyl)phenyl]-1,3-thiazol-5-yl |
| 2050 | pyridazin-4-yl | H | 2-[2-(trifluoromethyl)pyridin-3-yl]-1,3-thiazol-5-yl |
| 2051 | pyridazin-4-yl | H | 6-[(4,6-dimethoxy-1,3,5-triazin-2-yl)sulphanyl]pyridin-3-yl |
| 2052 | pyridazin-4-yl | H | 2-(2-chloropyridin-3-yl)-1,3-thiazol-5-yl |
| 2053 | pyridazin-4-yl | H | 1-(2,6-dichloropyridin-3-yl)-5-methyl-1H-pyrazol-4-yl |
| 2054 | pyridazin-4-yl | H | 1-(3-chloropyridin-2-yl)-5-(methoxymethyl)-1H-pyrazol-4-yl |
| 2055 | pyridazin-4-yl | H | 6-[(4-chlorophenyl)sulphanyl]pyridin-3-yl |
| 2056 | pyridazin-4-yl | H | 6-(piperidin-l-yl)pyridin-3-yl |
| 2057 | pyridazin-4-yl | H | 3-[(cyclopentylcarbonyl)amino]-4-methylphenyl |
| 2058 | pyridazin-4-yl | H | 6-ethoxypyridin-3-yl |
| 2059 | pyridazin-4-yl | H | 2-(2,6-dichlorophenyl)-1,3-thiazol-4-yl |
| 2060 | pyridazin-4-yl | H | 5-chloro-6-[(4-methylpyrimidin-2-yl)sulphanyl]pyridin-3-yl |
| 2061 | pyridazin-4-yl | H | 1-[4-chloro-2-(trifluoromethyl)phenyl]-5-(1-methoxyethyl)- |
| | | | 1H-pyrazol-4-yl |
| 2062 | pyridazin-4-yl | H | 6-(3-methylphenoxy)pyridin-3-yl |
| 2063 | pyridazin-4-yl | H | 4-[ethyl(methyl)carbamoyl]phenyl |
| 2064 | pyridazin-4-yl | H | 6-[3-(trifluoromethoxy)phenoxy]pyridin-3-yl |
| 2065 | pyridazin-4-yl | H | 5-(difluoromethyl)-1-(pyrimidin-2-yl)-1H-pyrazol-4-yl |
| 2066 | pyridazin-4-yl | H | 4-[(cyclopropylcarbonyl)amino]phenyl |
| 2067 | pyridazin-4-yl | H | 1-(3-bromopyridin-2-yl)-5-(1-methoxyethyl)-1H-pyrazol-4-yl |
| 2068 | pyridazin-4-yl | H | 3-[(cyclopropylcarbonyl)amino]-4-methoxyphenyl |
| 2069 | pyridazin-4-yl | H | 4-methyl-3-[(3-methylbut-2-enoyl)amino]phenyl |
| 2070 | pyridazin-4-yl | H | 1-[3-(methoxymethyl)pyridin-2-yl]-1H-pyrazol-4-yl |
| 2071 | pyridazin-4-yl | H | 4-[acetyl(methyl)amino]phenyl |
| 2072 | pyridazin-4-yl | H | 6-(naphthalen-2-yloxy)pyridin-3-yl |
| 2073 | pyridazin-4-yl | H | 1-(2-chlorophenyl)-5-(1-methoxyethyl)-1H-pyrazol-4-yl |
| 2074 | pyridazin-4-yl | H | 1-[2-(difluoromethyl)phenyl]-5-(1-methoxyethyl)-1H-pyrazol- |
| | | | 4-yl |
| 2075 | pyridazin-4-yl | H | 1-(3-chloropyridin-2-yl)-5-methyl-1H-pyrazol-4-yl |
| 2076 | pyridazin-4-yl | H | 4-{[3-(trifluoromethyl)-1H-1,2,4-triazol-1-yl]methyl}phenyl |
| 2077 | pyridazin-4-yl | H | 3-[(cyclopentylcarbonyl)amino]-4-methoxyphenyl |
| 2078 | pyridazin-4-yl | H | 1-(2,2,2-trifluoroethyl)-5-(trifluoromethyl)-1H-pyrazol-4-yl |
| 2079 | pyridazin-4-yl | H | 6-[4-(methylsulphanyl)phenoxy]pyridin-3-yl |
| 2080 | pyridazin-4-yl | H | 2-[3-(trifluoromethyl)pyridin-2-yl]-1,3-thiazol-5-yl |
| 2081 | pyridazin-4-yl | H | 4-{[3-(methoxycarbonyl)-1H-1,2,4-triazol-1-yl]methyl}phenyl |
| 2082 | pyridazin-4-yl | H | 1-[3-(1,1-difluoroethyl)pyridin-2-yl]-1H-pyrazol-4-yl |
| 2083 | pyridazin-4-yl | H | 6-{[3-(pyridin-2-yl)-1H-pyrazol-1-yl]methyl}pyridin-3-yl |
| 2084 | pyridazin-4-yl | H | 3-(acetylamino)phenyl |
| 2085 | pyridazin-4-yl | H | 1-(3-chloropyridin-2-yl)-5-(trifluoromethyl)-1H-pyrazol-4-yl |
| 2086 | pyridazin-4-yl | H | 2-{[3-(pyridin-2-yl)-1H-pyrazol-1-yl]methyl}pyridin-4-yl |
| 2087 | pyridazin-4-yl | H | 3-[(methoxyacetyl)amino]-4-methylphenyl |
| 2088 | pyridazin-4-yl | H | 1-(3-bromopyridin-2-yl)-5-(trifluoromethyl)-1H-pyrazol-4-yl |
| 2089 | pyridazin-4-yl | H | 4-[(methoxyimino)methyl]phenyl |
| 2090 | pyridazin-4-yl | H | 1-(pyrimidin-2-yl)-5-(trifluoromethyl)-1H-pyrazol-4-yl |
| 2091 | pyridazin-4-yl | H | 2-(2-chlorophenyl)-1,3-thiazol-4-yl |
| 2092 | pyridazin-4-yl | H | 4-methoxy-3-[(propan-2-ylsulphonyl)amino]phenyl |
| 2093 | pyridazin-4-yl | H | 6-(3-cyanophenoxy)pyridin-3-yl |
| 2094 | pyridazin-4-yl | H | 4-fluoro-3-[(2-methylpropanoyl)amino]phenyl |
| 2095 | pyridazin-4-yl | H | 1-(2-bromophenyl)-5-(difluoromethyl)-1H-pyrazol-4-yl |
| 2096 | pyridazin-4-yl | H | 1,3-dimethyl-1H-pyrazol-4-yl |
| 2097 | pyridazin-4-yl | H | 5-(1-methoxyethyl)-1-(2,2,2-trifluoroethyl)-1H-pyrazol-4-yl |
| 2098 | pyridazin-4-yl | H | 1-(2,6-dichlorophenyl)-5-(1-fluoroethyl)-1H-pyrazol-4-yl |
| 2099 | pyridazin-4-yl | H | 6-(pyrrolidin-l-yl)pyridin-3-yl |
| 2100 | pyridazin-4-yl | H | 3-(acetylamino)-5-(trifluoromethyl)phenyl |
| 2101 | pyridazin-4-yl | H | 2-[3-(methoxymethyl)pyridin-2-yl]-1,3-thiazol-4-yl |
| 2102 | pyridazin-4-yl | H | 2-[3-(1-methoxyethyl)pyridin-2-yl]-1,3-thiazol-5-yl |
| 2103 | pyridazin-4-yl | H | 1-[3-(dimethoxymethyl)pyridin-2-yl]-1H-pyrazol-4-yl |
| 2104 | pyridazin-4-yl | H | 5-(1-methoxyethyl)-1-(2-methylphenyl)-1H-pyrazol-4-yl |
| 2105 | pyridazin-4-yl | H | 1-methyl-1H-benzotriazol-5-yl |
| 2106 | pyridazin-4-yl | H | 4-[(butylsulphanyl)methyl]phenyl |
| 2107 | pyridazin-4-yl | H | 1H-pyrazol-4-yl |
| 2108 | pyridazin-4-yl | H | 1-(2-bromo-6-chlorophenyl)-5-(1-methoxyethyl)-1H-pyrazol- |
| | | | 4-yl |
| 2109 | pyridazin-4-yl | H | 5-chloro-6-[4-(methylsulphonyl)phenoxy]pyridin-3-yl |
| 2110 | pyridazin-4-yl | H | 1-[3-(1-methoxyethyl)pyridin-2-yl]-1H-pyrazol-4-yl |
| 2111 | pyridazin-4-yl | H | 6-[(4,6-dimethoxypyrimidin-2-yl)sulphanyl]pyridin-3-yl |
| 2112 | pyridazin-4-yl | H | 4-[(6-methylpyridin-3-yl)oxy]phenyl |
| 2113 | pyridazin-4-yl | H | 3-methoxy-1-methyl-1H-pyrazol-4-yl |
| 2114 | pyridazin-4-yl | H | 6-(4-tert-butylphenoxy)pyridin-3-yl |
| 2115 | pyridazin-4-yl | H | 1-(2,4-dichlorophenyl)-5-methyl-1H-pyrazol-4-yl |
| 2116 | pyridazin-4-yl | H | 3-(but-2-enoylamino)-4-methylphenyl |
| 2117 | pyridazin-4-yl | H | 4-{[4-(pyrimidin-2-yl)-1H-pyrazol-1-yl]methyl}phenyl |
| 2118 | pyridazin-4-yl | H | 4-{[6-(1H-pyrazol-1-yl)pyridin-2-yl]oxy}phenyl |
| 2119 | pyridazin-4-yl | H | 4-{[3,5-bis(trifluoromethyl)-1H-1,2,4-triazol-1-yl]methyl}-3- |
| | | | chlorophenyl |
| 2120 | pyridazin-4-yl | H | 4-[(2-methoxypyrimidin-4-yl)oxy]phenyl |
| 2121 | pyridazin-4-yl | H | 1-[4-chloro-2-(trifluoromethyl)phenyl]-5-(methoxymethyl)- |
| | | | 1H-pyrazol-4-yl |
| 2122 | pyridazin-4-yl | H | 5-(trifluoromethyl)-1-[2-(trifluoromethyl)phenyl]-1H-pyrazol- |
| | | | 4-yl |
| 2123 | pyridazin-4-yl | H | 6-(dimethylamino)pyridin-3-yl |
| 2124 | pyridazin-4-yl | H | 2-[5-chloro-3-(trifluoromethyl)pyridin-2-yl]-1,3-thiazol-5-yl |
| 2125 | pyridazin-4-yl | H | 4-{[4-(3-chloropyridin-2-yl)-1H-pyrazol-1-yl]methyl}phenyl |
| 2126 | pyridazin-4-yl | H | 4-[(2,2,3,3,4,4,4-heptafluorobutanoyl)(methyl)amino]phenyl |
| 2127 | pyridazin-4-yl | H | 4-[(trifluoromethyl)sulphanyl]phenyl |
| 2128 | pyridazin-4-yl | H | 6-[4-(methylsulphonyl)phenoxy]pyridin-3-yl |
| 2129 | pyridazin-4-yl | H | 1-(2-bromophenyl)-5-(methoxymethyl)-1H-pyrazol-4-yl |
| 2130 | pyridazin-4-yl | H | 6-[(4-bromo-1H-pyrazol-1-yl)methyl]pyridin-3-yl |
| 2131 | pyridazin-4-yl | H | 3-chloro-4-{[3-(trifluoromethyl)-1H-pyrazol-1- |
| | | | yl]methyl}phenyl |
| 2132 | pyridazin-4-yl | H | 6-{[4-(trifluoromethyl)-1H-pyrazol-1-yl]methyl}pyridin-3-yl |
| 2133 | pyridazin-4-yl | H | 1-(3-bromopyridin-2-yl)-5-(difluoromethyl)-1H-pyrazol-4-yl |
| 2134 | pyridazin-4-yl | CH₃ | 1-(2,6-dichloropyridin-3-yl)-5-(trifluoromethyl)-1H-pyrazol-4- |
| | | | yl |
| 2135 | pyridazin-4-yl | CH₃ | 5-(1-methoxyethyl)-1-[3-(trifluoromethyl)pyridin-2-yl]-1H- |
| | | | pyrazol-4-yl |
| 2136 | pyridazin-4-yl | CH₃ | 1-phenyl-1H-pyrazol-4-yl |
| 2137 | pyridazin-4-yl | CH₃ | 6-chloropyridin-3-yl |
| 2138 | pyridazin-4-yl | CH₃ | 2-[5-chloro-3-(trifluoromethyl)pyridin-2-yl]-1,3-thiazol-4-yl |
| 2139 | pyridazin-4-yl | CH₃ | 3-(trifluoromethyl)-1H-pyrazol-4-yl |
| 2140 | pyridazin-4-yl | CH₃ | 1-[3-(difluoromethyl)pyridin-2-yl]-1H-pyrazol-4-yl |
| 2141 | pyridazin-4-yl | CH₃ | 5-(trifluoromethyl)-1-[3-(trifluoromethyl)pyridin-2-yl]-1H- |
| | | | pyrazol-4-yl |
| 2142 | pyridazin-4-yl | CH₃ | 1-(2-bromophenyl)-5-(1-methoxyethyl)-1H-pyrazol-4-yl |
| 2143 | pyridazin-4-yl | CH₃ | 1-(1,3-thiazol-2-ylmethyl)-3-(trifluoromethyl)-1H-pyrazol-4-yl |
| 2144 | pyridazin-4-yl | CH₃ | 3-(trifluoromethyl)phenyl |
| 2145 | pyridazin-4-yl | CH₃ | 2-[3-(1-methoxyethyl)pyridin-2-yl]-1,3-thiazol-4-yl |
| 2146 | pyridazin-4-yl | CH₃ | 5-(1-fluoroethyl)-1-(pyrimidin-2-yl)-1H-pyrazol-4-yl |
| 2147 | pyridazin-4-yl | CH₃ | 6-(4-cyanophenoxy)pyridin-3-yl |
| 2148 | pyridazin-4-yl | CH₃ | 1-[2-(trifluoromethyl)phenyl]-1H-pyrazol-4-yl |
| 2149 | pyridazin-4-yl | CH₃ | 1-[3-chloro-2-(trifluoromethyl)phenyl]-5-(methoxymethyl)- |
| | | | 1H-pyrazol-4-yl |
| 2150 | pyridazin-4-yl | CH₃ | 4-[(2-cyanoethyl)(methyl)carbamoyl]phenyl |
| 2151 | pyridazin-4-yl | CH₃ | 4-[(trifluoroacetyl)amino]phenyl |
| 2152 | pyridazin-4-yl | CH₃ | 1-benzyl-3-(trifluoromethyl)-1H-pyrazol-4-yl |
| 2153 | pyridazin-4-yl | CH₃ | 6-(2,5-dichlorophenoxy)pyridin-3-yl |
| 2154 | pyridazin-4-yl | CH₃ | 2-(pyrimidin-2-yl)-1,3-thiazol-5-yl |
| 2155 | pyridazin-4-yl | CH₃ | 3-(but-2-enoylamino)-4-methoxyphenyl |
| 2156 | pyridazin-4-yl | CH₃ | 4-(dimethylcarbamoyl)phenyl |
| 2157 | pyridazin-4-yl | CH₃ | 4-chloro-3-[(cyclopropylcarbonyl)amino]phenyl |
| 2158 | pyridazin-4-yl | CH₃ | 1-(2,6-dichlorophenyl)-5-methyl-1H-pyrazol-4-yl |
| 2159 | pyridazin-4-yl | CH₃ | 1-(2-chloropyridin-3-yl)-5-(difluoromethyl)-1H-pyrazol-4-yl |
| 2160 | pyridazin-4-yl | CH₃ | 1-(3-chloropyridin-2-yl)-5-(dimethoxymethyl)-1H-pyrazol-4-yl |
| 2161 | pyridazin-4-yl | CH₃ | 5-(difluoromethyl)-1-(2,2,2-trifluoroethyl)-1H-pyrazol-4-yl |
| 2162 | pyridazin-4-yl | CH₃ | 4-[(propan-2-ylsulphanyl)methyl]phenyl |
| 2163 | pyridazin-4-yl | CH₃ | 1-(2-bromo-6-chlorophenyl)-5-(dimethoxymethyl)-1H- |
| | | | pyrazol-4-yl |
| 2164 | pyridazin-4-yl | CH₃ | 4-methoxy-3-[3-(pyridin-2-yl)-1H-pyrazol-1-yl]phenyl |
| 2165 | pyridazin-4-yl | CH₃ | 3-chloro-4-{[4-(trifluoromethyl)-1H-pyrazol-1- |
| | | | yl]methyl}phenyl |
| 2166 | pyridazin-4-yl | CH₃ | 3-[(4,6-dimethoxypyrimidin-2-yl)methoxy]phenyl |
| 2167 | pyridazin-4-yl | CH₃ | 4-methyl-3-[(2-methylbutanoyl)amino]phenyl |
| 2168 | pyridazin-4-yl | CH₃ | 4-{[chloro(difluoro)acetyl](methyl)amino}phenyl |
| 2169 | pyridazin-4-yl | CH₃ | 4-{[3-(3-chloropyridin-2-yl)-1H-pyrazol-1-yl]methyl}phenyl |
| 2170 | pyridazin-4-yl | CH₃ | 6-{[4-(pyridin-2-yl)-1H-pyrazol-1-yl]methyl}pyridin-3-yl |
| 2171 | pyridazin-4-yl | CH₃ | 4-methoxy-3-[3-(pyrimidin-2-yl)-1H-pyrazol-1-yl]phenyl |
| 2172 | pyridazin-4-yl | CH₃ | 6-{[4-(trifluoromethyl)phenyl]sulphanyl}pyridin-3-yl |
| 2173 | pyridazin-4-yl | CH₃ | 4-{[3-(pyridin-2-yl)-1H-pyrazol-1-yl]methyl}phenyl |
| 2174 | pyridazin-4-yl | CH₃ | 4-chloro-3-[(2,2-dimethylpropanoyl)amino]phenyl |
| 2175 | pyridazin-4-yl | CH₃ | 1-(2-chloropyridin-3-yl)-5-(1-methoxyethyl)-1H-pyrazol-4-yl |
| 2176 | pyridazin-4-yl | CH₃ | 2-(3,5-dichloropyridin-2-yl)-1,3-thiazol-4-yl |
| 2177 | pyridazin-4-yl | CH₃ | 6-(propylamino)pyridin-3-yl |
| 2178 | pyridazin-4-yl | CH₃ | 5-chloro-6-(4-cyanophenoxy)pyridin-3-yl |
| 2179 | pyridazin-4-yl | CH₃ | 6-(3,4-dichlorophenoxy)pyridin-3-yl |
| 2180 | pyridazin-4-yl | CH₃ | 6-[(2-chloropyrimidin-4-yl)oxy]pyridin-3-yl |
| 2181 | pyridazin-4-yl | CH₃ | 1-(3-chloropyridin-2-yl)-5-(1-methoxyethyl)-1H-pyrazol-4-yl |
| 2182 | pyridazin-4-yl | CH₃ | 2-[2-(trifluoromethyl)phenyl]-1,3-thiazol-4-yl |
| 2183 | pyridazin-4-yl | CH₃ | 3-(propan-2-ylcarbamoyl)phenyl |
| 2184 | pyridazin-4-yl | CH₃ | 4-(heptafluoropropyl)phenyl |
| 2185 | pyridazin-4-yl | CH₃ | 2-(2,6-dichloropyridin-3-yl)-1,3-thiazol-4-yl |
| 2186 | pyridazin-4-yl | CH₃ | 1-ethyl-5-(trifluoromethyl)-1H-pyrazol-4-yl |
| 2187 | pyridazin-4-yl | CH₃ | 1-[3-(difluoromethyl)pyridin-2-yl]-5-methyl-1H-pyrazol-4-yl |
| 2188 | pyridazin-4-yl | CH₃ | 1-(2,4-dichlorophenyl)-1H-pyrazol-4-yl |
| 2189 | pyridazin-4-yl | CH₃ | 3-fluoro-4-(methylsulphanyl)phenyl |
| 2190 | pyridazin-4-yl | CH₃ | 2-[4-chloro-2-(trifluoromethyl)phenyl]-1,3-thiazol-5-yl |
| 2191 | pyridazin-4-yl | CH₃ | 4-[(2-methylpyrimidin-4-yl)oxy]phenyl |
| 2192 | pyridazin-4-yl | CH₃ | 6-{[5-(trifluoromethyl)pyridin-2-yl]sulphanyl}pyridin-3-yl |
| 2193 | pyridazin-4-yl | CH₃ | 4-{[(propan-2-yloxy)imino]methyl}phenyl |
| 2194 | pyridazin-4-yl | CH₃ | 4-(diethylcarbamoyl)phenyl |
| 2195 | pyridazin-4-yl | CH₃ | 6-[(4-nitrophenyl)sulphanyl]pyridin-3-yl |
| 2196 | pyridazin-4-yl | CH₃ | 4-methyl-3-{[(propan-2-yloxy)carbonyl]amino}phenyl |
| 2197 | pyridazin-4-yl | CH₃ | 1-[4-fluoro-2-(trifluoromethyl)phenyl]-5-(methoxymethyl)-1H- |
| | | | pyrazol-4-yl |
| 2198 | pyridazin-4-yl | CH₃ | 5-(1,1-difluoroethyl)-1-(pyrimidin-2-yl)-1H-pyrazol-4-yl |
| 2199 | pyridazin-4-yl | CH₃ | 3-{[3,5-bis(trifluoromethyl)-1H-1,2,4-triazol-1- |
| | | | yl]methyl}phenyl |
| 2200 | pyridazin-4-yl | CH₃ | 4-{[3,5-bis(trifluoromethyl)-1H-pyrazol-1-yl]methyl}phenyl |
| 2201 | pyridazin-4-yl | CH₃ | 1-(2-chlorophenyl)-5-(1,1-dimethoxyethyl)-1H-pyrazol-4-yl |
| 2202 | pyridazin-4-yl | CH₃ | 1-(3-chloropyridin-2-yl)-5-(1,1-difluoroethyl)-1H-pyrazol-4-yl |
| 2203 | pyridazin-4-yl | CH₃ | 1-(2,6-dichloropyridin-3-yl)-5-(methoxymethyl)-1H-pyrazol-4- |
| | | | yl |
| 2204 | pyridazin-4-yl | CH₃ | 6-(quinolin-6-yloxy)pyridin-3-yl |
| 2205 | pyridazin-4-yl | CH₃ | 3-(4-chloro-1H-pyrazol-1-yl)-4-methoxyphenyl |
| 2206 | pyridazin-4-yl | CH₃ | 2-(2,4-dichlorophenyl)-1,3-thiazol-4-yl |
| 2207 | pyridazin-4-yl | CH₃ | 1-(2-chloropyridin-3-yl)-5-(1,1-difluoroethyl)-1H-pyrazol-4-yl |
| 2208 | pyridazin-4-yl | CH₃ | 6-{4-[4-(methylsulphanyl)phenoxy]phenoxy}pyridin-3-yl |
| 2209 | pyridazin-4-yl | CH₃ | 6-(quinolin-5-yloxy)pyridin-3-yl |
| 2210 | pyridazin-4-yl | CH₃ | 1-[4-chloro-2-(trifluoromethyl)phenyl]-5-(difluoromethyl)-1H- |
| | | | pyrazol-4-yl |
| 2211 | pyridazin-4-yl | CH₃ | 1-[2-(1-methoxyethyl)phenyl]-1H-pyrazol-4-yl |
| 2212 | pyridazin-4-yl | CH₃ | 4-methoxy-3-[(2-methylbutanoyl)amino]phenyl |
| 2213 | pyridazin-4-yl | CH₃ | 6-(2,4-dichlorophenoxy)pyridin-3-yl |
| 2214 | pyridazin-4-yl | CH₃ | 4-{[acetyl(2-methylpropyl)amino]methyl}phenyl |
| 2215 | pyridazin-4-yl | CH₃ | 6-[(methylsulphanyl)methyl]pyridin-3-yl |
| 2216 | pyridazin-4-yl | CH₃ | 2-(2,4-dichlorophenyl)-1,3-thiazol-5-yl |
| 2217 | pyridazin-4-yl | CH₃ | 6-[(4,5,6-trimethylpyrimidin-2-yl)sulphanyl]pyridin-3-yl |
| 2218 | pyridazin-4-yl | CH₃ | 4-[(4,6-dimethylpyrimidin-2-yl)sulphanyl]phenyl |
| 2219 | pyridazin-4-yl | CH₃ | 6-[2-chloro-4-(methylsulphanyl)phenoxy]pyridin-3-yl |
| 2220 | pyridazin-4-yl | CH₃ | 1-(2-bromo-6-chlorophenyl)-5-(difluoromethyl)-1H-pyrazol-4- |
| | | | yl |
| 2221 | pyridazin-4-yl | CH₃ | 4-(propan-2-yloxy)-3-[3-(trifluoromethyl)-1H-pyrazol-1- |
| | | | yl]phenyl |
| 2222 | pyridazin-4-yl | CH₃ | 3-[ethyl(methyl)carbamoyl]phenyl |
| 2223 | pyridazin-4-yl | CH₃ | 6-(3,5-dimethylphenoxy)pyridin-3-yl |
| 2224 | pyridazin-4-yl | CH₃ | 1-[4-chloro-2-(trifluoromethyl)phenyl]-5-methyl-1H-pyrazol- |
| | | | 4-yl |
| 2225 | pyridazin-4-yl | CH₃ | 4-fluoro-3-(trifluoromethyl)phenyl |
| 2226 | pyridazin-4-yl | CH₃ | 6-[4-(methoxycarbonyl)phenoxy]pyridin-3-yl |
| 2227 | pyridazin-4-yl | CH₃ | 3-[(cyclopropylcarbonyl)amino]-4-methylphenyl |
| 2228 | pyridazin-4-yl | CH₃ | 1-(4-chlorophenyl)-1H-pyrazol-4-yl |
| 2229 | pyridazin-4-yl | CH₃ | 2-[2-(difluoromethyl)phenyl]-1,3-thiazol-5-yl |
| 2230 | pyridazin-4-yl | CH₃ | 1-ethyl-3-(trifluoromethyl)-1H-pyrazol-4-yl |
| 2231 | pyridazin-4-yl | CH₃ | 5-chloro-6-[4-(trifluoromethyl)phenoxy]pyridin-3-yl |
| 2232 | pyridazin-4-yl | CH₃ | 4-methyl-3-(propanoylamino)phenyl |
| 2233 | pyridazin-4-yl | CH₃ | 2-(2-chloropyridin-3-yl)-1,3-thiazol-4-yl |
| 2234 | pyridazin-4-yl | CH₃ | 4-[(2-methylpropanoyl)amino]phenyl |
| 2235 | pyridazin-4-yl | CH₃ | 3-[(2,2-dimethylpropanoyl)amino]-4-methylphenyl |
| 2236 | pyridazin-4-yl | CH₃ | 4-methoxy-3-{[(propan-2-yloxy)carbonyl]amino}phenyl |
| 2237 | pyridazin-4-yl | CH₃ | 1-(2,3-dichlorophenyl)-5-(methoxymethyl)-1H-pyrazol-4-yl |
| 2238 | pyridazin-4-yl | CH₃ | 3-(hydroxymethyl)-1-methyl-1H-pyrazol-4-yl |
| 2239 | pyridazin-4-yl | CH₃ | 1-(propan-2-yl)-3-(trifluoromethyl)-1H-pyrazol-4-yl |
| 2240 | pyridazin-4-yl | CH₃ | 2-[3-(difluoromethyl)pyridin-2-yl]-1,3-thiazol-5-yl |
| 2241 | pyridazin-4-yl | CH₃ | 4-(pyrrolidin-l-ylcarbonyl)phenyl |
| 2242 | pyridazin-4-yl | CH₃ | 3-(methoxymethyl)-1-methyl-1H-pyrazol-4-yl |
| 2243 | pyridazin-4-yl | CH₃ | 2-(3,5-dichloropyridin-2-yl)-1,3-thiazol-5-yl |
| 2244 | pyridazin-4-yl | CH₃ | 1-(2,6-dichloropyridin-3-yl)-5-(difluoromethyl)-1H-pyrazol-4- |
| | | | yl |
| 2245 | pyridazin-4-yl | CH₃ | 6-[(4,6-dimethylpyrimidin-2-yl)oxy]pyridin-3-yl |
| 2246 | pyridazin-4-yl | CH₃ | 4-[(4,6-dimethoxy-1,3,5-triazin-2-yl)sulphanyl]phenyl |
| 2247 | pyridazin-4-yl | CH₃ | 4-(methylsulphanyl)phenyl |
| 2248 | pyridazin-4-yl | CH₃ | 4-(ethylsulphanyl)phenyl |
| 2249 | pyridazin-4-yl | CH₃ | 6-[4-(trifluoromethoxy)phenoxy]pyridin-3-yl |
| 2250 | pyridazin-4-yl | CH₃ | 5-chloro-6-(4-nitrophenoxy)pyridin-3-yl |
| 2251 | pyridazin-4-yl | CH₃ | 2-[2-(difluoromethyl)phenyl]-1,3-thiazol-4-yl |
| 2252 | pyridazin-4-yl | CH₃ | 1-(difluoromethyl)-3-(trifluoromethyl)-1H-pyrazol-4-yl |
| 2253 | pyridazin-4-yl | CH₃ | 6-[(6-chloropyridin-2-yl)oxy]pyridin-3-yl |
| 2254 | pyridazin-4-yl | CH₃ | 3-(butanoylamino)-4-methoxyphenyl |
| 2255 | pyridazin-4-yl | CH₃ | 4-(acetylamino)phenyl |
| 2256 | pyridazin-4-yl | CH₃ | 1-(2-bromophenyl)-5-(trifluoromethyl)-1H-pyrazol-4-yl |
| 2257 | pyridazin-4-yl | CH₃ | 6-(4-chlorophenoxy)pyridin-3-yl |
| 2258 | pyridazin-4-yl | CH₃ | 3-{[3-(trifluoromethyl)-1H-pyrazol-1-yl]methyl}phenyl |
| 2259 | pyridazin-4-yl | CH₃ | 5-(difluoromethyl)-1-(2-methylphenyl)-1H-pyrazol-4-yl |
| 2260 | pyridazin-4-yl | CH₃ | 2-[2-(1-methoxyethyl)phenyl]-1,3-thiazol-5-yl |
| 2261 | pyridazin-4-yl | CH₃ | 1-(2-chlorophenyl)-5-(1,1-difluoroethyl)-1H-pyrazol-4-yl |
| 2262 | pyridazin-4-yl | CH₃ | 1-(2-bromo-4-fluorophenyl)-1H-pyrazol-4-yl |
| 2263 | pyridazin-4-yl | CH₃ | 6-[(cyclopropylmethyl)amino]pyridin-3-yl |
| 2264 | pyridazin-4-yl | CH₃ | 6-[(2-methoxypyrimidin-4-yl)oxy]pyridin-3-yl |
| 2265 | pyridazin-4-yl | CH₃ | 4-(methoxycarbonyl)phenyl |
| 2266 | pyridazin-4-yl | CH₃ | 1-[2-(1,1-difluoroethyl)phenyl]-5-methyl-I H-pyrazol-4-yl |
| 2267 | pyridazin-4-yl | CH₃ | 3-{[3,5-bis(trifluoromethyl)-1H-1,2,4-triazol-1-yl]methyl}-4- |
| | | | chlorophenyl |
| 2268 | pyridazin-4-yl | CH₃ | 6-(morpholin-4-yl)pyridin-3-yl |
| 2269 | pyridazin-4-yl | CH₃ | 2-(pyrimidin-2-yl)-1,3-thiazol-4-yl |
| 2270 | pyridazin-4-yl | CH₃ | 4-[(4,6-dimethoxypyrimidin-2-yl)oxy]phenyl |
| 2271 | pyridazin-4-yl | CH₃ | 2-{[3-(trifluoromethyl)-1H-pyrazol-1-yl]methyl}pyridin-4-yl |
| 2272 | pyridazin-4-yl | CH₃ | 1-(2-chloropyridin-3-yl)-5-(trifluoromethyl)-1H-pyrazol-4-yl |
| 2273 | pyridazin-4-yl | CH₃ | 6-(4-bromophenoxy)pyridin-3-yl |
| 2274 | pyridazin-4-yl | CH₃ | 4-[(propylsulphanyl)methyl]phenyl |
| 2275 | pyridazin-4-yl | CH₃ | 6-(pyrimidin-2-ylsulphanyl)pyridin-3-yl |
| 2276 | pyridazin-4-yl | CH₃ | 1-(2-chloro-4-fluorophenyl)-1H-pyrazol-4-yl |
| 2277 | pyridazin-4-yl | CH₃ | 4-methoxy-3-[4-(pyridin-2-yl)-1H-pyrazol-1-yl]phenyl |
| 2278 | pyridazin-4-yl | CH₃ | 1-(2-chlorophenyl)-1H-pyrazol-4-yl |
| 2279 | pyridazin-4-yl | CH₃ | 6-(2,6-dichlorophenoxy)pyridin-3-yl |
| 2280 | pyridazin-4-yl | CH₃ | 3-[(4,6-dimethoxypyrimidin-2-yl)oxy]phenyl |
| 2281 | pyridazin-4-yl | CH₃ | 6-(4-nitrophenoxy)pyridin-3-yl |
| 2282 | pyridazin-4-yl | CH₃ | 1-(2-chlorophenyl)-5-methyl-1H-pyrazol-4-yl |
| 2283 | pyridazin-4-yl | CH₃ | 3-[ethyl(methyl)carbamoyl]-4-methoxyphenyl |
| 2284 | pyridazin-4-yl | CH₃ | 4-[(ethylsulphanyl)methyl]phenyl |
| 2285 | pyridazin-4-yl | CH₃ | 2-[2-(1-methoxyethyl)phenyl]-1,3-thiazol-4-yl |
| 2286 | pyridazin-4-yl | CH₃ | 6-{[6-(1H-pyrazol-1-yl)pyridin-2-yl]oxy}pyridin-3-yl |
| 2287 | pyridazin-4-yl | CH₃ | 2-[4-chloro-2-(trifluoromethyl)phenyl]-1,3-thiazol-4-yl |
| 2288 | pyridazin-4-yl | CH₃ | 4-methoxy-3-(1H-pyrazol-1-yl)phenyl |
| 2289 | pyridazin-4-yl | CH₃ | 3-(acetylamino)-4-methylphenyl |
| 2290 | pyridazin-4-yl | CH₃ | 2-{[4-(trifluoromethyl)-1H-pyrazol-1-yl]methyl}pyridin-4-yl |
| 2291 | pyridazin-4-yl | CH₃ | 1-(2,4-difluorophenyl)-1H-pyrazol-4-yl |
| 2292 | pyridazin-4-yl | CH₃ | 1-(2-methylphenyl)-5-(trifluoromethyl)-1H-pyrazol-4-yl |
| 2293 | pyridazin-4-yl | CH₃ | 2-[3-(difluoromethyl)pyridin-2-yl]-1,3-thiazol-4-yl |
| 2294 | pyridazin-4-yl | CH₃ | 6-(3,5-dichlorophenoxy)pyridin-3-yl |
| 2295 | pyridazin-4-yl | CH₃ | 1-(pyrimidin-2-ylmethyl)-3-(trifluoromethyl)-1H-pyrazol-4-yl |
| 2296 | pyridazin-4-yl | CH₃ | 6-methylpyridin-3-yl |
| 2297 | pyridazin-4-yl | CH₃ | 6-[(4-methylphenyl)sulphanyl]pyridin-3-yl |
| 2298 | pyridazin-4-yl | CH₃ | 1-(2,4-dichlorophenyl)-5-(methoxymethyl)-1H-pyrazol-4-yl |
| 2299 | pyridazin-4-yl | CH₃ | 6-{4-[4-(trifluoromethyl)phenoxy]phenoxy}pyridin-3-yl |
| 2300 | pyridazin-4-yl | CH₃ | 1-(2,6-dichlorophenyl)-5-(trifluoromethyl)-1H-pyrazol-4-yl |
| 2301 | pyridazin-4-yl | CH₃ | 4-[(4,6-dimethylpyrimidin-2-yl)oxy]phenyl |
| 2302 | pyridazin-4-yl | CH₃ | 6-(naphthalen-1-yloxy)pyridin-3-yl |
| 2303 | pyridazin-4-yl | CH₃ | 2-(2,6-dichloropyridin-3-yl)-1,3-thiazol-5-yl |
| 2304 | pyridazin-4-yl | CH₃ | 6-{[(4,6-dimethoxypyrimidin-2-yl)methyl]sulphanyl}pyridin- |
| | | | 3-yl |
| 2305 | pyridazin-4-yl | CH₃ | 1-[3-chloro-2-(trifluoromethyl)phenyl]-5-(1-methoxyethyl)- |
| | | | 1H-pyrazol-4-yl |
| 2306 | pyridazin-4-yl | CH₃ | 3,4-dimethoxyphenyl |
| 2307 | pyridazin-4-yl | CH₃ | 4-methoxy-3-[(methoxyacetyl)amino]phenyl |
| 2308 | pyridazin-4-yl | CH₃ | 3-{[4-(trifluoromethyl)-1H-pyrazol-1-yl]methyl}phenyl |
| 2309 | pyridazin-4-yl | CH₃ | 6-(2-cyanophenoxy)pyridin-3-yl |
| 2310 | pyridazin-4-yl | CH₃ | 3-[(methylsulphanyl)methyl]phenyl |
| 2311 | pyridazin-4-yl | CH₃ | 3-(dimethoxymethyl)-1-methyl-1H-pyrazol-4-yl |
| 2312 | pyridazin-4-yl | CH₃ | 1-(ethoxymethyl)-3-(trifluoromethyl)-1H-pyrazol-4-yl |
| 2313 | pyridazin-4-yl | CH₃ | 1-(3-bromopyridin-2-yl)-5-(methoxymethyl)-1H-pyrazol-4-yl |
| 2314 | pyridazin-4-yl | CH₃ | 6-(methylsulphanyl)pyridin-3-yl |
| 2315 | pyridazin-4-yl | CH₃ | 1-(2-bromo-6-chlorophenyl)-5-(trifluoromethyl)-1H-pyrazol-4- |
| | | | yl |
| 2316 | pyridazin-4-yl | CH₃ | 3-{[4-(trifluoromethyl)phenoxy]methyl}phenyl |
| 2317 | pyridazin-4-yl | CH₃ | 6-(heptafluoropropyl)pyridin-3-yl |
| 2318 | pyridazin-4-yl | CH₃ | 1-ethenyl-3-(trifluoromethyl)-1H-pyrazol-4-yl |
| 2319 | pyridazin-4-yl | CH₃ | 4-methyl-3-nitrophenyl |
| 2320 | pyridazin-4-yl | CH₃ | 3-(4-bromo-1H-pyrazol-1-yl)-4-methoxyphenyl |
| 2321 | pyridazin-4-yl | CH₃ | 5-methyl-1-(2-methylphenyl)-1H-pyrazol-4-yl |
| 2322 | pyridazin-4-yl | CH₃ | 1-[3-(difluoromethyl)pyridin-2-yl]-5-(methoxymethyl)-1H- |
| | | | pyrazol-4-yl |
| 2323 | pyridazin-4-yl | CH₃ | 6-{[2-(trifluoromethyl)pyrimidin-5-yl]oxy}pyridin-3-yl |
| 2324 | pyridazin-4-yl | CH₃ | 2-[2-(methoxymethyl)phenyl]-1,3-thiazol-5-yl |
| 2325 | pyridazin-4-yl | CH₃ | 1-(ethoxymethyl)-5-(trifluoromethyl)-1H-pyrazol-4-yl |
| 2326 | pyridazin-4-yl | CH₃ | 5-chloro-6-[(4,5,6-trimethylpyrimidin-2-yl)sulphanyl]pyridin- |
| | | | 3-yl |
| 2327 | pyridazin-4-yl | CH₃ | 6-[3,5-bis(trifluoromethyl)phenoxy]pyridin-3-yl |
| 2328 | pyridazin-4-yl | CH₃ | 4-{[4-(pyridin-2-yl)-1H-pyrazol-1-yl]methyl}phenyl |
| 2329 | pyridazin-4-yl | CH₃ | 1-[3-(1,1-difluoroethyl)pyridin-2-yl]-5-methyl-I H-pyrazol-4-yl |
| 2330 | pyridazin-4-yl | CH₃ | 1-(2,6-dichlorophenyl)-5-(difluoromethyl)-1H-pyrazol-4-yl |
| 2331 | pyridazin-4-yl | CH₃ | 1-[4-fluoro-2-(trifluoromethyl)phenyl]-5-methyl-1H-pyrazol-4- |
| | | | yl |
| 2332 | pyridazin-4-yl | CH₃ | 4-chloro-3-[(cyclobutylcarbonyl)amino]phenyl |
| 2333 | pyridazin-4-yl | CH₃ | 2-[2-(methoxymethyl)phenyl]-1,3-thiazol-4-yl |
| 2334 | pyridazin-4-yl | CH₃ | 4-(1H-pyrazol-1-ylmethyl)phenyl |
| 2335 | pyridazin-4-yl | CH₃ | 1-(2-bromophenyl)-5-methyl-1H-pyrazol-4-yl |
| 2336 | pyridazin-4-yl | CH₃ | 1-(2,3-difluorophenyl)-1H-pyrazol-4-yl |
| 2337 | pyridazin-4-yl | CH₃ | 6-{4-[(trifluoromethyl)sulphanyl]phenoxy}pyridin-3-yl |
| 2338 | pyridazin-4-yl | CH₃ | 4-{[3-(pentafluoroethyl)-1H-1,2,4-triazol-1-yl]methyl}phenyl |
| 2339 | pyridazin-4-yl | CH₃ | 4-{[3-(pyrimidin-2-yl)-1H-pyrazol-1-yl]methyl}phenyl |
| 2340 | pyridazin-4-yl | CH₃ | 1-(2-chlorophenyl)-5-(difluoromethyl)-1H-pyrazol-4-yl |
| 2341 | pyridazin-4-yl | CH₃ | 5-(difluoromethyl)-1-[2-(trifluoromethyl)phenyl]-1H-pyrazol- |
| | | | 4-yl |
| 2342 | pyridazin-4-yl | CH₃ | 6-(4-methoxyphenoxy)pyridin-3-yl |
| 2343 | pyridazin-4-yl | CH₃ | 1-[2-(dimethoxymethyl)phenyl]-1H-pyrazol-4-yl |
| 2344 | pyridazin-4-yl | CH₃ | 5-(methoxymethyl)-1-(pyrimidin-2-yl)-1H-pyrazol-4-yl |
| 2345 | pyridazin-4-yl | CH₃ | 6-tert-butoxypyridin-3-yl |
| 2346 | pyridazin-4-yl | CH₃ | 4-[(cyclopentylimino)methyl]phenyl |
| 2347 | pyridazin-4-yl | CH₃ | 1-(3-chlorophenyl)-1H-pyrazol-4-yl |
| 2348 | pyridazin-4-yl | CH₃ | 3-[(2-methylpropanoyl)amino]phenyl |
| 2349 | pyridazin-4-yl | CH₃ | 6-{[3,5-bis(trifluoromethyl)-1H-pyrazol-1-yl]methyl}pyridin- |
| | | | 3-yl |
| 2350 | pyridazin-4-yl | CH₃ | 4-chloro-3-[(2-methylpropanoyl)amino]phenyl |
| 2351 | pyridazin-4-yl | CH₃ | 4-(propylsulphanyl)phenyl |
| 2352 | pyridazin-4-yl | CH₃ | 1-[2-(difluoromethyl)phenyl]-5-(methoxymethyl)-1H-pyrazol- |
| | | | 4-yl |
| 2353 | pyridazin-4-yl | CH₃ | 4-methoxy-3-[4-(pyrimidin-2-yl)-1H-pyrazol-1-yl]phenyl |
| 2354 | pyridazin-4-yl | CH₃ | 4-{[(4,6-dimethylpyrimidin-2-yl)sulphanyl]methyl}phenyl |
| 2355 | pyridazin-4-yl | CH₃ | 1-(2-chlorophenyl)-5-(methoxymethyl)-1H-pyrazol-4-yl |
| 2356 | pyridazin-4-yl | CH₃ | 6-{[3,5-bis(trifluoromethyl)-1H-1,2,4-triazol-1- |
| | | | yl]methyl}pyridin-3-yl |
| 2357 | pyridazin-4-yl | CH₃ | 5-chloro-6-[4-(trifluoromethoxy)phenoxy]pyridin-3-yl |
| 2358 | pyridazin-4-yl | CH₃ | 4-[(tent-butoxyimino)methyl]phenyl |
| 2359 | pyridazin-4-yl | CH₃ | 2-(3-chloropyridin-2-yl)-1,3-thiazol-5-yl |
| 2360 | pyridazin-4-yl | CH₃ | 1-(2-fluorophenyl)-1H-pyrazol-4-yl |
| 2361 | pyridazin-4-yl | CH₃ | 4-(trifluoromethoxy)phenyl |
| 2362 | pyridazin-4-yl | CH₃ | 6-(2-chlorophenoxy)pyridin-3-yl |
| 2363 | pyridazin-4-yl | CH₃ | 3-[(2,2-dimethylpropanoyl)amino]-5-(trifluoromethyl)phenyl |
| 2364 | pyridazin-4-yl | CH₃ | 6-[(4-methylpyrimidin-2-yl)sulphanyl]pyridin-3-yl |
| 2365 | pyridazin-4-yl | CH₃ | 6-{[2-(morpholin-4-yl)ethyl]amino}pyridin-3-yl |
| 2366 | pyridazin-4-yl | CH₃ | 3-formyl-1-methyl-1H-pyrazol-4-yl |
| 2367 | pyridazin-4-yl | CH₃ | 1-(3-chlorophenyl)-5-(1,1-dimethoxyethyl)-1H-pyrazol-4-yl |
| 2368 | pyridazin-4-yl | CH₃ | 4-nitrophenyl |
| 2369 | pyridazin-4-yl | CH₃ | 6-{[5-(methoxycarbonyl)pyridin-3-yl]oxy}pyridin-3-yl |
| 2370 | pyridazin-4-yl | CH₃ | 3-{[4-(trifluoromethyl)benzyl]oxy}phenyl |
| 2371 | pyridazin-4-yl | CH₃ | 1-(2,4-dichlorophenyl)-5-(1,1-dimethoxyethyl)-1H-pyrazol-4- |
| | | | yl |
| 2372 | pyridazin-4-yl | CH₃ | 4-{[4-bromo-3-(trifluoromethyl)-1H-pyrazol-1- |
| | | | yl]methyl}phenyl |
| 2373 | pyridazin-4-yl | CH₃ | 3-[(cyclopropylcarbonyl)amino]-4-fluorophenyl |
| 2374 | pyridazin-4-yl | CH₃ | 6-[4-(dimethylsulphamoyl)phenoxy]pyridin-3-yl |
| 2375 | pyridazin-4-yl | CH₃ | 5-methyl-1-(2-methylpyridin-3-yl)-1H-pyrazol-4-yl |
| 2376 | pyridazin-4-yl | CH₃ | 1-(2,4-difluorophenyl)-5-methyl-1H-pyrazol-4-yl |
| 2377 | pyridazin-4-yl | CH₃ | 3-(methylsulphanyl)phenyl |
| 2378 | pyridazin-4-yl | CH₃ | 3-(acetylamino)-4-chlorophenyl |
| 2379 | pyridazin-4-yl | CH₃ | 4-[(4-bromo-1H-pyrazol-1-yl)methyl]phenyl |
| 2380 | pyridazin-4-yl | CH₃ | 4-methyl-3-[(propan-2-ylcarbamoyl)amino]phenyl |
| 2381 | pyridazin-4-yl | CH₃ | 1-(2,2-diethoxyethyl)-3-(trifluoromethyl)-1H-pyrazol-4-yl |
| 2382 | pyridazin-4-yl | CH₃ | 6-[(4,6-dimethoxypyrimidin-2-yl)oxy]pyridin-3-yl |
| 2383 | pyridazin-4-yl | CH₃ | 6-(1H-pyrazol-1-yl)pyridin-3-yl |
| 2384 | pyridazin-4-yl | CH₃ | 4-(1H-1,2,4-triazol-1-ylmethyl)phenyl |
| 2385 | pyridazin-4-yl | CH₃ | 5-(methoxymethyl)-1-[2-(trifluoromethyl)phenyl]-1H-pyrazol- |
| | | | 4-yl |
| 2386 | pyridazin-4-yl | CH₃ | 5-methylpyridin-3-yl |
| 2387 | pyridazin-4-yl | CH₃ | 5-chloro-6-[(4,6-dimethylpyrimidin-2-yl)sulphanyl]pyridin-3- |
| | | | yl |
| 2388 | pyridazin-4-yl | CH₃ | 3-[(2,2-dimethylpropanoyl)amino]-4-methoxyphenyl |
| 2389 | pyridazin-4-yl | CH₃ | 2-[2-(trifluoromethyl)pyridin-3-yl]-1,3-thiazol-4-yl |
| 2390 | pyridazin-4-yl | CH₃ | 3-methyl-I H-pyrazol-4-yl |
| 2391 | pyridazin-4-yl | CH₃ | 5-(methoxymethyl)-1-(2,2,2-trifluoroethyl)-1H-pyrazol-4-yl |
| 2392 | pyridazin-4-yl | CH₃ | 4-methoxy-3-(propanoylamino)phenyl |
| 2393 | pyridazin-4-yl | CH₃ | 4-methoxy-3-[(2-methylpropanoyl)amino]phenyl |
| 2394 | pyridazin-4-yl | CH₃ | 6-(quinolin-7-yloxy)pyridin-3-yl |
| 2395 | pyridazin-4-yl | CH₃ | 4-{[3-(trifluoromethyl)-1H-pyrazol-1-yl]methyl}phenyl |
| 2396 | pyridazin-4-yl | CH₃ | 3-[(4,6-dimethylpyrimidin-2-yl)oxy]phenyl |
| 2397 | pyridazin-4-yl | CH₃ | 4-{[3,5-bis(trifluoromethyl)-1H-1,2,4-triazol-1- |
| | | | yl]methyl}phenyl |
| 2398 | pyridazin-4-yl | CH₃ | 3-fluoro-4-{[4-(trifluoromethyl)-1H-pyrazol-1- |
| | | | yl]methyl}phenyl |
| 2399 | pyridazin-4-yl | CH₃ | 2,3-dihydro-1,4-benzodioxin-6-yl |
| 2400 | pyridazin-4-yl | CH₃ | 2-[3-(methoxymethyl)pyridin-2-yl]-1,3-thiazol-5-yl |
| 2401 | pyridazin-4-yl | CH₃ | 6-({6-[3-(trifluoromethyl)-1H-pyrazol-1-yl]pyridin-2- |
| | | | yl}oxy)pyridin-3-yl |
| 2402 | pyridazin-4-yl | CH₃ | 3-{[3-(pyridin-2-yl)-1H-pyrazol-1-yl]methyl}phenyl |
| 2403 | pyridazin-4-yl | CH₃ | 1-(2-bromo-4-chlorophenyl)-5-methyl-1H-pyrazol-4-yl |
| 2404 | pyridazin-4-yl | CH₃ | 2,2-difluoro-1,3-benzodioxo1-5-yl |
| 2405 | pyridazin-4-yl | CH₃ | 2-[3-(trifluoromethyl)pyridin-2-yl]-1,3-thiazol-4-yl |
| 2406 | pyridazin-4-yl | CH₃ | 4-(benzylcarbamoyl)phenyl |
| 2407 | pyridazin-4-yl | CH₃ | 4-[(2-chloropyrimidin-4-yl)oxy]phenyl |
| 2408 | pyridazin-4-yl | CH₃ | 1-[3-(trifluoromethyl)pyridin-2-yl]-1H-pyrazol-4-yl |
| 2409 | pyridazin-4-yl | CH₃ | 5-chloro-6-(4-chlorophenoxy)pyridin-3-yl |
| 2410 | pyridazin-4-yl | CH₃ | 3-cyano-4-(1H-1,2,4-triazol-1-yl)phenyl |
| 2411 | pyridazin-4-yl | CH₃ | 3-amino-4-(methylsulphonyl)phenyl |
| 2412 | pyridazin-4-yl | CH₃ | 4-methyl-3-[(2-methylpropanoyl)amino]phenyl |
| 2413 | pyridazin-4-yl | CH₃ | 1-(2,6-dibromophenyl)-5-(trifluoromethyl)-1H-pyrazol-4-yl |
| 2414 | pyridazin-4-yl | CH₃ | 3-{[3-(trifluoromethyl)-1H-1,2,4-triazol-1-yl]methyl}phenyl |
| 2415 | pyridazin-4-yl | CH₃ | 4-[(3,3,3-trifluoropropanoyl)amino]phenyl |
| 2416 | pyridazin-4-yl | CH₃ | 5-(pyrazin-2-yl)-1-(2,2,2-trifluoroethyl)-1H-pyrazol-4-yl |
| 2417 | pyridazin-4-yl | CH₃ | 1-(2,6-dichlorophenyl)-5-(dimethoxymethyl)-1H-pyrazol-4-yl |
| 2418 | pyridazin-4-yl | CH₃ | 1-(4-chlorophenyl)-5-(1,1-dimethoxyethyl)-1H-pyrazol-4-yl |
| 2419 | pyridazin-4-yl | CH₃ | 6-[3-(trifluoromethyl)phenoxy]pyridin-3-yl |
| 2420 | pyridazin-4-yl | CH₃ | 4-({6-[3-(trifluoromethyl)-1H-pyrazol-1-yl]pyridin-2- |
| | | | yl}oxy)phenyl |
| 2421 | pyridazin-4-yl | CH₃ | 4-methoxy-3-(propan-2-ylcarbamoyl)phenyl |
| 2422 | pyridazin-4-yl | CH₃ | 1-[2-(1-methoxyethyl)phenyl]-5-methyl-1H-pyrazol-4-yl |
| 2423 | pyridazin-4-yl | CH₃ | 4-[(4,6-dimethoxypyrimidin-2-yl)sulphanyl]phenyl |
| 2424 | pyridazin-4-yl | CH₃ | 6-(4-methylphenoxy)pyridin-3-yl |
| 2425 | pyridazin-4-yl | CH₃ | 6-(4-chloro-2-cyanophenoxy)pyridin-3-yl |
| 2426 | pyridazin-4-yl | CH₃ | 1-propyl-3-(trifluoromethyl)-1H-pyrazol-4-yl |
| 2427 | pyridazin-4-yl | CH₃ | 6-(2-methylphenoxy)pyridin-3-yl |
| 2428 | pyridazin-4-yl | CH₃ | 4-{[4-(trifluoromethyl)-1H-pyrazol-1-yl]methyl}phenyl |
| 2429 | pyridazin-4-yl | CH₃ | 6-[(6-methylpyridin-3-yl)oxy]pyridin-3-yl |
| 2430 | pyridazin-4-yl | CH₃ | 6-methoxypyridin-3-yl |
| 2431 | pyridazin-4-yl | CH₃ | 4-[(ethoxyimino)methyl]phenyl |
| 2432 | pyridazin-4-yl | CH₃ | 4-{[acetyl(cyclopentyl)amino]methyl}phenyl |
| 2433 | pyridazin-4-yl | CH₃ | 3-{[3,5-bis(trifluoromethyl)-1H-pyrazol-1-yl]methyl}phenyl |
| 2434 | pyridazin-4-yl | CH₃ | 1-(2-bromo-4-chlorophenyl)-1H-pyrazol-4-yl |
| 2435 | pyridazin-4-yl | CH₃ | 6-[4-(1H-1,2,4-triazol-1-yl)phenoxy]pyridin-3-yl |
| 2436 | pyridazin-4-yl | CH₃ | 6-[4-(trifluoromethyl)phenoxy]pyridin-3-yl |
| 2437 | pyridazin-4-yl | CH₃ | 1-(2-chlorophenyl)-5-(trifluoromethyl)-1H-pyrazol-4-yl |
| 2438 | pyridazin-4-yl | CH₃ | 4-methoxy-3-nitrophenyl |
| 2439 | pyridazin-4-yl | CH₃ | 1-(2,6-dibromophenyl)-5-(difluoromethyl)-1H-pyrazol-4-yl |
| 2440 | pyridazin-4-yl | CH₃ | 6-[(4-methoxyphenyl)sulphanyl]pyridin-3-yl |
| 2441 | pyridazin-4-yl | CH₃ | 1-(2,6-dichlorophenyl)-5-(1-methoxyethyl)-1H-pyrazol-4-yl |
| 2442 | pyridazin-4-yl | CH₃ | 4-[(6-chloropyridin-2-yl)oxy]phenyl |
| 2443 | pyridazin-4-yl | CH₃ | 3-fluoro-4-{[3-(trifluoromethyl)-1H-pyrazol-1- |
| | | | yl]methyl}phenyl |
| 2444 | pyridazin-4-yl | CH₃ | 4-{[(difluoromethyl)sulphanyl]methyl}phenyl |
| 2445 | pyridazin-4-yl | CH₃ | 6-{[3-(trifluoromethyl)-1H-pyrazol-1-yl]methyl}pyridin-3-yl |
| 2446 | pyridazin-4-yl | CH₃ | 4-{[2-(3,5-dichlorophenyl)propan-2-yl]carbamoyl}phenyl |
| 2447 | pyridazin-4-yl | CH₃ | 3-(1H-1,2,4-triazol-1-ylmethyl)phenyl |
| 2448 | pyridazin-4-yl | CH₃ | 2-(3-chloropyridin-2-yl)-1,3-thiazol-4-yl |
| 2449 | pyridazin-4-yl | CH₃ | 5-(1-methoxyethyl)-1-(pyrimidin-2-yl)-1H-pyrazol-4-yl |
| 2450 | pyridazin-4-yl | CH₃ | 1-(3-chloropyridin-2-yl)-5-(1,1-dimethoxyethyl)-1H-pyrazol- |
| | | | 4-yl |
| 2451 | pyridazin-4-yl | CH₃ | 1-(2-bromo-4-fluorophenyl)-5-methyl-1H-pyrazol-4-yl |
| 2452 | pyridazin-4-yl | CH₃ | 3-(butanoylamino)-4-methylphenyl |
| 2453 | pyridazin-4-yl | CH₃ | 5-(methoxymethyl)-1-[3-(trifluoromethyl)pyridin-2-yl]-1H- |
| | | | pyrazol-4-yl |
| 2454 | pyridazin-4-yl | CH₃ | 6-[3-(trifluoromethyl)-1H-pyrazol-1-yl]pyridin-3-yl |
| 2455 | pyridazin-4-yl | CH₃ | 6-(3-chlorophenoxy)pyridin-3-yl |
| 2456 | pyridazin-4-yl | CH₃ | 1-(2,6-dichloropyridin-3-yl)-5-(1-methoxyethyl)-1H-pyrazol- |
| | | | 4-yl |
| 2457 | pyridazin-4-yl | CH₃ | 1-[2-(difluoromethyl)phenyl]-5-methyl-1H-pyrazol-4-yl |
| 2458 | pyridazin-4-yl | CH₃ | 2-[2-(trifluoromethyl)phenyl]-1,3-thiazol-5-yl |
| 2459 | pyridazin-4-yl | CH₃ | 2-[2-(trifluoromethyl)pyridin-3-yl]-1,3-thiazol-5-yl |
| 2460 | pyridazin-4-yl | CH₃ | 6-[(4,6-dimethoxy-1,3,5-triazin-2-yl)sulphanyl]pyridin-3-yl |
| 2461 | pyridazin-4-yl | CH₃ | 2-(2-chloropyridin-3-yl)-1,3-thiazol-5-yl |
| 2462 | pyridazin-4-yl | CH₃ | 1-(2,6-dichloropyridin-3-yl)-5-methyl-1H-pyrazol-4-yl |
| 2463 | pyridazin-4-yl | CH₃ | 1-(3-chloropyridin-2-yl)-5-(methoxymethyl)-1H-pyrazol-4-yl |
| 2464 | pyridazin-4-yl | CH₃ | 6-[(4-chlorophenyl)sulphanyl]pyridin-3-yl |
| 2465 | pyridazin-4-yl | CH₃ | 6-(piperidin-l-yl)pyridin-3-yl |
| 2466 | pyridazin-4-yl | CH₃ | 3-[(cyclopentylcarbonyl)amino]-4-methylphenyl |
| 2467 | pyridazin-4-yl | CH₃ | 6-ethoxypyridin-3-yl |
| 2468 | pyridazin-4-yl | CH₃ | 2-(2,6-dichlorophenyl)-1,3-thiazol-4-yl |
| 2469 | pyridazin-4-yl | CH₃ | 5-chloro-6-[(4-methylpyrimidin-2-yl)sulphanyl]pyridin-3-yl |
| 2470 | pyridazin-4-yl | CH₃ | 1-[4-chloro-2-(trifluoromethyl)phenyl]-5-(1-methoxyethyl)- |
| | | | 1H-pyrazol-4-yl |
| 2471 | pyridazin-4-yl | CH₃ | 6-(3-methylphenoxy)pyridin-3-yl |
| 2472 | pyridazin-4-yl | CH₃ | 4-[ethyl(methyl)carbamoyl]phenyl |
| 2473 | pyridazin-4-yl | CH₃ | 6-[3-(trifluoromethoxy)phenoxy]pyridin-3-yl |
| 2474 | pyridazin-4-yl | CH₃ | 5-(difluoromethyl)-1-(pyrimidin-2-yl)-1H-pyrazol-4-yl |
| 2475 | pyridazin-4-yl | CH₃ | 4-[(cyclopropylcarbonyl)amino]phenyl |
| 2476 | pyridazin-4-yl | CH₃ | 1-(3-bromopyridin-2-yl)-5-(1-methoxyethyl)-1H-pyrazol-4-yl |
| 2477 | pyridazin-4-yl | CH₃ | 3-[(cyclopropylcarbonyl)amino]-4-methoxyphenyl |
| 2478 | pyridazin-4-yl | CH₃ | 4-methyl-3-[(3-methylbut-2-enoyl)amino]phenyl |
| 2479 | pyridazin-4-yl | CH₃ | 1-[3-(methoxymethyl)pyridin-2-yl]-1H-pyrazol-4-yl |
| 2480 | pyridazin-4-yl | CH₃ | 4-[acetyl(methyl)amino]phenyl |
| 2481 | pyridazin-4-yl | CH₃ | 6-(naphthalen-2-yloxy)pyridin-3-yl |
| 2482 | pyridazin-4-yl | CH₃ | 1-(2-chlorophenyl)-5-(1-methoxyethyl)-1H-pyrazol-4-yl |
| 2483 | pyridazin-4-yl | CH₃ | 1-[2-(difluoromethyl)phenyl]-5-(1-methoxyethyl)-1H-pyrazol- |
| | | | 4-yl |
| 2484 | pyridazin-4-yl | CH₃ | 1-(3-chloropyridin-2-yl)-5-methyl-1H-pyrazol-4-yl |
| 2485 | pyridazin-4-yl | CH₃ | 4-{[3-(trifluoromethyl)-1H-1,2,4-triazol-1-yl]methyl}phenyl |
| 2486 | pyridazin-4-yl | CH₃ | 3-[(cyclopentylcarbonyl)amino]-4-methoxyphenyl |
| 2487 | pyridazin-4-yl | CH₃ | 6-[4-(methylsulphanyl)phenoxy]pyridin-3-yl |
| 2488 | pyridazin-4-yl | CH₃ | 2-[3-(trifluoromethyl)pyridin-2-yl]-1,3-thiazol-5-yl |
| 2489 | pyridazin-4-yl | CH₃ | 4-{[3-(methoxycarbonyl)-1H-1,2,4-triazol-1-yl]methyl}phenyl |
| 2490 | pyridazin-4-yl | CH₃ | 1-[3-(1,1-difluoroethyl)pyridin-2-yl]-1H-pyrazol-4-yl |
| 2491 | pyridazin-4-yl | CH₃ | 6-{[3-(pyridin-2-yl)-1H-pyrazol-1-yl]methyl}pyridin-3-yl |
| 2492 | pyridazin-4-yl | CH₃ | 3-(acetylamino)phenyl |
| 2493 | pyridazin-4-yl | CH₃ | 1-(3-chloropyridin-2-yl)-5-(trifluoromethyl)-1H-pyrazol-4-yl |
| 2494 | pyridazin-4-yl | CH₃ | 2-{[3-(pyridin-2-yl)-1H-pyrazol-1-yl]methyl}pyridin-4-yl |
| 2495 | pyridazin-4-yl | CH₃ | 3-[(methoxyacetyl)amino]-4-methylphenyl |
| 2496 | pyridazin-4-yl | CH₃ | 1-(3-bromopyridin-2-yl)-5-(trifluoromethyl)-1H-pyrazol-4-yl |
| 2497 | pyridazin-4-yl | CH₃ | 4-[(methoxyimino)methyl]phenyl |
| 2498 | pyridazin-4-yl | CH₃ | 1-(pyrimidin-2-yl)-5-(trifluoromethyl)-1H-pyrazol-4-yl |
| 2499 | pyridazin-4-yl | CH₃ | 2-(2-chlorophenyl)-1,3-thiazol-4-yl |
| 2500 | pyridazin-4-yl | CH₃ | 4-methoxy-3-[(propan-2-ylsulphonyl)amino]phenyl |
| 2501 | pyridazin-4-yl | CH₃ | 6-(3-cyanophenoxy)pyridin-3-yl |
| 2502 | pyridazin-4-yl | CH₃ | 4-fluoro-3-[(2-methylpropanoyl)amino]phenyl |
| 2503 | pyridazin-4-yl | CH₃ | 1-(2-bromophenyl)-5-(difluoromethyl)-1H-pyrazol-4-yl |
| 2504 | pyridazin-4-yl | CH₃ | 1,3-dimethyl-1H-pyrazol-4-yl |
| 2505 | pyridazin-4-yl | CH₃ | 5-(1-methoxyethyl)-1-(2,2,2-trifluoroethyl)-1H-pyrazol-4-yl |
| 2506 | pyridazin-4-yl | CH₃ | 1-(2,6-dichlorophenyl)-5-(1-fluoroethyl)-1H-pyrazol-4-yl |
| 2507 | pyridazin-4-yl | CH₃ | 6-(pyrrolidin-l-yl)pyridin-3-yl |
| 2508 | pyridazin-4-yl | CH₃ | 3-(acetylamino)-5-(trifluoromethyl)phenyl |
| 2509 | pyridazin-4-yl | CH₃ | 2-[3-(methoxymethyl)pyridin-2-yl]-1,3-thiazol-4-yl |
| 2510 | pyridazin-4-yl | CH₃ | 2-[3-(1-methoxyethyl)pyridin-2-yl]-1,3-thiazol-5-yl |
| 2511 | pyridazin-4-yl | CH₃ | 1-[3-(dimethoxymethyl)pyridin-2-yl]-1H-pyrazol-4-yl |
| 2512 | pyridazin-4-yl | CH₃ | 5-(1-methoxyethyl)-1-(2-methylphenyl)-1H-pyrazol-4-yl |
| 2513 | pyridazin-4-yl | CH₃ | 1-methyl-1H-benzotriazol-5-yl |
| 2514 | pyridazin-4-yl | CH₃ | 4-[(butylsulphanyl)methyl]phenyl |
| 2515 | pyridazin-4-yl | CH₃ | 1H-pyrazol-4-yl |
| 2516 | pyridazin-4-yl | CH₃ | 1-(2-bromo-6-chlorophenyl)-5-(1-methoxyethyl)-1H-pyrazol- |
| | | | 4-yl |
| 2517 | pyridazin-4-yl | CH₃ | 5-chloro-6-[4-(methylsulphonyl)phenoxy]pyridin-3-yl |
| 2518 | pyridazin-4-yl | CH₃ | 1-[3-(1-methoxyethyl)pyridin-2-yl]-1H-pyrazol-4-yl |
| 2519 | pyridazin-4-yl | CH₃ | 6-[(4,6-dimethoxypyrimidin-2-yl)sulphanyl]pyridin-3-yl |
| 2520 | pyridazin-4-yl | CH₃ | 4-[(6-methylpyridin-3-yl)oxy]phenyl |
| 2521 | pyridazin-4-yl | CH₃ | 3-methoxy-1-methyl-1H-pyrazol-4-yl |
| 2522 | pyridazin-4-yl | CH₃ | 6-(4-tert-butylphenoxy)pyridin-3-yl |
| 2523 | pyridazin-4-yl | CH₃ | 1-(2,4-dichlorophenyl)-5-methyl-1H-pyrazol-4-yl |
| 2524 | pyridazin-4-yl | CH₃ | 3-(but-2-enoylamino)-4-methylphenyl |
| 2525 | pyridazin-4-yl | CH₃ | 4-{[4-(pyrimidin-2-yl)-1H-pyrazol-1-yl]methyl}phenyl |
| 2526 | pyridazin-4-yl | CH₃ | 4-{[6-(1H-pyrazol-1-yl)pyridin-2-yl]oxy}phenyl |
| 2527 | pyridazin-4-yl | CH₃ | 4-{[3,5-bis(trifluoromethyl)-1H-1,2,4-triazol-1-yl]methyl}-3- |
| | | | chlorophenyl |
| 2528 | pyridazin-4-yl | CH₃ | 4-[(2-methoxypyrimidin-4-yl)oxy]phenyl |
| 2529 | pyridazin-4-yl | CH₃ | 1-[4-chloro-2-(trifluoromethyl)phenyl]-5-(methoxymethyl)- |
| | | | 1H-pyrazol-4-yl |
| 2530 | pyridazin-4-yl | CH₃ | 5-(trifluoromethyl)-1-[2-(trifluoromethyl)phenyl]-1H-pyrazol- |
| | | | 4-yl |
| 2531 | pyridazin-4-yl | CH₃ | 6-(dimethylamino)pyridin-3-yl |
| 2532 | pyridazin-4-yl | CH₃ | 2-[5-chloro-3-(trifluoromethyl)pyridin-2-yl]-1,3-thiazol-5-yl |
| 2533 | pyridazin-4-yl | CH₃ | 4-{[4-(3-chloropyridin-2-yl)-1H-pyrazol-1-yl]methyl}phenyl |
| 2534 | pyridazin-4-yl | CH₃ | 4-[(2,2,3,3,4,4,4-heptafluorobutanoyl)(methyl)amino]phenyl |
| 2535 | pyridazin-4-yl | CH₃ | 4-[(trifluoromethyl)sulphanyl]phenyl |
| 2536 | pyridazin-4-yl | CH₃ | 6-[4-(methylsulphonyl)phenoxy]pyridin-3-yl |
| 2537 | pyridazin-4-yl | CH₃ | 1-(2-bromophenyl)-5-(methoxymethyl)-1H-pyrazol-4-yl |
| 2538 | pyridazin-4-yl | CH₃ | 6-[(4-bromo-1H-pyrazol-1-yl)methyl]pyridin-3-yl |
| 2539 | pyridazin-4-yl | CH₃ | 3-chloro-4-{[3-(trifluoromethyl)-1H-pyrazol-1- |
| | | | yl]methyl}phenyl |
| 2540 | pyridazin-4-yl | CH₃ | 6-{[4-(trifluoromethyl)-1H-pyrazol-1-yl]methyl}pyridin-3-yl |
| 2541 | pyridazin-4-yl | CH₃ | 1-(3-bromopyridin-2-yl)-5-(difluoromethyl)-1H-pyrazol-4-yl |

### USE EXAMPLES

### Example A

### Myzus Test (MYZUPE Spray Treatment)

Solvents: 78 parts by weight of acetone
1.5 parts by weight of dimethylformamide
   Emulsifier: 0.5 part by weight of alkylaryl polyglycol ether

An appropriate preparation of active ingredient is prepared by mixing 1 part by weight of active ingredient with the stated amounts of solvents and emulsifier and diluting the concentrate with emulsifier-containing water to the desired concentration.

Discs of Chinese cabbage (Brassica pekinensis) infested with all stages of the green peach aphid (Myzus persicae) are sprayed with a preparation of active ingredient at the desired concentration.

After the desired time, the effect in % is determined. Here, 100% means that all of the aphids have been killed; 0% means that no aphids have been killed.

In this test, for example, the following compounds from the Preparation Examples exhibit an effect of ≧80% at an application rate of 500 g/ha:

Ex. No.: I-257, I-258, I-259, I-260, I-261, I-262, I-263, I-264, I-265, I-266, I-268, I-269, I-270, I-271, I-272, I-273, I-274, I-275, I-276, I-277, I-278, I-279, I-280, I-281, I-282, I-283, I-285, I-286, I-287, I-288, I-289, I-290, I-291, I-292, I-293, I-294, I-295, I-297, I-298, I-299, I-300, I-301, I-302, I-303, I-305, I-307, I-308, I-310, I-311, I-312, I-314, I-315, I-316, I-317, I-318, I-319, I-320, I-321, I-322, I-323, I-324, I-326, I-327, I-329, I-330, I-331, I-332, I-333, I-334, I-335, I-336, I-337, I-336

In this test, for example, the following compounds from the Preparation Examples exhibit an effect of ≧80% at an application rate of 20 g/ha:

Ex. No.: I-2

In this test, for example, the following compounds from the Preparation Examples exhibit an effect of ≧80% at an application rate of 500 ppm:

Ex. No.: I-284, I-296

### Example B

### Myzus Test; Oral (MYZUPE O)

Solvent: 80 parts by weight of acetone

An appropriate preparation of active ingredient is prepared by mixing 1 part by weight of active ingredient with the stated amounts of solvent and diluting the concentrate with water to the desired concentration.

Pots are populated with all stages of the green peach aphid (Myzus persicae); treatment is by sucking on the preparation of active ingredient at the desired concentration.

After the desired time, the effect in % is determined. Here, 100% means that all of the aphids have been killed; 0% means that no aphids have been killed.

In this test, for example, the following compounds from the Preparation Examples exhibit an effect of ≧80% at an application rate of 200 ppm:

Ex. No.: I-304, I-306, I-309, I-325, I-328

### Example C

### Boophilus microplus Test (BOOPMI Injection)

Solvent: dimethyl sulphoxide

An appropriate preparation of active ingredient is prepared by mixing 1 part by weight of active ingredient with the stated amounts of solvent and diluting the concentrate with water to the desired concentration.

The solution of active ingredient is injected into the abdomen (Boophilus microplus) and the animals are transferred into dishes and kept in a controlled atmosphere. The effect is assessed on the basis of deposition of fertile eggs.

After the desired time, the effect in % is determined. Here, 100% means that none of the tics has laid fertile eggs. In this test, for example, the following compounds from the Preparation Examples exhibit an activity of ≧80% at an application rate of 20 μg/animal:

Ex. No.: I-8, I-101, I-135

### Example D

### Myzus Test (MYZUPE)

Solvent: 3 parts by weight of dimethylformamide
Emulsifier: 1 part by weight of polyoxyethylenealkyl phenyl ether

An appropriate preparation of active ingredient is prepared by mixing 1 part by weight of active ingredient with the stated amount of solvent and emulsifier and diluting the concentrate with water to the desired concentration.

Aubergine plants (Solanum melongena) infested by all stages of the green peach aphid (Myzus persicae) are sprayed with a preparation of active ingredient at the desired concentration.

After the desired time, the effect in % is determined; here, 100% means that all of the aphids have been killed. Conversely, 0% means that no aphids have been killed.

In this test, for example, the following compounds from the Preparation Examples exhibit an effect of ≧80% at an application rate of 500 ppm:

Ex. No.: I-5, I-6, I-8, I-9, I-12, I-13, I-14, I-15, I-16, I-17, I-18, I-20, I-21, I-22, I-23, I-25, I-26, I-27, I-28, I-29, I-30, I-32, I-33, I-34, I-35, I-36, I-37, I-38, I-39, I-40, I-41, I-42, I-43, I-47, I-48, I-49, I-50, I-51, I-52, I-53, I-54, I-55, I-56, I-57, I-58, I-59, I-60, I-61, I-62, I-63, I-64, I-66, I-67, I-68, I-69, I-70, I-71, I-73, I-74, I-75, I-76, I-77, I-78, I-79, I-80, I-81, I-82, I-83, I-84, I-88, I-89, I-90, I-91, I-92, I-93, I-96, I-97, I-98, I-99, I-100, I-101, I-102, I-103, I-104, I-105, I-106, I-107, I-108, I-109, I-110, I-112, I-113, I-114, I-115, I-116, I-117, I-118, I-119, I-123, I-124, I-128, I-130, I-131, I-132, I-133, I-134, I-135, I-136, I-137, I-138, I-141, I-142, I-144, I-145, I-146, I-147, I-148, I-149, I-152, I-154, I-155, I-156, I-157, I-159, I-161, I-163, I-164, I-165, I-166, I-167, I-168, I-169, I-170, I-171, I-172, I-173, I-174, I-175, I-176, I-177, I-178 I-179, I-180, I-181, I-182, I-183, I-184, I-185, I-186, I-187, I-188, I-189, I-190, I-191, I-192, I-193, I-194, I-195, I-196, I-197, I-200, I-205, I-207, I-208, I-209, I-210, I-212, I-217, I-218, I-219, I-220, I-221, I-222, I-223, I-224, I-225, I-226, I-227, I-228, I-229, I-230, I-231, I-232, I-233, I-234, I-236, I-238, I-241, I-244, I-245, I-246, I-247, I-248, I-250, I-251, I-252, I-253, I-254, I-255

In this test, for example, the following compounds from the Preparation Examples exhibit an effect of ≧80% at an application rate of 100 ppm:

Ex. No.: I-1, I-10, I-65, I-95

### Example E

### Spodoptera litura Test (PRODLI)

Solvent: 3 parts by weight of dimethylformamide
Emulsifier: 1 part by weight of polyoxyethylenealkyl phenyl ether

An appropriate preparation of active ingredient is prepared by mixing 1 part by weight of active ingredient with the stated amount of solvent and emulsifier and diluting the concentrate with water to the desired concentration.

Leaves of sweet potato (Ipomoea batatas) are immersed in the preparation of active ingredient at the desired concentration and then populated with larvae of the cotton leaf worm (Spodoptera litura).

After the desired time, the effect in % is determined; here, 100% means that all of the larvae have been killed. Conversely, 0% means that no larvae have been killed.

In this test, for example, the following compounds from the Preparation Examples exhibit an effect of ≧80% at an application rate of 500 ppm:

Ex. No.: I-86

### Example F

### Tetranychus Test (TETRUR)

Solvent: 3 parts by weight of dimethylformamide
Emulsifier: 1 part by weight of polyoxyethylenealkyl phenyl ether

An appropriate preparation of active ingredient is prepared by mixing 1 part by weight of active ingredient with the stated amount of solvent and emulsifier and diluting the concentrate with water to the desired concentration. French bean (Phaseolus vulgaris) plants are infested with a mixed population of the common spider mite (Tetranychus urticae) and sprayed with the preparation of active ingredient at the desired concentration. After the desired time, the effect in % is determined: here, 100% means that all of the spider mites have been killed. Conversely, 0% means that no spider mites have been killed.

In this test, for example, the following compounds from the Preparation Examples exhibit an effect of ≧80% at an application rate of 500 ppm: Ex. No.: I-127

1. A compound of the general formula (I), 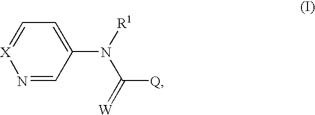
in which
R¹ is hydrogen or is optionally singly or multiply and identically or differently substituted C₁-C₆-alkyl, C₂-C₆-alkenyl, C₂-C₆-alkynyl or C₃-C₆-cycloalkyl, it being possible for the substituents to be selected independently of one another from halogen, cyano, nitro, hydroxyl, C₁-C₆-alkyl, C₁-C₆-alkoxy, C₃-C₆-cycloalkyl, C₁-C₆-haloalkoxy, C₁-C₆-alkylthio, aryl, hetaryl, arylalkyl and hetarylalkyl,
it being possible for the substituents aryl, hetaryl, arylalkyl, and hetarylalkyl to be optionally substituted singly or multiply and identically or differently by halogen, cyano, nitro, hydroxyl, C₁-C₆-alkyl, C₂-C₆-alkenyl, C₂-C₆-alkynyl, C₃-C₆-cycloalkyl, C₁-C₆-alkoxy, C₁-C₆-haloalkyl, C₁-C₆-haloalkoxy or C₁-C₆-alkylthio,
X is CH or N,
Q is an aromatic 5-6-membered ring, wherein said ring is optionally substituted by 1 to 3 heteroatoms chosen from the series N, S and O and wherein said ring is optionally, independently substituted by the radicals R²-R⁴ or and the radical A-Y,
with the proviso that when X is N:
Q is an aromatic 6-membered ring, wherein said ring is optionally substituted by 1 to 3 heteroatoms chosen from N, S and O and wherein said ring is independently, optionally substituted by the radicals R²-R⁴ or the radical A-Y;
or is an aromatic 5-membered ring, wherein said ring which is optionally substituted by 1 to 3 heteroatoms chosen from N, S and O, not more than one of the heteroatoms being N, and wherein said ring is optionally, independently substituted by the radicals R²-R⁴ or the radical A-Y;
or is a ring system selected from the group consisting of Q8a, Q8b, Q8c, and Q8d 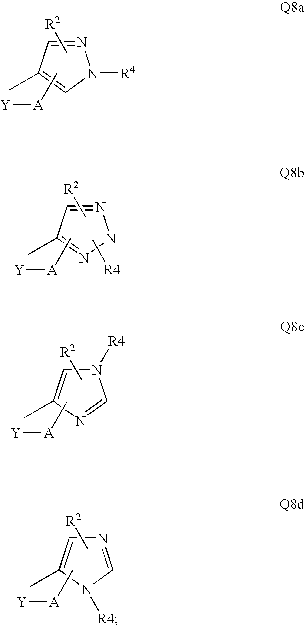
with the proviso that when X is CH:
W is O or S,
R² and R³ independently of one another are hydrogen, hydroxyl, halogen or nitro, or are optionally singly or multiply, identically or differently substituted amino, C₁-C₆-alkyl, C₁-C₆-alkoxy, C₁-C₆-alkoxy-C₁-C₆-alkyl, C₂-C₆-alkenyl, C₂-C₆-alkynyl or C₃-C₆-cycloalkyl,
it being possible for the substituents to be selected independently of one another from halogen, C₁-C₆-alkyl, C₁-C₆-alkylcarbonyl, C₁-C₆-alkoxy, C₁-C₆-haloalkoxy, C₃-C₆-cycloalkyl, C₃-C₆-cycloalkylcarbonyl, cyano, nitro, hydroxyl, C₁-C₆-alkylthio, C₁-C₆-alkylsulphinyl, C₁-C₆-alkylsulphonyl, (C₁-C₆-alkoxy)carbonyl, C₁-C₆-alkylamino, C₃-C₆-cycloalkylamino, (C₁-C₆-alkyl)C₃-C₆-cycloalkylamino, aryl, or hetaryl,
which may optionally be singly or multiply and identically or differently substituted by halogen, cyano, nitro, hydroxyl, C₁-C₆-alkyl, C₁-C₆-haloalkyl, C₂-C₆-alkenyl, C₂-C₆-alkynyl, C₃-C₆-cycloalkyl, C₁-C₆-alkoxy, C₁-C₆-haloalkoxy, C₁-C₆-alkylthio, C₁-C₆-alkylsulphinyl and C₁-C₆-alkylsulphonyl, or
R² and R³ taken together form a 5- to 7-membered heteroaromatic ring which contains 1-3 heteroatoms chosen from the series N, S and O and wherein said ring is optionally substituted by one or more C₁-C₆-alkyl,
R⁴ is hydrogen or is optionally singly or multiply, identically or differently substituted C₁-C₆-alkyl, C₁-C₆-alkoxy-C₁-C₆-alkyl, C₂-C₆-alkenyl, C₂-C₆-alkynyl, C₃-C₆-cycloalkyl, aryl or hetaryl,
it being possible for the substituents to be selected independently of one another from halogen, C₁-C₆-alkyl, C₁-C₆-alkylcarbonyl, C₁-C₆-alkoxy, C₁-C₆-haloalkoxy, C₃-C₆-cycloalkyl, C₃-C₆-cycloalkylcarbonyl, cyano, nitro, hydroxyl, C₁-C₆-alkylthio, C₁-C₆-alkylsulphinyl, C₁-C₆-alkylsulphonyl, (C₁-C₆-alkoxy)carbonyl, C₁-C₆-alkylamino, C₃-C₆-cycloalkylamino, (C₁-C₆-alkyl)C₃-C₆-cycloalkylamino, aryl, and hetaryl,
which may optionally be singly or multiply and identically or differently substituted by halogen, cyano, nitro, hydroxyl, C₁-C₆-alkyl, C₁-C₆-haloalkyl, C₂-C₆-alkenyl, C₂-C₆-alkynyl, C₃-C₆-cycloalkyl, C₁-C₆-alkoxy, C₁-C₆-haloalkoxy, C₁-C₆-alkylthio, C₁-C₆-alkylsulphinyl, or C₁-C₆-alkylsulphonyl,
A is a direct bond or is C₁-C₆-alkylene, C₁-C₆-alkyleneoxy, oxy(C₁-C₆)-alkylene, thio(C₁-C₆)-alkylene, C₁-C₆-alkylenethio, —O—, —S—, C₁-C₄-alkylenecarboxy, carboxy(C₁-C₄)-alkylene, —NR^{S}, C₁-C₄-alkylenecarbamido, carbamido(C₁-C₄)-alkylene, —NR⁵(C═O)O—, NR⁵(SO₂)—, —NR⁵(C═O)NR⁵— or —(SO₂)—,
R⁵ is C₁-C₆-alkyl, C₃-C₆-cycloalkyl or hydrogen, and
Y is hydrogen, optionally singly or multiply, identically or differently substituted C₁-C₆-alkyl, C₃-C₆-cycloalkyl, an optionally singly or multiply, identically or differently substituted, 5-10-membered, optionally fused ring system containing 0-4 heteroatoms, it being possible for the heteroatoms to be selected from the series N, S and O,
it being possible for the substituents to be selected from halogen, nitro, cyano, from optionally singly or multiply, identically or differently substituted amino, C₁-C₆-alkyl, C₁-C₆-alkoxy, C₁-C₆-alkoxy-C₁-C₆-alkyl, C₂-C₆-alkenyl, C₂-C₆-alkynyl, C₃-C₆-cycloalkyl, aryl, hetaryl, C₁-C₆-arylalkyl, C₁-C₆-hetarylalkyl, aryloxy, hetaryloxy, sulphonyl, C₁-C₆-thioalkyl, and C₁-C₆-carboxyalkyl,
it being possible for the substituents to be selected independently of one another from halogen, C₁-C₆-alkyl, C₁-C₆-alkylcarbonyl, C₁-C₆-haloalkyl, C₁-C₆-alkoxy, C₁-C₆-haloalkoxy, C₃-C₆-cycloalkyl, C₃-C₆-cycloalkylcarbonyl, cyano, nitro, C₁-C₆-alkylthio, C₁-C₆-alkylsulphinyl, C₁-C₆-alkylsulphonyl, (C₁-C₆-alkoxy)carbonyl, C₂-C₆-alkenyl, C₂-C₆-alkynyl, C₁-C₆-alkylamino, C₃-C₆-cycloalkylamino, (C₁-C₆-alkyl)C₃-C₆-cycloalkylamino, and di(C₁-C₄)alkylamino,
or a salt thereof.

2. The compound according to claim 1, in which
R¹ is hydrogen or is optionally singly or multiply, identically or differently substituted C₁-C₄-alkyl, C₂-C₄-alkenyl, C₂-C₄-alkynyl or C₃-C₄-cycloalkyl,
it being possible for the substituents to be selected independently of one another from halogen, cyano, nitro, hydroxyl, C₁-C₄-alkyl, C₁-C₄-alkoxy, C₃-C₄-cycloalkyl, C₁-C₄-haloalkoxy, aryl, hetaryl, arylalkyl and hetarylalkyl,
it being possible for the substituents aryl, hetaryl, arylalkyl, and hetarylalkyl to be optionally substituted singly or multiply, identically or differently by halogen, cyano, nitro, hydroxyl, C₁-C₄-alkyl, C₂-C₄-alkenyl, C₂-C₄-alkynyl, C₃-C₄-cycloalkyl, C₁-C₄-alkoxy, C₁-C₄-haloalkyl or C₁-C₄-haloalkoxy,
Q is a ring selected from the group Q1 to Q14: 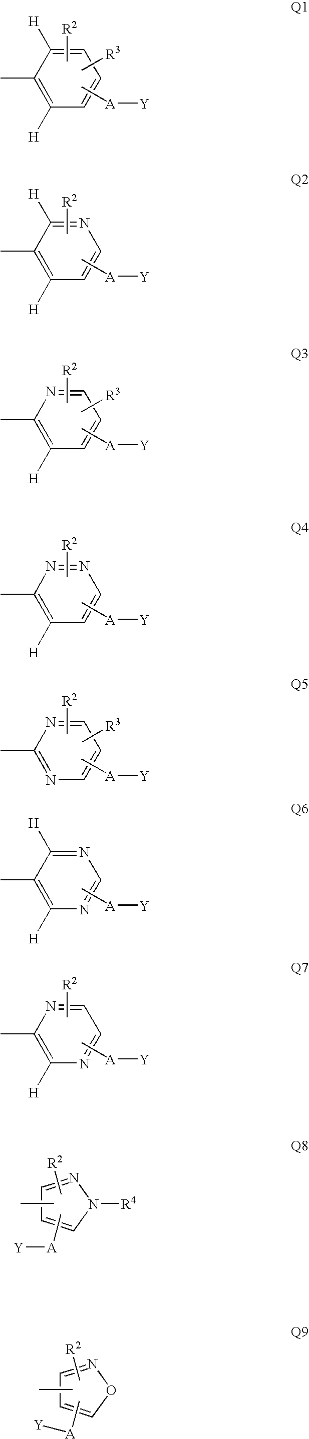 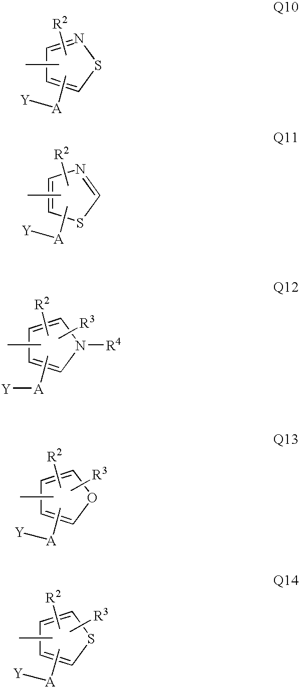
with the proviso that when X is N;
Q is a ring selected from the group Q1 to Q14: 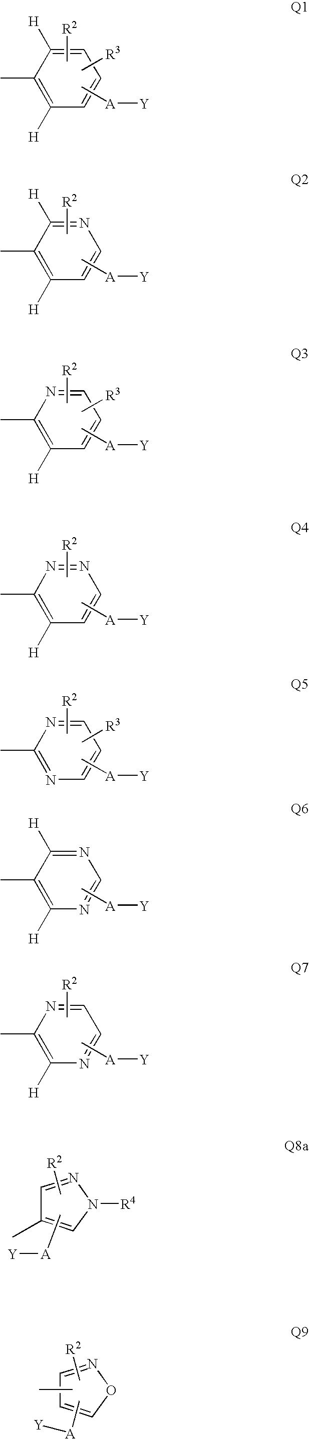 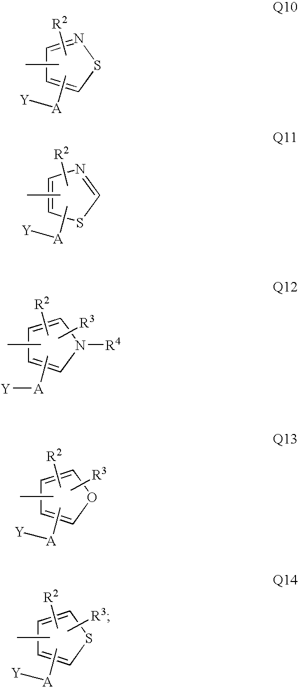
with the proviso that when X is CH:
R² and R³ independently of one another are hydrogen, hydroxyl, halogen, nitro, or are optionally singly or multiply, identically or differently substituted amino, C₁-C₄-alkyl, C₁-C₄-alkoxy, C₁-C₄-alkoxy-C₁-C₄-alkyl, C₂-C₄-alkenyl, C₂-C₄-alkynyl, or C₃-C₄-cycloalkyl,
it being possible for the substituents to be selected independently of one another from halogen, C₁-C₄-alkyl, C₁-C₄-alkylcarbonyl, C₁-C₄-alkoxy, C₁-C₄-haloalkoxy, C₃-C₄-cycloalkyl, C₃-C₄-cycloalkylcarbonyl, cyano, nitro, hydroxyl, C₁-C₄-alkylthio, C₁-C₄-alkylsulphinyl, C₁-C₄-alkylsulphonyl, (C₁-C₄-alkoxy)carbonyl, C₁-C₄-alkylamino, C₃-C₄-cycloalkylamino, (C₁-C₄-alkyl)C₃-C₄-cycloalkylamino, aryl, and hetaryl,
which may optionally be singly or multiply and identically or differently substituted by halogen, cyano, nitro, hydroxyl, C₁-C₄-alkyl, C₁-C₄-haloalkyl, C₂-C₄-alkenyl, C₂-C₄-alkynyl, C₃-C₄-cycloalkyl, C₁-C₄-alkoxy, C₁-C₄-haloalkoxy C₁-C₄-alkylthio, C₁-C₄-alkylsulphinyl or C₁-C₄-alkylsulphonyl, or
R² and R³ taken together form a 5-membered, N-containing heteroaromatic ring which is optionally singly or multiply substituted by C₁-C₆-alkyl,
R⁴ is hydrogen or is optionally singly or multiply, identically or differently substituted C₁-C₄-alkyl, C₁-C₄-alkoxy-C₁-C₄-alkyl, C₂-C₄-alkenyl, C₂-C₄-alkynyl, C₃-C₄-cycloalkyl, aryl or hetaryl,
it being possible for the substituents to be selected independently of one another from halogen, C₁-C₄-alkyl, C₁-C₄-alkoxy, C₁-C₄-haloalkoxy, C₃-C₄-cycloalkyl, cyano, nitro, C₁-C₄-alkylthio, (C₁-C₆-alkoxy)carbonyl, aryl, and hetaryl,
which may optionally be singly or multiply and identically or differently substituted by halogen, cyano, nitro, C₁-C₄-alkyl, C₁-C₄-haloalkyl, C₃-C₄-cycloalkyl, C₁-C₄-alkoxy or C₁-C₄-haloalkoxy,
A is a direct bond or is C₁-C₄-alkylene, C₁-C₄-alkyleneoxy, oxy(C₁-C₄)-alkylene, thio(C₁-C₄)-alkylene, C₁-C₄-alkylenethio, —O—, —S—, C₁-C₄-alkylenecarboxy, carboxy(C₁-C₄)-alkylene, —NR^{S}, —C═N—O—, —NR⁵(C═O)O—, NR⁵(SO₂)—, —NR⁵(C═O)NR⁵—, optionally R⁵-substituted carbamido, amidocarbonyl, C₁-C₄-alkylenecarbamido, carbamido(C₁-C₄)-alkylene or —(SO₂)—,
R⁵ is C₁-C₄-alkyl, C₃-C₆-cycloalkyl or hydrogen,
Y is hydrogen, optionally singly or multiply and identically or differently substituted C₁-C₄-alkyl, C₃-C₄-cycloalkyl, an optionally singly or multiply and identically or differently substituted 5-6-membered ring system containing 0-4 heteroatoms, it being possible for the heteroatoms to be selected from the series N and O, or is naphthalene or quinoline,
it being possible for the substituents to be selected from halogen, nitro, cyano, from optionally singly or multiply, identically or differently substituted amino, C₁-C₄-alkyl, C₁-C₄-alkoxy, C₁-C₄-alkoxy-C₁-C₄-alkyl, C₂-C₄-alkenyl, C₂-C₄-alkynyl, C₃-C₄-cycloalkyl, aryl, hetaryl, C₁-C₄-arylalkyl, C₁-C₄-hetarylalkyl, aryloxy, hetaryloxy, sulphonyl, C₁-C₄-thioalkyl and C₁-C₄-carboxyalkyl,
it being possible for the substituents to be selected independently of one another from halogen, C₁-C₄-alkyl, C₁-C₄-alkylcarbonyl, C₁-C₄-haloalkyl, C₁-C₄-alkoxy, C₁-C₄-haloalkoxy, C₃-C₄-cycloalkyl, C₃-C₄-cycloalkylcarbonyl, cyano, nitro, (C₁-C₄-alkoxy)carbonyl, C₁-C₄-alkylthio and di(C₁-C₄)alkylamino,
X is CH or N, and
W is O or S.

3. The compound according to claim 1, in which
R¹ is hydrogen or is optionally singly or multiply, identically or differently substituted C₁-C₄-alkyl, C₂-C₄-alkenyl or C₂-C₄-alkynyl,
it being possible for the substituents to be selected independently of one another from halogen, C₁-C₄-alkyl, aryl and arylalkyl,
it being possible for the substituents aryl and arylalkyl to be optionally singly or multiply, identically or differently substituted by halogen, C₁-C₄-alkyl, C₁-C₄-haloalkyl, or C₁-C₄-haloalkoxy,
Q is a ring selected from the group consisting of 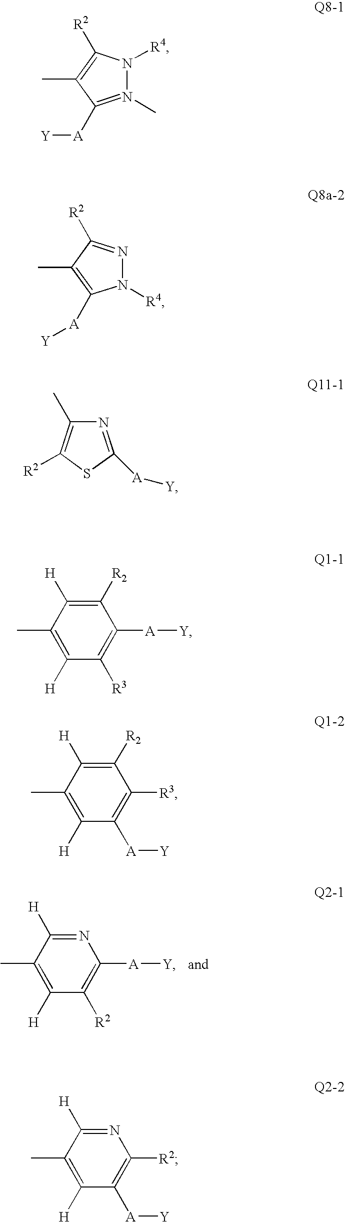
with the proviso that when X is N:
Q is a ring selected from the group consisting of 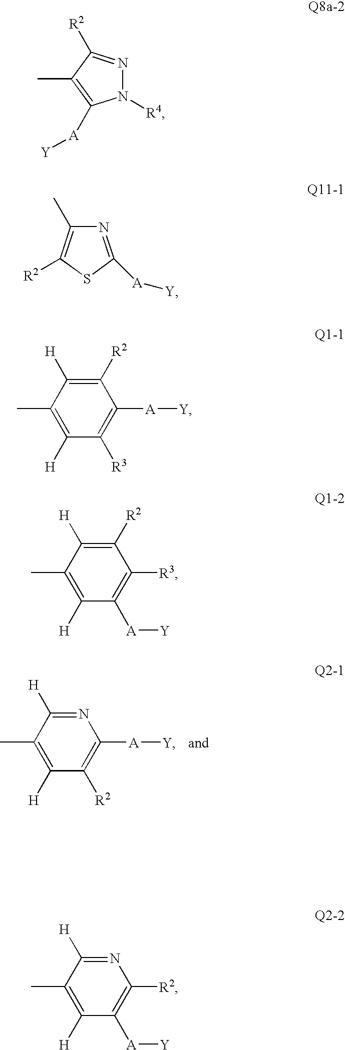
with the proviso that when X is CH:
R² and R³ independently of one another are hydrogen, halogen or nitro, or are optionally singly or multiply, identically or differently substituted amino, C₁-C₄-alkyl, C₁-C₄-alkoxy, C₁-C₄-alkoxy-C₁-C₄-alkyl or C₃-C₄-cycloalkyl,
it being possible for the substituents to be selected independently of one another from halogen, C₁-C₄-alkyl, C₁-C₄-alkoxy, C₁-C₄-haloalkoxy, C₃-C₄-cycloalkyl, cyano and nitro,
R⁴ is hydrogen or is optionally singly or multiply, identically or differently substituted, C₁-C₄-alkyl, C₁-C₄-alkoxy-C₁-C₄-alkyl, C₂-C₄-alkenyl, C₂-C₄-alkynyl, C₃-C₄-cycloalkyl, aryl, or hetaryl,
it being possible for the substituents to be selected independently of one another from halogen, C₁-C₄-alkyl, C₁-C₄-alkoxy, C₁-C₄-haloalkoxy, C₃-C₄-cycloalkyl, cyano, nitro, (C₁-C₄-alkoxy)carbonyl, aryl, and hetaryl,
which may optionally be singly or multiply and identically or differently substituted by halogen, cyano, nitro, C₁-C₄-alkyl, C₁-C₄ haloalkyl, C₁-C₄-alkoxy, or C₁-C₄-haloalkoxy,
A is a direct bond or is C₁-C₄-alkylene, —CH₂—O—, CH₂—S—, —O—, —S—, —O—CH₂, —S—CH₂—, —C(═O)O—, OC(═O)—, —NR^{S}—, —C═N—O—, —R⁵NC(═O)—, —(CH₂)R⁵NC(═O)—, —C(═O)NR⁵—, —NR⁵(C═O)O—, NR⁵(SO₂)—, —NR⁵(C═O)NR⁵— or —(SO₂)—,
R⁵ is methyl, ethyl, cyclopentyl, isobutyl or hydrogen,
Y is optionally singly or multiply, identically or differently substituted C₁-C₄-alkyl, C₃-C₄-cycloalkyl, or is an optionally singly or multiply, identically or differently substituted, 5-6-membered ring containing 0-3 nitrogen atoms, or is naphthalene or quinoline,
it being possible for the substituents to be selected from halogen, nitro, and cyano, from optionally singly or multiply, identically or differently substituted amino, C₁-C₄-alkyl, C₁-C₄-alkoxy, C₁-C₄-alkoxy-C₁-C₄-alkyl, C₃-C₄-cycloalkyl, aryl, hetaryl, C₁-C₄-arylalkyl, C₁-C₄-hetarylalkyl, aryloxy, hetaryloxy, sulphonyl, C₁-C₄-thioalkyl, and C₁-C₄-carboxyalkyl,
it being possible for the substituents to be selected independently of one another from halogen, C₁-C₄-alkyl, C₁-C₄-haloalkyl, C₁-C₄-alkoxy, C₁-C₄-haloalkoxy, C₁-C₄-alkylthio, C₃-C₄-cycloalkyl and di(C₁-C₄)alkylamino,
X is CH or N, and
W is O.

4. An agrochemical composition, comprising at least one compound according to claim 1, one or more extenders and one or more surface-active substances.

5. A method of controlling animal pests, comprising contacting said animal pests or their habitat, or seed with at least one compound according to claim 1.

6. A process for preparing an agrochemical composition, comprising mixing at least one compound according to claim 1 with one or more extenders, or one or more surface-active substances, and combinations thereof.

7. The method according to claim 5, wherein said 1 animal pests are controlled in crop protection, in materials protection, in the hygiene sector, or in the veterinary sector.
